(19) United States
(12) Patent Application Publication
(10) Pub. No.: US 2021/0388390 A1
(43) Pub. Date: Dec. 16, 2021

(54) INTRACELLULAR DELIVERY OF BIOMOLECULES TO ENHANCE ANTIGEN PRESENTING CELL FUNCTION

(71) Applicant: SQZ Biotechnologies Company, Watertown, MA (US)

(72) Inventors: Howard BERNSTEIN, Watertown, MA (US); Matt BOOTY, Watertown, MA (US); Luke CASSEREAU, San Bruno, CA (US); Jonathan CHOW, Watertown, MA (US); Kelan HLAVATY, Belmont, MA (US); Scott LOUGHHEAD, Watertown, MA (US); Melissa MYINT, Watertown, MA (US); Scott SAUER, Watertown, MA (US); Armon SHAREI, Watertown, MA (US); Vidhya VIJAYAKUMAR, Watertown, MA (US)

(21) Appl. No.: 17/282,709

(22) PCT Filed: Oct. 3, 2019
(86) PCT No.: PCT/US2019/054586

§ 371 Date: Apr. 2, 2021

Related U.S. Application Data
(60) Provisional application No. 62/741,491, filed on Oct. 4, 2018.
(60) Provisional application No. 62/794,518, filed on Jan. 18, 2019.
(60) Provisional application No. 62/898,935, filed on Sep. 11, 2019.

(51) Int. Cl. C12N 15/87; A61P 37/04; A61K 39/00; C12N 5/0784; C12N 5/0786; A61K 35/15

(52) U.S. Cl. CPC C12N 15/87; A61P 37/04; A61K 39/0011; C12N 5/0639; A61K 2039/5158; A61K 35/15; A61K 2039/5154; C12N 2527/00; C12N 5/0645

(57) ABSTRACT

The present application provides enhanced antigen presenting cells comprising an agent that enhances the viability and/or function of the antigen presenting cell and/or an antigen and/or an adjuvant, methods of manufacturing such modified antigen presenting cells, and methods of using such modified antigen presenting cells, such as for modulating an immune response in an individual.

### CROSS-REFERENCE TO RELATED APPLICATIONS

This application claims priority to U.S. Provisional Application No. 62/741,491, filed Oct. 4, 2019, U.S. Provisional Application No. 62/794,518, filed Jan. 18, 2019, and U.S. Provisional Application No. 62/898,935, filed Sep. 11, 2019. The contents of each of which is hereby incorporated by reference in its entirety.

### FIELD OF THE INVENTION

The present disclosure relates generally to antigen presenting cells comprising an agent that enhances the viability and/or function of the antigen presenting cell, methods of manufacturing such enhanced antigen presenting cells, and methods of using such enhanced antigen presenting cells, such as for modulating an immune response in an individual.

### BACKGROUND OF THE INVENTION

Immunotherapy can be divided into two main types of interventions, either passive or active. Passive protocols include administration of pre-activated and/or engineered cells, disease-specific therapeutic antibodies, and/or cytokines. Active immunotherapy strategies are directed at stimulating immune system effector functions in vivo. Several current active protocols include vaccination strategies with disease-associated peptides, lysates, or allogeneic whole cells, infusion of autologous DCs as vehicles for tumor antigen delivery, and infusion of immune checkpoint modulators. See Papaioannou, Nikos E., et al. Annals of translational medicine 4.14 (2016).

All references cited herein, including patent applications and publications, are incorporated by reference in their entirety.

### BRIEF SUMMARY OF THE INVENTION

In some aspects, the invention provides a method for enhancing tumor homing of an antigen presenting cell, the method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances tumor homing of the antigen presenting cell to pass into the antigen presenting cell; and b) incubating the perturbed input antigen presenting cell with the agent that enhances tumor homing of the antigen presenting cell for a sufficient time to allow the agent to enter the perturbed input antigen presenting cell, thereby generating an enhanced antigen presenting cell. In some embodiments, wherein the agent that enhances tumor homing of the antigen presenting cell upregulates expression of one or more of CXCR3, CCR5, VLA-4 or LFA-1. In some embodiments, the agent that upregulates expression of one or more of CXCR3, CCR5, VLA-4 or LFA-1 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with. In some embodiments, the nucleic acid-protein complex is a gene-editing complex with an ssODN for homologous recombination.

In some aspects, the invention provides a method for enhancing the viability and/or function of an antigen presenting cell, the method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an anti-apoptotic agent to pass into the antigen presenting cell; and b) incubating the perturbed input antigen presenting cell with the anti-apoptotic agent for a sufficient time to allow the agent to enter the perturbed input antigen presenting cell, thereby generating an enhanced antigen presenting cell. In some embodiments, the anti-apoptotic agent upregulates expression of one or more of XIAP, cIAP1/2, survivin, livin, cFLIP, Hsp72, or Hsp90. In some embodiments, the agent that upregulates expression of one or more of XIAP, cIAP1/2, survivin, livin, cFLIP, Hsp72 or Hsp90 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for enhancing the function of an antigen presenting cell, the method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing to pass into the antigen presenting cell; and b) incubating the perturbed input antigen presenting cell with the agent that enhances antigen processing for a sufficient time to allow the agent to enter the perturbed input antigen presenting cell, thereby generating an enhanced antigen presenting cell. In some embodiments, the agent that enhances antigen processing upregulates expression of one or more of LMP2, LMP7, MECL-1 or β5t. In some embodiments, the agent that upregulates expression of one or more of LMP2, LMP7, MECL-1 or β5t is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for enhancing the function of an antigen presenting cell, the method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing and/or loading onto MHC molecules to pass into the antigen presenting cell; and b) incubating the perturbed input antigen presenting cell with the agent that enhances antigen processing and/or loading onto MHC molecules for a sufficient time to allow the agent to enter the perturbed input antigen presenting cell, thereby generating an enhanced antigen presenting cell. In some embodiments, the agent that enhances antigen processing and/or loading onto MHC molecules upregulates expression of one or more of TAP, Tapasin, ERAAP, Calreticulin, Erp57 or PDI. In some embodiments, the agent that upregulates expression of one or more of TAP, Tapasin, ERAAP, Calreticulin, Erp57 or PDI is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for modulating immune activity of an antigen presenting cell, the method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that modulates immune activity to pass into the antigen presenting cell; and b) incubating the perturbed input antigen presenting cell with the agent that modulates immune activity for a sufficient time to allow the agent to enter the perturbed input antigen presenting cell, thereby generating an enhanced antigen presenting cell. In some embodiments, the agent that modulates immune activity upregulates expression of one or more of type I interferon, type II interferon, type III interferon and Shp2. In some embodiments, the agent that upregulates expression of one or more of type I interferon, type II interferon, type III interferon and Shp2 is a nucleic acid, a protein or a nucleic acid-protein complex. In some embodiments, the nucleic acid is a DNA, an mRNA, an siRNA, an shRNA or an miRNA. In some embodiments, the agent that modulates immune activity downregulates expression of interferon beta. In some embodiments, the agent that downregulates expression of interferon beta is a nucleic acid, a protein, a peptide, a nucleic acid-protein complex or a small molecule. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for enhancing the viability of an antigen presenting cell, the method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances viability of the antigen presenting cell to pass into the antigen presenting cell; and b) incubating the perturbed input antigen presenting cell with the agent that enhances viability of the antigen presenting cell for a sufficient time to allow the agent to enter the perturbed input antigen presenting cell, thereby generating an enhanced antigen presenting cell. In some embodiments, the agent that enhances viability of the antigen presenting cell upregulates expression of a serpin. In some embodiments, the agent that upregulates expression a serpin is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for enhancing the function of an antigen presenting cell, the method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances homing and/or triggers alternative homing to pass into the antigen presenting cell; and b) incubating the perturbed input antigen presenting cell with the agent that enhances homing and/or triggers alternative homing for a sufficient time to allow the agent to enter the perturbed input antigen presenting cell, thereby generating an enhanced antigen presenting cell. In some embodiments, the agent that enhances homing and/or triggers alternative homing upregulates expression of a CCL2. In some embodiments, the agent that upregulates expression of CCL2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for enhancing the viability and/or function of an antigen presenting cell, the method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that activates T cells to pass into the antigen presenting cell; and b) incubating the perturbed input antigen presenting cell with the agent that activates T cells for a sufficient time to allow the agent to enter the perturbed input antigen presenting cell, thereby generating an enhanced antigen presenting cell. In some embodiments, the agent that activates T cells upregulates expression of one or more of CD27, CD28, CD40, CD122, 4-1BB (CD137), OX40(CD134)/OX40L(CD252), GITR or ICOS. In some embodiments, the agent that upregulates expression of one or more of CD27, CD28, CD40, CD122, 4-1BB (CD137), OX40(CD134)/OX40L(CD252), GITR or ICOS is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for enhancing the viability and/or function of an antigen presenting cell, the method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that downregulates T cell inhibition to pass into the antigen presenting cell; and b) incubating the perturbed input antigen presenting cell with the agent that downregulates T cell inhibition for a sufficient time to allow the agent to enter the perturbed input antigen presenting cell, thereby generating an enhanced antigen presenting cell. In some embodiments, the agent that downregulates T cell inhibition downregulates expression of one or more of LAG3, VISTA, TIM1, B7-H4 (VTCN1) or BTLA. In some embodiments, the agent that downregulates expression of one or more of LAG3, VISTA, TIM1, B7-H4 (VTCN1) or BTLA is a nucleic acid, a protein, a peptide, a nucleic acid-protein complex or a small molecule. In some embodiments, the nucleic acid is an siRNA, an shRNA or an miRNA. In some embodiments, the nucleic acid-protein complex is a gene-editing complex with or without an ssODN for homologous recombination.

In some aspects, the invention provides a method for promoting DC formation from a monocyte or monocyte-dendritic progenitor cell, the method comprising: a) passing a cell suspension comprising the monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that promotes formation of DCs to pass into the monocyte or monocyte-dendritic progenitor cell; and b) incubating the perturbed input monocyte with the agent that promotes formation of DCs for a sufficient time to allow the agent to enter the perturbed input monocyte or monocyte-dendritic progenitor cell. In some embodiments, the agent that promotes formation of DCs upregulates expression of one or more of PU.1, Flt3, Flt3L or GMCSF. In some embodiments, the agent that upregulates expression of one or more of PU.1, Flt3, Flt3L or GMCSF is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for promoting plasmacytoid DC (pDC) formation from a monocyte or monocyte-dendritic progenitor cell, the method comprising: a) passing a cell suspension comprising the monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of pDCs to pass into the monocyte or monocyte-dendritic progenitor cell; and b) incubating the perturbed input monocyte or monocyte-dendritic progenitor cell with the agent that promotes formation of pDCs for a sufficient time to allow the agent to enter the perturbed input monocyte or monocyte-dendritic progenitor cell. In some embodiments, the agent that promotes formation of pDCs upregulates expression of E2-2. In some embodiments, the agent that upregulates expression of E2-2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for promoting CD8a+/CD10+ DC formation from a monocyte or monocyte-dendritic progenitor cell, the method comprising: a) passing a cell suspension comprising the monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that promotes formation of CD8a+/CD10+ DCs to pass into the monocyte; and b) incubating the perturbed input monocyte or monocyte-dendritic progenitor cell with the agent that promotes formation of CD8a+/CD10+ DCs for a sufficient time to allow the agent to enter the perturbed input monocyte or monocyte-dendritic progenitor cell. In some embodiments, the agent that promotes formation of CD8a+/CD10+ DCs upregulates expression of one or more of Batf3, IRF8 or Id2. In some embodiments, the agent that upregulates expression of one or more of Batf3, IRF8 or Id2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provides a method for promoting CD11 b+ DC formation from a monocyte or monocyte-dendritic progenitor cell, the method comprising: a) passing a cell suspension comprising the monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of CD11b+ DCs to pass into the monocyte or monocyte-dendritic progenitor cell; and b) incubating the perturbed input monocyte or monocyte-dendritic progenitor cell with the agent that promotes formation of CD11b+ DCs for a sufficient time to allow the agent to enter the perturbed input monocyte or monocyte-dendritic progenitor cell. In some embodiments, the agent that promotes formation of CD11b+ DCs upregulates expression of one or more of IRF4, RBJ, MgI or Mtg16. In some embodiments, the agent that upregulates expression of one or more of IRF4, RBJ, MgI or Mtg16 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some aspects, the invention provide a method for inhibiting formation of pDCs and classical DCs from a monocyte or monocyte-dendritic progenitor cell, the method comprising:

a) passing a cell suspension comprising the monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that inhibits formation of pDCs and classical DCs to pass into the monocyte or monocyte-dendritic progenitor cell; and b) incubating the perturbed input monocyte or monocyte-dendritic progenitor cell with the agent that inhibits formation of pDCs and classical DCs for a sufficient time to allow the agent to enter the perturbed input monocyte or monocyte-dendritic progenitor cell. In some embodiments, the agent that inhibits formation of pDCs and classical DCs downregulates expression of STAT3 and/or Xbp1. In some embodiments, the agent that downregulates expression of STAT3 and/or Xbp1 is a nucleic acid, a protein, a peptide, a nucleic acid-protein complex or a small molecule. In some embodiments, the nucleic acid is an siRNA, an shRNA or an miRNA. In some embodiments, the nucleic acid-protein complex is a gene-editing complex. In some embodiments, the nucleic acid-protein complex is a gene-editing complex with an ssODN for homologous recombination.

In some embodiments of the above aspects, the antigen presenting cell further comprises an antigen. In some embodiments, the antigen is delivered before, at the same time, or after the agent that enhances the viability and/or function of the antigen presenting cell is delivered to the cell. In some embodiments, the antigen is delivered to the antigen presenting cell by a method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for the antigen to pass into the antigen presenting cell; and b) incubating the perturbed input antigen presenting cell with the antigen for a sufficient time to allow the antigen to enter the perturbed input antigen presenting cell.

In some embodiments of the above aspects and embodiments, the antigen presenting cell further comprises an adjuvant. In some embodiments, the adjuvant is delivered before, at the same time, or after the antigen is delivered to the cell and/or before, at the same time, or after the agent that enhances the viability and/or function of the antigen presenting cell is delivered to the cell. In some embodiments, the adjuvant is delivered to the antigen presenting cell by a method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for the adjuvant to pass into the antigen presenting cell; and b) incubating the perturbed input antigen presenting cell with the adjuvant for a sufficient time to allow the adjuvant to enter the perturbed input antigen presenting cell. In some embodiments, the adjuvant is a CpG ODN, IFN-α, STING agonists, RIG-I agonists, poly I:C, imiquimod, and/or resiquimod. In some embodiments, the antigen is capable of being processed into an MHC class I-restricted peptide and/or an MHC class II-restricted peptide.

In some embodiments of the above aspects, the diameter of the constriction is less than the diameter of the input antigen presenting cell. In some embodiments, the diameter of the constriction is about 20% to about 99% of the diameter of the input antigen presenting cell. In some embodiments, the diameter of the constriction is about 20% to about 60% of the diameter of the input antigen presenting cell.

In some embodiments, the antigen and/or adjuvant are present in the cytosol and/or a vesicle of the antigen presenting cell. In some embodiments, the antigen is bound to the surface of the antigen presenting cell. In some embodiments, the antigen is a disease associated antigen. In some embodiments, the antigen is a tumor antigen. In some embodiments, the antigen is derived from a lysate. In some embodiments, the lysate is a tumor lysate.

In some embodiments, the antigen presenting cell is a peripheral blood mononuclear cell (PBMC). In some embodiments, the antigen presenting cell is in a mixed population of cells. In some embodiments, the mixed population of cells is a population of PBMCs. In some embodiments, the PBMC is a T cell, a B cell, an NK cells, a monocyte, a macrophage and/or a dendritic cell. In some embodiments, the PBMC is engineered to present an antigen.

In some embodiments of the above aspects and embodiments, the monocyte, or monocyte-dendritic progenitor or DC further comprises an antigen. In some embodiments, the antigen is delivered before, at the same time, or after the agent that promotes or inhibits DC formation is delivered to the cell. In some embodiments, the antigen is delivered to the monocyte, or monocyte-dendritic progenitor or DC by a method comprising: a) passing a cell suspension comprising the monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the antigen to pass into the monocyte, or monocyte-dendritic progenitor or DC; and b) incubating the perturbed input monocyte, or monocyte-dendritic progenitor or DC with the antigen for a sufficient time to allow the antigen to enter the perturbed input monocyte, or monocyte-dendritic progenitor or DC.

In some embodiments of the above aspects and embodiments, the monocyte, or monocyte-dendritic progenitor or DC further comprises an adjuvant. In some embodiments, the adjuvant is delivered before, at the same time, or after the antigen is delivered to the cell and/or before, at the same time, or after the agent that promotes DC formation is delivered to the cell. In some embodiments, the adjuvant is delivered to the monocyte, or monocyte-dendritic progenitor or DC by a method comprising: a) passing a cell suspension comprising the monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the adjuvant to pass into the monocyte, or monocyte-dendritic progenitor or DC; and b) incubating the perturbed input monocyte, or monocyte-dendritic progenitor or DC with the adjuvant for a sufficient time to allow the adjuvant to enter the perturbed input monocyte, or monocyte-dendritic progenitor or DC. In some embodiments, the adjuvant is a CpG ODN, IFN-α, STING agonists, RIG-I agonists, poly I:C, imiquimod, and/or resiquimod. In some embodiments, the antigen is capable of being processed into an MHC class I-restricted peptide and/or an MHC class II-restricted peptide.

In some embodiments, the diameter of the constriction is less than the diameter of the input monocyte, or monocyte-dendritic progenitor or DC. In some embodiments, the diameter of the constriction is about 20% to about 99% of the diameter of the input monocyte, or monocyte-dendritic progenitor or DC. In some embodiments, the diameter of the constriction is about 20% to about 60% of the diameter of the input monocyte, or monocyte-dendritic progenitor or DC.

In some embodiments, the antigen and/or adjuvant are present in the cytosol and/or a vesicle of the monocyte, or monocyte-dendritic progenitor or DC. In some embodiments, the antigen is bound to the surface of the monocyte, or monocyte-dendritic progenitor or DC. In some embodiments, the antigen is a disease associated antigen. In some embodiments, the antigen is a tumor antigen. In some embodiments, the antigen is derived from a lysate. In some embodiments, the lysate is a tumor lysate.

In some aspects, the invention provides a modified antigen presenting cell comprising an agent that enhances the viability and/or function of an antigen presenting cell, wherein the cell is prepared by any of the methods described herein. In some aspects, the invention provides a modified monocyte, or monocyte-dendritic progenitor or DC, wherein the monocyte, or monocyte-dendritic progenitor or DC is prepared by any of the methods described herein.

In some aspects, the invention provides a method for modulating an immune response in an individual, comprising: administering to the individual an antigen presenting cell, wherein the antigen presenting cell is prepared by a process according to any one of the methods described herein. In some aspects, the invention provides a method for modulating an immune response in an individual, comprising: administering to the individual a dendritic cell, wherein the dendritic cell is prepared by a process according to of any one of the methods described herein.

### BRIEF DESCRIPTION OF THE DRAWINGS

FIG. 1A shows a representative schematic of an experiment to evaluate whether overexpression of costimulatory molecules in antigen presenting cells (APCs) could enhance the ability of the APCs to induce in vitro an antigen-specific T cell response. FIG. 1B shows the results of the induction of IFN-γ secretion by antigen-loaded APCs with or without co-delivery of costimulatory molecules.

FIG. 2A shows a representative schematic of an experiment to evaluate whether overexpression of costimulatory molecules in APCs could enhance the ability of the APCs to induce in vivo CD8+ T cell response. FIG. 2B shows the results of the induction of IFN-γ production in CD8+ T cells by antigen-loaded APCs with or without co-delivery of costimulatory molecules.

FIG. 3A shows a representative schematic of an experiment to compare the antigen-specific CD8+ T cell response when APCs SQZ-loaded with the antigen were administered intravenously or intranodally. FIG. 3B shows the results of the induction of IFN-γ production in CD8+ T cells by antigen-loaded APCs administered intravenously or intranodally.

FIG. 4A shows a representative schematic of an experiment to evaluate whether SQZ-mediated loading can be used to enhance the levels of homing molecules in APCs. FIG. 4B shows the surface levels of CD62L expression in APCs 4 hours and 24 hours after being SQZ-loaded with mRNA encoding CD62L. FIG. 4C shows the surface levels of CCR7 expression in APCs 4 hours and 24 hours after being SQZ-loaded with mRNA encoding CCR7.

FIG. 5A shows the percentage of each subset of PBMCs expressing CD86 on cell surface 4 hours subsequent to SQZ-mediated loading of CD86-encoding mRNA in human PBMCs. FIG. 5B shows the percentage of each subset of PBMCs expressing IFNα2 4 hours subsequent to SQZ-mediated loading of IFNα2-encoding mRNA in human PBMCs.

FIG. 6A shows the percentage of the T cell subset of PBMCs expressing CD86 on cell surface at the indicated time point subsequent to SQZ-mediated loading of CD86-encoding mRNA in human PBMCs. FIG. 6B shows the percentage of the T cell subset of PBMCs expressing 4-1BBL on cell surface at the indicated time point subsequent to SQZ-mediated loading of 4-1BBL-encoding mRNA in human PBMCs.

FIG. 7 shows the GFP mean fluorescence intensity (MFI) in the T cell subset of PBMCs 4 hours subsequent to SQZ-mediated loading of mRNA encoding unmodified eGFP or eGFP modified with 5-metoxyuridine backbone (5moU) respectively in human PBMCs, at the indicated concentration of mRNA used for SQZ-loading.

FIG. 8A shows the levels of IL-12 in culture supernatant after human PBMCs were SQZ-loaded with IL-12a- and IL-12b-encoding mRNAs and incubated at 37 C for 4 hours. FIG. 8B shows the levels of IFNα in culture supernatant after human PBMCs were SQZ-loaded with IFNα encoding mRNAs and incubated at 37° C. for 4 hours. FIG. 8C shows the levels of IFNα in culture supernatant after human PBMCs were SQZ-loaded with IFNα encoding mRNA and incubated at 37° C. for 4 hours. FIG. 8C shows the levels of IL-2 in culture supernatant after human PBMCs were SQZ-loaded with IL-2 encoding mRNA and incubated at 37° C. for 4 hours.

### DETAILED DESCRIPTION OF THE INVENTION

Antigen presenting cells (APCs) play a key role in inducing endogenous activation of CTLs. In this work, the implementation of the CellSqueeze® platform to enhance the viability and/or function of an antigen presenting cell is described. The engineered antigen presenting cells can be used for modulating an immune response to various indications, including cancer and infectious disease. By enabling efficient cytosolic delivery of agents that enhances the viability and/or function of the antigen presenting cell, this platform has demonstrated the ability to enhance the viability and/or function of an antigen presenting cell. In some embodiments, enhanced viability and/or function of the antigen presenting cell includes, but is not limited to increased persistence, circulation time or in vivo lifespan.

The present application in some aspects provides a method of enhancing the viability and/or function of an antigen presenting cell comprising a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances the viability and/or function of the antigen presenting cell to pass into the antigen presenting cell; and b) incubating the perturbed input antigen presenting cell with the agent that enhances the viability and/or function of the antigen presenting cell for a sufficient time to allow the agent to enter the perturbed input antigen presenting cell, thereby generating an enhanced antigen presenting cell. In some embodiments, the enhanced antigen presenting cell is further contacted with an additional agent that modulates in vitro maintenance and/or function of an antigen presenting cell.

In other aspects, there is provided a modified antigen presenting cell comprising an agent that enhances the viability and/or function of the antigen presenting cell, wherein the modified antigen presenting cell is prepared by a process comprising the steps of: a) passing a cell suspension comprising an input antigen presenting through a cell-deforming constriction, wherein a diameter of the constriction is a function of a diameter of the antigen presenting cell in the suspension, thereby causing perturbations of the input antigen presenting cell large enough for the agent that enhances the viability and/or function of the antigen to pass through to form a perturbed input antigen presenting cell; and b) incubating the perturbed input antigen presenting cell cell with the agent that enhances the viability and/or function of the antigen for a sufficient time to allow the antigen and the agent to enter the perturbed input antigen presenting cell; thereby generating the modified antigen presenting cell comprising the agent that enhances the viability and/or function of the antigen.

In yet other aspects, there is provided a method for modulating an immune response in an individual, comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances the viability and/or function of the antigen presenting cell to pass into the antigen presenting cell; and b) incubating the perturbed input antigen presenting cell with the agent that enhances the viability and/or function of the antigen presenting cell for a sufficient time to allow the agent to enter the perturbed input antigen presenting cell, thereby generating an enhanced antigen presenting cell; and c) administering the modified antigen presenting cell to the individual.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 2011).

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

The term “antigen presenting cell” or “APC” as used herein refers to a cell that presents antigen on an MHC complex that can elicit an antigen-specific T cell response. An antigen presenting cell can be a classical antigen presenting cell but in some embodiments, the antigen presenting cell can be any cell engineered to present an antigen. In a non-limiting example, a T cell engineered to present an antigen on an MHC complex is an antigen presenting cell.

In some embodiments, antigen presenting cells are isolated from an individual. In some embodiments, the antigen presenting cells are autologous to an individual, where the cells are derived from a particular individual, manipulated by any of the methods described herein, and returned to the particular individual. In some embodiments, the antigen presenting cells are allogeneic, where the population is derived from one individual, manipulated by any of the methods described herein, and administered to a second individual.

As used herein, “peripheral blood mononuclear cells” or “PBMCs” refers to a heterogeneous population of blood cells having a round nucleus. Examples of cells that may be found in a population of PBMCs include lymphocytes such as T cells, B cells, NK cells, monocytes, macrophages and dendritic cells. A “population of PBMCs” or a “plurality of PBMCs” as used herein refers to a preparation of PBMCs comprising cells of at least two types of blood cells. In some embodiments, a plurality of PBMCs comprises two or more of T cells, B cells, NK cells, monocytes, macrophages or dendritic cells. In some embodiments, a plurality of PBMCs comprises three or more of T cells, B cells, NK cells, monocytes, macrophages or dendritic cells. In some embodiments, a plurality of PBMCs comprises four or more of T cells, B cells, NK cells, monocytes, macrophages or dendritic cells. In some embodiments, a plurality of PBMCs comprises T cells, B cells, NK cells, monocytes, macrophages and dendritic cells.

PBMCs can be isolated by means known in the art. For example, PBMCs can be derived from peripheral blood of an individual based on density of PBMCs compared to other blood cells. In some embodiments, PBMCs are derived from peripheral blood of an individual using Ficoll (e.g., a ficoll gradient). In some embodiments, PBMCs are derived from peripheral blood of an individual using ELUTRA® cell separation system.

The term “pore” as used herein refers to an opening, including without limitation, a hole, tear, cavity, aperture, break, gap, or perforation within a material. In some examples, (where indicated) the term refers to a pore within a surface of the present disclosure. In other examples, (where indicated) a pore can refer to a pore in a cell membrane.

The term “membrane” as used herein refers to a selective barrier or sheet containing pores. The term includes a pliable sheetlike structure that acts as a boundary or lining. In some examples, the term refers to a surface or filter containing pores. This term is distinct from the term “cell membrane”.

The term “filter” as used herein refers to a porous article that allows selective passage through the pores. In some examples the term refers to a surface or membrane containing pores.

The term “heterogeneous” as used herein refers to something which is mixed or not uniform in structure or composition. In some examples the term refers to pores having varied sizes, shapes or distributions within a given surface.

The term “homogeneous” as used herein refers to something which is consistent or uniform in structure or composition throughout. In some examples the term refers to pores having consistent sizes, shapes, or distribution within a given surface.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

The term “heterologous” as it relates to amino acid sequences such as peptide sequences and polypeptide sequences, denotes sequences that are not normally joined together, and/or are not normally associated with a particular cell. Thus, a “heterologous” region of a peptide sequence is a segment of amino acids within or attached to another amino acid molecule that is not found in association with the other molecule in nature. For example, a heterologous region of a peptide construct could include the amino acid sequence of the peptide flanked by sequences not found in association with the amino acid sequence of the peptide in nature. Another example of a heterologous peptide sequence is a construct where the peptide sequence itself is not found in nature (e.g., synthetic sequences having amino acids different as coded from the native gene). Similarly, a cell transformed with a vector that expresses an amino acid construct which is not normally present in the cell would be considered heterologous for purposes of this invention. Allelic variation or naturally occurring mutational events do not give rise to heterologous peptides, as used herein.

The term “exogenous” when used in reference to an agent, such as an antigen or an adjuvant, with relation to a cell refers to an agent delivered from outside the cell (that is, from outside the cell). The cell may or may not have the agent already present, and may or may not produce the agent after the exogenous agent has been delivered.

As used herein, the term “inhibit” may refer to the act of blocking, reducing, eliminating, or otherwise antagonizing the presence, or an activity of, a particular target. Inhibition may refer to partial inhibition or complete inhibition. For example, inhibiting an immune response may refer to any act leading to a blockade, reduction, elimination, or any other antagonism of an immune response. In other examples, inhibition of the expression of a nucleic acid may include, but not limited to reduction in the transcription of a nucleic acid, reduction of mRNA abundance (e.g., silencing mRNA transcription), degradation of mRNA, inhibition of mRNA translation, and so forth.

As used herein, the term “suppress” may refer to the act of decreasing, reducing, prohibiting, limiting, lessening, or otherwise diminishing the presence, or an activity of, a particular target. In some examples, the term “suppress” may refer to the act of decreasing, reducing, prohibiting, limiting, lessening, or otherwise diminishing a general immune response. Suppression may refer to partial suppression or complete suppression. For example, suppressing an immune response may refer to any act leading to decreasing, reducing, prohibiting, limiting, lessening, or otherwise diminishing an immune response. In other examples, suppression of the expression of a nucleic acid may include, but is not limited to, reduction in the transcription of a nucleic acid, reduction of mRNA abundance (e.g., silencing mRNA transcription), degradation of mRNA, inhibition of mRNA translation, and so forth.

As used herein, the term “enhance” may refer to the act of improving, boosting, heightening, or otherwise increasing the presence, or an activity of, a particular target. In some examples, the term “enhance” may refer to the act of improving, boosting, heightening, or otherwise increasing a general immune response. For example, enhancing an immune response may refer to any act leading to improving, boosting, heightening, or otherwise increasing an immune response. In one exemplary example, enhancing an immune response may refer to employing an antigen and/or adjuvant to improve, boost, heighten, or otherwise increase an immune response. In other examples, enhancing the expression of a nucleic acid may include, but not limited to an increase in the transcription of a nucleic acid, increase in mRNA abundance (e.g., increasing mRNA transcription), decrease in degradation of mRNA, increase in mRNA translation, and so forth.

As used herein, the term “modulate” may refer to the act of changing, altering, varying, or otherwise modifying the presence, or an activity of, a particular target. For example, modulating an immune response may refer to any act leading to changing, altering, varying, or otherwise modifying an immune response. In other examples, modulating the expression of a nucleic acid may include, but not limited to a change in the transcription of a nucleic acid, a change in mRNA abundance (e.g., increasing mRNA transcription), a corresponding change in degradation of mRNA, a change in mRNA translation, and so forth.

As used herein, the term “induce” may refer to the act of initiating, prompting, stimulating, establishing, or otherwise producing a result. For example, inducing an immune response may refer to any act leading to initiating, prompting, stimulating, establishing, or otherwise producing a desired immune response. In other examples, inducing the expression of a nucleic acid may include, but not limited to initiation of the transcription of a nucleic acid, initiation of mRNA translation, and so forth.

The term “homologous” as used herein refers to a molecule which is derived from the same organism. In some examples the term refers to a nucleic acid or protein which is normally found or expressed within the given organism.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and phosphorothioates, and thus can be an oligodeoxynucleoside phosphoramidate (P-NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, the term “adjuvant” refers to a substance which modulates and/or engenders an immune response. Generally, the adjuvant is administered in conjunction with an antigen to effect enhancement of an immune response to the antigen as compared to antigen alone. Various adjuvants are described herein.

The terms “CpG oligodeoxynucleotide” and “CpG ODN” refer to DNA molecules containing a dinucleotide of cytosine and guanine separated by a phosphate (also referred to herein as a “CpG” dinucleotide, or “CpG”). The CpG ODNs of the present disclosure contain at least one unmethylated CpG dinucleotide. That is, the cytosine in the CpG dinucleotide is not methylated (i.e., is not 5-methylcytosine). CpG ODNs may have a partial or complete phosphorothioate (PS) backbone.

As used herein, the term “antibody” refers to immunoglobulin molecules and antigen-binding portions or fragments of immunoglobulin (Ig) molecules, i.e., molecules that contain an antigen binding site that specifically binds an antigen. The term antibody encompasses not only intact polyclonal or monoclonal antibodies, but also fragments thereof, such as dAb, Fab, Fab′, F(ab′)2, Fv), single chain (scFv) or single domain antibody (sdAb). Typically, an “antigen-binding fragment” contains at least one CDR of an immunoglobulin heavy and/or light chain that binds to at least one epitope of the antigen of interest. In this regard, an antigen-binding fragment may comprise 1, 2, 3, 4, 5, or all 6 CDRs of a variable heavy chain (VH) and variable light chain (VL) sequence from antibodies that bind the antigen, such as generally six CDRs for an antibody containing a VH and a VL (“CDR1,” “CDR2” and “CDR3” for each of a heavy and light chain), or three CDRs for an antibody containing a single variable domain. Antibody fragments or antigen binding fragments include single domain antibodies, such as those only containing a VH or only containing a VL, including, for example, camelid antibody (VHH), shark antibody (VNAR), a nanobody or engineered VH or VK domains.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

For any of the structural and functional characteristics described herein, methods of determining these characteristics are known in the art.

Methods for Enhancing the Viability and/or Function of an Antigen Presenting Cell

In certain aspects, there is provided a method for enhancing the viability and/or function of an antigen presenting cell, the method comprising: a) passing a cell suspension comprising an input antigen presenting cell through a cell-deforming constriction, wherein a diameter of the constriction is a function of a diameter of the antigen presenting cell in the suspension, thereby causing perturbations of the input antigen presenting cell large enough for an agent that enhances the viability and/or function of the antigen presenting cell to pass into the antigen presenting cell; and b) incubating the perturbed input antigen presenting cell with the agent that enhances the viability and/or function of the antigen presenting cell for a sufficient time to allow the agent to enter the perturbed input antigen presenting cell, thereby generating an enhanced antigen presenting cell.

In some embodiments according to any of the methods for enhancing the viability and/or function of an antigen presenting cell described herein, the agent comprises a protein or polypeptide. In some embodiments, the agent is a protein or polypeptide. In some embodiments, the protein or polypeptide is a therapeutic protein, antibody, fusion protein, antigen, synthetic protein, reporter marker, or selectable marker. In some embodiments, the protein is a gene-editing protein or nuclease such as a zinc-finger nuclease (ZFN), transcription activator-like effector nuclease (TALEN), mega nuclease, or CRE recombinase. In some embodiments, the gene-editing protein or nuclease is Cas9. In further embodiments, the agent comprises Cas9 with or without an ssODN for homologous recombination or homology directed repair. In some embodiments, the fusion proteins can include, without limitation, chimeric protein drugs such as antibody drug conjugates or recombinant fusion proteins such as proteins tagged with OST or streptavidin. In some embodiments, the agent is a transcription factor. In some embodiments, the agent comprises a nucleic acid. In some embodiments, the agent is a nucleic acid. Exemplary nucleic acids include, without limitation, recombinant nucleic acids, DNA, recombinant DNA, cDNA, genomic DNA, RNA, siRNA, mRNA, saRNA, miRNA, lncRNA, tRNA, and shRNA. In some embodiments, the nucleic acid is homologous to a nucleic acid in the cell. In some embodiments, the nucleic acid is heterologous to a nucleic acid in the cell. In some embodiments, the agent is a plasmid. In some embodiments, the agent is a nucleic acid-protein complex. In some embodiments, the nucleic acid-protein complex is a gene-editing complex with or without an ssODN for homologous recombination. In some embodiments, the nucleic acid-protein complex comprises Cas9 and guide RNA, with or without an ssODN for homologous recombination or homology directed repair.

In some embodiments according to any of the methods for enhancing the viability and/or function of the antigen presenting cell described herein, the antigen presenting cell is a peripheral blood mononuclear cell (PBMC). In some embodiments, the antigen presenting cell is a mixed population of cells. In some embodiments, the antigen presenting cell is a mixed population of cells contained within PBMCs. In some embodiments, wherein the enhanced antigen presenting cell comprises an agent that enhances the viability and/or function of the antigen presenting cell and wherein the input antigen presenting cell is a PBMC, the agent modulates immune activity. In further embodiments, the agent that modulates immune activity upregulates the expression of one or more of IL-2, IL-7, IL-12a IL-12b, or IL-15. In some embodiments, the agent that modulates immune activity modulates the expression of one or more of the interferon-regulatory factors (IRFs), such as IRF3 or IRF5. In some embodiments, the agent that modulates immune activity modulates the expression of one or more of the toll-like receptors (TLRs), such as TLR-4. In some embodiments, the agent that modulates immune activity modulates the expression and/or activity of one or more of the toll-like receptors (TLRs), such as TLR-4 and/or TLR-9. In some embodiments, the agent that modulates immune activity modulates the expression of one or more of pattern recognition receptors (PRRs). In some embodiments, the agent that modulates immune activity modulates the activity of one or more of pattern recognition receptors (PRRs). In some embodiments, the agent that modulates immune activity modulates the expression and/or activity of one or more of STING, RIG-I, AIM2, LRRF1P1 or NLPR3. In some embodiments, wherein the enhanced antigen presenting cell comprises an agent that enhances the viability and/or function of the antigen presenting cell and wherein the input antigen presenting cell is a PBMC, the agent enhances antigen presentation. In some embodiments, the agent that enhances antigen presentation upregulates the expression of MHC-I and/or MHC-II. In some embodiments, the agent that enhances antigen presentation upregulates the expression of T-cell Receptor (TCR). In some embodiments, wherein the enhanced antigen presenting cell comprises an agent that enhances the viability and/or function of the antigen presenting cell and wherein the input antigen presenting cell is a PBMC, the agent enhances activation of the antigen presenting cell. In some embodiments, the agent that enhances activation of the antigen presenting cell modulates the expression of one or more of CD25, KLRG1, CD80, or CD86. In some embodiments, the agent that enhances activation of the antigen presenting cell modulates the expression of CD80 and/or CD86. In some embodiments, wherein the enhanced antigen presenting cell comprises an agent that enhances the viability and/or function of the antigen presenting cell and wherein the input antigen presenting cell is a PBMC, the agent enhances homing of the antigen presenting cell. In some embodiments, the agent that enhances homing of the antigen presenting cell modulates the expression of one or more of CD62L, CCR2, CCR7, CX3CR1, or CXCR5. In some embodiments, wherein the enhanced antigen presenting cell comprises an agent that enhances the viability and/or function of the antigen presenting cell and wherein the input antigen presenting cell is a PBMC, the agent is an anti-apoptotic agent. In some embodiments, the anti-apoptotic agent modulates the expression of one or more of Bcl-2, Bcl-3, or Bcl-xL. In some embodiments, wherein the enhanced antigen presenting cell comprises an agent that enhances the viability and/or function of the antigen presenting cell and wherein the input antigen presenting cell is a PBMC, the agent induces alteration in cell fate or phenotype. In some embodiments, the agent that induces alteration in cell fate or phenotype modulates the expression of one or more of Oct4, Sox2, c-Myc, Klf-4, Nanog, Lin28, Lin28B, T-bet, or GATA3. In some embodiments, the agent is a protein, a nucleic acid or a nucleic acid-protein complex. In some embodiments, the nucleic acid is a DNA or an mRNA. In some embodiments, the nucleic acid is a siRNA, shRNA or miRNA. In some embodiments, the nucleic acid-protein complex is a gene-editing complex.

In some embodiments that can be combined with any of the methods described herein, the agent enhances homing of the antigen presenting cell to a site for T cell activation. In some embodiments, the agent enhances homing of the antigen presenting cell to lymph nodes. In some embodiments, the agent that enhances homing of the antigen presenting cell modulates the expression of one or more of CD62L, CCR2, CCR7, CX3CR1, or CXCR5. In some embodiments, the agent is a protein, a nucleic acid or a nucleic acid-protein complex. In some embodiments, the nucleic acid is a DNA or an mRNA. In some embodiments, the nucleic acid is a siRNA, shRNA or miRNA. In some embodiments, the nucleic acid-protein complex is a gene-editing complex. In some embodiments, the agent that enhances homing of the antigen presenting cell comprises one or more mRNAs encoding one or more of: CD62L, CCR2, CCR7, CX3CR1, or CXCR5. In some embodiments, the expression of one or more of CD62L, CCR2, CCR7, CX3CR1, or CXCR5 is increased by about any one of: 5%, 10%, 20%, 30%, 40%, 50%, 60%, 70%, 80%, 90%, 95%, 99%, or 100%. In some embodiments, the expression of one or more of CD62L, CCR2, CCR7, CX3CR1, or CXCR5 is increased by about any one of: 2-fold, 3-fold, 5-fold, 10-fold, 50-fold, 100 fold, 500-fold, or 1000-fold. In some embodiments, the homing of an antigen presenting cell comprising the agent to a site for T cell activation is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the homing of an antigen presenting cell comprising the agent to a site for T cell activation is increased by about any one of: 2-fold, 3-fold, 5-fold, 10-fold, 50-fold, 100 fold, 500-fold, or 1000-fold compared to an antigen presenting cell that does not comprise the agent. In some embodiments, the antigen presenting cell is a dendritic cell.

In certain aspects, there is provided a method for enhancing the viability and/or function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances viability and/or function of the antigen presenting cell to pass into the antigen presenting cell; and; b) incubating the perturbed input antigen presenting cell with the agent that enhances viability and/or function of the antigen presenting cell for a sufficient time to allow the agent to enter the perturbed input antigen presenting cell, thereby generating an antigen presenting cell with enhanced viability and/or function. In some embodiments, the agent that enhances viability and/or function of the antigen presenting cell upregulates expression of one or more of IL-2, IL-7, IL-12a IL-12b, IL-15, IL-18 or IL-21. In further embodiments, the agent that upregulates expression of one or more of IL-2, IL-7, IL-12a IL-12b, IL-15, IL-18 or IL-21 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances viability and/or function of the antigen presenting cell comprises one or more mRNAs encoding one or more of: IL-2, IL-7, IL-12a IL-12b, IL-15, IL-18 or IL-21. In some embodiments, the expression of one or more of IL-2, IL-7, IL-12a IL-12b, IL-15, IL-18 or IL-21 is increased by about any one of: 5%, 10%, 20%, 30%, 40%, 50%, 60%, 70%, 80%, 90%, 95%, 99%, or 100%. In some embodiments, the expression of one or more of IL-2, IL-7, IL-12a IL-12b, IL-15, IL-18 or IL-21 is increased by about any one of: 2-fold, 3-fold, 5-fold, 10-fold, 50-fold, 100 fold, 500-fold, 1000-fold, or more. In some embodiments, the circulating half-life and/or in vivo persistence of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the circulating half-life and/or in vivo persistence of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell. In some embodiments that can be combined with any other embodiments, the one or more of IL-2, IL-7, IL-12a IL-12b, IL-15, IL-18 or IL-21 comprise endogenous nucleotide or protein sequences. In some embodiments, the one or more of: IL-2, IL-7, IL-12a IL-12b, IL-15, IL-18 or IL-21 comprise modified nucleotide or protein sequences. In some embodiments, the one or more of: IL-2, IL-7, IL-12a IL-12b, IL-15, IL-18 or IL-21 are membrane-bound, such as bound to the membrane of the modified antigen presenting cell. In some embodiments, the one or more of: IL-2, IL-7, IL-12a IL-12b, IL-15, IL-18 or IL-21 are bound to membrane by GPI anchor. In some embodiments, the one or more of: IL-2, IL-7, IL-12a IL-12b, IL-15, IL-18 or IL-21 comprise a transmembrane domain sequence. In some embodiments, the one or more of: IL-2, IL-7, IL-12a IL-12b, IL-15, IL-18 or IL-21 comprise a GPI-anchor signal sequence. In some embodiments, the one or more of: IL-2, IL-7, IL-12a IL-12b, IL-15, IL-18 or IL-21 comprise the transmembrane domain and cytoplasmic tail of murine B7-1 (B7TM). In some embodiments, the one or more of: IL-2, IL-7, IL-12a IL-12b, IL-15, IL-18 or IL-21 comprising modified sequences do not bind to IL-2Rα chain (CD25) and/or do not bind IL-15Rα (CD215). In some embodiments, the one or more of: IL-2, IL-7, IL-12a IL-12b, IL-15, IL-18 or IL-21 comprising modified sequences bind to IL-2Rβ [symbol] _{c} with higher affinity than the respective natural counterpart, such as but not limited to affinity that is higher than the natural counterpart by 5%, 10%, 20%, 30%, 40%, 50%, 60%, 70%, 80%, 90%, 95%, 99%, 100%, 2-fold, 3-fold, 5-fold, 10-fold, 50-fold, 100 fold, 500-fold, 1000-fold or more. In some embodiments, the one or more of: IL-2, IL-7, IL-12a IL-12b, IL-15, IL-18 or IL-21 comprising modified amino acid sequence display about any one of: 80%, 81%, 82%, 83%, 84%, 85%, 86%, 87%, 88%, 89%, 90%, 91%, 92%, 93%, 94%, 95%, 96%, 97%, 98%, or 99% similarity as the respective wild type amino acid sequence. In some embodiments, the one or more of: IL-2, IL-7, IL-12a IL-12b, IL-15, IL-18 or IL-21 comprising modified nucleotide sequence display about any one of: 80%, 81%, 82%, 83%, 84%, 85%, 86%, 87%, 88%, 89%, 90%, 91%, 92%, 93%, 94%, 95%, 96%, 97%, 98%, or 99% similarity as the respective wild type nucleotide sequence. In some embodiments, the agent comprises one or more mimics of: IL-2, IL-7, IL-12a IL-12b, IL-15, IL-18 or IL-21, wherein the mimic comprises nucleotide or protein sequence that displays about any one of: 80%, 81%, 82%, 83%, 84%, 85%, 86%, 87%, 88%, 89%, 90%, 91%, 92%, 93%, 94%, 95%, 96%, 97%, 98%, or 99% similarity as the respective wild type sequence of IL-2, IL-7, IL-12a IL-12b, IL-15, IL-18 or IL-21. In some embodiments, the agent comprises an IL-2 mimic. In some embodiments, the agent comprises Neoleukin-2/15 (Neo-2/15).

In certain aspects, there is provided a method for enhancing the tumor homing of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances tumor homing of the antigen presenting cell to pass into the antigen presenting cell; and; b) incubating the perturbed input antigen presenting cell with the agent that enhances tumor homing of the antigen presenting cell for a sufficient time to allow the agent to enter the perturbed input antigen presenting cell, thereby generating an antigen presenting cell with enhanced tumor homing. In some embodiments, the agent that enhances tumor homing of the antigen presenting cell upregulates expression of one or more of CXCR3, CCR5, VLA-4 or LFA-1. In further embodiments, the agent that upregulates expression of one or more of CXCR3, CCR5, VLA-4 or LFA-1 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances tumor homing of the antigen presenting cell comprises one or more mRNAs encoding one or more of: CXCR3, CCR5, VLA-4 or LFA-1. In some embodiments, the expression of one or more of CXCR3, CCR5, VLA-4 or LFA-1 is increased by about any one of: 5%, 10%, 20%, 30%, 40%, 50%, 60%, 70%, 80%, 90%, 95%, 99%, or 100%. In some embodiments, the expression of one or more of CXCR3, CCR5, VLA-4 or LFA-1 is increased by about any one of: 2-fold, 3-fold, 5-fold, 10-fold, 50-fold, 100 fold, 500-fold, 1000-fold, or more. In some embodiments, the tumor homing of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the tumor homing of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for enhancing the viability and/or function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an anti-apoptotic agent to pass into the antigen presenting cell; and b) incubating the perturbed input antigen presenting cell with the anti-apoptotic agent for a sufficient time to allow the agent to enter the perturbed input antigen presenting cell, thereby generating an enhanced antigen presenting cell. In some embodiments, the anti-apoptotic agent upregulates expression of one or more of XIAP, cIAP1/2, survivin, livin, cFLIP, Hsp72, or Hsp90. In further embodiments, the agent that upregulates expression of one or more of XIAP, cIAP1/2, survivin, livin, cFLIP, Hsp72 or Hsp90 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances viability and/or function of an antigen presenting cell comprises one or more mRNAs encoding one or more of: XIAP, cIAP1/2, survivin, livin, cFLIP, Hsp72, or Hsp90. In some embodiments, the expression of one or more of XIAP, cIAP1/2, survivin, livin, cFLIP, Hsp72, or Hsp90 is increased by about any one of: 5%, 10%, 20%, 30%, 40%, 50%, 60%, 70%, 80%, 90%, 95%, 99%, or 100%. In some embodiments, the expression of one or more of XIAP, cIAP1/2, survivin, livin, cFLIP, Hsp72, or Hsp90 is increased by about any one of: 2-fold, 3-fold, 5-fold, 10-fold, 50-fold, 100 fold, 500-fold, 1000-fold, or more. In some embodiments, the circulating half-life and/or in vivo persistence of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the circulating half-life and/or in vivo persistence of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for enhancing the function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing to pass into the antigen presenting cell; and b) incubating the perturbed input antigen presenting cell with the agent that enhances antigen processing for a sufficient time to allow the agent to enter the perturbed input antigen presenting cell, thereby generating an enhanced antigen presenting cell. In some embodiments, the agent that enhances antigen processing upregulates expression of one or more of LMP2, LMP7, MECL-1 or β5t. In further embodiments, the agent that upregulates expression of one or more of LMP2, LMP7, MECL-1 or β5t is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances antigen processing comprises one or more mRNAs encoding one or more of: LMP2, LMP7, MECL-1 or β5t. In some embodiments, the expression of one or more of LMP2, LMP7, MECL-1 or β5t is increased by about any one of: 5%, 10%, 20%, 30%, 40%, 50%, 60%, 70%, 80%, 90%, 95%, 99%, or 100%. In some embodiments, the expression of one or more of LMP2, LMP7, MECL-1 or β5t is increased by about any one of: 2-fold, 3-fold, 5-fold, 10-fold, 50-fold, 100 fold, 500-fold, 1000-fold, or more. In some embodiments, the antigen processing in an antigen presenting cell comprising the agent is enhanced by about any one of: 5%, 10%, 20%, 30%, 40%, 50%, 60%, 70%, 80%, 90%, 95%, 99%, or 100% compared to an antigen presenting cell that does not comprise the agent. In some embodiments, the antigen processing in an antigen presenting cell comprising the agent is enhanced by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for enhancing the function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing and/or loading onto MHC molecules to pass into the antigen presenting cell; and b) incubating the perturbed input antigen presenting cell with the agent that enhances antigen processing and/or loading onto MHC molecules for a sufficient time to allow the agent to enter the perturbed input antigen presenting cell, thereby generating an enhanced antigen presenting cell. In some embodiments, the agent that enhances antigen processing and/or loading onto MHC molecules upregulates expression of one or more of TAP, Tapasin, ERAAP, Calreticulin, Erp57 or PDI. In further embodiments, the agent that upregulates expression of one or more of TAP, Tapasin, ERAAP, Calreticulin, Erp57 or PDI is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances antigen processing and/or loading comprises one or more mRNAs encoding one or more of: TAP, Tapasin, ERAAP, Calreticulin, Erp57 or PDI. In some embodiments, the expression of one or more of TAP, Tapasin, ERAAP, Calreticulin, Erp57 or PDI is increased by about any one of: 5%, 10%, 20%, 30%, 40%, 50%, 60%, 70%, 80%, 90%, 95%, 99%, or 100%. In some embodiments, the expression of one or more of TAP, Tapasin, ERAAP, Calreticulin, Erp57 or PDI is increased by about any one of: 2-fold, 3-fold, 5-fold, 10-fold, 50-fold, 100 fold, 500-fold, 1000-fold, or more. In some embodiments, the antigen processing and/or loading in an antigen presenting cell comprising the agent is enhanced by about any one of: 5%, 10%, 20%, 30%, 40%, 50%, 60%, 70%, 80%, 90%, 95%, 99%, or 100% compared to an antigen presenting cell that does not comprise the agent. In some embodiments, the antigen processing and/or loading in an antigen presenting cell comprising the agent is enhanced by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating immune activity of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that modulates immune activity to pass into the antigen presenting cell; and b) incubating the perturbed input antigen presenting cell with the agent that modulates immune activity for a sufficient time to allow the agent to enter the perturbed input antigen presenting cell, thereby generating a modified antigen presenting cell, such as an enhanced antigen presenting cell. In some embodiments, the agent that modulates immune activity upregulates expression of one or more of type I interferons, type II interferons, type III interferons and Shp2. In further embodiments, the agent that upregulates expression of one or more of type I interferon, type II interferon, type III interferon and Shp2 is a nucleic acid, a protein or a nucleic acid-protein complex. In some embodiments, the agent that modulates immune activity upregulates expression of one or more of type I interferons, type II interferons, or type III interferons. In further embodiments, the agent that upregulates expression of one or more of type I interferon, type II interferon, or type III interferon is a nucleic acid, a protein or a nucleic acid-protein complex. In some embodiments, the nucleic acid is a DNA, an mRNA, an siRNA, an shRNA or an miRNA. In some embodiments, the agent that modulates immune activity downregulates expression of interferon-beta. In further embodiments, the agent that downregulates expression of interferon-beta is a nucleic acid, a protein, a nucleic acid-protein complex or a small molecule. In some embodiments, the nucleic acid is a DNA, an mRNA, an siRNA, an shRNA or an miRNA. In some embodiments, the nucleic acid-protein complex is a gene-editing complex with or without an ssODN for homologous recombination.

In certain aspects, there is provided a method for enhancing the function and/or maturation of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances the function and/or maturation of an antigen presenting cell to pass into the antigen presenting cell; and b) incubating the perturbed input antigen presenting cell with the agent that enhances the function and/or maturation of an antigen presenting cell for a sufficient time to allow the agent to enter the perturbed input antigen presenting cell, thereby generating an enhanced antigen presenting cell. In some embodiments, the agent that enhances the function and/or maturation of an antigen presenting cell of the antigen presenting cell upregulates expression of one or more of type I interferons, type II interferons, or type III interferons. In some embodiments, the agent that enhances the function and/or maturation of an antigen presenting cell of the antigen presenting cell upregulates expression of one or more of: IFN-α2, IFN-β, IFN-γ, IFN-λ1, IFN-λ2, or IFN-λ3. In some embodiments, the agent that enhances expression of homing receptors in antigen presenting cell comprises one or more mRNAs encoding one or more of: IFN-α2, IFN-β, IFN-γ, IFN-λ1, IFN-λ2, or IFN-λ3. In some embodiments, the expression of one or more of IFN-α2, IFN-β, IFN-γ, IFN-λ1, IFN-λ2, or IFN-λ3 is increased by about any one of: 5%, 10%, 20%, 30%, 40%, 50%, 60%, 70%, 80%, 90%, 95%, 99%, or 100%. In some embodiments, the expression of one or more of IFN-α2, IFN-β, IFN-γ, IFN-λ1, IFN-λ2, or IFN-λ3 is increased by about any one of: 2-fold, 3-fold, 5-fold, 10-fold, 50-fold, 100 fold, 500-fold, 1000-fold, or more. In some embodiments, the maturation of an antigen presenting cell comprising the agent is enhanced by about any one of: 5%, 10%, 20%, 30%, 40%, 50%, 60%, 70%, 80%, 90%, 95%, 99%, or 100% compared to an antigen presenting cell that does not comprise the agent. In some embodiments, the maturation of an antigen presenting cell comprising the agent is enhanced by about any one of: 2-fold, 3-fold, 5-fold, 10-fold, 50-fold, 100 fold, 500-fold, 1000-fold or more compared to an antigen presenting cell that does not comprise the agent.

In certain aspects, there is provided a method for enhancing the viability of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances viability of the antigen presenting cell to pass into the antigen presenting cell; and b) incubating the perturbed input antigen presenting cell with the agent that enhances viability of the antigen presenting cell for a sufficient time to allow the agent to enter the perturbed input antigen presenting cell, thereby generating an enhanced antigen presenting cell. In some embodiments, the agent that enhances viability of the antigen presenting cell upregulates expression of a serpin. In further embodiments, the agent that upregulates expression a serpin is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances viability of the antigen presenting cell comprises one or more mRNAs encoding one or more serpins. In some embodiments, the expression of one or more serpins is increased by about any one of: 5%, 10%, 20%, 30%, 40%, 50%, 60%, 70%, 80%, 90%, 95%, 99%, or 100%. In some embodiments, the expression of one or more serpins is increased by about any one of: 2-fold, 3-fold, 5-fold, 10-fold, 50-fold, 100 fold, 500-fold, 1000-fold, or more. In some embodiments, the circulating half-life and/or in vivo persistence of an antigen presenting cell of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the circulating half-life and/or in vivo persistence of an antigen presenting cell of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent.

In certain aspects, there is provided a method for enhancing the function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances homing and/or triggers alternative homing to pass into the antigen presenting cell; and b) incubating the perturbed input antigen presenting cell with the agent that enhances homing and/or triggers alternative homing for a sufficient time to allow the agent to enter the perturbed input antigen presenting cell, thereby generating an enhanced antigen presenting cell. In some embodiments, the agent that enhances homing receptors of the antigen presenting cell upregulates expression of CCL2. In further embodiments, the agent that upregulates expression of CCL2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances homing receptors of the antigen presenting cell upregulates expression of one or more of: CD62L, CCR2, CCR7, CX3CR1, or CXCR5. In further embodiments, the agent that upregulates expression of one or more of: CD62L, CCR2, CCR7, CX3CR1, or CXCR5 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent enhances homing of the enhanced antigen presenting cell to lymph nodes. In some embodiments, the antigen presenting cell is a dendritic cell. In some embodiments, the agent that enhances homing receptors of the antigen presenting cell upregulates expression of CCL2. In further embodiments, the agent that upregulates expression of CCL2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances expression of homing receptors in antigen presenting cell comprises one or more mRNAs encoding one or more of: CCL2, CD62L, CCR2, CCR7, CX3CR1, or CXCR5. In some embodiments, the expression of one or more of CCL2, CD62L, CCR2, CCR7, CX3CR1, or CXCR5 is increased by about any one of: 5%, 10%, 20%, 30%, 40%, 50%, 60%, 70%, 80%, 90%, 95%, 99%, or 100%. In some embodiments, the expression of one or more of CCL2, CD62L, CCR2, CCR7, CX3CR1, or CXCR5 is increased by about any one of: 2-fold, 3-fold, 5-fold, 10-fold, 50-fold, 100 fold, 500-fold, 1000-fold, or more. In some embodiments, the expression of homing receptors in an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the expression of homing receptors in an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent.

In certain aspects, there is provided a method for enhancing the function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances homing and/or triggers alternative homing to pass into the antigen presenting cell; and b) incubating the perturbed input antigen presenting cell with the agent that enhances homing and/or triggers alternative homing for a sufficient time to allow the agent to enter the perturbed input antigen presenting cell, thereby generating an enhanced antigen presenting cell. In some embodiments, the agent that enhances homing receptors of the antigen presenting cell upregulates expression of CCL2. In further embodiments, the agent that upregulates expression of CCL2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances homing and/or triggers alternative homing comprises one or more mRNAs encoding CCL2. In some embodiments, the expression of CCL2 is increased by about any one of: 5%, 10%, 20%, 30%, 40%, 50%, 60%, 70%, 80%, 90%, 95%, 99%, or 100%. In some embodiments, the expression of CCL2 is increased by about any one of: 2-fold, 3-fold, 5-fold, 10-fold, 50-fold, 100 fold, 500-fold, 1000-fold, or more. In some embodiments, the homing and/or alternative homing of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the homing and/or alternative homing of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for enhancing the viability and/or function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that activates T cells to pass into the antigen presenting cell; and b) incubating the perturbed input antigen presenting cell with the agent that activates T cells for a sufficient time to allow the agent to enter the perturbed input antigen presenting cell, thereby generating an enhanced antigen presenting cell. In some embodiments, the agent that activates T cells upregulates expression of one or more of CD27, CD28, CD40, CD122, 4-1BB (CD137), OX40(CD134)/OX40L(CD252), GITR or ICOS. In further embodiments, the agent that upregulates expression of one or more of CD27, CD28, CD40, CD122, 4-1BB (CD137), OX40(CD134)/OX40L(CD252), GITR or ICOS is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances T cell activation comprises one or more mRNAs encoding one or more of: CD27, CD28, CD40, CD122, 4-1BB (CD137), OX40(CD134)/OX40L(CD252), GITR or ICOS.

In some embodiments, the expression of one or more of CD27, CD28, CD40, CD122, 4-1BB (CD137), OX40(CD134)/OX40L(CD252), GITR or ICOS is increased by about any one of: 5%, 10%, 20%, 30%, 40%, 50%, 60%, 70%, 80%, 90%, 95%, 99%, or 100%. In some embodiments, the expression of one or more of CD27, CD28, CD40, CD122, 4-1BB (CD137), OX40(CD134)/OX40L(CD252), GITR or ICOS is increased by about any one of: 2-fold, 3-fold, 5-fold, 10-fold, 50-fold, 100 fold, 500-fold, 1000-fold, or more. In some embodiments, the T cell activation by an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the T cell activation by an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for enhancing the viability and/or function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that activates T cells to pass into the antigen presenting cell; and b) incubating the perturbed input antigen presenting cell with the agent that activates T cells for a sufficient time to allow the agent to enter the perturbed input antigen presenting cell, thereby generating an enhanced antigen presenting cell. In some embodiments, the agent that activates T cells upregulates expression of one or more of CD70, CD80, CD86, CD40L, 4-1BBL (CD137L), OX40L(CD252), GITRL or ICOSL. In further embodiments, the agent that upregulates expression of one or more of CD70, CD80, CD86, CD40L, 4-1BBL (CD137L), OX40L(CD252), GITRL or ICOSL is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances T cell activation comprises one or more mRNAs encoding one or more of: CD70, CD80, CD86, CD40L, 4-1BBL (CD137L), OX40L(CD252), GITRL or ICOSL. In some embodiments, the expression of one or more of CD70, CD80, CD86, CD40L, 4-1BBL (CD137L), OX40L(CD252), GITRL or ICOSL is increased by about any one of: 5%, 10%, 20%, 30%, 40%, 50%, 60%, 70%, 80%, 90%, 95%, 99%, or 100%. In some embodiments, the expression of one or more of CD70, CD80, CD86, CD40L, 4-1BBL (CD137L), OX40L(CD252), GITRL or ICOSL is increased by about any one of: 2-fold, 3-fold, 5-fold, 10-fold, 50-fold, 100 fold, 500-fold, 1000-fold, or more. In some embodiments, the T cell activation by an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the T cell activation by an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for enhancing the viability and/or function of an antigen presenting T cell, the method comprising: a) passing a cell suspension comprising an input antigen presenting T cell through a cell-deforming constriction, wherein a diameter of the constriction is a function of a diameter of the input antigen presenting T cell in the suspension, thereby causing perturbations of the input antigen presenting T cell large enough for an agent that activates T cells to pass into the antigen presenting T cell; and b) incubating the perturbed input antigen presenting T cell with the agent that activates T cells for a sufficient time to allow the agent to enter the perturbed input antigen presenting T cell, thereby generating an enhanced antigen presenting T cell. In some embodiments, the agent that activates T cells upregulates expression of one or more of CD27, CD28, CD40, CD122, 4-1BB (CD137), OX40(CD134)/OX40L(CD252), GITR or ICOS. In further embodiments, the agent that upregulates expression of one or more of CD27, CD28, CD40, CD122, 4-1BB (CD137), OX40(CD134)/OX40L(CD252), GITR or ICOS is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances T cell activation comprises one or more mRNAs encoding one or more of: CD27, CD28, CD40, CD122, 4-1BB (CD137), OX40(CD134)/OX40L(CD252), GITR or ICOS.

In some embodiments, the expression of one or more of CD27, CD28, CD40, CD122, 4-1BB (CD137), OX40(CD134)/OX40L(CD252), GITR or ICOS is increased by about any one of: 5%, 10%, 20%, 30%, 40%, 50%, 60%, 70%, 80%, 90%, 95%, 99%, or 100%. In some embodiments, the expression of one or more of CD27, CD28, CD40, CD122, 4-1BB (CD137), OX40(CD134)/OX40L(CD252), GITR or ICOS is increased by about any one of: 2-fold, 3-fold, 5-fold, 10-fold, 50-fold, 100 fold, 500-fold, 1000-fold, or more. In some embodiments, the T cell activation induced by an antigen presenting T cell comprising the agent is increased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the T cell activation induced by an antigen presenting T cell comprising the agent is increased by about any one of: 2-fold, 3-fold, 5-fold, 10-fold, 50-fold, 100 fold, 500-fold, 1000-fold or more compared to an antigen presenting T cell that does not comprise the agent. In some embodiments, the activation of an antigen presenting T cell comprising the agent is increased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the activation of an antigen presenting T cell comprising the agent is increased by about any one of: 2-fold, 3-fold, 5-fold, 10-fold, 50-fold, 100 fold, 500-fold, 1000-fold or more compared to an antigen presenting T cell that does not comprise the agent.

In certain aspects, there is provided a method for enhancing the viability and/or function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that downregulates T cell inhibition to pass into the antigen presenting cell; and b) incubating the perturbed input antigen presenting cell with the agent that downregulates T cell inhibition for a sufficient time to allow the agent to enter the perturbed input antigen presenting cell, thereby generating an enhanced antigen presenting cell. In some embodiments, the agent that downregulates T cell inhibition downregulates expression of one or more of LAG3, VISTA, TIM1, B7-H4 (VTCN1) or BTLA. In further embodiments, the agent that downregulates expression of one or more of LAG3, VISTA, TIM1, B7-H4 (VTCN1) or BTLA is a nucleic acid, a protein, a peptide, a nucleic acid-protein complex or a small molecule. In some embodiments, the nucleic acid is an siRNA, an shRNA or an miRNA. In some embodiments, the nucleic acid-protein complex is a gene-editing complex with or without an ssODN for homologous recombination. In some embodiments, the agent that downregulates T cell inhibition comprises one or more Cas9-gRNA RNP complexes targeting one or more of: LAG3, VISTA, TIM1, B7-H4 (VTCN1) or BTLA. In some embodiments, the expression of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the expression of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, 1000-fold, or more. In some embodiments, the agent that downregulates T cell inhibition comprises one or more small molecules targeting one or more of: LAG3, VISTA, TIM1, B7-H4 (VTCN1) or BTLA. In some embodiments, the agent that downregulates T cell inhibition comprises one or more antibodies or fragments thereof targeting one or more of: LAG3, VISTA, TIM1, B7-H4 (VTCN1) or BTLA. In some embodiments, the activity of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the activity of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, or 1000-fold. In some embodiments, the T cell inhibition by an antigen presenting cell comprising the agent is decreased by about any one of: 5%, 10%, 20%, 30%, 40%, 50%, 60%, 70%, 80%, 90%, 95%, 99%, or 100% compared to an antigen presenting cell that does not comprise the agent. In some embodiments, the T cell inhibition by an antigen presenting cell comprising the agent is decreased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for enhancing the viability and/or function of an antigen presenting T cell, the method comprising: a) passing a cell suspension comprising an input antigen presenting T cell through a cell-deforming constriction, wherein a diameter of the constriction is a function of a diameter of the input antigen presenting T cell in the suspension, thereby causing perturbations of the input antigen presenting T cell large enough for an agent that downregulates T cell inhibition to pass into the antigen presenting T cell; and b) incubating the perturbed input antigen presenting T cell with the agent that downregulates T cell inhibition for a sufficient time to allow the agent to enter the perturbed input antigen presenting T cell, thereby generating an enhanced antigen presenting T cell. In some embodiments, the agent that downregulates T cell inhibition downregulates expression of one or more of LAG3, VISTA, TIM1, B7-H4 (VTCN1) or BTLA. In further embodiments, the agent that downregulates expression of one or more of LAG3, VISTA, TIM1, B7-H4 (VTCN1) or BTLA is a nucleic acid, a protein, a peptide, a nucleic acid-protein complex or a small molecule. In some embodiments, the nucleic acid is an siRNA, an shRNA or an miRNA. In some embodiments, the nucleic acid-protein complex is a gene-editing complex with or without an ssODN for homologous recombination. In some embodiments, the agent that downregulates T cell inhibition comprises one or more Cas9-gRNA RNP complexes targeting one or more of: LAG3, VISTA, TIM1, B7-H4 (VTCN1) or BTLA. In some embodiments, the expression of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the expression of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, 1000-fold, or more. In some embodiments, the agent that downregulates T cell inhibition comprises one or more small molecules targeting one or more of: LAG3, VISTA, TIM1, B7-H4 (VTCN1) or BTLA. In some embodiments, the agent that downregulates T cell inhibition comprises one or more antibodies or fragments thereof targeting one or more of: LAG3, VISTA, TIM1, B7-H4 (VTCN1) or BTLA. In some embodiments, the activity of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the activity of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, 1000-fold, or more. In some embodiments, the agent that downregulates T cell inhibition comprises one or more small molecules targeting one or more of: LAG3, VISTA, TIM1, B7-H4 (VTCN1) or BTLA. In some embodiments, the function of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the function of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, or 1000-fold. In some embodiments, the T cell inhibition induced by the antigen presenting T cell comprising the agent is decreased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the T cell inhibition induced by the antigen presenting T cell comprising the agent is decreased by about any one of: 2-fold, 3-fold, 5-fold, 10-fold, 50-fold, 100 fold, 500-fold, 1000-fold or more compared to an antigen presenting T cell that does not comprise the agent. In some embodiments, the inhibition of the antigen presenting T cell comprising the agent is decreased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the inhibition of the antigen presenting T cell comprising the agent is decreased by about any one of: 2-fold, 3-fold, 5-fold, 10-fold, 50-fold, 100 fold, 500-fold, 1000-fold or more compared to an antigen presenting T cell that does not comprise the agent.

In certain aspects, there is provided a method for promoting DC formation from a monocyte or monocyte-dendritic progenitor cell, the method comprising: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of DCs to pass into the monocyte or monocyte-dendritic progenitor cell; and b) incubating the perturbed input monocyte or monocyte-dendritic progenitor cell with the agent that promotes formation of DCs for a sufficient time to allow the agent to enter the perturbed input monocyte or monocyte-dendritic progenitor cell. In some embodiments, the agent that promotes formation of DCs upregulates expression of one or more of PU.1, Flt3, Flt3L or GMCSF. In further embodiments, the agent that upregulates expression of one or more of PU.1, Flt3, Flt3L or GMCSF is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes DC formation from a monocyte or monocyte-dendritic progenitor cell comprises one or more mRNAs encoding one or more of: PU.1, Flt3, Flt3L or GMCSF. In some embodiments, the expression of one or more of PU.1, Flt3, Flt3L or GMCSF is increased by about any one of: 5%, 10%, 20%, 30%, 40%, 50%, 60%, 70%, 80%, 90%, 95%, 99%, or 100%. In some embodiments, the expression of one or more of PU.1, Flt3, Flt3L or GMCSF is increased by about any one of: 2-fold, 3-fold, 5-fold, 10-fold, 50-fold, 100 fold, 500-fold, 1000-fold, or more. In some embodiments, DC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, DC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In certain aspects, there is provided a method for promoting plasmacytoid DC (pDC) formation from a monocyte or monocyte-dendritic progenitor cell, the method comprising: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of pDCs to pass into the monocyte or monocyte-dendritic progenitor cell; and b) incubating the perturbed input monocyte or monocyte-dendritic progenitor cell with the agent that promotes formation of pDCs for a sufficient time to allow the agent to enter the perturbed input monocyte or monocyte-dendritic progenitor cell. In some embodiments, the agent that promotes formation of pDCs upregulates expression of E2-2. In further embodiments, the agent that upregulates expression of E2-2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes pDC formation from a monocyte or monocyte-dendritic progenitor cell comprises one or more mRNAs encoding E2-2. In some embodiments, the expression of E2-2 is increased by about any one of: 5%, 10%, 20%, 30%, 40%, 50%, 60%, 70%, 80%, 90%, 95%, 99%, or 100%. In some embodiments, the expression of E2-2 is increased by about any one of: 2-fold, 3-fold, 5-fold, 10-fold, 50-fold, 100 fold, 500-fold, 1000-fold, or more. In some embodiments, pDC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, pDC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In certain aspects, there is provided a method for method for promoting CD8a+/CD10+ DC formation from a monocyte or monocyte-dendritic progenitor cell, the method comprising: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of CD8a+/CD10+ DCs to pass into the monocyte or monocyte-dendritic progenitor cell; and b) incubating the perturbed input monocyte or monocyte-dendritic progenitor cell with the agent that promotes formation of CD8a+/CD10+ DCs for a sufficient time to allow the agent to enter the perturbed input monocyte or monocyte-dendritic progenitor cell. In some embodiments, the agent that promotes formation of CD8a+/CD10+ DCs upregulates expression of one or more of Batf3, IRF8 or Id2. In further embodiments, the agent that upregulates expression of one or more of Batf3, IRF8 or Id2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes CD8a+/CD10+ DC formation from a monocyte or monocyte-dendritic progenitor cell comprises one or more mRNAs encoding one or more of: Batf3, IRF8 or Id2. In some embodiments, the expression of one or more of Batf3, IRF8 or Id2 is increased by about any one of: 5%, 10%, 20%, 30%, 40%, 50%, 60%, 70%, 80%, 90%, 95%, 99%, or 100%. In some embodiments, the expression of one or more of Batf3, IRF8 or Id2 is increased by about any one of: 2-fold, 3-fold, 5-fold, 10-fold, 50-fold, 100 fold, 500-fold, 1000-fold, or more. In some embodiments, CD8a+/CD10+ DC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, CD8a+/CD10+ DC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In certain aspects, there is provided a method for promoting CD11b+ DC formation from a monocyte or monocyte-dendritic progenitor cell, the method comprising: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of CD11b+ DCs to pass into the monocyte or monocyte-dendritic progenitor cell; and b) incubating the perturbed input monocyte or monocyte-dendritic progenitor cell with the agent that promotes formation of CD11b+ DCs for a sufficient time to allow the agent to enter the perturbed input monocyte or monocyte-dendritic progenitor cell. In some embodiments, the agent that promotes formation of CD11b+ DCs upregulates expression of one or more of IRF4, RBJ, MgI or Mtg16. In further embodiments, the agent that upregulates expression of one or more of IRF4, RBJ, MgI or Mtg16 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes CD11b+ DC formation from a monocyte or monocyte-dendritic progenitor cell comprises one or more mRNAs encoding one or more of: IRF4, RBJ, MgI or Mtg16. In some embodiments, the expression of one or more of IRF4, RBJ, MgI or Mtg16 is increased by about any one of: 5%, 10%, 20%, 30%, 40%, 50%, 60%, 70%, 80%, 90%, 95%, 99%, or 100%. In some embodiments, the expression of one or more of IRF4, RBJ, MgI or Mtg16 is increased by about any one of: 2-fold, 3-fold, 5-fold, 10-fold, 50-fold, 100 fold, 500-fold, 1000-fold, or more. In some embodiments, CD11b+ DC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, CD11b+ DC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In certain aspects, there is provided a method for inhibiting formation of pDCs and classical DCs from a monocyte or monocyte-dendritic progenitor cell, the method comprising: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that inhibits formation of pDCs and classical DCs to pass into the monocyte or monocyte-dendritic progenitor cell; and b) incubating the perturbed input monocyte or monocyte-dendritic progenitor cell with the agent that inhibits formation of pDCs and classical DCs for a sufficient time to allow the agent to enter the perturbed input monocyte or monocyte-dendritic progenitor cell. In some embodiments, the agent that inhibits formation of pDCs and classical DCs downregulates expression of STAT3 and/or Xbp1. In further embodiments, the agent that downregulates expression of STAT3 and/or Xbp1 is a nucleic acid, a protein, a peptide, a nucleic acid-protein complex or a small molecule. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that inhibits formation of pDCs and classical DCs from a monocyte or monocyte-dendritic progenitor cell comprises one or more Cas9-gRNA RNP complexes targeting STAT3 and/or Xbp1. In some embodiments, the expression of STAT3 and/or Xbp1 is decreased by about any one of: 5%, 10%, 20%, 30%, 40%, 50%, 60%, 70%, 80%, 90%, 95%, 99%, or 100%. In some embodiments, the expression of STAT3 and/or Xbp1 is decreased by about any one of: 2-fold, 3-fold, 5-fold, 10-fold, 50-fold, 100 fold, 500-fold, or 1000-fold. In some embodiments, formation of pDCs and classical DCs from a monocyte or monocyte-dendritic progenitor cell comprising the agent is decreased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, formation of pDCs and classical DCs from a monocyte or monocyte-dendritic progenitor cell comprising the agent is decreased by about any one of: 2-fold, 3-fold, 5-fold, 10-fold, 50-fold, 100 fold, 500-fold, or 1000-fold compared to respective monocyte or monocyte-dendritic progenitor cell that does not comprise the agent.

In some embodiments, according to any of the methods for enhancing the viability and/or function of an antigen presenting cell described herein, the modified antigen presenting cell comprises two or more agents that enhance the viability and/or function of the antigen presenting cell is delivered to the antigen presenting cell. In further embodiments, according to the modified antigen presenting cells described above, the two or more agents that enhance the viability and/or function of the antigen presenting cell are chosen from one or more of a tumor homing agent, an anti-apoptotic agent, a T cell activating agent, an antigen processing agent, an immune activity modulating agent, a homing receptor, or an agent that downregulates T cell inhibition.

In some embodiments, according to any of the methods for enhancing the viability and/or function of an antigen presenting cell described herein, the agent that enhances the viability and/or function of the antigen presenting cell is an agent that alters cell fate or cell phenotype. In some embodiments, the agent that alters cell fate or phenotype is a somatic cell reprogramming factor. In some embodiments, the agent that alters cell fate or phenotype is a dedifferentiation factor. In some embodiments, the agent that alters cell fate or phenotype is a trans-differentiation factor. In some embodiments, the agent that alters cell phenotype is a differentiation factor. In further embodiments, the agent that alters cell fate or phenotype is one or more of OCT4, SOX2, C-MYC, KLF-4, NANOG, LIN28 or LIN28B. In some embodiments, the agent that alters cell fate or phenotype is one or more of T-bet, GATA3. In some embodiments, the agent that alters cell fate or phenotype is one or more of EOMES, RUNX1, ERG, LCOR, HOXA5, or HOXA9. In some embodiments, the agent that alters cell fate or phenotype is one or more of GM-CSF, M-CSF, or RANKL. In some embodiments, the agent that alters cell fate or cell phenotype comprises one or more mRNAs encoding one or more of: OCT4, SOX2, C-MYC, KLF-4, NANOG, LIN28, LIN28B, T-bet, GATA3, EOMES, RUNX1, ERG, LCOR, HOXA5, HOXA9, GM-CSF, M-CSF, or RANKL. In some embodiments, the expression of one or more of OCT4, SOX2, C-MYC, KLF-4, NANOG, LIN28, LIN28B, T-bet, GATA3, EOMES, RUNX1, ERG, LCOR, HOXA5, HOXA9, GM-CSF, M-CSF, or RANKL is increased by about any one of: 5%, 10%, 20%, 30%, 40%, 50%, 60%, 70%, 80%, 90%, 95%, 99%, or 100%. In some embodiments, the expression of one or more of OCT4, SOX2, C-MYC, KLF-4, NANOG, LIN28, LIN28B, T-bet, GATA3, EOMES, RUNX1, ERG, LCOR, HOXA5, HOXA9, GM-CSF, M-CSF, or RANKL is increased by about any one of: 2-fold, 3-fold, 5-fold, 10-fold, 50-fold, 100 fold, 500-fold, 1000-fold, or more.

In some embodiments, according to any of the methods for enhancing the viability and/or function of an antigen presenting cell described herein, the modified antigen presenting cell further comprises an antigen. In some embodiments, the antigen is delivered before, at the same time, or after the agent that enhances the viability and/or function of the antigen presenting cell is delivered to the cell. In some embodiments, the antigen is delivered to the antigen presenting cell by a method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for the antigen to pass into the antigen presenting cell; and b) incubating the perturbed input antigen presenting cell with the antigen for a sufficient time to allow the antigen to enter the perturbed input antigen presenting cell.

In some embodiments, according to any of the methods for enhancing the viability and/or function of an antigen presenting cell described herein, the modified antigen presenting cell further comprises an adjuvant. In some embodiments, the adjuvant is delivered before, at the same time, or after the antigen is delivered to the cell and/or before, at the same time, or after the agent that enhances the viability and/or function of the antigen presenting cell is delivered to the cell. In some embodiments, the adjuvant is delivered to the antigen presenting cell by a method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for the adjuvant to pass into the antigen presenting cell; and b) incubating the perturbed input antigen presenting cell with the adjuvant for a sufficient time to allow the adjuvant to enter the perturbed input antigen presenting cell.

In some embodiments, the invention provides methods for enhancing the viability and/or function of an antigen presenting cell described herein, wherein any agent that enhances the viability and/or function of an antigen presenting cell as described herein is delivered to the cell by means other than by passing the cell through a constriction or is delivered to the cell extracellularly. In some embodiments, the invention provides methods for enhancing the viability and/or function of an antigen presenting cell described herein, wherein any agent that enhances the viability and/or function of an antigen presenting cell as described herein is delivered to the cell by means other than by passing the cell through a constriction or is delivered to the cell extracellularly and where the antigen is delivered to the antigen presenting cell by a method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for the antigen to pass into the antigen presenting cell; and b) incubating the perturbed input antigen presenting cell with the antigen for a sufficient time to allow the antigen to enter the perturbed input antigen presenting cell.

In some embodiments, the invention provides methods for enhancing the viability and/or function of an antigen presenting cell described herein, wherein any agent that enhances the viability and/or function of an antigen presenting cell as described herein is delivered to the cell by means other than by passing the cell through a constriction or is delivered to the cell extracellularly and wherein the antigen presenting cell comprises an adjuvant, wherein the adjuvant is delivered to the antigen presenting cell by a method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the adjuvant to pass into the antigen presenting cell; and b) incubating the perturbed input antigen presenting cell with the adjuvant for a sufficient time to allow the adjuvant to enter the perturbed input antigen presenting cell.

In some embodiments, the invention provides methods for enhancing the viability and/or function of an antigen presenting cell described herein, wherein any agent that enhances the viability and/or function of an antigen presenting cell as described herein is delivered to the cell by means other than by passing the cell through a constriction or is delivered to the cell extracellularly and wherein the antigen and an adjuvant is delivered to the antigen presenting cell by a method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the antigen and adjuvant to pass into the antigen presenting cell; and b) incubating the perturbed input antigen presenting cell with the antigen and the adjuvant for a sufficient time to allow the antigen and the adjuvant to enter the perturbed input antigen presenting cell.

In some embodiments, the invention provides methods for enhancing the viability and/or function of an antigen presenting cell described herein, wherein the antigen presenting cell comprises an antigen and/or an adjuvant, the agent is delivered to the antigen presenting cell by a method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the agent to pass into the antigen presenting cell; and b) incubating the perturbed input antigen presenting cell with the antigen and the adjuvant for a sufficient time to allow the agent to enter the perturbed input antigen presenting cell. In some embodiments, the antigen and/or adjuvant is delivered to the cell by means other than by passing the cell through a constriction or is delivered to the cell extracellularly.

In some embodiments according to any one of the embodiments described herein, the antigen, adjuvant and/or agent that enhances the viability and/or function of an antigen is delivered into an antigen presenting cell in a method comprising: passing an input antigen presenting cell through an energy field. In some embodiments, the energy field is one or more of: an optical field, an acoustic field, a magnetic field or an electric field. In some embodiments, the antigen, adjuvant and/or agent that enhances the viability and/or function of an antigen is delivered into an antigen presenting cell in a method comprising: passing an input antigen presenting cell through an electric field. In some embodiments, the electric field is between about 0.1 kV/m to about 100 MV/m, or any number or range of numbers therebetween. In some embodiments according to any one of the embodiments described herein, the antigen, adjuvant and/or agent that enhances the viability and/or function of an antigen is delivered into an antigen presenting cell by electroporation.

Therefore in some embodiments, according to any of the methods for enhancing the viability and/or function of an antigen presenting cell described herein, the modified antigen presenting cell further comprises an antigen and/or an adjuvant. In some embodiments, the antigen is exogenous to the modified antigen presenting cell and comprises an immunogenic epitope, and the adjuvant is present intracellularly. Exogenous antigens are one or more antigens from a source outside the antigen presenting cell introduced into a cell to be modified. Exogenous antigens can include antigens that may be present in the antigen presenting cell (i.e. also present from an endogenous source), either before or after introduction of the exogenous antigen, and as such can thus be produced by the antigen presenting cell (e.g., encoded by the genome of the antigen presenting cell). For example, in some embodiments, the modified antigen presenting cell further comprises two pools of an antigen, a first pool comprising an endogenous source of the antigen, and a second pool comprising an exogenous source of the antigen produced outside of and introduced into the antigen presenting cell to be modified. In some embodiments, the antigen is ectopically expressed or overexpressed in a disease cell in an individual, and the modified antigen presenting cell is derived from the individual and comprises an exogenous source of the antigen, or an immunogenic epitope contained therein, produced outside of and introduced into the antigen presenting cell to be modified. In some embodiments, the antigen is a neoantigen (e.g., an altered-self protein or portion thereof) comprising a neoepitope, and the modified antigen presenting cell comprises an exogenous source of the antigen, or a fragment thereof comprising the neoepitope, produced outside of and introduced into the antigen presenting cell to be modified. In some embodiments, the adjuvant is exogenous to the modified antigen presenting cell. In some embodiments, the antigen and/or the adjuvant are present in multiple compartments of the modified antigen presenting cell. In some embodiments, the antigen and/or adjuvant are present in the cytosol and/or a vesicle of the modified antigen presenting cell. In some embodiments, the vesicle is an endosome. In some embodiments, the antigen or immunogenic epitope, and/or the adjuvant is bound to the surface of the antigen presenting cell.

In some embodiments, according to any of the methods for enhancing the viability and/or function of an antigen presenting cell described herein, the antigen is present in multiple compartments of the modified antigen presenting cell. In some embodiments, the antigen is present in the cytosol and/or a vesicle of the modified antigen presenting cell. In some embodiments, the vesicle is an endosome. In some embodiments, the antigen is bound to the surface of the modified antigen presenting cell. In some embodiments, the antigen or an immunogenic epitope contained therein is bound to the surface of the modified antigen presenting cell. In some embodiments, the antigen presenting cell is a PBMC. In some embodiments, the antigen presenting cell is a mixed population of cells. In some embodiments, the antigen presenting cell is in a mixed population of cells, wherein the mixed population of cells is a population of PBMCs. In some embodiments, the PBMC includes one or more of a T cell, a B cell, an NK cells or, a monocyte, a macrophage or a dendritic cell. In some embodiments, the modified antigen presenting cell further comprises an adjuvant. In some embodiments, the antigen and/or the adjuvant are present in the cytosol and/or a vesicle of the antigen presenting cell.

In some embodiments, according to any of the methods for enhancing the viability and/or function of an antigen presenting cell described herein, the adjuvant is present in multiple compartments of the modified antigen presenting cell. In some embodiments, the adjuvant is present in the cytosol and/or a vesicle of the modified antigen presenting cell. In some embodiments, the vesicle is an endosome. In some embodiments, the adjuvant is bound to the surface of the modified antigen presenting cell. In some embodiments, the antigen presenting cell is a PBMC. In some embodiments, the antigen presenting cell is a mixed population of cells. In some embodiments, the antigen presenting cell is in a mixed population of cells, wherein the mixed population of cells is a population of PBMCs. In some embodiments, the PBMC includes one or more of a T cell, a B cell, an NK cells or, a monocyte, a macrophage or a dendritic cell. In some embodiments, the modified antigen presenting cell further comprises an antigen. In some embodiments, the antigen and/or the adjuvant are present in the cytosol and/or a vesicle of the antigen presenting cell.

In some embodiments, according to any of the methods for enhancing the viability and/or function of an antigen presenting cell described herein, the modified antigen presenting cell further comprises an adjuvant. In some embodiments, the adjuvant is a CpG oligodeoxynucleotide (ODN), IFN-α, STING agonists, RIG-I agonists, poly I:C, imiquimod, and/or resiquimod. In some embodiments, the adjuvant is a CpG ODN. In some embodiments, the CpG ODN is no greater than about 50 (such as no greater than about any of 45, 40, 35, 30, 25, 20, or fewer) nucleotides in length. In some embodiments, the CpG ODN is a Class A CpG ODN, a Class B CpG ODN, or a Class C CpG ODN. In some embodiments, the CpG ODN comprises the nucleotide sequences as disclosed in US provisional application U.S. 62/641,987, incorporated herein by reference in its entirety. In some embodiments, the modified antigen presenting cell comprises a plurality of different CpG ODNs. For example, in some embodiments, the modified antigen presenting cell comprises a plurality of different CpG ODNs selected from among Class A, Class B, and Class C CpG ODNs.

In some embodiments, according to any of the methods for enhancing the viability and/or function of an antigen presenting cell described herein, the antigen is a disease-associated antigen. In further embodiments, the antigen is a tumor antigen. In some embodiments, the antigen is derived from a lysate. In some embodiments, the lysate is derived from a biopsy of an individual. In some embodiments, the lysate is derived from a biopsy of an individual being infected by a pathogen, such as a bacteria or a virus. In some embodiments, the lysate is derived from a biopsy of an individual bearing tumors (i.e. tumor biopsy lysates). Thus in some embodiments, the lysate is a tumor lysate.

In some embodiments, according to any of the methods for enhancing the viability and/or function of an antigen presenting cell described herein, the modified antigen presenting cell comprises an antigen comprising an immunogenic epitope. In some embodiments, the immunogenic epitope is derived from a disease-associated antigen. In some embodiments, the immunogenic epitope is derived from peptides or mRNA isolated from a diseased cell. In some embodiments, the immunogenic epitope is derived from a protein ectopically expressed or overexpressed in a diseased cell. In some embodiments, the immunogenic epitope is derived from a neoantigen, e.g., a cancer-associated neoantigen. In some embodiments, the immunogenic epitope comprises a neoepitope, e.g., a cancer-associated neoepitope. In some embodiments, the immunogenic epitope is derived from a non-self antigen. In some embodiments, the immunogenic epitope is derived from a mutated or otherwise altered self antigen. In some embodiments, the immunogenic epitope is derived from a tumor antigen, viral antigen, bacterial antigen, or fungal antigen. In some embodiments, the antigen comprises an immunogenic epitope fused to heterologous peptide sequences. In some embodiments, the antigen comprises a plurality of immunogenic epitopes. In some embodiments, some of the plurality of immunogenic epitopes are derived from the same source. For example, in some embodiments, some of the plurality of immunogenic epitopes are derived from the same viral antigen. In some embodiments, all of the plurality of immunogenic epitopes are derived from the same source. In some embodiments, none of the plurality of immunogenic epitopes are derived from the same source. In some embodiments, the modified antigen presenting cell comprises a plurality of different antigens.

In some embodiments, according to any of the methods for enhancing the viability and/or function of an antigen presenting cell described herein, the modified antigen presenting cell further comprises an antigen, wherein the antigen comprises an immunogenic epitope. In some embodiments, the antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the immunogenic peptide epitope fused to the N-terminal flanking polypeptide and/or the C-terminal flanking polypeptide is a non-naturally occurring sequence. In some embodiments, the N-terminal and/or C-terminal flanking polypeptides are derived from an immunogenic synthetic long peptide (SLP). In some embodiments, the N-terminal and/or C-terminal flanking polypeptides are derived from a disease-associated immunogenic SLP. In some embodiments, the immunogenic peptide epitope fused to the N-terminal flanking polypeptide and/or the C-terminal flanking polypeptide is heterologous to the cell to which it is delivered.

In some embodiments, according to any of the methods for enhancing the viability and/or function of an antigen presenting cell described herein, the modified antigen presenting cell further comprises an antigen, wherein the antigen is capable of being processed into an MHC class I-restricted peptide and/or an MHC class II-restricted peptide. In some embodiments, the antigen is capable of being processed into an MHC class I-restricted peptide. In some embodiments, the antigen is capable of being processed into an MHC class II-restricted peptide. In some embodiments, the antigen comprises a plurality of immunogenic epitopes, and is capable of being processed into an MHC class I-restricted peptide and an MHC class II-restricted peptide. In some embodiments, some of the plurality of immunogenic epitopes are derived from the same source. In some embodiments, all of the plurality of immunogenic epitopes are derived from the same source. In some embodiments, none of the plurality of immunogenic epitopes are derived from the same source.

In some embodiments, according to any of the methods for enhancing the viability and/or function of an antigen presenting cell described herein, the modified antigen presenting cell comprises a plurality of antigens that comprise a plurality of immunogenic epitopes. In some embodiments, following administration to an individual of the modified antigen presenting cell comprising the plurality of antigens that comprise the plurality of immunogenic epitopes, none of the plurality of immunogenic epitopes decreases an immune response in the individual to any of the other immunogenic epitopes.

In some embodiments, according to any of the methods for enhancing the viability and/or function of an antigen presenting cell described herein, the modified antigen presenting cell comprises an agent that enhances the viability and/or function of the modified antigen presenting cell. In some embodiments, the modified antigen presenting cell further comprises an antigen and/or an adjuvant. In some embodiments, the modified antigen presenting cell comprises the agent that enhances the viability and/or function of the modified antigen presenting cell at a concentration between about 1 pM and about 10 mM. In some embodiments, the modified antigen presenting cell comprises the antigen at a concentration between about 1 pM and about 10 mM. In some embodiments, the modified antigen presenting cell comprises the adjuvant at a concentration between about 1 pM and about 10 mM. In some embodiments, the modified antigen presenting cell comprises the agent that enhances the viability and/or function of the modified antigen presenting cell at a concentration between about 0.1 μM and about 10 mM. In some embodiments, the modified antigen presenting cell comprises the antigen at a concentration between about 0.1 μM and about 10 mM. In some embodiments, the modified antigen presenting cell comprises the adjuvant at a concentration between about 0.1 μM and about 10 mM. For example, in some embodiments, the concentration of the agent that enhances the viability and/or function of the modified antigen presenting cell in the modified antigen presenting cell is any of less than about 1 pM, about 10 pM, about 100 pM, about 1 nM, about 10 nM, about 100 nM, about 1 μM, about 10 μM, about 100 μM, about 1 mM or about 10 mM. In some embodiments, the concentration of the agent that enhances the viability and/or function of the modified antigen presenting cell in the modified antigen presenting cell is greater than about 10 mM. In some embodiments, the concentration of adjuvant in the modified antigen presenting cell is any of less than about 1 pM, about 10 pM, about 100 pM, about 1 nM, about 10 nM, about 100 nM, about 1 μM, about 10 μM, about 100 μM, about 1 mM or about 10 mM. In some embodiments, the concentration of adjuvant in the modified antigen presenting cell is greater than about 10 mM. In some embodiments, the concentration of antigen in the modified antigen presenting cell is any of less than about 1 pM, about 10 pM, about 100 pM, about 1 nM, about 10 nM, about 100 nM, about 1 about 10 about 100 about 1 mM or about 10 mM. In some embodiments, the concentration of antigen in the modified antigen presenting cell is greater than about 10 mM. In some embodiments, the concentration of the agent that enhances the viability and/or function of the modified antigen presenting cell in the modified antigen presenting cell is any of between about 1 pM and about 10 pM, between about 10 pM and about 100 pM, between about 100 pM and about 1 nM, between about 1 nM and about 10 nM, between about 10 nM and about 100 nM, between about 100 nM and about 1 between about 1 μM and about 10 between about 10 μM and about 100 between about 100 μM and about 1 mM, or between 1 mM and about 10 mM.

In some embodiments, according to any of the methods for enhancing the viability and/or function of an antigen presenting cell described herein, the molar ratio of the agent that enhances the viability and/or function of the modified antigen presenting cell to antigen in the modified antigen presenting cell is any of between about 10000:1 to about 1:10000. For example, in some embodiments, the molar ratio of the agent that enhances the viability and/or function of the modified antigen presenting cell to antigen in the modified antigen presenting cell is about any of 10000:1, about 1000:1, about 100:1, about 10:1, about 1:1, about 1:10, about 1:100, about 1:1000, or about 1:10000. In some embodiments, the molar ratio of the agent that enhances the viability and/or function of the modified antigen presenting cell to antigen in the modified antigen presenting cell is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the molar ratio of the agent that enhances the viability and/or function of the modified antigen presenting cell to adjuvant in the modified antigen presenting cell is any of between about 10000:1 to about 1:10000. For example, in some embodiments, the molar ratio of the agent to adjuvant in the modified antigen presenting cell is about any of 10000:1, about 1000:1, about 100:1, about 10:1, about 1:1, about 1:10, about 1:100, about 1:1000, or about 1:10000. In some embodiments, the molar ratio of the agent that enhances the viability and/or function of the modified antigen presenting cell to adjuvant in the modified antigen presenting cell is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the modified antigen presenting cell comprises a complex comprising: a) the agent that enhances the viability and/or function of the modified antigen presenting cell, b) the agent and at least another agent, c) the agent and the antigen, d) the agent and the adjuvant, and/or e) the agent, the antigen and the adjuvant.

In some embodiments, according to any of the methods for enhancing the viability and/or function of an antigen presenting cell described herein, the modified antigen presenting cell further comprises an additional agent that enhances the viability and/or function of the modified antigen presenting cell as compared to a corresponding modified antigen presenting cell that does not comprise the additional agent. In some embodiments, the additional agent is a stabilizing agent or a co-factor. In some embodiments, the agent is albumin. In some embodiments, the albumin is mouse, bovine, or human albumin. In some embodiments, the additional agent is a divalent metal cation, glucose, ATP, potassium, glycerol, trehalose, D-sucrose, PEG1500, L-arginine, L-glutamine, or EDTA.

In some embodiments, according to any of the methods for enhancing the viability and/or function of an antigen presenting cell described herein, the modified antigen presenting cell comprises a further modification. In some embodiments, the modified antigen presenting cell comprises a further modification to modulate MHC class I expression. In some embodiments, the modified antigen presenting cell comprises a further modification to decrease MHC class I expression. In some embodiments, the modified antigen presenting cell comprises a further modification to increase MHC class I expression. In some embodiments, the modified T cell comprises a further modification to modulate MHC class II expression. In some embodiments, the modified antigen presenting cell comprises a further modification to decrease MHC class II expression. In some embodiments, the modified antigen presenting cell comprises a further modification to increase MHC class II expression. In some embodiments, an innate immune response mounted in an individual in response to administration, in an allogeneic context, of the modified antigen presenting cells is reduced compared to an innate immune response mounted in an individual in response to administration, in an allogeneic context, of corresponding modified antigen presenting cells that do not comprise the further modification. In some embodiments, the circulating half-life and/or in vivo persistence of the modified antigen presenting cells in an individual to which they were administered is increased compared to the circulating half-life and/or in vivo persistence of corresponding modified T cells that do not comprise the further modification in an individual to which they were administered.

In certain aspects, there is provided a method for enhancing the viability and/or function of an antigen presenting cell, the method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the agent that enhances the viability and/or function of the antigen presenting cell, an antigen and an adjuvant to pass through to form a perturbed input antigen presenting cell; and b) incubating the perturbed input antigen presenting cell with the agent that enhances the viability and/or function of the antigen presenting cell, the antigen and the adjuvant for a sufficient time to allow the agent, the antigen and the adjuvant to enter the perturbed input antigen presenting cell; thereby generating the modified antigen presenting cell comprising the agent that enhances the viability and/or function of the antigen presenting cell, the antigen and the adjuvant. In some embodiments, the concentration of the agent that enhances the viability and/or function of the antigen presenting cell incubated with the perturbed input antigen presenting cell is between about 1 pM-10 mM, the concentration of the antigen incubated with the perturbed input antigen presenting cell is between about 1 pM-10 mM and the concentration of the adjuvant incubated with the perturbed input antigen presenting cell is between about 1 pM-10 mM. In some embodiments, the concentration of the agent that enhances the viability and/or function of the antigen presenting cell incubated with the perturbed input antigen presenting cell is between about 0.1 μM-10 mM, the concentration of the antigen incubated with the perturbed input antigen presenting cell is between about 0.1 μM-10 mM and the concentration of the adjuvant incubated with the perturbed input antigen presenting cell is between about 0.1 μM-10 mM. In some embodiments, the ratio of the agent to the antigen incubated with the perturbed input antigen presenting cell is between about 10000:1 to about 1:10000. In some embodiments, the ratio of the agent to the adjuvant incubated with the perturbed input antigen presenting cell is between about 10000:1 to about 1:10000. In some embodiments, the ratio of the antigen to the adjuvant incubated with the perturbed input antigen presenting cell is between about 10000:1 to about 1:10000.

In some embodiments, the method for enhancing the viability and/or function of an antigen presenting cell described herein comprises a process employing a cell-deforming constriction through which an input antigen presenting cell is passed. In some embodiments, the diameter of the constriction is less than the diameter of the input antigen presenting cell. In some embodiments, the diameter of the constriction is about 20% to about 99% of the diameter of the input antigen presenting cell. In some embodiments, the diameter of the constriction is about 20% to about 60% of the diameter of the input antigen presenting cell. In some embodiments, the cell-deforming constriction is contained in a microfluidic channel, such as any of the microfluidic channels described herein. The microfluidic channel may be contained in any of the microfluidic devices described herein, such as described in the section titled Microfluidic Devices below. Thus, in some embodiments, according to any of the methods described herein prepared by a process employing a microfluidic channel including a cell-deforming constriction through which an input antigen presenting cell is passed, the process comprises passing the input antigen presenting cell through a microfluidic channel including a cell-deforming constriction contained in any of the microfluidic systems described herein. In some embodiments, a deforming force is applied to the input antigen presenting cell as it passes through the constriction, thereby causing the perturbations of the input antigen presenting cell.

Input antigen presenting cells can be obtained from a number of sources, including peripheral blood mononuclear cells (PBMCs), bone marrow, lymph node tissue, cord blood, thymus tissue, tissue from a site of infection, ascites, pleural effusion, spleen tissue, and tumors. In some embodiments, according to any of the methods for enhancing the viability and/or function of an antigen presenting cell described herein, the input antigen presenting cell is a PBMC. In some embodiments, the antigen presenting cell is a mixed population of cells. In some embodiments, the antigen presenting cell is in a mixed population of cells, wherein the mixed population of cells is a population of PBMCs. In some embodiments, the PBMC is a T cell, a B cell, an NK cells, a monocyte, a macrophage and/or a dendritic cell. In some embodiments of the present invention, any number of cell lines of PBMC subtype population available in the art may be used, such as T cell lines or B cell lines. In some embodiments of the present invention, various subtype populations of PBMCs can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, CD45RO⁺ T cells, and γδ-T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least one, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process (negative selection). “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD11b, CD 16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/mL is used. In some embodiments, a concentration of about 1 billion cells/mL is used. In some embodiments, greater than about 100 million cells/mL is used. In some embodiments, a concentration of cells of about any of 10, 15, 20, 25, 30, 35, 40, 45, or 50 million cells/mL is used. In some embodiments, a concentration of cells of about any of 75, 80, 85, 90, 95, or 100 million cells/mL is used. In some embodiments, a concentration of about 125 or about 150 million cells/mL is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments, according to any of the methods for enhancing the viability and/or function of an antigen presenting cell described herein, wherein the modified antigen presenting cell comprises an agent that enhances the viability and/or function of the modified antigen presenting cell, the input antigen presenting cell is a peripheral blood mononuclear cell (PBMC). In some embodiments, the antigen presenting cell is a mixed population of cells. In some embodiments, the antigen presenting cell is in a mixed population of cells, wherein the mixed population of cells is a population of PBMCs. In some embodiments, the PBMC is a T cell, a B cell, an NK cells, a monocyte, a macrophage and/or a dendritic cell. In some embodiments, the PBMC is engineered to present an antigen. In some embodiments, the agent enhances tumor homing of the antigen presenting cell. In some embodiments, the agent is an anti-apoptotic agent. In some embodiments, the agent enhances T-cell activation. In some embodiments, the agent enhances antigen processing. In some embodiments, the agent enhances antigen processing and loading into MHC-1. In some embodiments, the agent modulates immune activity. In some embodiments, the agent is a homing receptor. In some embodiments, the agent downregulates T cell inhibition.

In some embodiments, according to any of the methods for modulating the function of monocyte, or monocyte-dendritic progenitor or DC described herein, the modified monocyte, or monocyte-dendritic progenitor or DC further comprises an antigen. In some embodiments, the antigen is delivered before, at the same time, or after the agent that promotes or inhibits DC formation is delivered to the cell. In some embodiments, the antigen is delivered to the monocyte, or monocyte-dendritic progenitor or DC by a method comprising: a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the antigen to pass into the monocyte, or monocyte-dendritic progenitor or DC; and b) incubating the perturbed input monocyte, or monocyte-dendritic progenitor or DC with the antigen for a sufficient time to allow the antigen to enter the perturbed input monocyte, or monocyte-dendritic progenitor or DC.

In some embodiments, according to any of the methods for modulating the function of monocyte, or monocyte-dendritic progenitor or DC described herein, the modified monocyte, or monocyte-dendritic progenitor or DC further comprises an adjuvant. In some embodiments, the adjuvant is delivered before, at the same time, or after the antigen is delivered to the cell and/or before, at the same time, or after the agent that promotes or inhibits DC formation of the monocyte, or monocyte-dendritic progenitor or DC is delivered to the cell. In some embodiments, the adjuvant is delivered to the monocyte, or monocyte-dendritic progenitor or DC by a method comprising: a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the adjuvant to pass into the monocyte, or monocyte-dendritic progenitor or DC; and b) incubating the perturbed input monocyte, or monocyte-dendritic progenitor or DC with the adjuvant for a sufficient time to allow the adjuvant to enter the perturbed input monocyte, or monocyte-dendritic progenitor or DC.

Therefore in some embodiments, according to any of the methods for modulating the function of monocyte, or monocyte-dendritic progenitor or DC described herein, the modified monocyte, or monocyte-dendritic progenitor or DC further comprises an antigen and/or an adjuvant. In some embodiments, the antigen is exogenous to the modified monocyte, or monocyte-dendritic progenitor or DC and comprises an immunogenic epitope, and the adjuvant is present intracellularly. Exogenous antigens are one or more antigens from a source outside the monocyte, or monocyte-dendritic progenitor or DC introduced into a cell to be modified. Exogenous antigens can include antigens that may be present in the monocyte, or monocyte-dendritic progenitor or DC (i.e. also present from an endogenous source), either before or after introduction of the exogenous antigen, and as such can thus be produced by the monocyte, or monocyte-dendritic progenitor or DC (e.g., encoded by the genome of the monocyte, or monocyte-dendritic progenitor or DC). For example, in some embodiments, the modified monocyte, or monocyte-dendritic progenitor or DC further comprises two pools of an antigen, a first pool comprising an endogenous source of the antigen, and a second pool comprising an exogenous source of the antigen produced outside of and introduced into the monocyte, or monocyte-dendritic progenitor or DC to be modified. In some embodiments, the antigen is ectopically expressed or overexpressed in a disease cell in an individual, and the modified monocyte, or monocyte-dendritic progenitor or DC is derived from the individual and comprises an exogenous source of the antigen, or an immunogenic epitope contained therein, produced outside of and introduced into the monocyte, or monocyte-dendritic progenitor or DC to be modified. In some embodiments, the antigen is a neoantigen (e.g., an altered-self protein or portion thereof) comprising a neoepitope, and the modified monocyte, or monocyte-dendritic progenitor or DC comprises an exogenous source of the antigen, or a fragment thereof comprising the neoepitope, produced outside of and introduced into the monocyte, or monocyte-dendritic progenitor or DC to be modified. In some embodiments, the adjuvant is exogenous to the modified monocyte, or monocyte-dendritic progenitor or DC. In some embodiments, the antigen and/or the adjuvant are present in multiple compartments of the modified monocyte, or monocyte-dendritic progenitor or DC. In some embodiments, the antigen and/or adjuvant are present in the cytosol and/or a vesicle of the modified monocyte, or monocyte-dendritic progenitor or DC. In some embodiments, the vesicle is an endosome. In some embodiments, the antigen or immunogenic epitope, and/or the adjuvant is bound to the surface of the modified monocyte, or monocyte-dendritic progenitor or DC.

In some embodiments, according to any of the methods for modulating the function of monocyte, or monocyte-dendritic progenitor or DC described herein, the antigen is present in multiple compartments of the modified monocyte, or monocyte-dendritic progenitor or DC. In some embodiments, the antigen is present in the cytosol and/or a vesicle of the modified monocyte, or monocyte-dendritic progenitor or DC. In some embodiments, the vesicle is an endosome. In some embodiments, the antigen is bound to the surface of the modified monocyte, or monocyte-dendritic progenitor or DC. In some embodiments, the antigen or an immunogenic epitope contained therein is bound to the surface of the modified monocyte, or monocyte-dendritic progenitor or DC. In some embodiments, the antigen and/or the adjuvant are present in the cytosol and/or a vesicle of the monocyte, or monocyte-dendritic progenitor or DC.

In some embodiments, according to any of the methods for modulating the function of monocyte, or monocyte-dendritic progenitor or DC described herein, the adjuvant is present in multiple compartments of the modified monocyte, or monocyte-dendritic progenitor or DC. In some embodiments, the adjuvant is present in the cytosol and/or a vesicle of the modified monocyte, or monocyte-dendritic progenitor or DC. In some embodiments, the vesicle is an endosome. In some embodiments, the adjuvant is bound to the surface of the modified monocyte, or monocyte-dendritic progenitor or DC. In some embodiments, the modified monocyte, or monocyte-dendritic progenitor or DC further comprises an antigen. In some embodiments, the antigen and/or the adjuvant are present in the cytosol and/or a vesicle of the monocyte, or monocyte-dendritic progenitor or DC.

In some embodiments, according to any of the methods for modulating the function of monocyte, or monocyte-dendritic progenitor or DC described herein, the modified monocyte, or monocyte-dendritic progenitor or DC further comprises an adjuvant. In some embodiments, the adjuvant is a CpG oligodeoxynucleotide (ODN), IFN-α, STING agonists, RIG-I agonists, poly I:C, imiquimod, and/or resiquimod. In some embodiments, the adjuvant is a CpG ODN. In some embodiments, the CpG ODN is no greater than about 50 (such as no greater than about any of 45, 40, 35, 30, 25, 20, or fewer) nucleotides in length. In some embodiments, the CpG ODN is a Class A CpG ODN, a Class B CpG ODN, or a Class C CpG ODN. In some embodiments, the CpG ODN comprises the nucleotide sequences as disclosed in US provisional application U.S. 62/641,987, incorporated herein by reference in its entirety. In some embodiments, the modified monocyte, or monocyte-dendritic progenitor or DC comprises a plurality of different CpG ODNs. For example, in some embodiments, the modified monocyte, or monocyte-dendritic progenitor or DC comprises a plurality of different CpG ODNs selected from among Class A, Class B, and Class C CpG ODNs.

In some embodiments, according to any of the methods for modulating the function of monocyte, or monocyte-dendritic progenitor or DC described herein, the antigen is a disease-associated antigen. In further embodiments, the antigen is a tumor antigen. In some embodiments, the antigen is derived from a lysate. In some embodiments, the lysate is derived from a biopsy of an individual. In some embodiments, the lysate is derived from a biopsy of an individual being infected by a pathogen, such as a bacteria or a virus. In some embodiments, the lysate is derived from a biopsy of an individual bearing tumors (i.e. tumor biopsy lysates). Thus in some embodiments, the lysate is a tumor lysate.

In some embodiments, according to any of the methods for modulating the function of monocyte, or monocyte-dendritic progenitor or DC described herein, the modified monocyte, or monocyte-dendritic progenitor or DC comprises an antigen comprising an immunogenic epitope. In some embodiments, the immunogenic epitope is derived from a disease-associated antigen. In some embodiments, the immunogenic epitope is derived from peptides or mRNA isolated from a diseased cell. In some embodiments, the immunogenic epitope is derived from a protein ectopically expressed or overexpressed in a diseased cell. In some embodiments, the immunogenic epitope is derived from a neoantigen, e.g., a cancer-associated neoantigen. In some embodiments, the immunogenic epitope comprises a neoepitope, e.g., a cancer-associated neoepitope. In some embodiments, the immunogenic epitope is derived from a non-self antigen.

In some embodiments, the immunogenic epitope is derived from a mutated or otherwise altered self antigen. In some embodiments, the immunogenic epitope is derived from a tumor antigen, viral antigen, bacterial antigen, or fungal antigen. In some embodiments, the antigen comprises an immunogenic epitope fused to heterologous peptide sequences. In some embodiments, the antigen comprises a plurality of immunogenic epitopes. In some embodiments, some of the plurality of immunogenic epitopes are derived from the same source. For example, in some embodiments, some of the plurality of immunogenic epitopes are derived from the same viral antigen. In some embodiments, all of the plurality of immunogenic epitopes are derived from the same source. In some embodiments, none of the plurality of immunogenic epitopes are derived from the same source. In some embodiments, the modified monocyte, or monocyte-dendritic progenitor or DC comprises a plurality of different antigens.

In some embodiments, according to any of the methods for modulating the function of monocyte, or monocyte-dendritic progenitor or DC described herein, the modified monocyte, or monocyte-dendritic progenitor or DC further comprises an antigen, wherein the antigen comprises an immunogenic epitope. In some embodiments, the antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the immunogenic peptide epitope fused to the N-terminal flanking polypeptide and/or the C-terminal flanking polypeptide is a non-naturally occurring sequence. In some embodiments, the N-terminal and/or C-terminal flanking polypeptides are derived from an immunogenic synthetic long peptide (SLP). In some embodiments, the N-terminal and/or C-terminal flanking polypeptides are derived from a disease-associated immunogenic SLP.

In some embodiments, according to any of the methods for modulating the function of monocyte, or monocyte-dendritic progenitor or DC described herein, the modified monocyte, or monocyte-dendritic progenitor or DC further comprises an antigen, wherein the antigen is capable of being processed into an MHC class I-restricted peptide and/or an MHC class II-restricted peptide. In some embodiments, the antigen is capable of being processed into an MHC class I-restricted peptide. In some embodiments, the antigen is capable of being processed into an MHC class II-restricted peptide. In some embodiments, the antigen comprises a plurality of immunogenic epitopes, and is capable of being processed into an MHC class I-restricted peptide and an MHC class II-restricted peptide. In some embodiments, some of the plurality of immunogenic epitopes are derived from the same source. In some embodiments, all of the plurality of immunogenic epitopes are derived from the same source. In some embodiments, none of the plurality of immunogenic epitopes are derived from the same source.

In some embodiments, according to any of the methods for enhancing the viability and/or function of monocyte, or monocyte-dendritic progenitor or DC described herein, the modified monocyte, or monocyte-dendritic progenitor or DC comprises a plurality of antigens that comprise a plurality of immunogenic epitopes. In some embodiments, following administration to an individual of the modified monocyte, or monocyte-dendritic progenitor or DC comprising the plurality of antigens that comprise the plurality of immunogenic epitopes, none of the plurality of immunogenic epitopes decreases an immune response in the individual to any of the other immunogenic epitopes.

In some embodiments, the method for enhancing modulating the function of monocyte, or monocyte-dendritic progenitor or DC described herein comprises a process employing a cell-deforming constriction through which an input monocyte, or monocyte-dendritic progenitor or DC is passed. In some embodiments, the diameter of the constriction is less than the diameter of the input monocyte, or monocyte-dendritic progenitor or DC. In some embodiments, the diameter of the constriction is about 20% to about 99% of the diameter of the input monocyte, or monocyte-dendritic progenitor or DC. In some embodiments, the diameter of the constriction is about 20% to about 60% of the diameter of the input monocyte, or monocyte-dendritic progenitor or DC. In some embodiments, the cell-deforming constriction is contained in a microfluidic channel, such as any of the microfluidic channels described herein. The microfluidic channel may be contained in any of the microfluidic devices described herein, such as described in the section titled Microfluidic Devices below. Thus, in some embodiments, according to any of the methods described herein prepared by a process employing a microfluidic channel including a cell-deforming constriction through which an input monocyte, or monocyte-dendritic progenitor or DC is passed, the process comprises passing the input monocyte, or monocyte-dendritic progenitor or DC through a microfluidic channel including a cell-deforming constriction contained in any of the microfluidic systems described herein. In some embodiments, a deforming force is applied to the input monocyte, or monocyte-dendritic progenitor or DC as it passes through the constriction, thereby causing the perturbations of the input monocyte, or monocyte-dendritic progenitor or DC.

In some embodiments, there is provided a modified antigen presenting cell comprising an agent that enhances the viability and/or function of an antigen presenting cell, wherein the cell is prepared by any of the methods described herein.

In some embodiments, there is a provided a modified monocyte, or monocyte-dendritic progenitor or DC, wherein the monocyte, or monocyte-dendritic progenitor or DC is prepared by the any of the methods described herein.

In some embodiments, there is provided a method for modulating an immune response in an individual, comprising: administering to the individual an antigen presenting cell, wherein the antigen presenting cell is prepared by a process according to any of the methods described herein.

In some embodiments, there is provided a method for modulating an immune response in an individual, comprising: administering to the individual a dendritic cell, wherein the dendritic cell is prepared by a process according to of any that is prepared by a process according to any of the methods described herein.

### Modified Antigen Presenting Cells

In certain aspects, there is provided a modified antigen presenting cell comprising an agent that enhances the viability and/or function of the antigen presenting cell, wherein the modified antigen presenting cell is prepared by a process comprising the steps of: a) passing a cell suspension comprising an input antigen presenting cell through a cell-deforming constriction, wherein a diameter of the constriction is a function of a diameter of the antigen presenting cell in the suspension, thereby causing perturbations of the input antigen presenting cell large enough for the agent that enhances the viability and/or function of the antigen to pass through to form a perturbed input antigen presenting cell; and b) incubating the perturbed input antigen presenting cell with the agent that enhances the viability and/or function of the antigen for a sufficient time to allow the antigen and the agent to enter the perturbed input antigen presenting cell; thereby generating the modified antigen presenting cell comprising the agent that enhances the viability and/or function of the antigen presenting cell.

In some embodiments according to any of the modified antigen presenting cells described herein, the agent comprises a protein or polypeptide. In some embodiments, the agent is a protein or polypeptide. In some embodiments, the protein or polypeptide is a therapeutic protein, antibody, fusion protein, antigen, synthetic protein, reporter marker, or selectable marker. In some embodiments, the protein is a gene-editing protein or nuclease such as a zinc-finger nuclease (ZFN), transcription activator-like effector nuclease (TALEN), mega nuclease, or CRE recombinase. In some embodiments, the gene-editing protein or nuclease is Cas 9. In further embodiments, the agent comprises Cas9 with or without an ssODN for homologous recombination or homology directed repair. In some embodiments, the fusion proteins can include, without limitation, chimeric protein drugs such as antibody drug conjugates or recombinant fusion proteins such as proteins tagged with OST or streptavidin. In some embodiments, the agent is a transcription factor. In some embodiments, the agent comprises a nucleic acid. In some embodiments, the agent is a nucleic acid. Exemplary nucleic acids include, without limitation, recombinant nucleic acids, DNA, recombinant DNA, cDNA, genomic DNA, RNA, siRNA, mRNA, saRNA, miRNA, lncRNA, tRNA, and shRNA. In some embodiments, the nucleic acid is homologous to a nucleic acid in the cell. In some embodiments, the nucleic acid is heterologous to a nucleic acid in the cell. In some embodiments, the agent is a plasmid. In some embodiments, the agent is a nucleic acid-protein complex. In some embodiments, the nucleic acid-protein complex is a gene-editing complex with or without an ssODN for homologous recombination. In some embodiments, the nucleic acid-protein complex comprises Cas9 and guide RNA, with or without an ssODN for homologous recombination or homology directed repair.

In some embodiments according to any of the modified antigen presenting cells described herein, the antigen presenting cell is a peripheral blood mononuclear cell (PBMC). In some embodiments, the antigen presenting cell is a mixed population of cells. In some embodiments, the antigen presenting cell is a mixed population of cells contained within PBMCs. In some embodiments, wherein the enhanced antigen presenting cell comprises an agent that enhances the viability and/or function of the antigen presenting cell and wherein the input antigen presenting cell is a PBMC, the agent modulates immune activity. In further embodiments, the agent that modulates immune activity upregulates the expression of one or more of IL-2, IL-7, IL-12a IL-12b, or IL-15. In some embodiments, the agent that modulates immune activity modulates the expression of one or more of the interferon-regulatory factors (IRFs), such as IRF3 or IRF5. In some embodiments, the agent that modulates immune activity modulates the expression of one or more of the toll-like receptors (TLRs), such as TLR-4. In some embodiments, the agent that modulates immune activity modulates the expression and/or activity of one or more of the toll-like receptors (TLRs), such as TLR-4 and/or TLR-9. In some embodiments, the agent that modulates immune activity modulates the expression of one or more of pattern recognition receptors (PRRs). In some embodiments, the agent that modulates immune activity modulates the activity of one or more of pattern recognition receptors (PRRs). In some embodiments, the agent that modulates immune activity modulates the expression and/or activity of one or more of STING, RIG-I, AIM2, LRRF1P1 or NLPR3. In some embodiments, wherein the enhanced antigen presenting cell comprises an agent that enhances the viability and/or function of the antigen presenting cell and wherein the input antigen presenting cell is a PBMC, the agent enhances antigen presentation. In some embodiments, the agent that enhances antigen presentation upregulates the expression of MHC-I and/or MHC-II. In some embodiments, the agent that enhances antigen presentation upregulates the expression of T-cell Receptor (TCR). In some embodiments, wherein the enhanced antigen presenting cell comprises an agent that enhances the viability and/or function of the antigen presenting cell and wherein the input antigen presenting cell is a PBMC, the agent enhances activation of the antigen presenting cell. In some embodiments, the agent that enhances activation of the antigen presenting cell modulates the expression of one or more of CD25, KLRG1, CD80, or CD86. In some embodiments, the agent that enhances activation of the antigen presenting cell modulates the expression of CD80 and/or CD86. In some embodiments, wherein the enhanced antigen presenting cell comprises an agent that enhances the viability and/or function of the antigen presenting cell and wherein the input antigen presenting cell is a PBMC, the agent enhances homing of the antigen presenting cell. In some embodiments, the agent that enhances homing of the antigen presenting cell modulates the expression of one or more of CD62L, CCR2, CCR7, CX3CR1, or CXCR5. In some embodiments, wherein the enhanced antigen presenting cell comprises an agent that enhances the viability and/or function of the antigen presenting cell and wherein the input antigen presenting cell is a PBMC, the agent is an anti-apoptotic agent. In some embodiments, the anti-apoptotic agent modulates the expression of one or more of Bcl-2, Bcl-3, or Bcl-xL. In some embodiments, wherein the enhanced antigen presenting cell comprises an agent that enhances the viability and/or function of the antigen presenting cell and wherein the input antigen presenting cell is a PBMC, the agent induces alteration in cell fate or phenotype. In some embodiments, the agent that induces alteration in cell fate or phenotype modulates the expression of one or more of Oct4, Sox2, c-Myc, Klf-4, Nanog, Lin28, Lin28B, T-bet, or GATA3. In some embodiments, the agent is a nucleic acid or a nucleic acid-protein complex. In some embodiments, the nucleic acid is a DNA or an mRNA. In some embodiments, the nucleic acid is a siRNA, shRNA or miRNA. In some embodiments, the nucleic acid-protein complex is a gene-editing complex, with or without an ssODN for homologous recombination or homology directed repair.

In some embodiments according to any of the modified antigen presenting cells described herein, the agent enhances homing of the antigen presenting cell to a site for T cell activation. In some embodiments, the agent enhances homing of the antigen presenting cell to lymph nodes. In some embodiments, the agent that enhances homing of the antigen presenting cell modulates the expression of one or more of CD62L, CCR2, CCR7, CX3CR1, or CXCR5. In some embodiments, the agent is a protein, a nucleic acid or a nucleic acid-protein complex. In some embodiments, the nucleic acid is a DNA or an mRNA. In some embodiments, the nucleic acid is a siRNA, shRNA or miRNA. In some embodiments, the nucleic acid-protein complex is a gene-editing complex. In some embodiments, the agent that enhances homing of the antigen presenting cell comprises one or more mRNAs encoding one or more of: CD62L, CCR2, CCR7, CX3CR1, or CXCR5. In some embodiments, the expression of one or more of CD62L, CCR2, CCR7, CX3CR1, or CXCR5 is increased by about any one of: 5%, 10%, 20%, 30%, 40%, 50%, 60%, 70%, 80%, 90%, 95%, 99%, or 100%. In some embodiments, the expression of one or more of CD62L, CCR2, CCR7, CX3CR1, or CXCR5 is increased by about any one of: 2-fold, 3-fold, 5-fold, 10-fold, 50-fold, 100 fold, 500-fold, or 1000-fold. In some embodiments, the homing of the modified antigen presenting cell comprising the agent to a site for T cell activation is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the homing of the modified antigen presenting cell comprising the agent to a site for T cell activation is increased by about any one of: 2-fold, 3-fold, 5-fold, 10-fold, 50-fold, 100 fold, 500-fold, or 1000-fold compared to an antigen presenting cell that does not comprise the agent. In some embodiments, the antigen presenting cell is a dendritic cell.

In certain aspects, there is provided a modified antigen presenting cell comprising an agent that enhances viability and/or function of an antigen presenting cell, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances viability and/or function of the antigen presenting cell to pass into the antigen presenting cell; and; b) incubating the perturbed input antigen presenting cell with the agent that enhances viability and/or function of the antigen presenting cell for a sufficient time to allow the agent to enter the perturbed input antigen presenting cell, thereby generating an antigen presenting cell with enhanced viability and/or function. In some embodiments, the agent that enhances viability and/or function of the antigen presenting cell upregulates expression of one or more of IL-2, IL-7, IL-12a IL-12b, IL-15, IL-18 or IL-21. In further embodiments, the agent that upregulates expression of one or more of IL-2, IL-7, IL-12a IL-12b, IL-15, IL-18 or IL-21 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances viability and/or function of the antigen presenting cell comprises one or more mRNAs encoding one or more of: IL-2, IL-7, IL-12a IL-12b, IL-15, IL-18 or IL-21. In some embodiments, the expression of one or more of IL-2, IL-7, IL-12a IL-12b, IL-15, IL-18 or IL-21 is increased by about any one of: 5%, 10%, 20%, 30%, 40%, 50%, 60%, 70%, 80%, 90%, 95%, 99%, or 100%. In some embodiments, the expression of one or more of IL-2, IL-7, IL-12a IL-12b, IL-15, IL-18 or IL-21 is increased by about any one of: 2-fold, 3-fold, 5-fold, 10-fold, 50-fold, 100 fold, 500-fold, 1000-fold, or more. In some embodiments, the circulating half-life and/or in vivo persistence of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the circulating half-life and/or in vivo persistence of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell. In some embodiments that can be combined with any other embodiments, the one or more of IL-2, IL-7, IL-12a IL-12b, IL-15, IL-18 or IL-21 comprise endogenous nucleotide or protein sequences. In some embodiments, the one or more of: IL-2, IL-7, IL-12a IL-12b, IL-15, IL-18 or IL-21 comprise modified nucleotide or protein sequences. In some embodiments, the one or more of: IL-2, IL-7, IL-12a IL-12b, IL-15, IL-18 or IL-21 are membrane-bound, such as bound to the membrane of the modified antigen presenting cell. In some embodiments, the one or more of: IL-2, IL-7, IL-12a IL-12b, IL-15, IL-18 or IL-21 are bound to membrane by GPI anchor. In some embodiments, the one or more of: IL-2, IL-7, IL-12a IL-12b, IL-15, IL-18 or IL-21 comprise a transmembrane domain sequence. In some embodiments, the one or more of: IL-2, IL-7, IL-12a IL-12b, IL-15, IL-18 or IL-21 comprise a GPI-anchor signal sequence. In some embodiments, the one or more of: IL-2, IL-7, IL-12a IL-12b, IL-15, IL-18 or IL-21 comprise the transmembrane domain and cytoplasmic tail of murine B7-1 (B7TM). In some embodiments, the one or more of: IL-2, IL-7, IL-12a IL-12b, IL-15, IL-18 or IL-21 comprising modified sequences do not bind to IL-2Rα chain (CD25) and/or do not bind IL-15Rα (CD215). In some embodiments, the one or more of: IL-2, IL-7, IL-12a IL-12b, IL-15, IL-18 or IL-21 comprising modified sequences bind to IL-2Rβ [symbol] _{c} with higher affinity than the respective natural counterpart, such as but not limited to affinity that is higher than the natural counterpart by 5%, 10%, 20%, 30%, 40%, 50%, 60%, 70%, 80%, 90%, 95%, 99%, 100%, 2-fold, 3-fold, 5-fold, 10-fold, 50-fold, 100 fold, 500-fold, 1000-fold or more. In some embodiments, the one or more of: IL-2, IL-7, IL-12a IL-12b, IL-15, IL-18 or IL-21 comprising modified amino acid sequence display about any one of: 80%, 81%, 82%, 83%, 84%, 85%, 86%, 87%, 88%, 89%, 90%, 91%, 92%, 93%, 94%, 95%, 96%, 97%, 98%, or 99% similarity as the respective wild type amino acid sequence. In some embodiments, the one or more of: IL-2, IL-7, IL-12a IL-12b, IL-15, IL-18 or IL-21 comprising modified nucleotide sequence display about any one of: 80%, 81%, 82%, 83%, 84%, 85%, 86%, 87%, 88%, 89%, 90%, 91%, 92%, 93%, 94%, 95%, 96%, 97%, 98%, or 99% similarity as the respective wild type nucleotide sequence. In some embodiments, the agent comprises one or more mimics of: IL-2, IL-7, IL-12a IL-12b, IL-15, IL-18 or IL-21, wherein the mimic comprises nucleotide or protein sequence that displays about any one of: 80%, 81%, 82%, 83%, 84%, 85%, 86%, 87%, 88%, 89%, 90%, 91%, 92%, 93%, 94%, 95%, 96%, 97%, 98%, or 99% similarity as the respective wild type sequence of IL-2, IL-7, IL-12a IL-12b, IL-15, IL-18 or IL-21. In some embodiments, the one or more of: IL-2, IL-7, IL-12a IL-12b, IL-15, IL-18 or IL-21 comprising modified sequence or the mimic of one or more of IL-2, IL-7, IL-12a IL-12b, IL-15, IL-18 or IL-21 display structural modifications compare to respective wild type counterparts. In some embodiments, the agent comprises an IL-2 mimic. In some embodiments, the agent comprises Neoleukin-2/15 (Neo-2/15).

In certain aspects, there is provided a modified antigen presenting cell comprising an agent that enhances tumor homing, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances tumor homing of the antigen presenting cell to pass into the antigen presenting cell; and; b) incubating the perturbed input antigen presenting cell with the agent that enhances tumor homing of the antigen presenting cell for a sufficient time to allow the agent to enter the perturbed input antigen presenting cell, thereby generating the modified antigen presenting cell, such as an enhanced antigen presenting cell. In some embodiments, the agent that enhances tumor homing of the antigen presenting cell upregulates expression of one or more of CXCR3, CCR5, VLA-4 or LFA-1. In further embodiments, the agent that upregulates expression of one or more of CXCR3, CCR5, VLA-4 or LFA-1 is a nucleic acid, a protein or a nucleic acid-protein complex. In some embodiments, the nucleic acid is a DNA, an mRNA, an siRNA, an shRNA or an miRNA. In some embodiments, t the nucleic acid-protein complex is a gene-editing complex with or without an ssODN for homologous recombination. In some embodiments, the agent that enhances tumor homing of the antigen presenting cell comprises one or more mRNAs encoding one or more of: CXCR3, CCR5, VLA-4 or LFA-1. In some embodiments, the expression of one or more of CXCR3, CCR5, VLA-4 or LFA-1 is increased by about any one of: 5%, 10%, 20%, 30%, 40%, 50%, 60%, 70%, 80%, 90%, 95%, 99%, or 100%. In some embodiments, the expression of one or more of CXCR3, CCR5, VLA-4 or LFA-1 is increased by about any one of: 2-fold, 3-fold, 5-fold, 10-fold, 50-fold, 100 fold, 500-fold, 1000-fold, or more. In some embodiments, the tumor homing of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the tumor homing of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a modified antigen presenting cell comprising an anti-apoptotic agent, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an anti-apoptotic agent to pass into the antigen presenting cell; and b) incubating the perturbed input antigen presenting cell with the anti-apoptotic agent for a sufficient time to allow the agent to enter the perturbed input antigen presenting cell, thereby generating the modified antigen presenting cell, such as an enhanced antigen presenting cell. In some embodiments, the anti-apoptotic agent upregulates expression of one or more of XIAP, cIAP1/2, survivin, livin, cFLIP, Hsp72, or Hsp90. In further embodiments, the agent that upregulates expression of one or more of XIAP, cIAP1/2, survivin, livin, cFLIP, Hsp72 or Hsp90 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances viability of an antigen presenting cell comprises one or more mRNAs encoding one or more of: XIAP, cIAP1/2, survivin, livin, cFLIP, Hsp72, or Hsp90. In some embodiments, the expression of one or more of XIAP, cIAP1/2, survivin, livin, cFLIP, Hsp72, or Hsp90 is increased by about any one of: 5%, 10%, 20%, 30%, 40%, 50%, 60%, 70%, 80%, 90%, 95%, 99%, or 100%. In some embodiments, the expression of one or more of XIAP, cIAP1/2, survivin, livin, cFLIP, Hsp72, or Hsp90 is increased by about any one of: 2-fold, 3-fold, 5-fold, 10-fold, 50-fold, 100 fold, 500-fold, 1000-fold, or more. In some embodiments, the circulating half-life and/or in vivo persistence of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the circulating half-life and/or in vivo persistence of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a modified antigen presenting cell comprising an agent that enhances antigen processing, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing to pass into the antigen presenting cell; and b) incubating the perturbed input antigen presenting cell with the agent that enhances antigen processing for a sufficient time to allow the agent to enter the perturbed input antigen presenting cell, thereby generating the modified antigen presenting cell, such as an enhanced antigen presenting cell. In some embodiments, the agent that enhances antigen processing upregulates expression of one or more of LMP2, LMP7, MECL-1 or β5t. In further embodiments, the agent that upregulates expression of one or more of LMP2, LMP7, MECL-1 or β5t is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances antigen processing comprises one or more mRNAs encoding one or more of: LMP2, LMP7, MECL-1 or β5t. In some embodiments, the expression of one or more of LMP2, LMP7, MECL-1 or β5t is increased by about any one of: 5%, 10%, 20%, 30%, 40%, 50%, 60%, 70%, 80%, 90%, 95%, 99%, or 100%. In some embodiments, the expression of one or more of LMP2, LMP7, MECL-1 or β5t is increased by about any one of: 2-fold, 3-fold, 5-fold, 10-fold, 50-fold, 100 fold, 500-fold, 1000-fold, or more. In some embodiments, the antigen processing in an antigen presenting cell comprising the agent is enhanced by about any one of: 5%, 10%, 20%, 30%, 40%, 50%, 60%, 70%, 80%, 90%, 95%, 99%, or 100% compared to an antigen presenting cell that does not comprise the agent. In some embodiments, the antigen processing in an antigen presenting cell comprising the agent is enhanced by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a modified antigen presenting cell comprising an agent that enhances antigen processing and/or loading onto MHC molecules, wherein the modified antigen presenting cell is prepared by a process comprising the steps of: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing and/or loading onto MHC molecules to pass into the antigen presenting cell; and b) incubating the perturbed input antigen presenting cell with the agent that enhances antigen processing and/or loading onto MHC molecules for a sufficient time to allow the agent to enter the perturbed input antigen presenting cell, thereby generating the modified antigen presenting cell, such as an enhanced antigen presenting cell. In some embodiments, the agent that enhances antigen processing and/or loading onto WIC molecules upregulates expression of one or more of TAP, Tapasin, ERAAP, Calreticulin, Erp57 or PDI. In further embodiments, the agent that upregulates expression of one or more of TAP, Tapasin, ERAAP, Calreticulin, Erp57 or PDI is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances antigen processing and/or loading comprises one or more mRNAs encoding one or more of: TAP, Tapasin, ERAAP, Calreticulin, Erp57 or PDI. In some embodiments, the expression of one or more of TAP, Tapasin, ERAAP, Calreticulin, Erp57 or PDI is increased by about any one of: 5%, 10%, 20%, 30%, 40%, 50%, 60%, 70%, 80%, 90%, 95%, 99%, or 100%. In some embodiments, the expression of one or more of TAP, Tapasin, ERAAP, Calreticulin, Erp57 or PDI is increased by about any one of: 2-fold, 3-fold, 5-fold, 10-fold, 50-fold, 100 fold, 500-fold, 1000-fold, or more. In some embodiments, the antigen processing and/or loading in an antigen presenting cell comprising the agent is enhanced by about any one of: 5%, 10%, 20%, 30%, 40%, 50%, 60%, 70%, 80%, 90%, 95%, 99%, or 100% compared to an antigen presenting cell that does not comprise the agent. In some embodiments, the antigen processing and/or loading in an antigen presenting cell comprising the agent is enhanced by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a modified antigen presenting cell comprising an agent that modulates immune activity, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that modulates immune activity to pass into the antigen presenting cell; and b) incubating the perturbed input antigen presenting cell with the agent that modulates immune activity for a sufficient time to allow the agent to enter the perturbed input antigen presenting cell, thereby generating the modified antigen presenting cell, such as an enhanced antigen presenting cell. In some embodiments, the agent that modulates immune activity upregulates expression of one or more of type I interferon, type II interferon, type III interferon and Shp2. In further embodiments, the agent that upregulates expression of one or more of type I interferon, type II interferon, type III interferon and Shp2 is a nucleic acid, a protein or a nucleic acid-protein complex. In some embodiments, the agent that modulates immune activity upregulates expression of one or more of type I interferon, type II interferon, or type III interferon. In further embodiments, the agent that upregulates expression of one or more of type I interferon, type II interferon, or type III interferon is a nucleic acid, a protein or a nucleic acid-protein complex. In some embodiments, the nucleic acid is a DNA, an mRNA, an siRNA, an shRNA or an miRNA. In some embodiments, the agent that modulates immune activity downregulates expression of interferon-beta. In further embodiments, the agent that downregulates expression of interferon-beta is a nucleic acid, a protein, a nucleic acid-protein complex or a small molecule. In some embodiments, the nucleic acid is a DNA, an mRNA, an siRNA, an shRNA or an miRNA. In some embodiments, the nucleic acid-protein complex is a gene-editing complex with or without an ssODN for homologous recombination.

In certain aspects, there is provided a modified antigen presenting cell comprising an agent that enhances the function and/or maturation of an antigen presenting cell, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances the function and/or maturation of an antigen presenting cell to pass into the antigen presenting cell; and b) incubating the perturbed input antigen presenting cell with the agent that enhances the function and/or maturation of an antigen presenting cell for a sufficient time to allow the agent to enter the perturbed input antigen presenting cell, thereby generating the modified antigen presenting cell. In some embodiments, the agent that enhances the function and/or maturation of an antigen presenting cell of the antigen presenting cell upregulates expression of one or more of type I interferons, type II interferons, or type III interferons. In some embodiments, the agent that enhances the function and/or maturation of an antigen presenting cell of the antigen presenting cell upregulates expression of one or more of: IFN-α2, IFN-β, IFN-γ, IFN-λ1, IFN-λ2, or IFN-λ3. In some embodiments, the agent that enhances expression of homing receptors in antigen presenting cell comprises one or more mRNAs encoding one or more of: IFN-α2, IFN-β, IFN-γ, IFN-λ1, IFN-λ2, or IFN-λ3. In some embodiments, the expression of one or more of IFN-α2, IFN-β, IFN-γ, IFN-λ1, IFN-λ2, or IFN-λ3 is increased by about any one of: 5%, 10%, 20%, 30%, 40%, 50%, 60%, 70%, 80%, 90%, 95%, 99%, or 100%. In some embodiments, the expression of one or more of IFN-α2, IFN-β, IFN-γ, IFN-λ1, IFN-λ2, or IFN-λ3 is increased by about any one of: 2-fold, 3-fold, 5-fold, 10-fold, 50-fold, 100 fold, 500-fold, 1000-fold, or more. In some embodiments, the maturation of an antigen presenting cell comprising the agent is enhanced by about any one of: 5%, 10%, 20%, 30%, 40%, 50%, 60%, 70%, 80%, 90%, 95%, 99%, or 100% compared to an antigen presenting cell that does not comprise the agent. In some embodiments, the maturation of an antigen presenting cell comprising the agent is enhanced by about any one of: 2-fold, 3-fold, 5-fold, 10-fold, 50-fold, 100 fold, 500-fold, 1000-fold or more compared to an antigen presenting cell that does not comprise the agent.

In certain aspects, there is provided a modified antigen presenting cell comprising an agent that enhances viability of the antigen presenting cell, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances viability of the antigen presenting cell to pass into the antigen presenting cell; and b) incubating the perturbed input antigen presenting cell with the agent that enhances viability of the antigen presenting cell for a sufficient time to allow the agent to enter the perturbed input antigen presenting cell, thereby generating a modified antigen presenting cell, such as an enhanced antigen presenting cell. In some embodiments, the agent that enhances viability of the antigen presenting cell upregulates expression of a serpin. In further embodiments, the agent that upregulates expression a serpin is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances viability of the antigen presenting cell comprises one or more mRNAs encoding one or more serpins. In some embodiments, the expression of one or more serpins is increased by about any one of: 5%, 10%, 20%, 30%, 40%, 50%, 60%, 70%, 80%, 90%, 95%, 99%, or 100%. In some embodiments, the expression of one or more serpins is increased by about any one of: 2-fold, 3-fold, 5-fold, 10-fold, 50-fold, 100 fold, 500-fold, 1000-fold, or more. In some embodiments, the circulating half-life and/or in vivo persistence of an antigen presenting cell of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the circulating half-life and/or in vivo persistence of an antigen presenting cell of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent.

In certain aspects, there is provided a modified antigen presenting cell comprising an agent that enhances homing and/or triggers alternative homing, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances homing and/or triggers alternative homing to pass into the antigen presenting cell; and b) incubating the perturbed input antigen presenting cell with the agent that enhances homing and/or triggers alternative homing for a sufficient time to allow the agent to enter the perturbed input antigen presenting cell, thereby generating the modified antigen presenting cell, such as an enhanced antigen presenting cell. In some embodiments, the agent that enhances homing receptors of the antigen presenting cell upregulates expression of CCL2. In further embodiments, the agent that upregulates expression of CCL2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances homing and/or triggers alternative homing comprises one or more mRNAs encoding CCL2. In some embodiments, the expression of CCL2 is increased by about any one of: 5%, 10%, 20%, 30%, 40%, 50%, 60%, 70%, 80%, 90%, 95%, 99%, or 100%. In some embodiments, the expression of CCL2 is increased by about any one of: 2-fold, 3-fold, 5-fold, 10-fold, 50-fold, 100 fold, 500-fold, 1000-fold, or more. In some embodiments, the homing and/or alternative homing of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the homing and/or alternative homing of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a modified antigen presenting cell comprising an agent that activates T cells, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that activates T cells to pass into the antigen presenting cell; and b) incubating the perturbed input antigen presenting cell with the agent that activates T cells for a sufficient time to allow the agent to enter the perturbed input antigen presenting cell, thereby generating the modified antigen presenting cell, such as an enhanced antigen presenting cell. In some embodiments, the agent that activates T cells upregulates expression of one or more of CD27, CD28, CD40, CD122, 4-1BB (CD137), OX40(CD134)/OX40L(CD252), GITR or ICOS. In further embodiments, the agent that upregulates expression of one or more of CD27, CD28, CD40, CD122, 4-1BB (CD137), OX40(CD134)/OX40L(CD252), GITR or ICOS is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances T cell activation comprises one or more mRNAs encoding one or more of: CD27, CD28, CD40, CD122, 4-1BB (CD137), OX40(CD134)/OX40L(CD252), GITR or ICOS. In some embodiments, the expression of one or more of CD27, CD28, CD40, CD122, 4-1BB (CD137), OX40(CD134)/OX40L(CD252), GITR or ICOS is increased by about any one of: 5%, 10%, 20%, 30%, 40%, 50%, 60%, 70%, 80%, 90%, 95%, 99%, or 100%. In some embodiments, the expression of one or more of CD27, CD28, CD40, CD122, 4-1BB (CD137), OX40(CD134)/OX40L(CD252), GITR or ICOS is increased by about any one of: 2-fold, 3-fold, 5-fold, 10-fold, 50-fold, 100 fold, 500-fold, 1000-fold, or more. In some embodiments, the T cell activation by an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the T cell activation by an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a modified antigen presenting cell comprising an agent that activates T cells, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that activates T cells to pass into the antigen presenting cell; and b) incubating the perturbed input antigen presenting cell with the agent that activates T cells for a sufficient time to allow the agent to enter the perturbed input antigen presenting cell, thereby generating the modified antigen presenting cell, such as an enhanced antigen presenting cell. In some embodiments, the agent that activates T cells upregulates expression of one or more of CD70, CD80, CD86, CD40L, 4-1BBL (CD137L), OX40L(CD252), GITRL or ICOSL. In further embodiments, the agent that upregulates expression of one or more of CD70, CD80, CD86, CD40L, 4-1BBL (CD137L), OX40L(CD252), GITRL or ICOSL is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances T cell activation comprises one or more mRNAs encoding one or more of: CD70, CD80, CD86, CD40L, 4-1BBL (CD137L), OX40L(CD252), GITRL or ICOSL. In some embodiments, the expression of one or more of CD70, CD80, CD86, CD40L, 4-1BBL (CD137L), OX40L(CD252), GITRL or ICOSL is increased by about any one of: 5%, 10%, 20%, 30%, 40%, 50%, 60%, 70%, 80%, 90%, 95%, 99%, or 100%. In some embodiments, the expression of one or more of CD70, CD80, CD86, CD40L, 4-1BBL (CD137L), OX40L(CD252), GITRL or ICOSL is increased by about any one of: 2-fold, 3-fold, 5-fold, 10-fold, 50-fold, 100 fold, 500-fold, 1000-fold, or more. In some embodiments, the T cell activation by an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the T cell activation by an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a modified antigen presenting T cell comprising an agent that activates T cells, wherein the modified antigen presenting T cell is prepared by a process comprising the steps of: a) passing a cell suspension comprising an input antigen presenting T cell through a cell-deforming constriction, wherein a diameter of the constriction is a function of a diameter of the input antigen presenting T cell in the suspension, thereby causing perturbations of the input antigen presenting T cell large enough for an agent that activates T cells to pass into the antigen presenting T cell; and b) incubating the perturbed input antigen presenting T cell with the agent that activates T cells for a sufficient time to allow the agent to enter the perturbed input antigen presenting T cell, thereby generating the modified antigen presenting T cell, such as an enhanced antigen presenting T cell. In some embodiments, the agent that activates T cells upregulates expression of one or more of CD27, CD28, CD40, CD122, 4-1BB (CD137), OX40(CD134)/OX40L(CD252), GITR or ICOS. In further embodiments, the agent that upregulates expression of one or more of CD27, CD28, CD40, CD122, 4-1BB (CD137), OX40(CD134)/OX40L(CD252), GITR or ICOS is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances T cell activation comprises one or more mRNAs encoding one or more of: CD27, CD28, CD40, CD122, 4-1BB (CD137), OX40(CD134)/OX40L(CD252), GITR or ICOS. In some embodiments, the expression of one or more of CD27, CD28, CD40, CD122, 4-1BB (CD137), OX40(CD134)/OX40L(CD252), GITR or ICOS is increased by about any one of: 5%, 10%, 20%, 30%, 40%, 50%, 60%, 70%, 80%, 90%, 95%, 99%, or 100%. In some embodiments, the expression of one or more of CD27, CD28, CD40, CD122, 4-1BB (CD137), OX40(CD134)/OX40L(CD252), GITR or ICOS is increased by about any one of: 2-fold, 3-fold, 5-fold, 10-fold, 50-fold, 100 fold, 500-fold, 1000-fold, or more. In some embodiments, the T cell activation induced by an antigen presenting T cell comprising the agent is increased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the T cell activation induced by an antigen presenting T cell comprising the agent is increased by about any one of: 2-fold, 3-fold, 5-fold, 10-fold, 50-fold, 100 fold, 500-fold, 1000-fold or more compared to an antigen presenting T cell that does not comprise the agent. In some embodiments, the activation of an antigen presenting T cell comprising the agent is increased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the activation of an antigen presenting T cell comprising the agent is increased by about any one of: 2-fold, 3-fold, 5-fold, 10-fold, 50-fold, 100 fold, 500-fold, 1000-fold or more compared to an antigen presenting T cell that does not comprise the agent.

In certain aspects, there is provided a modified antigen presenting cell, comprising an agent that downregulates T cell inhibition, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that downregulates T cell inhibition to pass into the antigen presenting cell; and b) incubating the perturbed input antigen presenting cell with the agent that downregulates T cell inhibition for a sufficient time to allow the agent to enter the perturbed input antigen presenting cell, thereby generating the modified antigen presenting cell, such as an enhanced antigen presenting cell. In some embodiments, the agent that downregulates T cell inhibition downregulates expression of one or more of LAG3, VISTA, TIM1, B7-H4 (VTCN1) or BTLA. In further embodiments, the agent that downregulates expression of one or more of LAG3, VISTA, TIM1, B7-H4 (VTCN1) or BTLA is a nucleic acid, a protein, a peptide, a nucleic acid-protein complex or a small molecule. In some embodiments, the nucleic acid is an siRNA, an shRNA or an miRNA. In some embodiments, the nucleic acid-protein complex is a gene-editing complex with or without an ssODN for homologous recombination. In some embodiments, the agent that downregulates T cell inhibition comprises one or more Cas9-gRNA RNP complexes targeting one or more of: LAG3, VISTA, TIM1, B7-H4 (VTCN1) or BTLA. In some embodiments, the expression of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the expression of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, 1000-fold, or more. In some embodiments, the agent that downregulates T cell inhibition comprises one or more small molecules targeting one or more of: LAG3, VISTA, TIM1, B7-H4 (VTCN1) or BTLA. In some embodiments, the agent that downregulates T cell inhibition comprises one or more antibodies or fragments thereof targeting one or more of: LAG3, VISTA, TIM1, B7-H4 (VTCN1) or BTLA. In some embodiments, the activity of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the activity of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, or 1000-fold, or more. In some embodiments, the T cell inhibition by an antigen presenting cell comprising the agent is decreased by about any one of: 5%, 10%, 20%, 30%, 40%, 50%, 60%, 70%, 80%, 90%, 95%, 99%, or 100% compared to an antigen presenting cell that does not comprise the agent. In some embodiments, the T cell inhibition by an antigen presenting cell comprising the agent is decreased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a modified antigen presenting T cell, comprising an agent that downregulates T cell inhibition, wherein the modified antigen presenting T cell is prepared by a process comprising the steps of: a) passing a cell suspension comprising an input antigen presenting T cell through a cell-deforming constriction, wherein a diameter of the constriction is a function of a diameter of the input antigen presenting T cell in the suspension, thereby causing perturbations of the input antigen presenting T cell large enough for an agent that downregulates T cell inhibition to pass into the antigen presenting T cell; and b) incubating the perturbed input antigen presenting T cell with the agent that downregulates T cell inhibition for a sufficient time to allow the agent to enter the perturbed input antigen presenting T cell, thereby generating the modified antigen presenting T cell, such as an enhanced antigen presenting T cell. In some embodiments, the agent that downregulates T cell inhibition downregulates expression of one or more of LAG3, VISTA, TIM1, B7-H4 (VTCN1) or BTLA. In further embodiments, the agent that downregulates expression of one or more of LAG3, VISTA, TIM1, B7-H4 (VTCN1) or BTLA is a nucleic acid, a protein, a peptide, a nucleic acid-protein complex or a small molecule. In some embodiments, the nucleic acid is an siRNA, an shRNA or an miRNA. In some embodiments, the nucleic acid-protein complex is a gene-editing complex with or without an ssODN for homologous recombination. In some embodiments, the agent that downregulates T cell inhibition comprises one or more Cas9-gRNA RNP complexes targeting one or more of: LAG3, VISTA, TIM1, B7-H4 (VTCN1) or BTLA. In some embodiments, the expression of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the expression of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, 1000-fold, or more. In some embodiments, the agent that downregulates T cell inhibition comprises one or more small molecules targeting one or more of: LAG3, VISTA, TIM1, B7-H4 (VTCN1) or BTLA. In some embodiments, the agent that downregulates T cell inhibition comprises one or more antibodies or fragments thereof targeting one or more of: LAG3, VISTA, TIM1, B7-H4 (VTCN1) or BTLA. In some embodiments, the activity of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the activity of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, or 1000-fold, or more. In some embodiments, the T cell inhibition induced by the antigen presenting T cell comprising the agent is decreased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the T cell inhibition induced by the antigen presenting T cell comprising the agent is decreased by about any one of: 2-fold, 3-fold, 5-fold, 10-fold, 50-fold, 100 fold, 500-fold, 1000-fold or more compared to an antigen presenting T cell that does not comprise the agent. In some embodiments, the inhibition of the antigen presenting T cell comprising the agent is decreased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the inhibition of the antigen presenting T cell comprising the agent is decreased by about any one of: 2-fold, 3-fold, 5-fold, 10-fold, 50-fold, 100 fold, 500-fold, 1000-fold or more compared to an antigen presenting T cell that does not comprise the agent.

In certain aspects, there is provided a modified monocyte or monocyte-dendritic progenitor cell comprising an agent that promotes formation of DCs, wherein the modified monocyte or monocyte-dendritic progenitor cell is prepared by a process comprising the steps of: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that promotes formation of DCs to pass into the monocyte or monocyte-dendritic progenitor cell; and b) incubating the perturbed input monocyte or monocyte-dendritic progenitor cell with the agent that promotes formation of DCs for a sufficient time to allow the agent to enter the perturbed input monocyte or monocyte-dendritic progenitor cell, thereby generating the modified monocyte or monocyte-dendritic progenitor cell. In some embodiments, the agent that promotes formation of DCs upregulates expression of one or more of PU.1, Flt3, Flt3L or GMCSF. In further embodiments, the agent that upregulates expression of one or more of PU.1, Flt3, Flt3L or GMCSF is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes DC formation from a monocyte or monocyte-dendritic progenitor cell comprises one or more mRNAs encoding one or more of: PU.1, Flt3, Flt3L or GMCSF. In some embodiments, the expression of one or more of PU.1, Flt3, Flt3L or GMCSF is increased by about any one of: 5%, 10%, 20%, 30%, 40%, 50%, 60%, 70%, 80%, 90%, 95%, 99%, or 100%. In some embodiments, the expression of one or more of PU.1, Flt3, Flt3L or GMCSF is increased by about any one of: 2-fold, 3-fold, 5-fold, 10-fold, 50-fold, 100 fold, 500-fold, 1000-fold, or more. In some embodiments, DC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, DC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In certain aspects, there is provided a modified monocyte or monocyte-dendritic progenitor cell comprising an agent that an agent that promotes formation of plasmacytoid DCs (pDCs), wherein the modified monocyte or monocyte-dendritic progenitor cell is prepared by a process comprising the steps of: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of pDCs to pass into the monocyte or monocyte-dendritic progenitor cell; and b) incubating the perturbed input monocyte or monocyte-dendritic progenitor cell with the agent that promotes formation of pDCs for a sufficient time to allow the agent to enter the perturbed input monocyte or monocyte-dendritic progenitor cell, thereby generating the modified monocyte or monocyte-dendritic progenitor cell. In some embodiments, the agent that promotes formation of pDCs upregulates expression of E2-2. In further embodiments, the agent that upregulates expression of E2-2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination.

In certain aspects, there is provided a modified monocyte or monocyte-dendritic progenitor cell comprising an agent that promotes formation of CD8a+/CD10+ DCs, wherein the modified antigen presenting cell is prepared by a process comprising the steps of: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of CD8a+/CD10+ DCs to pass into the monocyte or monocyte-dendritic progenitor cell; and b) incubating the perturbed input monocyte or monocyte-dendritic progenitor cell with the agent that promotes formation of CD8a+/CD10+ DCs for a sufficient time to allow the agent to enter the perturbed input monocyte or monocyte-dendritic progenitor cell, thereby generating the modified monocyte or monocyte-dendritic progenitor cell. In some embodiments, the agent that promotes formation of CD8a+/CD10+ DCs upregulates expression of one or more of Batf3, IRF8 or Id2. In further embodiments, the agent that upregulates expression of one or more of Batf3, IRF8 or Id2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes CD8a+/CD10+ DC formation from a monocyte or monocyte-dendritic progenitor cell comprises one or more mRNAs encoding one or more of: Batf3, IRF8 or Id2. In some embodiments, the expression of one or more of Batf3, IRF8 or Id2 is increased by about any one of: 5%, 10%, 20%, 30%, 40%, 50%, 60%, 70%, 80%, 90%, 95%, 99%, or 100%. In some embodiments, the expression of one or more of Batf3, IRF8 or Id2 is increased by about any one of: 2-fold, 3-fold, 5-fold, 10-fold, 50-fold, 100 fold, 500-fold, 1000-fold, or more. In some embodiments, CD8a+/CD10+ DC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, CD8a+/CD10+ DC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In certain aspects, there is provided a modified monocyte or monocyte-dendritic progenitor comprising agent that promotes formation of CD11b+ DCs, wherein the modified monocyte or monocyte-dendritic progenitor cell is prepared by a process comprising the steps of: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of CD11b+ DCs to pass into the monocyte or monocyte-dendritic progenitor cell; and b) incubating the perturbed input monocyte or monocyte-dendritic progenitor cell with the agent that promotes formation of CD11b+ DCs for a sufficient time to allow the agent to enter the perturbed input monocyte or monocyte-dendritic progenitor cell, thereby generating modified monocyte or monocyte-dendritic progenitor cell. In some embodiments, the agent that promotes formation of CD11b+ DCs upregulates expression of one or more of IRF4, RBJ, MgI or Mtg16. In further embodiments, the agent that upregulates expression of one or more of IRF4, RBJ, MgI or Mtg16 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination.

In certain aspects, there is provided a modified monocyte or monocyte-dendritic progenitor cell comprising an agent that inhibits formation of pDCs and classical DCs, wherein the modified monocyte or monocyte-dendritic progenitor cell is prepared by a process comprising the steps of: a) passing a cell suspension comprising the monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that inhibits formation of pDCs and classical DCs to pass into the monocyte or monocyte-dendritic progenitor cell; and b) incubating the perturbed input monocyte or monocyte-dendritic progenitor cell with the agent that inhibits formation of pDCs and classical DCs for a sufficient time to allow the agent to enter the perturbed input monocyte or monocyte-dendritic progenitor cell, thereby generating the modified monocyte or monocyte-dendritic progenitor cell. In some embodiments, the agent that inhibits formation of pDCs and classical DCs downregulates expression of STAT3 and/or Xbp1. In further embodiments, the agent that downregulates expression of STAT3 and/or Xbp1 is a nucleic acid, a protein, a peptide, a nucleic acid-protein complex or a small molecule. In some embodiments, the nucleic acid is a DNA, an mRNA, an siRNA, an shRNA or an miRNA. In some embodiments, the nucleic acid-protein complex is a gene-editing complex with or without an ssODN for homologous recombination.

In some embodiments, according to any of the modified antigen presenting cells described herein, the modified antigen presenting cell comprises two or more agents that enhance the viability and/or function of the antigen presenting cell is delivered to the antigen presenting cell. In further embodiments, according to the modified antigen presenting cells described above, the two or more agents that enhance the viability and/or function of the antigen presenting cell are chosen from one or more of a tumor homing agent, an anti-apoptotic agent, a T cell activating agent, an antigen processing agent, an immune activity modulating agent, a homing receptor, or an agent that down regulates T cell inhibition.

In some embodiments, according to any of the modified antigen presenting cells described herein, the agent that enhances the viability and/or function of the antigen presenting cell is an agent that alters cell fate or cell phenotype. In some embodiments, the agent that alters cell fate or phenotype is a somatic cell reprogramming factor. In some embodiments, the agent that alters cell fate or phenotype is a dedifferentiation factor. In some embodiments, the agent that alters cell fate or phenotype is a trans-differentiation factor. In some embodiments, the agent that alters cell phenotype is a differentiation factor. In further embodiments, the agent that alters cell fate or phenotype is one or more of OCT4, SOX2, C-MYC, KLF-4, NANOG, LIN28 or LIN28B. In some embodiments, the agent that alters cell fate or phenotype is one or more of T-bet, GATA3. In some embodiments, the agent that alters cell fate or phenotype is one or more of EOMES, RUNX1, ERG, LCOR, HOXA5, or HOXA9. In some embodiments, the agent that alters cell fate or phenotype is one or more of GM-CSF, M-CSF, or RANKL. In some embodiments, the agent that alters cell fate or cell phenotype comprises one or more mRNAs encoding one or more of: OCT4, SOX2, C-MYC, KLF-4, NANOG, LIN28, LIN28B, T-bet, GATA3, EOMES, RUNX1, ERG, LCOR, HOXA5, HOXA9, GM-CSF, M-CSF, or RANKL. In some embodiments, the expression of one or more of OCT4, SOX2, C-MYC, KLF-4, NANOG, LIN28, LIN28B, T-bet, GATA3, EOMES, RUNX1, ERG, LCOR, HOXA5, HOXA9, GM-CSF, M-CSF, or RANKL is increased by about any one of: 5%, 10%, 20%, 30%, 40%, 50%, 60%, 70%, 80%, 90%, 95%, 99%, or 100%. In some embodiments, the expression of one or more of OCT4, SOX2, C-MYC, KLF-4, NANOG, LIN28, LIN28B, T-bet, GATA3, EOMES, RUNX1, ERG, LCOR, HOXA5, HOXA9, GM-CSF, M-CSF, or RANKL is increased by about any one of: 2-fold, 3-fold, 5-fold, 10-fold, 50-fold, 100 fold, 500-fold, 1000-fold, or more.

In some embodiments, according to any of the modified antigen presenting cells described herein, the modified antigen presenting cell further comprises an antigen. In some embodiments, the antigen is delivered before, at the same time, or after the agent that enhances the viability and/or function of the antigen presenting cell is delivered to the cell. In some embodiments, the antigen is delivered to the antigen presenting cell by a method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the antigen to pass into the antigen presenting cell; and b) incubating the perturbed input antigen presenting cell with the antigen for a sufficient time to allow the antigen to enter the perturbed input antigen presenting cell.

In some embodiments, according to any of the modified antigen presenting cells described herein, the modified antigen presenting cell further comprises an adjuvant. In some embodiments, the adjuvant is delivered before, at the same time, or after the antigen is delivered to the cell and/or before, at the same time, or after the agent that enhances the viability and/or function of the antigen presenting cell is delivered to the cell. In some embodiments, the adjuvant is delivered to the antigen presenting cell by a method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the adjuvant to pass into the antigen presenting cell; and b) incubating the perturbed input antigen presenting cell with the adjuvant for a sufficient time to allow the adjuvant to enter the perturbed input antigen presenting cell.

Therefore in some embodiments, according to any of the modified antigen presenting cells described herein, the modified antigen presenting cell further comprises an antigen and/or an adjuvant. In some embodiments, the antigen is exogenous to the modified antigen presenting cell and comprises an immunogenic epitope, and the adjuvant is present intracellularly. Exogenous antigens are one or more antigens from a source outside the antigen presenting cell introduced into a cell to be modified. Exogenous antigens can include antigens that may be present in the antigen presenting cell (i.e. also present from an endogenous source), either before or after introduction of the exogenous antigen, and as such can thus be produced by the antigen presenting cell (e.g., encoded by the genome of the antigen presenting cell). For example, in some embodiments, the modified antigen presenting cell further comprises two pools of an antigen, a first pool comprising an endogenous source of the antigen, and a second pool comprising an exogenous source of the antigen produced outside of and introduced into the antigen presenting cell to be modified. In some embodiments, the antigen is ectopically expressed or overexpressed in a disease cell in an individual, and the modified antigen presenting cell is derived from the individual and comprises an exogenous source of the antigen, or an immunogenic epitope contained therein, produced outside of and introduced into the antigen presenting cell to be modified. In some embodiments, the antigen is a neoantigen (e.g., an altered-self protein or portion thereof) comprising a neoepitope, and the modified antigen presenting cell comprises an exogenous source of the antigen, or a fragment thereof comprising the neoepitope, produced outside of and introduced into the antigen presenting cell to be modified. In some embodiments, the adjuvant is exogenous to the modified antigen presenting cell. In some embodiments, the antigen and/or the adjuvant are present in multiple compartments of the modified antigen presenting cell. In some embodiments, the antigen and/or adjuvant are present in the cytosol and/or a vesicle of the modified T cell. In some embodiments, the vesicle is an endosome. In some embodiments, the antigen or immunogenic epitope, and/or the adjuvant is bound to the surface of the modified T cell.

In some embodiments, according to any of the modified antigen presenting cells described herein, the antigen is present in multiple compartments of the modified antigen presenting cell. In some embodiments, the antigen is present in the cytosol and/or a vesicle of the modified antigen presenting cell. In some embodiments, the vesicle is an endosome. In some embodiments, the antigen is bound to the surface of the modified antigen presenting cell. In some embodiments, the antigen or an immunogenic epitope contained therein is bound to the surface of the modified antigen presenting cell. In some embodiments, the antigen presenting cell is a PBMC. In some embodiments, the antigen presenting cell is a mixed population of cells. In some embodiments, the antigen presenting cell is in a mixed population of cells, wherein the mixed population of cells is a population of PBMCs. In some embodiments, the PBMC includes one or more of a T cell, a B cell, an NK cells or, a monocyte, a macrophage or a dendritic cell. In some embodiments, the modified antigen presenting cell further comprises an adjuvant. In some embodiments, the antigen and/or the adjuvant are present in the cytosol and/or a vesicle of the antigen presenting cell.

In some embodiments, according to any of the modified antigen presenting cells described herein, the adjuvant is present in multiple compartments of the modified antigen presenting cell. In some embodiments, the adjuvant is present in the cytosol and/or a vesicle of the modified antigen presenting cell. In some embodiments, the vesicle is an endosome. In some embodiments, the adjuvant is bound to the surface of the modified antigen presenting cell. In some embodiments, the antigen presenting cell is a PBMC. In some embodiments, the antigen presenting cell is a mixed population of cells. In some embodiments, the antigen presenting cell is in a mixed population of cells, wherein the mixed population of cells is a population of PBMCs. In some embodiments, the PBMC includes one or more of a T cell, a B cell, an NK cells or, a monocyte, a macrophage or a dendritic cell. In some embodiments, the modified antigen presenting cell further comprises an antigen. In some embodiments, the antigen and/or the adjuvant are present in the cytosol and/or a vesicle of the antigen presenting cell.

In some embodiments, according to any of the modified antigen presenting cells described herein, the modified antigen presenting cell further comprises an adjuvant. In some embodiments, the adjuvant is a CpG oligodeoxynucleotide (ODN), IFN-α, STING agonists, RIG-I agonists, poly I:C, imiquimod, and/or resiquimod. In some embodiments, the adjuvant is a CpG ODN. In some embodiments, the CpG ODN is no greater than about 50 (such as no greater than about any of 45, 40, 35, 30, 25, 20, or fewer) nucleotides in length. In some embodiments, the CpG ODN is a Class A CpG ODN, a Class B CpG ODN, or a Class C CpG ODN. In some embodiments, the CpG ODN comprises the nucleotide sequences as disclosed in US provisional application U.S. 62/641,987. In some embodiments, the modified antigen presenting cell comprises a plurality of different CpG ODNs. For example, in some embodiments, the modified antigen presenting cell comprises a plurality of different CpG ODNs selected from among Class A, Class B, and Class C CpG ODNs.

In some embodiments, according to any of the modified antigen presenting cells described herein, the antigen is a disease-associated antigen. In further embodiments, the antigen is a tumor antigen. In some embodiments, the antigen is derived from a lysate. In some embodiments, the lysate is derived from a biopsy of an individual. In some embodiments, the lysate is derived from a biopsy of an individual being infected by a pathogen, such as a bacteria or a virus. In some embodiments, the lysate is derived from a biopsy of an individual bearing tumors (i.e. tumor biopsy lysates). Thus in some embodiments, the lysate is a tumor lysate.

In some embodiments, according to any of the modified antigen presenting cells described herein, the modified antigen presenting cell comprises an antigen comprising an immunogenic epitope. In some embodiments, the immunogenic epitope is derived from a disease-associated antigen. In some embodiments, the immunogenic epitope is derived from peptides or mRNA isolated from a diseased cell. In some embodiments, the immunogenic epitope is derived from a protein ectopically expressed or overexpressed in a diseased cell. In some embodiments, the immunogenic epitope is derived from a neoantigen, e.g., a cancer-associated neoantigen. In some embodiments, the immunogenic epitope comprises a neoepitope, e.g., a cancer-associated neoepitope. In some embodiments, the immunogenic epitope is derived from a non-self antigen. In some embodiments, the immunogenic epitope is derived from a mutated or otherwise altered self antigen. In some embodiments, the immunogenic epitope is derived from a tumor antigen, viral antigen, bacterial antigen, or fungal antigen. In some embodiments, the antigen comprises an immunogenic epitope fused to heterologous peptide sequences. In some embodiments, the antigen comprises a plurality of immunogenic epitopes. In some embodiments, some of the plurality of immunogenic epitopes are derived from the same source. For example, in some embodiments, some of the plurality of immunogenic epitopes are derived from the same viral antigen. In some embodiments, all of the plurality of immunogenic epitopes are derived from the same source. In some embodiments, none of the plurality of immunogenic epitopes are derived from the same source. In some embodiments, the modified antigen presenting cell comprises a plurality of different antigens.

In some embodiments, according to any of the modified antigen presenting cells described herein, the modified antigen presenting cell further comprises an antigen, wherein the antigen comprises an immunogenic epitope. In some embodiments, the antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the immunogenic peptide epitope fused to the N-terminal flanking polypeptide and/or the C-terminal flanking polypeptide is a non-naturally occurring sequence. In some embodiments, the N-terminal and/or C-terminal flanking polypeptides are derived from an immunogenic synthetic long peptide (SLP). In some embodiments, the N-terminal and/or C-terminal flanking polypeptides are derived from a disease-associated immunogenic SLP.

In some embodiments, according to any of the modified antigen presenting cells described herein, the modified antigen presenting cell further comprises an antigen, wherein the antigen is capable of being processed into an MHC class I-restricted peptide and/or an MHC class II-restricted peptide. In some embodiments, the antigen is capable of being processed into an MHC class I-restricted peptide. In some embodiments, the antigen is capable of being processed into an MHC class II-restricted peptide. In some embodiments, the antigen comprises a plurality of immunogenic epitopes, and is capable of being processed into an MHC class I-restricted peptide and an MHC class II-restricted peptide. In some embodiments, some of the plurality of immunogenic epitopes are derived from the same source. In some embodiments, all of the plurality of immunogenic epitopes are derived from the same source. In some embodiments, none of the plurality of immunogenic epitopes are derived from the same source.

In some embodiments, according to any of the modified antigen presenting cells described herein, the modified antigen presenting cell comprises a plurality of antigens that comprise a plurality of immunogenic epitopes. In some embodiments, following administration to an individual of the modified antigen presenting cell comprising the plurality of antigens that comprise the plurality of immunogenic epitopes, none of the plurality of immunogenic epitopes decreases an immune response in the individual to any of the other immunogenic epitopes.

In some embodiments, according to any of the modified antigen presenting cells described herein, the modified antigen presenting cell comprises an agent that enhances the viability and/or function of the modified antigen presenting cell. In some embodiments, the modified antigen presenting cell further comprises an antigen and/or an adjuvant. In some embodiments, the modified antigen presenting cell comprises the agent that enhances the viability and/or function of the modified antigen presenting cell at a concentration between about 1 pM and about 10 mM. In some embodiments, the modified antigen presenting cell comprises the antigen at a concentration between about 1 pM and about 10 mM. In some embodiments, the modified antigen presenting cell comprises the adjuvant at a concentration between about 1 pM and about 10 mM. In some embodiments, the modified antigen presenting cell comprises the agent that enhances the viability and/or function of the modified antigen presenting cell at a concentration between about 0.1 μM and about 10 mM. In some embodiments, the modified antigen presenting cell comprises the antigen at a concentration between about 0.1 μM and about 10 mM. In some embodiments, the modified antigen presenting cell comprises the adjuvant at a concentration between about 0.1 μM and about 10 mM. For example, in some embodiments, the concentration of the agent that enhances the viability and/or function of the modified antigen presenting cell in the modified antigen presenting cell is any of less than about 1 pM, about 10 pM, about 100 pM, about 1 nM, about 10 nM, about 100 nM, about 1 about 10 about 100 about 1 mM or about 10 mM. In some embodiments, the concentration of the agent that enhances the viability and/or function of the modified antigen presenting cell in the modified antigen presenting cell is greater than about 10 mM. In some embodiments, the concentration of adjuvant in the modified antigen presenting cell is any of less than about 1 pM, about 10 pM, about 100 pM, about 1 nM, about 10 nM, about 100 nM, about 1 about 10 about 100 about 1 mM or about 10 mM. In some embodiments, the concentration of adjuvant in the modified antigen presenting cell is greater than about 10 mM. In some embodiments, the concentration of antigen in the modified antigen presenting cell is any of less than about 1 pM, about 10 pM, about 100 pM, about 1 nM, about 10 nM, about 100 nM, about 1 about 10 about 100 about 1 mM or about 10 mM. In some embodiments, the concentration of antigen in the modified antigen presenting cell is greater than about 10 mM. In some embodiments, the concentration of the agent that enhances the viability and/or function of the modified antigen presenting cell in the modified antigen presenting cell is any of between about 1 pM and about 10 pM, between about 10 pM and about 100 pM, between about 100 pM and about 1 nM, between about 1 nM and about 10 nM, between about 10 nM and about 100 nM, between about 100 nM and about 1 between about 1 μM and about 10 between about 10 μM and about 100 between about 100 μM and about 1 mM, or between 1 mM and about 10 mM.

In some embodiments, the molar ratio of the agent that enhances the viability and/or function of the modified antigen presenting cell to antigen in the modified antigen presenting cell is any of between about 10000:1 to about 1:10000. For example, in some embodiments, the molar ratio of the agent that enhances the viability and/or function of the modified antigen presenting cell to antigen in the modified antigen presenting cell is about any of 10000:1, about 1000:1, about 100:1, about 10:1, about 1:1, about 1:10, about 1:100, about 1:1000, or about 1:10000. In some embodiments, the molar ratio of the agent that enhances the viability and/or function of the modified antigen presenting cell to antigen in the modified antigen presenting cell is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the molar ratio of the agent that enhances the viability and/or function of the modified antigen presenting cell to adjuvant in the modified antigen presenting cell is any of between about 10000:1 to about 1:10000. For example, in some embodiments, the molar ratio of the agent to adjuvant in the modified antigen presenting cell is about any of 10000:1, about 1000:1, about 100:1, about 10:1, about 1:1, about 1:10, about 1:100, about 1:1000, or about 1:10000. In some embodiments, the molar ratio of the agent that enhances the viability and/or function of the modified antigen presenting cell to adjuvant in the modified antigen presenting cell is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the modified antigen presenting cell comprises a complex comprising: a) the agent that enhances the viability and/or function of the modified antigen presenting cell, b) the agent and at least another agent, c) the agent and the antigen, d) the agent and the adjuvant, and/or e) the agent, the antigen and the adjuvant.

In some embodiments, according to any of the modified antigen presenting cells described herein, the modified antigen presenting cell further comprises an additional agent that enhances the viability and/or function of the modified antigen presenting cell as compared to a corresponding modified antigen presenting cell that does not comprise the additional agent. In some embodiments, the additional agent is a stabilizing agent or a co-factor. In some embodiments, the agent is albumin. In some embodiments, the albumin is mouse, bovine, or human albumin. In some embodiments, the additional agent is a divalent metal cation, glucose, ATP, potassium, glycerol, trehalose, D-sucrose, PEG1500, L-arginine, L-glutamine, or EDTA.

In some embodiments, according to any of the modified antigen presenting cells described herein, the modified antigen presenting cell comprises a further modification. In some embodiments, the modified antigen presenting cell comprises a further modification to modulate MHC class I expression. In some embodiments, the modified antigen presenting cell comprises a further modification to decrease MHC class I expression. In some embodiments, the modified antigen presenting cell comprises a further modification to increase MHC class I expression. In some embodiments, the modified T cell comprises a further modification to modulate MHC class II expression. In some embodiments, the modified antigen presenting cell comprises a further modification to decrease MHC class II expression. In some embodiments, the modified antigen presenting cell comprises a further modification to increase MHC class II expression. In some embodiments, an innate immune response mounted in an individual in response to administration, in an allogeneic context, of the modified antigen presenting cells is reduced compared to an innate immune response mounted in an individual in response to administration, in an allogeneic context, of corresponding modified antigen presenting cells that do not comprise the further modification. In some embodiments, the circulating half-life and/or in vivo persistence of the modified antigen presenting cells in an individual to which they were administered is increased compared to the circulating half-life and/or in vivo persistence of corresponding modified T cells that do not comprise the further modification in an individual to which they were administered.

In certain aspects, there is provided a modified antigen presenting cell comprising an agent that enhances the viability and/or function of the antigen presenting cell, an antigen and an adjuvant, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the agent that enhances the viability and/or function of the antigen presenting cell, the antigen and the adjuvant to pass through to form a perturbed input antigen presenting cell; and b) incubating the perturbed input antigen presenting cell with the agent that enhances the viability and/or function of the antigen presenting cell, the antigen and the adjuvant for a sufficient time to allow the antigen and the adjuvant to enter the perturbed input antigen presenting cell; thereby generating the modified antigen presenting cell comprising the agent that enhances the viability and/or function of the antigen presenting cell, the antigen and the adjuvant. In some embodiments, the concentration of the agent that enhances the viability and/or function of the antigen presenting cell incubated with the perturbed input antigen presenting cell is between about 1 pM-10 mM, the concentration of the antigen incubated with the perturbed input antigen presenting cell is between about 1 pM-10 mM and the concentration of the adjuvant incubated with the perturbed input antigen presenting cell is between about 1 pM-10 mM. In some embodiments, the concentration of the agent that enhances the viability and/or function of the antigen presenting cell incubated with the perturbed input antigen presenting cell is between about 0.1 μM-10 mM, the concentration of the antigen incubated with the perturbed input antigen presenting cell is between about 0.1 μM-10 mM and the concentration of the adjuvant incubated with the perturbed input antigen presenting cell is between about 0.1 μM-10 mM. In some embodiments, the ratio of the agent to the antigen incubated with the perturbed input antigen presenting cell is between about 10000:1 to about 1:10000. In some embodiments, the ratio of the agent to the adjuvant incubated with the perturbed input antigen presenting cell is between about 10000:1 to about 1:10000. In some embodiments, the ratio of the antigen to the adjuvant incubated with the perturbed input antigen presenting cell is between about 10000:1 to about 1:10000.

The modified antigen presenting cells described herein in some embodiments are prepared by a process employing a cell-deforming constriction through which an input antigen presenting cell is passed. In some embodiments, according to any of the modified antigen presenting cells described herein, the diameter of the constriction is less than the diameter of the input antigen presenting cell. In some embodiments, the diameter of the constriction is about 20% to about 99% of the diameter of the input antigen presenting cell. In some embodiments, the diameter of the constriction is about 20% to about 60% of the diameter of the input antigen presenting cell. In some embodiments, the cell-deforming constriction is contained in a microfluidic channel, such as any of the microfluidic channels described herein. The microfluidic channel may be contained in any of the microfluidic devices described herein, such as described in the section titled Microfluidic Devices below. Thus, in some embodiments, according to any of the modified antigen presenting cell s described herein prepared by a process employing a microfluidic channel including a cell-deforming constriction through which an input antigen presenting cell is passed, the process comprises passing the input antigen presenting cell through a microfluidic channel including a cell-deforming constriction contained in any of the microfluidic systems described herein. In some embodiments, a deforming force is applied to the input antigen presenting cell as it passes through the constriction, thereby causing the perturbations of the input antigen presenting cell.

Input antigen presenting cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the input antigen presenting cell is a peripheral blood mononuclear cell (PBMC). In some embodiments, the antigen presenting cell is a mixed population of cells. In some embodiments, the antigen presenting cell is in a mixed population of cells, wherein the mixed population of cells is a population of PBMCs. In some embodiments, the PBMC is a T cell, a B cell, an NK cells or a monocyte. In some embodiments of the present invention, any number of cell lines of PBMC subtype population available in the art may be used, such as T cell lines or B cell lines. In some embodiments of the present invention, various subtype populations of PBMCs can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, CD45RO⁺ T cells, and γδ-T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least one, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process (negative selection). “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD11b, CD 16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/mL is used. In some embodiments, a concentration of about 1 billion cells/mL is used. In some embodiments, greater than about 100 million cells/mL is used. In some embodiments, a concentration of cells of about any of 10, 15, 20, 25, 30, 35, 40, 45, or 50 million cells/mL is used. In some embodiments, a concentration of cells of about any of 75, 80, 85, 90, 95, or 100 million cells/mL is used. In some embodiments, a concentration of about 125 or about 150 million cells/mL is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments, according to any of the modified antigen presenting cells described herein, wherein the modified antigen presenting cell comprises an agent that enhances the viability and/or function of the modified antigen presenting cell, the input antigen presenting cell is a peripheral blood mononuclear cell (PBMC). In some embodiments, the PBMC is a T cell, a B cell, an NK cells or a monocyte. In some embodiments, the PBMC is engineered to present an antigen. In some embodiments, the agent enhances tumor homing of the antigen presenting cell. In some embodiments, the agent is an anti-apoptotic agent. In some embodiments, the agent enhances T-cell activation. In some embodiments, the agent enhances antigen processing. In some embodiments, the agent enhances antigen processing and loading into MHC-1. In some embodiments, the agent modulates immune activity. In some embodiments, the agent is a homing receptor. In some embodiments, the agent downregulates T cell inhibition.

In some embodiments, according to any of the modified monocytes, or monocyte-dendritic progenitors or DCs described herein, the modified monocyte, or monocyte-dendritic progenitor or DC further comprises an antigen. In some embodiments, the antigen is delivered before, at the same time, or after the agent that promotes or inhibits DC formation is delivered to the cell. In some embodiments, the antigen is delivered to the monocyte, or monocyte-dendritic progenitor or DC by a method comprising: a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the antigen to pass into the monocyte, or monocyte-dendritic progenitor or DC; and b) incubating the perturbed input monocyte, or monocyte-dendritic progenitor or DC with the antigen for a sufficient time to allow the antigen to enter the perturbed input monocyte, or monocyte-dendritic progenitor or DC.

In some embodiments, according to any of the modified monocytes, or monocyte-dendritic progenitors or DCs described herein, the modified monocyte, or monocyte-dendritic progenitor or DC further comprises an adjuvant. In some embodiments, the adjuvant is delivered before, at the same time, or after the antigen is delivered to the cell and/or before, at the same time, or after the agent that promotes or inhibits DC formation of the monocyte, or monocyte-dendritic progenitor or DC is delivered to the cell. In some embodiments, the adjuvant is delivered to the monocyte, or monocyte-dendritic progenitor or DC by a method comprising: a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the adjuvant to pass into the monocyte, or monocyte-dendritic progenitor or DC; and b) incubating the perturbed input monocyte, or monocyte-dendritic progenitor or DC with the adjuvant for a sufficient time to allow the adjuvant to enter the perturbed input monocyte, or monocyte-dendritic progenitor or DC.

Therefore in some embodiments, according to any of the modified monocytes, or monocyte-dendritic progenitors or DCs described herein, the modified monocyte, or monocyte-dendritic progenitor or DC further comprises an antigen and/or an adjuvant. In some embodiments, the antigen is exogenous to the modified monocyte, or monocyte-dendritic progenitor or DC and comprises an immunogenic epitope, and the adjuvant is present intracellularly. Exogenous antigens are one or more antigens from a source outside the monocyte, or monocyte-dendritic progenitor or DC introduced into a cell to be modified. Exogenous antigens can include antigens that may be present in the monocyte, or monocyte-dendritic progenitor or DC (i.e. also present from an endogenous source), either before or after introduction of the exogenous antigen, and as such can thus be produced by the monocyte, or monocyte-dendritic progenitor or DC (e.g., encoded by the genome of the monocyte, or monocyte-dendritic progenitor or DC). For example, in some embodiments, the modified monocyte, or monocyte-dendritic progenitor or DC further comprises two pools of an antigen, a first pool comprising an endogenous source of the antigen, and a second pool comprising an exogenous source of the antigen produced outside of and introduced into the monocyte, or monocyte-dendritic progenitor or DC to be modified. In some embodiments, the antigen is ectopically expressed or overexpressed in a disease cell in an individual, and the modified monocyte, or monocyte-dendritic progenitor or DC is derived from the individual and comprises an exogenous source of the antigen, or an immunogenic epitope contained therein, produced outside of and introduced into the monocyte, or monocyte-dendritic progenitor or DC to be modified. In some embodiments, the antigen is a neoantigen (e.g., an altered-self protein or portion thereof) comprising a neoepitope, and the modified monocyte, or monocyte-dendritic progenitor or DC comprises an exogenous source of the antigen, or a fragment thereof comprising the neoepitope, produced outside of and introduced into the monocyte, or monocyte-dendritic progenitor or DC to be modified. In some embodiments, the adjuvant is exogenous to the modified monocyte, or monocyte-dendritic progenitor or DC. In some embodiments, the antigen and/or the adjuvant are present in multiple compartments of the modified monocyte, or monocyte-dendritic progenitor or DC. In some embodiments, the antigen and/or adjuvant are present in the cytosol and/or a vesicle of the modified monocyte, or monocyte-dendritic progenitor or DC. In some embodiments, the vesicle is an endosome. In some embodiments, the antigen or immunogenic epitope, and/or the adjuvant is bound to the surface of the modified monocyte, or monocyte-dendritic progenitor or DC.

In some embodiments, according to any of the modified monocytes, or monocyte-dendritic progenitors or DCs described herein, the antigen is present in multiple compartments of the modified monocyte, or monocyte-dendritic progenitor or DC. In some embodiments, the antigen is present in the cytosol and/or a vesicle of the modified monocyte, or monocyte-dendritic progenitor or DC. In some embodiments, the vesicle is an endosome. In some embodiments, the antigen is bound to the surface of the modified monocyte, or monocyte-dendritic progenitor or DC. In some embodiments, the antigen or an immunogenic epitope contained therein is bound to the surface of the modified monocyte, or monocyte-dendritic progenitor or DC. In some embodiments, the antigen and/or the adjuvant are present in the cytosol and/or a vesicle of the monocyte, or monocyte-dendritic progenitor or DC.

In some embodiments, according to any of the modified monocytes, or monocyte-dendritic progenitors or DCs described herein, the adjuvant is present in multiple compartments of the modified monocyte, or monocyte-dendritic progenitor or DC. In some embodiments, the adjuvant is present in the cytosol and/or a vesicle of the modified monocyte, or monocyte-dendritic progenitor or DC. In some embodiments, the vesicle is an endosome. In some embodiments, the adjuvant is bound to the surface of the modified monocyte, or monocyte-dendritic progenitor or DC. In some embodiments, the modified monocyte, or monocyte-dendritic progenitor or DC further comprises an antigen. In some embodiments, the antigen and/or the adjuvant are present in the cytosol and/or a vesicle of the monocyte, or monocyte-dendritic progenitor or DC.

In some embodiments, according to any of the modified monocytes, or monocyte-dendritic progenitors or DCs described herein, the modified monocyte, or monocyte-dendritic progenitor or DC further comprises an adjuvant. In some embodiments, the adjuvant is a CpG oligodeoxynucleotide (ODN), IFN-α, STING agonists, RIG-I agonists, poly I:C, imiquimod, and/or resiquimod. In some embodiments, the adjuvant is a CpG ODN. In some embodiments, the CpG ODN is no greater than about 50 (such as no greater than about any of 45, 40, 35, 30, 25, 20, or fewer) nucleotides in length. In some embodiments, the CpG ODN is a Class A CpG ODN, a Class B CpG ODN, or a Class C CpG ODN. In some embodiments, the CpG ODN comprises the nucleotide sequences as disclosed in US provisional application U.S. 62/641,987. In some embodiments, the modified monocyte, or monocyte-dendritic progenitor or DC comprises a plurality of different CpG ODNs. For example, in some embodiments, the modified monocyte, or monocyte-dendritic progenitor or DC comprises a plurality of different CpG ODNs selected from among Class A, Class B, and Class C CpG ODNs.

In some embodiments, according to any of the modified monocytes, or monocyte-dendritic progenitors or DCs described herein, the antigen is a disease-associated antigen. In further embodiments, the antigen is a tumor antigen. In some embodiments, the antigen is derived from a lysate. In some embodiments, the lysate is derived from a biopsy of an individual. In some embodiments, the lysate is derived from a biopsy of an individual being infected by a pathogen, such as a bacteria or a virus. In some embodiments, the lysate is derived from a biopsy of an individual bearing tumors (i.e. tumor biopsy lysates). Thus in some embodiments, the lysate is a tumor lysate.

In some embodiments, according to any of the modified monocytes, or monocyte-dendritic progenitors or DCs described herein, the modified monocyte, or monocyte-dendritic progenitor or DC comprises an antigen comprising an immunogenic epitope. In some embodiments, the immunogenic epitope is derived from a disease-associated antigen. In some embodiments, the immunogenic epitope is derived from peptides or mRNA isolated from a diseased cell. In some embodiments, the immunogenic epitope is derived from a protein ectopically expressed or overexpressed in a diseased cell. In some embodiments, the immunogenic epitope is derived from a neoantigen, e.g., a cancer-associated neoantigen. In some embodiments, the immunogenic epitope comprises a neoepitope, e.g., a cancer-associated neoepitope. In some embodiments, the immunogenic epitope is derived from a non-self antigen. In some embodiments, the immunogenic epitope is derived from a mutated or otherwise altered self antigen. In some embodiments, the immunogenic epitope is derived from a tumor antigen, viral antigen, bacterial antigen, or fungal antigen. In some embodiments, the antigen comprises an immunogenic epitope fused to heterologous peptide sequences. In some embodiments, the antigen comprises a plurality of immunogenic epitopes. In some embodiments, some of the plurality of immunogenic epitopes are derived from the same source. For example, in some embodiments, some of the plurality of immunogenic epitopes are derived from the same viral antigen. In some embodiments, all of the plurality of immunogenic epitopes are derived from the same source. In some embodiments, none of the plurality of immunogenic epitopes are derived from the same source. In some embodiments, the modified monocyte, or monocyte-dendritic progenitor or DC comprises a plurality of different antigens.

In some embodiments, according to any of the modified monocytes, or monocyte-dendritic progenitors or DCs described herein, the modified monocyte, or monocyte-dendritic progenitor or DC further comprises an antigen, wherein the antigen comprises an immunogenic epitope. In some embodiments, the antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the immunogenic peptide epitope fused to the N-terminal flanking polypeptide and/or the C-terminal flanking polypeptide is a non-naturally occurring sequence. In some embodiments, the N-terminal and/or C-terminal flanking polypeptides are derived from an immunogenic synthetic long peptide (SLP). In some embodiments, the N-terminal and/or C-terminal flanking polypeptides are derived from a disease-associated immunogenic SLP.

In some embodiments, according to any of the modified monocytes, or monocyte-dendritic progenitors or DCs described herein, the modified monocyte, or monocyte-dendritic progenitor or DC further comprises an antigen, wherein the antigen is capable of being processed into an MHC class I-restricted peptide and/or an MHC class II-restricted peptide. In some embodiments, the antigen is capable of being processed into an MHC class I-restricted peptide. In some embodiments, the antigen is capable of being processed into an MHC class II-restricted peptide. In some embodiments, the antigen comprises a plurality of immunogenic epitopes, and is capable of being processed into an MHC class I-restricted peptide and an MHC class II-restricted peptide. In some embodiments, some of the plurality of immunogenic epitopes are derived from the same source. In some embodiments, all of the plurality of immunogenic epitopes are derived from the same source. In some embodiments, none of the plurality of immunogenic epitopes are derived from the same source.

In some embodiments, according to any of the modified monocytes, or monocyte-dendritic progenitors or DCs described herein, the modified monocyte, or monocyte-dendritic progenitor or DC comprises a plurality of antigens that comprise a plurality of immunogenic epitopes. In some embodiments, following administration to an individual of the modified monocyte, or monocyte-dendritic progenitor or DC comprising the plurality of antigens that comprise the plurality of immunogenic epitopes, none of the plurality of immunogenic epitopes decreases an immune response in the individual to any of the other immunogenic epitopes.

In some embodiments, according to any of the modified monocytes, or monocyte-dendritic progenitors or DCs described herein, the method for modulating the function of monocyte, or monocyte-dendritic progenitor or DC comprises a process employing a cell-deforming constriction through which an input monocyte, or monocyte-dendritic progenitor or DC is passed. In some embodiments, the diameter of the constriction is less than the diameter of the input monocyte, or monocyte-dendritic progenitor or DC. In some embodiments, the diameter of the constriction is about 20% to about 99% of the diameter of the input monocyte, or monocyte-dendritic progenitor or DC. In some embodiments, the diameter of the constriction is about 20% to about 60% of the diameter of the input monocyte, or monocyte-dendritic progenitor or DC. In some embodiments, the cell-deforming constriction is contained in a microfluidic channel, such as any of the microfluidic channels described herein. The microfluidic channel may be contained in any of the microfluidic devices described herein, such as described in the section titled Microfluidic Devices below. Thus, in some embodiments, according to any of the methods described herein prepared by a process employing a microfluidic channel including a cell-deforming constriction through which an input monocyte, or monocyte-dendritic progenitor or DC is passed, the process comprises passing the input monocyte, or monocyte-dendritic progenitor or DC through a microfluidic channel including a cell-deforming constriction contained in any of the microfluidic systems described herein. In some embodiments, a deforming force is applied to the input monocyte, or monocyte-dendritic progenitor or DC as it passes through the constriction, thereby causing the perturbations of the input monocyte, or monocyte-dendritic progenitor or DC.

In some embodiments, there is provided a method for modulating an immune response in an individual, comprising: administering to the individual any of the modified antigen presenting cells described herein.

In some embodiments, there is provided a method for modulating an immune response in an individual, comprising: administering to the any of the modified dendritic cells described herein.

### Compositions

In certain aspects, there is provided a composition (e.g., a pharmaceutical composition) comprising a modified antigen presenting cell comprising an agent that enhances the viability and/or function of the antigen presenting cell according to any of the embodiments described herein. In some embodiments, the modified antigen presenting cell further comprises an antigen and/or an adjuvant. In some embodiments, the composition is a pharmaceutical composition comprising the modified antigen presenting cell and a pharmaceutically acceptable carrier.

### Methods for Modulating an Immune Response

In certain aspects, there is provided a method for modulating an immune response in an individual, comprising administering to the individual a modified antigen presenting cell according to any of the embodiments described herein, a composition according to any of the embodiments described herein, or a pharmaceutical composition according to any of the embodiments described herein.

In certain aspects, there is provided a method for modulating an immune response in an individual, comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances the viability and/or function of the antigen presenting cell to pass into the antigen presenting cell; and b) incubating the perturbed input antigen presenting cell with the agent that enhances the viability and/or function of the antigen presenting cell for a sufficient time to allow the agent to enter the perturbed input antigen presenting cell, thereby generating a modified antigen presenting cell; and c) administering the modified antigen presenting cell to the individual.

In certain aspects, there is provided a method for modulating an immune response in an individual, comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances the viability and/or function of the antigen presenting cell to pass into the antigen presenting cell; and b) incubating the perturbed input antigen presenting cell with the agent that enhances the viability and/or function of the antigen presenting cell for a sufficient time to allow the agent to enter the perturbed input antigen presenting cell, thereby generating a modified antigen presenting cell; and c) administering the modified antigen presenting cell to the individual. In some embodiments, the modified antigen presenting cell further comprises an antigen and/or an adjuvant. In some embodiments, the concentration of the antigen incubated with the perturbed input antigen presenting cell is between about 1 pM-10 mM. In some embodiments, the antigen is encapsulated in a nanoparticle. In some embodiments, the concentration of the agent that enhances the viability and/or function of the antigen presenting cell incubated with the perturbed input antigen presenting cell is between about 1 pM-10 mM. In some embodiments, the agent is encapsulated in a nanoparticle. In some embodiments, the concentration of the adjuvant incubated with the perturbed input antigen presenting cell is between about 1 pM-10 mM. In some embodiments, the adjuvant is encapsulated in a nanoparticle.

In some embodiments, according to any of the methods for modulating an immune response in an individual described herein, the agent comprises a protein or polypeptide. In some embodiments, the agent is a protein or polypeptide. In some embodiments, the protein or polypeptide is a therapeutic protein, antibody, fusion protein, antigen, synthetic protein, reporter marker, or selectable marker. In some embodiments, the protein is a gene-editing protein or nuclease such as a zinc-finger nuclease (ZFN), transcription activator-like effector nuclease (TALEN), mega nuclease, or CRE recombinase. In some embodiments, the gene-editing protein or nuclease is CRISPR. In further embodiments, the agent comprises CRISPR with or without with or without an ssODN for homologous recombination. In some embodiments, the fusion proteins can include, without limitation, chimeric protein drugs such as antibody drug conjugates or recombinant fusion proteins such as proteins tagged with OST or streptavidin. In some embodiments, the agent is a transcription factor. In some embodiments, the agent comprises a nucleic acid. In some embodiments, the agent is a nucleic acid. Exemplary nucleic acids include, without limitation, recombinant nucleic acids, DNA, recombinant DNA, cDNA, genomic DNA, RNA, siRNA, mRNA, saRNA, miRNA, lncRNA, tRNA, and shRNA. In some embodiments, the nucleic acid is homologous to a nucleic acid in the cell. In some embodiments, the nucleic acid is heterologous to a nucleic acid in the cell. In some embodiments, the agent is a plasmid. In some embodiments, the agent is a nucleic acid-protein complex. In some embodiments, the nucleic acid-protein complex is a gene-editing complex with or without an ssODN for homologous recombination.

In some embodiments according to any of the methods for modulating an immune response in an individual described herein, the antigen presenting cell is a peripheral blood mononuclear cell (PBMC). In some embodiments, wherein the modified antigen presenting cell or enhanced antigen presenting cell comprises an agent that enhances the viability and/or function of the antigen presenting cell and wherein the input antigen presenting cell is a PBMC, the agent modulates immune activity. In further embodiments, the agent that modulates immune activity upregulates the expression of one or more of IL-2, IL-7, IL-12a IL-12b, or IL-15. In some embodiments, the agent that modulates immune activity modulates the expression of one or more of the interferon-regulatory factors (IRFs), such as IRF3 or IRF5. In some embodiments, the agent that modulates immune activity modulates the expression of one or more of the toll-like receptors (TLRs), such as TLR-4. In some embodiments, the agent that modulates immune activity modulates the expression and/or activity of one or more of the toll-like receptors (TLRs), such as TLR-4 and/or TLR-9. In some embodiments, the agent that modulates immune activity modulates the expression of one or more of pattern recognition receptors (PRRs). In some embodiments, the agent that modulates immune activity modulates the activity of one or more of pattern recognition receptors (PRRs). In some embodiments, the agent that modulates immune activity modulates the expression and/or activity of one or more of STING, RIG-I, AIM2, LRRF1P1 or NLPR3. In some embodiments, wherein the enhanced antigen presenting cell comprises an agent that enhances the viability and/or function of the antigen presenting cell and wherein the input antigen presenting cell is a PBMC, the agent enhances antigen presentation. In some embodiments, the agent that enhances antigen presentation upregulates the expression of MHC-I and/or MHC-II. In some embodiments, the agent that enhances antigen presentation upregulates the expression of T-cell Receptor (TCR). In some embodiments, wherein the enhanced antigen presenting cell comprises an agent that enhances the viability and/or function of the antigen presenting cell and wherein the input antigen presenting cell is a PBMC, the agent enhances activation of the antigen presenting cell. In some embodiments, the agent that enhances activation of the antigen presenting cell modulates the expression of one or more of CD25, KLRG1, CD80, or CD86. In some embodiments, the agent that enhances activation of the antigen presenting cell modulates the expression of CD80 and/or CD86. In some embodiments, wherein the enhanced antigen presenting cell comprises an agent that enhances the viability and/or function of the antigen presenting cell and wherein the input antigen presenting cell is a PBMC, the agent enhances activation of the antigen presenting cell. In some embodiments, the agent that enhances activation of the antigen presenting cell modulates the expression of one or more of CD25, KLRG1, CD80, or CD86. In some embodiments, wherein the enhanced antigen presenting cell comprises an agent that enhances the viability and/or function of the antigen presenting cell and wherein the input antigen presenting cell is a PBMC, the agent enhances homing of the antigen presenting cell. In some embodiments, the agent that enhances homing of the antigen presenting cell modulates the expression of one or more of CD62L, CCR2, CCR7, CX3CR1, or CXCR5. In some embodiments, wherein the enhanced antigen presenting cell comprises an agent that enhances the viability and/or function of the antigen presenting cell and wherein the input antigen presenting cell is a PBMC, the agent is an anti-apoptotic agent. In some embodiments, the anti-apoptotic agent modulates the expression of one or more of Bcl-2, Bcl-3, or Bcl-xL. In some embodiments, wherein the enhanced antigen presenting cell comprises an agent that enhances the viability and/or function of the antigen presenting cell and wherein the input antigen presenting cell is a PBMC, the agent induces alteration in cell fate or phenotype. In some embodiments, the agent that induces alteration in cell fate or phenotype modulates the expression of one or more of Oct4, Sox2, c-Myc, Klf-4, Nanog, Lin28, Lin28B, T-bet, or GATA3. In some embodiments, the agent is a nucleic acid or a nucleic acid-protein complex. In some embodiments, the nucleic acid is a DNA or an mRNA. In some embodiments, the nucleic acid is a siRNA, shRNA or miRNA. In some embodiments, the nucleic acid-protein complex is a gene-editing complex. In some embodiments, the nucleic acid-protein complex comprises Cas9 and guide RNA, with or without an ssODN for homologous recombination or homology directed repair.

In some embodiments according to any of the methods for modulating an immune response in an individual described herein, the agent enhances homing of the antigen presenting cell to a site for T cell activation. In some embodiments, the agent enhances homing of the antigen presenting cell to lymph nodes. In some embodiments, the agent that enhances homing of the antigen presenting cell modulates the expression of one or more of CD62L, CCR2, CCR7, CX3CR1, or CXCR5. In some embodiments, the agent is a protein, a nucleic acid or a nucleic acid-protein complex. In some embodiments, the nucleic acid is a DNA or an mRNA. In some embodiments, the nucleic acid is a siRNA, shRNA or miRNA. In some embodiments, the nucleic acid-protein complex is a gene-editing complex. In some embodiments, the agent that enhances homing of the antigen presenting cell comprises one or more mRNAs encoding one or more of: CD62L, CCR2, CCR7, CX3CR1, or CXCR5. In some embodiments, the expression of one or more of CD62L, CCR2, CCR7, CX3CR1, or CXCR5 is increased by about any one of: 5%, 10%, 20%, 30%, 40%, 50%, 60%, 70%, 80%, 90%, 95%, 99%, or 100%. In some embodiments, the expression of one or more of CD62L, CCR2, CCR7, CX3CR1, or CXCR5 is increased by about any one of: 2-fold, 3-fold, 5-fold, 10-fold, 50-fold, 100 fold, 500-fold, 1000-fold or more. In some embodiments, the homing of the modified antigen presenting cell comprising the agent to a site for T cell activation is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the homing of the modified antigen presenting cell comprising the agent to a site for T cell activation is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating an immune response in an individual, comprising: administering to the individual a modified antigen presenting cell, wherein the modified antigen presenting cell is prepared by a process comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances viability and/or function of the antigen presenting cell to pass into the antigen presenting cell; and; b) incubating the perturbed input antigen presenting cell with the agent that enhances viability and/or function of the antigen presenting cell for a sufficient time to allow the agent to enter the perturbed input antigen presenting cell, thereby generating the modified antigen presenting cell with enhanced viability and/or function. In some embodiments, the agent that enhances viability and/or function of the antigen presenting cell upregulates expression of one or more of IL-2, IL-7, IL-12a IL-12b, IL-15, IL-18 or IL-21. In further embodiments, the agent that upregulates expression of one or more IL-2, IL-7, IL-12a IL-12b, IL-15, IL-18 or IL-21 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances viability and/or function of the antigen presenting cell comprises one or more mRNAs encoding one or more of: IL-2, IL-7, IL-12a IL-12b, IL-15, IL-18 or IL-21. In some embodiments, the expression of one or more of IL-2, IL-7, IL-12a IL-12b, IL-15, IL-18 or IL-21 is increased by about any one of: 5%, 10%, 20%, 30%, 40%, 50%, 60%, 70%, 80%, 90%, 95%, 99%, or 100%. In some embodiments, the expression of one or more of IL-2, IL-7, IL-12a IL-12b, IL-15, IL-18 or IL-21 is increased by about any one of: 2-fold, 3-fold, 5-fold, 10-fold, 50-fold, 100 fold, 500-fold, 1000-fold, or more. In some embodiments, the circulating half-life and/or in vivo persistence of an antigen presenting cell of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the circulating half-life and/or in vivo persistence of an antigen presenting cell of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell. In some embodiments that can be combined with any other embodiments, the one or more of IL-2, IL-7, IL-12a IL-12b, IL-15, IL-18 or IL-21 comprise endogenous nucleotide or protein sequences. In some embodiments, the one or more of: IL-2, IL-7, IL-12a IL-12b, IL-15, IL-18 or IL-21 comprise modified nucleotide or protein sequences. In some embodiments, the one or more of: IL-2, IL-7, IL-12a IL-12b, IL-15, IL-18 or IL-21 are membrane-bound, such as bound to the membrane of the modified antigen presenting cell. In some embodiments, the one or more of: IL-2, IL-7, IL-12a IL-12b, IL-15, IL-18 or IL-21 are bound to membrane by GPI anchor. In some embodiments, the one or more of: IL-2, IL-7, IL-12a IL-12b, IL-15, IL-18 or IL-21 comprise a transmembrane domain sequence. In some embodiments, the one or more of: IL-2, IL-7, IL-12a IL-12b, IL-15, IL-18 or IL-21 comprise a GPI-anchor signal sequence. In some embodiments, the one or more of: IL-2, IL-7, IL-12a IL-12b, IL-15, IL-18 or IL-21 comprise the transmembrane domain and cytoplasmic tail of murine B7-1 (B7TM). In some embodiments, the one or more of: IL-2, IL-7, IL-12a IL-12b, IL-15, IL-18 or IL-21 comprising modified sequences do not bind to IL-2Rα chain (CD25) and/or do not bind IL-15Rα (CD215). In some embodiments, the one or more of: IL-2, IL-7, IL-12a IL-12b, IL-15, IL-18 or IL-21 comprising modified sequences bind to IL-2Rβ [symbol] _{c} with higher affinity than the respective natural counterpart, such as but not limited to affinity that is higher than the natural counterpart by 5%, 10%, 20%, 30%, 40%, 50%, 60%, 70%, 80%, 90%, 95%, 99%, 100%, 2-fold, 3-fold, 5-fold, 10-fold, 50-fold, 100 fold, 500-fold, 1000-fold or more. In some embodiments, the one or more of: IL-2, IL-7, IL-12a IL-12b, IL-15, IL-18 or IL-21 comprising modified amino acid sequence display about any one of: 80%, 81%, 82%, 83%, 84%, 85%, 86%, 87%, 88%, 89%, 90%, 91%, 92%, 93%, 94%, 95%, 96%, 97%, 98%, or 99% similarity as the respective wild type amino acid sequence. In some embodiments, the one or more of: IL-2, IL-7, IL-12a IL-12b, IL-15, IL-18 or IL-21 comprising modified nucleotide sequence display about any one of: 80%, 81%, 82%, 83%, 84%, 85%, 86%, 87%, 88%, 89%, 90%, 91%, 92%, 93%, 94%, 95%, 96%, 97%, 98%, or 99% similarity as the respective wild type nucleotide sequence. In some embodiments, the agent comprises one or more mimics of: IL-2, IL-7, IL-12a IL-12b, IL-15, IL-18 or IL-21, wherein the mimic comprises nucleotide or protein sequence that displays about any one of: 80%, 81%, 82%, 83%, 84%, 85%, 86%, 87%, 88%, 89%, 90%, 91%, 92%, 93%, 94%, 95%, 96%, 97%, 98%, or 99% similarity as the respective wild type sequence of IL-2, IL-7, IL-12a IL-12b, IL-15, IL-18 or IL-21. In some embodiments, the one or more of: IL-2, IL-7, IL-12a IL-12b, IL-15, IL-18 or IL-21 comprising modified sequence or the mimic of one or more of IL-2, IL-7, IL-12a IL-12b, IL-15, IL-18 or IL-21 display structural modifications compare to respective wild type counterparts. In some embodiments, the agent comprises an IL-2 mimic. In some embodiments, the agent comprises Neoleukin-2/15 (Neo-2/15).

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances tumor homing of the antigen presenting cell to pass into the antigen presenting cell; and; b) incubating the perturbed input antigen presenting cell with the agent that enhances tumor homing of the antigen presenting cell for a sufficient time to allow the agent to enter the perturbed input antigen presenting cell, thereby generating the modified antigen presenting cell with enhanced tumor homing. In some embodiments, the agent that enhances tumor homing of the antigen presenting cell upregulates expression of one or more of CXCR3, CCR5, VLA-4 or LFA-1. In further embodiments, the agent that upregulates expression of one or more of CXCR3, CCR5, VLA-4 or LFA-1 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances tumor homing of the antigen presenting cell comprises one or more mRNAs encoding one or more of: CXCR3, CCR5, VLA-4 or LFA-1. In some embodiments, the expression of one or more of CXCR3, CCR5, VLA-4 or LFA-1 is increased by about any one of: 5%, 10%, 20%, 30%, 40%, 50%, 60%, 70%, 80%, 90%, 95%, 99%, or 100%. In some embodiments, the expression of one or more of CXCR3, CCR5, VLA-4 or LFA-1 is increased by about any one of: 2-fold, 3-fold, 5-fold, 10-fold, 50-fold, 100 fold, 500-fold, 1000-fold, or more. In some embodiments, the tumor homing of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the tumor homing of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an anti-apoptotic agent to pass into the antigen presenting cell; and b) incubating the perturbed input antigen presenting cell with the anti-apoptotic agent for a sufficient time to allow the agent to enter the perturbed input antigen presenting cell, thereby generating an enhanced antigen presenting cell. In some embodiments, the anti-apoptotic agent upregulates expression of one or more of XIAP, cIAP1/2, survivin, livin, cFLIP, Hsp72, or Hsp90. In further embodiments, the agent that upregulates expression of one or more of XIAP, cIAP1/2, survivin, livin, cFLIP, Hsp72 or Hsp90 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances viability of an antigen presenting cell comprises one or more mRNAs encoding one or more of: XIAP, cIAP1/2, survivin, livin, cFLIP, Hsp72, or Hsp90. In some embodiments, the expression of one or more of XIAP, cIAP1/2, survivin, livin, cFLIP, Hsp72, or Hsp90 is increased by about any one of: 5%, 10%, 20%, 30%, 40%, 50%, 60%, 70%, 80%, 90%, 95%, 99%, or 100%. In some embodiments, the expression of one or more of XIAP, cIAP1/2, survivin, livin, cFLIP, Hsp72, or Hsp90 is increased by about any one of: 2-fold, 3-fold, 5-fold, 10-fold, 50-fold, 100 fold, 500-fold, 1000-fold, or more. In some embodiments, the circulating half-life and/or in vivo persistence of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the circulating half-life and/or in vivo persistence of an antigen presenting cell of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing to pass into the antigen presenting cell; and b) incubating the perturbed input antigen presenting cell with the agent that enhances antigen processing for a sufficient time to allow the agent to enter the perturbed input antigen presenting cell, thereby generating the modified antigen presenting cell. In some embodiments, the agent that enhances antigen processing upregulates expression of one or more of LMP2, LMP7, MECL-1 or β5t. In further embodiments, the agent that upregulates expression of one or more of LMP2, LMP7, MECL-1 or β5t is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances antigen processing comprises one or more mRNAs encoding one or more of: LMP2, LMP7, MECL-1 or β5t. In some embodiments, the expression of one or more of LMP2, LMP7, MECL-1 or β5t is increased by about any one of: 5%, 10%, 20%, 30%, 40%, 50%, 60%, 70%, 80%, 90%, 95%, 99%, or 100%. In some embodiments, the expression of one or more of LMP2, LMP7, MECL-1 or β5t is increased by about any one of: 2-fold, 3-fold, 5-fold, 10-fold, 50-fold, 100 fold, 500-fold, 1000-fold, or more. In some embodiments, the antigen processing in an antigen presenting cell comprising the agent is enhanced by about any one of: 5%, 10%, 20%, 30%, 40%, 50%, 60%, 70%, 80%, 90%, 95%, 99%, or 100% compared to an antigen presenting cell that does not comprise the agent. In some embodiments, the antigen processing in an antigen presenting cell comprising the agent is enhanced by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing and/or loading onto MHC molecules to pass into the antigen presenting cell; and b) incubating the perturbed input antigen presenting cell with the agent that enhances antigen processing and/or loading onto MHC molecules for a sufficient time to allow the agent to enter the perturbed input antigen presenting cell, thereby generating the modified antigen presenting cell. In some embodiments, the agent that enhances antigen processing and/or loading onto MHC molecules upregulates expression of one or more of TAP, Tapasin, ERAAP, Calreticulin, Erp57 or PDI. In further embodiments, the agent that upregulates expression of one or more of TAP, Tapasin, ERAAP, Calreticulin, Erp57 or PDI is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances antigen processing and/or loading comprises one or more mRNAs encoding one or more of: TAP, Tapasin, ERAAP, Calreticulin, Erp57 or PDI. In some embodiments, the expression of one or more of TAP, Tapasin, ERAAP, Calreticulin, Erp57 or PDI is increased by about any one of: 5%, 10%, 20%, 30%, 40%, 50%, 60%, 70%, 80%, 90%, 95%, 99%, or 100%. In some embodiments, the expression of one or more of TAP, Tapasin, ERAAP, Calreticulin, Erp57 or PDI is increased by about any one of: 2-fold, 3-fold, 5-fold, 10-fold, 50-fold, 100 fold, 500-fold, 1000-fold, or more. In some embodiments, the antigen processing and/or loading in an antigen presenting cell comprising the agent is enhanced by about any one of: 5%, 10%, 20%, 30%, 40%, 50%, 60%, 70%, 80%, 90%, 95%, 99%, or 100% compared to an antigen presenting cell that does not comprise the agent. In some embodiments, the antigen processing and/or loading in an antigen presenting cell comprising the agent is enhanced by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that modulates immune activity to pass into the antigen presenting cell; and b) incubating the perturbed input antigen presenting cell with the agent that modulates immune activity for a sufficient time to allow the agent to enter the perturbed input antigen presenting cell, thereby generating the modified antigen presenting cell. In some embodiments, the agent that modulates immune activity upregulates expression of one or more of type I interferon, type II interferon, type III interferon and Shp2. In further embodiments, the agent that upregulates expression of one or more of type I interferon, type II interferon, type III interferon and Shp2 is a nucleic acid, a protein or a nucleic acid-protein complex. In some embodiments, the agent that modulates immune activity upregulates expression of one or more of type I interferon, type II interferon, or type III interferon. In further embodiments, the agent that upregulates expression of one or more of type I interferon, type II interferon, or type III interferon is a nucleic acid, a protein or a nucleic acid-protein complex. In some embodiments, the nucleic acid is a DNA, an mRNA, an siRNA, an shRNA or an miRNA. In some embodiments, the agent that modulates immune activity downregulates expression of interferon-beta. In further embodiments, the agent that downregulates expression of interferon-beta is a nucleic acid, a protein, a nucleic acid-protein complex or a small molecule. In some embodiments, the nucleic acid is a DNA, an mRNA, an siRNA, an shRNA or an miRNA. In some embodiments, the nucleic acid-protein complex is a gene-editing complex with or without an ssODN for homologous recombination.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances the function and/or maturation of an antigen presenting cell to pass into the antigen presenting cell; and b) incubating the perturbed input antigen presenting cell with the agent that enhances the function and/or maturation of an antigen presenting cell for a sufficient time to allow the agent to enter the perturbed input antigen presenting cell, thereby generating the modified antigen presenting cell. In some embodiments, the agent that enhances the function and/or maturation of an antigen presenting cell of the antigen presenting cell upregulates expression of one or more of type I interferons, type II interferons, or type III interferons. In some embodiments, the agent that enhances the function and/or maturation of an antigen presenting cell of the antigen presenting cell upregulates expression of one or more of: IFN-α2, IFN-β, IFN-γ, IFN-λ1, IFN-λ2, or IFN-λ3. In some embodiments, the agent that enhances expression of homing receptors in antigen presenting cell comprises one or more mRNAs encoding one or more of: IFN-α2, IFN-β, IFN-γ, IFN-λ1, IFN-λ2, or IFN-λ3. In some embodiments, the expression of one or more of IFN-α2, IFN-β, IFN-γ, IFN-λ1, IFN-λ2, or IFN-λ3 is increased by about any one of: 5%, 10%, 20%, 30%, 40%, 50%, 60%, 70%, 80%, 90%, 95%, 99%, or 100%. In some embodiments, the expression of one or more of IFN-α2, IFN-β, IFN-γ, IFN-λ1, IFN-λ2, or IFN-λ3 is increased by about any one of: 2-fold, 3-fold, 5-fold, 10-fold, 50-fold, 100 fold, 500-fold, 1000-fold, or more. In some embodiments, the maturation of an antigen presenting cell comprising the agent is enhanced by about any one of: 5%, 10%, 20%, 30%, 40%, 50%, 60%, 70%, 80%, 90%, 95%, 99%, or 100% compared to an antigen presenting cell that does not comprise the agent. In some embodiments, the maturation of an antigen presenting cell comprising the agent is enhanced by about any one of: 2-fold, 3-fold, 5-fold, 10-fold, 50-fold, 100 fold, 500-fold, 1000-fold or more compared to an antigen presenting cell that does not comprise the agent.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances viability of the antigen presenting cell to pass into the antigen presenting cell; and b) incubating the perturbed input antigen presenting cell with the agent that enhances viability of the antigen presenting cell for a sufficient time to allow the agent to enter the perturbed input antigen presenting cell, thereby generating the modified antigen presenting cell. In some embodiments, the agent that enhances viability of the antigen presenting cell upregulates expression of a serpin. In further embodiments, the agent that upregulates expression a serpin is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances viability of the antigen presenting cell comprises one or more mRNAs encoding one or more serpins. In some embodiments, the expression of one or more serpins is increased by about any one of: 5%, 10%, 20%, 30%, 40%, 50%, 60%, 70%, 80%, 90%, 95%, 99%, or 100%. In some embodiments, the expression of one or more serpins is increased by about any one of: 2-fold, 3-fold, 5-fold, 10-fold, 50-fold, 100 fold, 500-fold, 1000-fold, or more. In some embodiments, the circulating half-life and/or in vivo persistence of an antigen presenting cell of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the circulating half-life and/or in vivo persistence of an antigen presenting cell of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances homing receptors of the antigen presenting cell to pass into the antigen presenting cell; and b) incubating the perturbed input antigen presenting cell with the agent that enhances homing receptors of the antigen presenting cell for a sufficient time to allow the agent to enter the perturbed input antigen presenting cell, thereby generating the modified antigen presenting cell. In some embodiments, the agent that enhances homing receptors of the antigen presenting cell upregulates expression of CCL2. In further embodiments, the agent that upregulates expression of CCL2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances homing and/or triggers alternative homing comprises one or more mRNAs encoding CCL2. In some embodiments, the expression of CCL2 is increased by about any one of: 5%, 10%, 20%, 30%, 40%, 50%, 60%, 70%, 80%, 90%, 95%, 99%, or 100%. In some embodiments, the expression of CCL2 is increased by about any one of: 2-fold, 3-fold, 5-fold, 10-fold, 50-fold, 100 fold, 500-fold, 1000-fold, or more. In some embodiments, the homing and/or alternative homing of an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the homing and/or alternative homing of an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that activates T cells to pass into the antigen presenting cell; and b) incubating the perturbed input antigen presenting cell with the agent that activates T cells for a sufficient time to allow the agent to enter the perturbed input antigen presenting cell, thereby generating the modified antigen presenting cell. In some embodiments, the agent that activates T cells upregulates expression of one or more of CD27, CD28, CD40, CD122, 4-1BB (CD137), OX40(CD134)/OX40L(CD252), GITR or ICOS. In further embodiments, the agent that upregulates expression of one or more of CD27, CD28, CD40, CD122, 4-1BB (CD137), OX40(CD134)/OX40L(CD252), GITR or ICOS is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances T cell activation comprises one or more mRNAs encoding one or more of: CD27, CD28, CD40, CD122, 4-1BB (CD137), OX40(CD134)/OX40L(CD252), GITR or ICOS. In some embodiments, the expression of one or more of CD27, CD28, CD40, CD122, 4-1BB (CD137), OX40(CD134)/OX40L(CD252), GITR or ICOS is increased by about any one of: 5%, 10%, 20%, 30%, 40%, 50%, 60%, 70%, 80%, 90%, 95%, 99%, or 100%. In some embodiments, the expression of one or more of CD27, CD28, CD40, CD122, 4-1BB (CD137), OX40(CD134)/OX40L(CD252), GITR or ICOS is increased by about any one of: 2-fold, 3-fold, 5-fold, 10-fold, 50-fold, 100 fold, 500-fold, 1000-fold, or more. In some embodiments, the T cell activation by an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the T cell activation by an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that activates T cells to pass into the antigen presenting cell; and b) incubating the perturbed input antigen presenting cell with the agent that activates T cells for a sufficient time to allow the agent to enter the perturbed input antigen presenting cell, thereby generating the modified antigen presenting cell. In some embodiments, the agent that activates T cells upregulates expression of one or more of CD70, CD80, CD86, CD40L, 4-1BBL (CD137L), OX40L(CD252), GITRL or ICOSL. In further embodiments, the agent that upregulates expression of one or more of CD70, CD80, CD86, CD40L, 4-1BBL (CD137L), OX40L(CD252), GITRL or ICOSL is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances T cell activation comprises one or more mRNAs encoding one or more of: CD70, CD80, CD86, CD40L, 4-1BBL (CD137L), OX40L(CD252), GITRL or ICOSL. In some embodiments, the expression of one or more of CD70, CD80, CD86, CD40L, 4-1BBL (CD137L), OX40L(CD252), GITRL or ICOSL is increased by about any one of: 5%, 10%, 20%, 30%, 40%, 50%, 60%, 70%, 80%, 90%, 95%, 99%, or 100%. In some embodiments, the expression of one or more of CD70, CD80, CD86, CD40L, 4-1BBL (CD137L), OX40L(CD252), GITRL or ICOSL is increased by about any one of: 2-fold, 3-fold, 5-fold, 10-fold, 50-fold, 100 fold, 500-fold, 1000-fold, or more. In some embodiments, the T cell activation by an antigen presenting cell comprising the agent is increased by about any one of: 5%, 10%, 20%, 30%, 40%, 50%, 60%, 70%, 80%, 90%, 95%, 99%, or 100% compared to an antigen presenting cell that does not comprise the agent. In some embodiments, the T cell activation by an antigen presenting cell comprising the agent is increased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating an immune response in an individual, comprising: administering to the individual a modified antigen presenting T cell, wherein the modified antigen presenting T cell is prepared by a process comprising: a) passing a cell suspension comprising an input antigen presenting T cell through a cell-deforming constriction, wherein a diameter of the constriction is a function of a diameter of the input antigen presenting T cell in the suspension, thereby causing perturbations of the input antigen presenting T cell large enough for an agent that activates T cells to pass into the antigen presenting T cell; and b) incubating the perturbed input antigen presenting T cell with the agent that activates T cells for a sufficient time to allow the agent to enter the perturbed input antigen presenting T cell, thereby generating the modified antigen presenting T cell. In some embodiments, the agent that activates T cells upregulates expression of one or more of CD27, CD28, CD40, CD122, 4-1BB (CD137), OX40(CD134)/OX40L(CD252), GITR or ICOS. In further embodiments, the agent that upregulates expression of one or more of CD27, CD28, CD40, CD122, 4-1BB (CD137), OX40(CD134)/OX40L(CD252), GITR or ICOS is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that enhances T cell activation comprises one or more mRNAs encoding one or more of: CD27, CD28, CD40, CD122, 4-1BB (CD137), OX40(CD134)/OX40L(CD252), GITR or ICOS. In some embodiments, the expression of one or more of CD27, CD28, CD40, CD122, 4-1BB (CD137), OX40(CD134)/OX40L(CD252), GITR or ICOS is increased by about any one of: 5%, 10%, 20%, 30%, 40%, 50%, 60%, 70%, 80%, 90%, 95%, 99%, or 100%. In some embodiments, the expression of one or more of CD27, CD28, CD40, CD122, 4-1BB (CD137), OX40(CD134)/OX40L(CD252), GITR or ICOS is increased by about any one of: 2-fold, 3-fold, 5-fold, 10-fold, 50-fold, 100 fold, 500-fold, 1000-fold, or more. In some embodiments, the T cell activation induced by an antigen presenting T cell comprising the agent is increased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the T cell activation induced by an antigen presenting T cell comprising the agent is increased by about any one of: 2-fold, 3-fold, 5-fold, 10-fold, 50-fold, 100 fold, 500-fold, 1000-fold or more compared to an antigen presenting T cell that does not comprise the agent. In some embodiments, the activation of an antigen presenting T cell comprising the agent is increased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the activation of an antigen presenting T cell comprising the agent is increased by about any one of: 2-fold, 3-fold, 5-fold, 10-fold, 50-fold, 100 fold, 500-fold, 1000-fold or more compared to an antigen presenting T cell that does not comprise the agent.

In certain aspects, there is provided a method for modulating an immune response in an individual, comprising: administering to the individual a modified antigen presenting cell, wherein the modified antigen presenting cell is prepared by a process comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that downregulates T cell inhibition to pass into the antigen presenting cell; and b) incubating the perturbed input antigen presenting cell with the agent that downregulates T cell inhibition for a sufficient time to allow the agent to enter the perturbed input antigen presenting cell, thereby generating the modified antigen presenting cell. In some embodiments, the agent that downregulates T cell inhibition downregulates expression of one or more of LAG3, VISTA, TIM1, B7-H4 (VTCN1) or BTLA. In further embodiments, the agent that downregulates expression of one or more of LAG3, VISTA, TIM1, B7-H4 (VTCN1) or BTLA is a nucleic acid, a protein, a peptide, a nucleic acid-protein complex or a small molecule. In some embodiments, the nucleic acid is an siRNA, an shRNA or an miRNA. In some embodiments, the nucleic acid-protein complex is a gene-editing complex with or without an ssODN for homologous recombination. In some embodiments, the agent that downregulates T cell inhibition comprises one or more Cas9-gRNA RNP complexes targeting one or more of: LAG3, VISTA, TIM1, B7-H4 (VTCN1) or BTLA. In some embodiments, the expression of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the expression of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, 1000-fold, or more. In some embodiments, the agent that downregulates T cell inhibition comprises one or more small molecules targeting one or more of: LAG3, VISTA, TIM1, B7-H4 (VTCN1) or BTLA. In some embodiments, the agent that downregulates T cell inhibition comprises one or more antibodies or fragments thereof targeting one or more of: LAG3, VISTA, TIM1, B7-H4 (VTCN1) or BTLA. In some embodiments, the activity of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the activity of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, or 1000-fold, or more. In some embodiments, the T cell inhibition induced by the antigen presenting cell comprising the agent is decreased by about any one of: 5%, 10%, 20%, 30%, 40%, 50%, 60%, 70%, 80%, 90%, 95%, 99%, or 100% compared to an antigen presenting cell that does not comprise the agent. In some embodiments, the T cell inhibition induced by the antigen presenting cell comprising the agent is decreased by about any one of: 2-fold, 3-fold, 5-fold, 10-fold, 50-fold, 100 fold, 500-fold, 1000-fold or more compared to an antigen presenting cell that does not comprise the agent. In some embodiments, the antigen presenting cell is a dendritic cell.

In certain aspects, there is provided a method for modulating an immune response in an individual, comprising: administering to the individual a modified antigen presenting T cell, wherein the modified antigen presenting T cell is prepared by a process comprising: a) passing a cell suspension comprising an input antigen presenting T cell through a cell-deforming constriction, wherein a diameter of the constriction is a function of a diameter of the input antigen presenting T cell in the suspension, thereby causing perturbations of the input antigen presenting T cell large enough for an agent that downregulates T cell inhibition to pass into the antigen presenting T cell; and b) incubating the perturbed input antigen presenting T cell with the agent that downregulates T cell inhibition for a sufficient time to allow the agent to enter the perturbed input antigen presenting T cell, thereby generating the modified antigen presenting T cell. In some embodiments, the agent that downregulates T cell inhibition downregulates expression of one or more of LAG3, VISTA, TIM1, B7-H4 (VTCN1) or BTLA. In further embodiments, the agent that downregulates expression of one or more of LAG3, VISTA, TIM1, B7-H4 (VTCN1) or BTLA is a nucleic acid, a protein, a peptide, a nucleic acid-protein complex or a small molecule. In some embodiments, the nucleic acid is an siRNA, an shRNA or an miRNA. In some embodiments, the nucleic acid-protein complex is a gene-editing complex with or without an ssODN for homologous recombination. In some embodiments, the agent that downregulates T cell inhibition comprises one or more Cas9-gRNA RNP complexes targeting one or more of: LAG3, VISTA, TIM1, B7-H4 (VTCN1) or BTLA. In some embodiments, the expression of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the expression of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, 1000-fold, or more. In some embodiments, the agent that downregulates T cell inhibition comprises one or more small molecules targeting one or more of: LAG3, VISTA, TIM1, B7-H4 (VTCN1) or BTLA. In some embodiments, the agent that downregulates T cell inhibition comprises one or more antibodies or fragments thereof targeting one or more of: LAG3, VISTA, TIM1, B7-H4 (VTCN1) or BTLA. In some embodiments, the activity of one or more of LAG3, VISTA, TIM1, B7-H4 (VTCN1) or BTLA, GITR or ICOS is decreased by about any one of: 5%, 10%, 20%, 30%, 40%, 50%, 60%, 70%, 80%, 90%, 95%, 99%, or 100%. In some embodiments, the activity of one or more of LAG3, VISTA, TIM1, B7-H4 (VTCN1) or BTLA is decreased by about any one of: 2-fold, 3-fold, 5-fold, 10-fold, 50-fold, 100 fold, 500-fold, 1000-fold, or more. In some embodiments, the T cell inhibition induced by the antigen presenting T cell comprising the agent is decreased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the T cell inhibition induced by the antigen presenting T cell comprising the agent is decreased by about any one of: 2-fold, 3-fold, 5-fold, 10-fold, 50-fold, 100 fold, 500-fold, 1000-fold or more compared to an antigen presenting T cell that does not comprise the agent. In some embodiments, the inhibition of the antigen presenting T cell comprising the agent is decreased by about any one of: 5%, 10%, 20%, 30%, 40%, 50%, 60%, 70%, 80%, 90%, 95%, 99%, or 100% compared to an antigen presenting T cell that does not comprise the agent. In some embodiments, the inhibition of the antigen presenting T cell comprising the agent is decreased by about any one of: 2-fold, 3-fold, 5-fold, 10-fold, 50-fold, 100 fold, 500-fold, 1000-fold or more compared to an antigen presenting T cell that does not comprise the agent.

In certain aspects, there is provided a method for modulating an immune response in an individual, comprising: administering to the individual a modified monocyte or monocyte-dendritic progenitor cell, wherein the modified monocyte or monocyte-dendritic progenitor cell is prepared by a process comprising: a) passing a cell suspension comprising an input monocyte or monocyte-dendritic progenitor through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of DCs to pass into the monocyte or monocyte-dendritic progenitor cell; and b) incubating the perturbed input monocyte or monocyte-dendritic progenitor cell with the agent that promotes formation of DCs for a sufficient time to allow the agent to enter the perturbed input monocyte or monocyte-dendritic progenitor cell, thereby generating the modified monocyte or monocyte-dendritic progenitor cell. In some embodiments, the agent that promotes formation of DCs upregulates expression of one or more of PU.1, Flt3, Flt3L or GMCSF. In further embodiments, the agent that upregulates expression of one or more of PU.1, Flt3, Flt3L or GMCSF is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes DC formation from a monocyte or monocyte-dendritic progenitor cell comprises one or more mRNAs encoding one or more of: PU.1, Flt3, Flt3L or GMCSF. In some embodiments, the expression of one or more of PU.1, Flt3, Flt3L or GMCSF is increased by about any one of: 5%, 10%, 20%, 30%, 40%, 50%, 60%, 70%, 80%, 90%, 95%, 99%, or 100%. In some embodiments, the expression of one or more of PU.1, Flt3, Flt3L or GMCSF is increased by about any one of: 2-fold, 3-fold, 5-fold, 10-fold, 50-fold, 100 fold, 500-fold, 1000-fold, or more. In some embodiments, DC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, DC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In certain aspects, there is provided a method for modulating an immune response in an individual, comprising: administering to the individual a modified monocyte or monocyte-dendritic progenitor cell, wherein the modified monocyte or monocyte-dendritic progenitor cell is prepared by a process comprising: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of pDCs to pass into the monocyte or monocyte-dendritic progenitor cell; and b) incubating the perturbed input monocyte or monocyte-dendritic progenitor cell with the agent that promotes formation of pDCs for a sufficient time to allow the agent to enter the perturbed input monocyte or monocyte-dendritic progenitor cell, thereby generating the modified monocyte or monocyte-dendritic progenitor cell. In some embodiments, the agent that promotes formation of pDCs upregulates expression of E2-2. In further embodiments, the agent that upregulates expression of E2-2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes pDC formation from a monocyte or monocyte-dendritic progenitor cell comprises one or more mRNAs encoding E2-2. In some embodiments, the expression of E2-2 is increased by about any one of: 5%, 10%, 20%, 30%, 40%, 50%, 60%, 70%, 80%, 90%, 95%, 99%, or 100%. In some embodiments, the expression of E2-2 is increased by about any one of: 2-fold, 3-fold, 5-fold, 10-fold, 50-fold, 100 fold, 500-fold, 1000-fold, or more. In some embodiments, pDC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, pDC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In certain aspects, there is provided a method for modulating an immune response in an individual, comprising: administering to the individual a modified monocyte or monocyte-dendritic progenitor cell, wherein the modified monocyte or monocyte-dendritic progenitor cell is prepared by a process comprising: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that promotes formation of CD8a+/CD10+ DCs to pass into the monocyte or monocyte-dendritic progenitor cell; and b) incubating the perturbed input monocyte or monocyte-dendritic progenitor cell with the agent that promotes formation of CD8a+/CD10+ DCs for a sufficient time to allow the agent to enter the perturbed input monocyte or monocyte-dendritic progenitor cell, thereby generating the modified monocyte or monocyte-dendritic progenitor cell. In some embodiments, the agent that promotes formation of CD8a+/CD10+ DCs upregulates expression of one or more of Batf3, IRF8 or Id2. In further embodiments, the agent that upregulates expression of one or more of Batf3, IRF8 or Id2 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes formation of CD8a+/CD10+ DCs from a monocyte or monocyte-dendritic progenitor cell comprises one or more mRNAs encoding one or more of: Batf3, IRF8 or Id2. In some embodiments, the expression of one or more of Batf3, IRF8 or Id2 is increased by about any one of: 5%, 10%, 20%, 30%, 40%, 50%, 60%, 70%, 80%, 90%, 95%, 99%, or 100%. In some embodiments, the expression of one or more of Batf3, IRF8 or Id2 is increased by about any one of: 2-fold, 3-fold, 5-fold, 10-fold, 50-fold, 100 fold, 500-fold, 1000-fold, or more. In some embodiments, CD8a+/CD10+ DC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, CD8a+/CD10+ DC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In c certain aspects, there is provided a method for modulating an immune response in an individual, comprising: administering to the individual a modified monocyte or monocyte-dendritic progenitor cell, wherein the modified monocyte or monocyte-dendritic progenitor cell is prepared by a process comprising: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of CD11b+ DCs to pass into the monocyte or monocyte-dendritic progenitor cell; and b) incubating the perturbed input monocyte or monocyte-dendritic progenitor cell with the agent that promotes formation of CD11b+ DCs for a sufficient time to allow the agent to enter the perturbed input monocyte or monocyte-dendritic progenitor cell, thereby generating the modified monocyte or monocyte-dendritic progenitor cell. In some embodiments, the agent that promotes formation of CD11b+ DCs upregulates expression of one or more of IRF4, RBJ, MgI or Mtg16. In further embodiments, the agent that upregulates expression of one or more of IRF4, RBJ, MgI or Mtg16 is a nucleic acid, a protein or a nucleic acid-protein complex. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that promotes formation of CD11b+ DCs comprises one or more mRNAs encoding one or more of: IRF4, RBJ, MgI or Mtg16. In some embodiments, the expression of one or more of IRF4, RBJ, MgI or Mtg16 is increased by about any one of: 5%, 10%, 20%, 30%, 40%, 50%, 60%, 70%, 80%, 90%, 95%, 99%, or 100%. In some embodiments, the expression of one or more of IRF4, RBJ, MgI or Mtg16 is increased by about any one of: 2-fold, 3-fold, 5-fold, 10-fold, 50-fold, 100 fold, 500-fold, 1000-fold, or more. In some embodiments, CD11b+ DC formation from a monocyte or monocyte-dendritic progenitor cell comprising the agent is increased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, CD11b+ DC formation from a monocyte or monocyte-dendritic progenitor cell comprising the agent is increased by about any one of: 2-fold, 3-fold, 5-fold, 10-fold, 50-fold, 100 fold, 500-fold, 1000-fold or more compared to respective monocyte or monocyte-dendritic progenitor cell that does not comprise the agent.

In certain aspects, there is provided a method for modulating an immune response in an individual, comprising: administering to the individual a modified monocyte or monocyte-dendritic progenitor cell, wherein the modified monocyte or monocyte-dendritic progenitor cell is prepared by a process comprising: a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that inhibits formation of pDCs and classical DCs to pass into the monocyte or monocyte-dendritic progenitor cell; and b) incubating the perturbed input monocyte or monocyte-dendritic progenitor cell with the agent that inhibits formation of pDCs and classical DCs for a sufficient time to allow the agent to enter the perturbed input monocyte or monocyte-dendritic progenitor cell, thereby generating the modified monocyte or monocyte-dendritic progenitor cell. In some embodiments, the agent that inhibits formation of pDCs and classical DCs downregulates expression of STAT3 and/or Xbp1. In further embodiments, the agent that downregulates expression of STAT3 and/or Xbp1 is a nucleic acid, a protein, a peptide, a nucleic acid-protein complex or a small molecule. In some embodiments, the nucleic acid is a DNA, an mRNA, an siRNA, an shRNA or an miRNA. In some embodiments, the nucleic acid-protein complex is a gene-editing complex with or without an ssODN for homologous recombination. In some embodiments, the agent that inhibits formation of pDCs and classical DCs from a monocyte or monocyte-dendritic progenitor cell comprises one or more Cas9-gRNA RNP complexes targeting STAT3 and/or Xbp1. In some embodiments, the expression of STAT3 and/or Xbp1 is decreased by about any one of: 5%, 10%, 20%, 30%, 40%, 50%, 60%, 70%, 80%, 90%, 95%, 99%, or 100%. In some embodiments, the expression of STAT3 and/or Xbp1 is decreased by about any one of: 2-fold, 3-fold, 5-fold, 10-fold, 50-fold, 100 fold, 500-fold, or 1000-fold. In some embodiments, formation of pDCs and classical DCs from a monocyte or monocyte-dendritic progenitor cell comprising the agent is decreased by about any one of: 5%, 10%, 20%, 30%, 40%, 50%, 60%, 70%, 80%, 90%, 95%, 99%, or 100% compared to respective monocyte or monocyte-dendritic progenitor cell that does not comprise the agent. In some embodiments, formation of pDCs and classical DCs from a monocyte or monocyte-dendritic progenitor cell comprising the agent is decreased by about any one of: 2-fold, 3-fold, 5-fold, 10-fold, 50-fold, 100 fold, 500-fold, or 1000-fold compared to respective monocyte or monocyte-dendritic progenitor cell that does not comprise the agent.

In some embodiments, according to any of the methods for modulating an immune response in an individual described herein, the modified antigen presenting cell comprises two or more agents that enhance the viability and/or function of the antigen presenting cell is delivered to the antigen presenting cell. In further embodiments, according to the modified antigen presenting cells described above, the two or more agents that enhance the viability and/or function of the antigen presenting cell are chosen from one or more of a tumor homing agent, an anti-apoptotic agent, a T cell activating agent, an antigen processing agent, an immune activity modulating agent, a homing receptor, or an agent that downregulates T cell inhibition.

In some embodiments, according to any of the methods for modulating an immune response in an individual described herein, the agent that enhances the viability and/or function of the antigen presenting cell is an agent that alters cell fate or cell phenotype. In some embodiments, the agent that alters cell fate or phenotype is a somatic cell reprogramming factor. In some embodiments, the agent that alters cell fate or phenotype is a dedifferentiation factor. In some embodiments, the agent that alters cell fate or phenotype is a trans-differentiation factor. In some embodiments, the agent that alters cell phenotype is a differentiation factor. In further embodiments, the agent that alters cell fate or phenotype is one or more of OCT4, SOX2, C-MYC, KLF-4, NANOG, LIN28 or LIN28B. In some embodiments, the agent that alters cell fate or phenotype is one or more of T-bet, GATA3. In some embodiments, the agent that alters cell fate or phenotype is one or more of EOMES, RUNX1, ERG, LCOR, HOXA5, or HOXA9. In some embodiments, the agent that alters cell fate or phenotype is one or more of GM-CSF, M-CSF, or RANKL. In some embodiments, the agent that alters cell fate or cell phenotype comprises one or more mRNAs encoding one or more of: OCT4, SOX2, C-MYC, KLF-4, NANOG, LIN28, LIN28B, T-bet, GATA3, EOMES, RUNX1, ERG, LCOR, HOXA5, HOXA9, GM-CSF, M-CSF, or RANKL. In some embodiments, the expression of one or more of OCT4, SOX2, C-MYC, KLF-4, NANOG, LIN28, LIN28B, T-bet, GATA3, EOMES, RUNX1, ERG, LCOR, HOXA5, HOXA9, GM-CSF, M-CSF, or RANKL is increased by about any one of: 5%, 10%, 20%, 30%, 40%, 50%, 60%, 70%, 80%, 90%, 95%, 99%, or 100%. In some embodiments, the expression of one or more of OCT4, SOX2, C-MYC, KLF-4, NANOG, LIN28, LIN28B, T-bet, GATA3, EOMES, RUNX1, ERG, LCOR, HOXA5, HOXA9, GM-CSF, M-CSF, or RANKL is increased by about any one of: 2-fold, 3-fold, 5-fold, 10-fold, 50-fold, 100 fold, 500-fold, 1000-fold, or more.

In certain aspects, there is provided a method for modulating an immune response in an individual, comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances the viability and/or function of the antigen presenting cell to pass into the antigen presenting cell; and b) incubating the perturbed input antigen presenting cell with the agent that enhances the viability and/or function of the antigen presenting cell for a sufficient time to allow the agent to enter the perturbed input antigen presenting cell, thereby generating an enhanced antigen presenting cell; and c) administering the modified antigen presenting cell to the individual. In some embodiments, the concentration of the agent that enhances the viability and/or function of the antigen presenting cell incubated with the perturbed input antigen presenting cell is between about 1 pM-10 mM. In some embodiments, the agent that enhances the viability and/or function of the antigen presenting cell is encapsulated in a nanoparticle.

In some embodiments, according to any of the methods for modulating an immune response in an individual described herein, the modified antigen presenting cell further comprises an antigen. In some embodiments, the antigen is delivered before, at the same time, or after the agent that enhances the viability and/or function of the antigen presenting cell is delivered to the cell. In some embodiments, the antigen is delivered to the antigen presenting cell by a method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the antigen to pass into the antigen presenting cell; and b) incubating the perturbed input antigen presenting cell with the antigen for a sufficient time to allow the antigen to enter the perturbed input antigen presenting cell.

In some embodiments, according to any of the methods for modulating an immune response in an individual described herein, the modified antigen presenting cell further comprises an adjuvant. In some embodiments, the adjuvant is delivered before, at the same time, or after the antigen is delivered to the cell and/or before, at the same time, or after the agent that enhances the viability and/or function of the antigen presenting cell is delivered to the cell. In some embodiments, the adjuvant is delivered to the antigen presenting cell by a method comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the adjuvant to pass into the antigen presenting cell; and b) incubating the perturbed input antigen presenting cell with the adjuvant for a sufficient time to allow the adjuvant to enter the perturbed input antigen presenting cell.

In some embodiments, according to any of the methods for modulating an immune response in an individual described herein, the method comprises administering a modified antigen presenting cell and an adjuvant. In some embodiments, the adjuvant is administered concurrently or simultaneously with the modified antigen presenting cell. In some embodiments, the adjuvant and the modified antigen presenting cell are administered sequentially. In some embodiments, the adjuvant is administered prior to administration of the modified antigen presenting cell. In some embodiments, the adjuvant is administered following administration of the modified antigen presenting cell. In some embodiments, the adjuvant is administered systemically, e.g., intravenously. In some embodiments, the adjuvant is administered locally, e.g., intratumorally. In some embodiments, the adjuvant is not contained in a cell, e.g., the adjuvant is free in solution. In some embodiments, the adjuvant is contained in a cell, such as an antigen presenting cell. In some embodiments, the adjuvant is delivered into the antigen presenting cell according to any of the methods of intracellular delivery described herein. In some embodiments, the modified antigen presenting cell comprising the agent that enhances the viability and/or function of the antigen presenting cell is prepared by a process comprising the steps of a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for an agent that enhances the viability and/or function of the antigen presenting cell to pass into the antigen presenting cell; and b) incubating the perturbed input antigen presenting cell with the agent that enhances the viability and/or function of the antigen presenting cell for a sufficient time to allow the agent to enter the perturbed input antigen presenting cell, thereby generating an enhanced antigen presenting cell. In some embodiments, the concentration of the agent that enhances the viability and/or function of the antigen presenting cell incubated with the perturbed input antigen presenting cell is between about 1 pM-10 mM. In some embodiments, the agent that enhances the viability and/or function of the antigen presenting cell is encapsulated in a nanoparticle. In some embodiments, the modified antigen presenting cell further comprises an antigen and/or an adjuvant. Thus in some embodiments, the antigen and/or the adjuvant are delivered to the antigen presenting cell by a method comprising: a) passing a cell suspension comprising the antigen presenting cell through a cell-deforming constriction, wherein a diameter of the constriction is a function of a diameter of the input antigen presenting cell in the suspension, thereby causing perturbations of the input antigen presenting cell large enough for the antigen and/or adjuvant to pass into the antigen presenting cell; and b) incubating the perturbed input antigen presenting cell with the antigen and/or the adjuvant for a sufficient time to allow the adjuvant to enter the perturbed input antigen presenting cell, thereby generating an antigen presenting cell comprising the antigen and/or adjuvant. In some embodiments, the adjuvant contained in the modified antigen presenting cell and the adjuvant of step b) are the same compound. In some embodiments, the adjuvant contained in the modified antigen presenting cell and the adjuvant of step b) are different compounds.

In certain aspects, there is provided a method for modulating an immune response in an individual, comprising: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances the viability and/or function of the antigen presenting cell to pass into the antigen presenting cell; and b) incubating the perturbed input antigen presenting cell with the agent that enhances the viability and/or function of the antigen presenting cell for a sufficient time to allow the agent to enter the perturbed input antigen presenting cell, thereby generating a modified antigen presenting cell, such as an enhanced antigen presenting cell; c) administering the modified antigen presenting cell to the individual; and d) administering an adjuvant to the individual. In some embodiments, the adjuvant is administered concurrently or simultaneously with the modified antigen presenting cell. In some embodiments, the adjuvant and the modified antigen presenting cell are administered sequentially. In some embodiments, the adjuvant is administered prior to administration of the modified antigen presenting cell. In some embodiments, the adjuvant is administered following administration of the modified antigen presenting cell. In some embodiments, the adjuvant is administered systemically, e.g., intravenously. In some embodiments, the adjuvant is administered locally, e.g., intratumorally. In some embodiments, the adjuvant is not contained in a cell, e.g., the adjuvant is free in solution. In some embodiments, the adjuvant is contained in a cell, such as an antigen presenting cell. In some embodiments, the adjuvant is delivered into the antigen presenting cell according to any of the methods of intracellular delivery described herein. In some embodiments, the concentration of the adjuvant incubated with the perturbed input antigen presenting cell is between about 1 pM-10 mM. In some embodiments, the adjuvant is encapsulated in a nanoparticle.

In some embodiments, according to any of the methods for modulating an immune response in an individual described herein, the immune response is enhanced. In some embodiments, the enhanced immune response is directed towards the antigen.

In some embodiments, according to any of the methods for modulating an immune response in an individual described herein, the method employs a cell-deforming constriction through which an input antigen presenting cell is passed. In some embodiments, the diameter of the constriction is less than the diameter of the input antigen presenting cell. In some embodiments, the diameter of the constriction is about 20% to about 99% of the diameter of the input antigen presenting cell. In some embodiments, the diameter of the constriction is about 20% to about 60% of the diameter of the input antigen presenting cell. In some embodiments, the cell-deforming constriction is contained in a microfluidic channel, such as any of the microfluidic channels described herein. The microfluidic channel may be contained in any of the microfluidic devices described herein, such as described in the section titled Microfluidic Devices below. Thus, in some embodiments, according to any of the modified antigen presenting cell described herein, the modified antigen presenting cells are prepared by a process employing a microfluidic channel including a cell-deforming constriction through which an input antigen presenting cell is passed, the process comprises passing the input antigen presenting cell through a microfluidic channel including a cell-deforming constriction contained in any of the microfluidic systems described herein. In some embodiments, a deforming force is applied to the input antigen presenting cell as it passes through the constriction, thereby causing the perturbations of the input anteing presenting cell.

In some embodiments, according to any of the methods for modulating an immune response in an individual described herein, the antigen is present in multiple compartments of the modified antigen presenting cell. In some embodiments, the antigen is present in the cytosol and/or a vesicle of the modified antigen presenting cell. In some embodiments, the vesicle is an endosome. In some embodiments, the antigen or an immunogenic epitope contained therein is bound to the surface of the modified antigen presenting cell. In some embodiment, the antigen presenting cell is a PBMC. In some embodiments, the antigen presenting cell is a mixed population of cells. In some embodiments, the antigen presenting cell is in a mixed population of cells, wherein the mixed population of cells is a population of PBMCs. In some embodiments, the PBMC includes one or more of a T cell, a B cell, an NK cells or, a monocyte, a macrophage or a dendritic cell. In some embodiments, the modified antigen presenting cell further comprises an adjuvant.

In some embodiments, according to any of the methods for modulating an immune response in an individual described herein, the adjuvant is present in multiple compartments of the modified antigen presenting cell. In some embodiments, the adjuvant is present in the cytosol and/or a vesicle of the modified antigen presenting cell. In some embodiments, the vesicle is an endosome. In some embodiments, the adjuvant contained therein is bound to the surface of the modified antigen presenting cell. In some embodiment, the antigen presenting cell is a PBMC. In some embodiments, the antigen presenting cell is a mixed population of cells. In some embodiments, the antigen presenting cell is in a mixed population of cells, wherein the mixed population of cells is a population of PBMCs. In some embodiments, the PBMC includes one or more of a T cell, a B cell, an NK cells or, a monocyte, a macrophage or a dendritic cell. In some embodiments, the modified antigen presenting cell further comprises an antigen. In some embodiments, the antigen and/or the adjuvant are present in the cytosol and/or a vesicle of the antigen presenting cell.

In some embodiments, according to any of the methods for modulating an immune response in an individual described herein, the method employs a modified antigen presenting cell comprising an adjuvant. In some embodiments, the adjuvant is a CpG oligodeoxynucleotide (ODN), IFN-α, STING agonists, RIG-I agonists, poly I:C, imiquimod, and/or resiquimod. In some embodiments, the adjuvant is a CpG ODN. In some embodiments, the adjuvant is a CpG ODN. In some embodiments, the CpG ODN is no greater than about 50 (such as no greater than about any of 45, 40, 35, 30, 25, 20, or fewer) nucleotides in length. In some embodiments, the CpG ODN is a Class A CpG ODN, a Class B CpG ODN, or a Class C CpG ODN. In some embodiments, the CpG ODN comprises the nucleotide sequences as disclosed in US provisional application U.S. 62/641,987. In some embodiments, the modified antigen presenting cell comprises a plurality of different CpG ODNs. For example, in some embodiments, the modified antigen presenting cell comprises a plurality of different CpG ODNs selected from among Class A, Class B, and Class C CpG ODNs.

In some embodiments, according to any of the methods for modulating an immune response in an individual described herein, the antigen is a disease-associated antigen. In further embodiments, the antigen is a tumor antigen. In some embodiments, the antigen is derived from a lysate. In some embodiments, the lysate is derived from a biopsy of an individual. In some embodiments, the lysate is derived from a biopsy of an individual being infected by a pathogen, such as a bacteria or a virus. In some embodiments, the lysate is derived from a biopsy of an individual bearing tumors (i.e. tumor biopsy lysates). Thus in some embodiments, the lysate is a tumor lysate.

In some embodiments, according to any of the methods for modulating an immune response in an individual described herein, the method employs a modified antigen presenting cell further comprising an antigen. In some embodiments, according to any of the methods for modulating an immune response in an individual described herein, the method employs a modified antigen presenting cell comprising an antigen comprising an immunogenic epitope. In some embodiments, the immunogenic epitope is derived from a disease-associated antigen. In some embodiments, the immunogenic epitope is derived from peptides or mRNA isolated from a diseased cell. In some embodiments, the immunogenic epitope is derived from a protein ectopically expressed or overexpressed in a disease cell. In some embodiments, the immunogenic epitope is derived from a neoantigen, e.g., a cancer-associated neoantigen. In some embodiments, the immunogenic epitope comprises a neoepitope, e.g., a cancer-associated neoepitope. In some embodiments, the immunogenic epitope is derived from a non-self antigen. In some embodiments, the immunogenic epitope is derived from a mutated or otherwise altered self antigen. In some embodiments, the immunogenic epitope is derived from a tumor antigen, viral antigen, bacterial antigen, or fungal antigen. In some embodiments, the antigen comprises a plurality of immunogenic epitopes. In some embodiments, some of the plurality of immunogenic epitopes are derived from the same source. For example, in some embodiments, some of the plurality of immunogenic epitopes are derived from the same viral antigen. In some embodiments, all of the plurality of immunogenic epitopes are derived from the same source. In some embodiments, none of the plurality of immunogenic epitopes are derived from the same source. In some embodiments, the modified antigen presenting cell comprises a plurality of different antigens.

In some embodiments, according to any of the methods for modulating an immune response in an individual described herein, the modified antigen presenting cell further comprises an antigen. In some embodiments, the antigen comprises an immunogenic epitope, the antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the immunogenic peptide epitope fused to the N-terminal flanking polypeptide and/or the C-terminal flanking polypeptide is a non-naturally occurring sequence. In some embodiments, the N-terminal and/or C-terminal flanking polypeptides are derived from an immunogenic synthetic long peptide (SLP). In some embodiments, the N-terminal and/or C-terminal flanking polypeptides are derived from a disease-associated immunogenic SLP.

In some embodiments, according to any of the methods for modulating an immune response in an individual described herein employing a modified antigen presenting cell further comprising an antigen, the antigen is capable of being processed into an MHC class I-restricted peptide and/or an MHC class II-restricted peptide. In some embodiments, the antigen is capable of being processed into an MHC class I-restricted peptide. In some embodiments, the antigen is capable of being processed into an MHC class II-restricted peptide. In some embodiments, the antigen comprises a plurality of immunogenic epitopes, and is capable of being processed into an MHC class I-restricted peptide and an MHC class II-restricted peptide. In some embodiments, some of the plurality of immunogenic epitopes are derived from the same source. In some embodiments, all of the plurality of immunogenic epitopes are derived from the same source. In some embodiments, none of the plurality of immunogenic epitopes are derived from the same source.

In some embodiments, according to any of the methods for modulating an immune response in an individual described herein employing a modified antigen presenting cell, the modified antigen presenting cell comprises a plurality of antigens that comprise a plurality of immunogenic epitopes. In some embodiments, none of the plurality of immunogenic epitopes decreases an immune response in the individual to any of the other immunogenic epitopes.

In some embodiments, according to any of the methods for modulating an immune response in an individual described herein, the modified antigen presenting cell comprises an agent that enhances the viability and/or function of the modified antigen presenting cell. In some embodiments, the modified antigen presenting cell further comprises an antigen and/or an adjuvant. In some embodiments, the modified antigen presenting cell comprises the agent that enhances the viability and/or function of the modified antigen presenting cell at a concentration between about 1 pM and about 10 mM. In some embodiments, the modified antigen presenting cell comprises the antigen at a concentration between about 1 pM and about 10 mM. In some embodiments, the modified antigen presenting cell comprises the adjuvant at a concentration between about 1 pM and about 10 mM. In some embodiments, the modified antigen presenting cell comprises the agent that enhances the viability and/or function of the modified antigen presenting cell at a concentration between about 0.1 μM and about 10 mM. In some embodiments, the modified antigen presenting cell comprises the antigen at a concentration between about 0.1 μM and about 10 mM. In some embodiments, the modified antigen presenting cell comprises the agent that enhances the viability and/or function of the modified antigen presenting cell at a concentration between about 0.1 μM and about 10 mM. For example, in some embodiments, the concentration of the agent that enhances the viability and/or function of the modified antigen presenting cell in the modified antigen presenting cell is any of less than about 1 pM, about 10 pM, about 100 pM, about 1 nM, about 10 nM, about 100 nM, about 1 about 10 about 100 about 1 mM or about 10 mM. In some embodiments, the concentration of the agent that enhances the viability and/or function of the modified antigen presenting cell in the modified antigen presenting cell is greater than about 10 mM. In some embodiments, the concentration of adjuvant in the modified antigen presenting cell is any of less than about 1 pM, about 10 pM, about 100 pM, about 1 nM, about 10 nM, about 100 nM, about 1 about 10 about 100 about 1 mM or about 10 mM. In some embodiments, the concentration of adjuvant in the modified antigen presenting cell is greater than about 10 mM. In some embodiments, the concentration of antigen in the modified antigen presenting cell is any of less than about 1 pM, about 10 pM, about 100 pM, about 1 nM, about 10 nM, about 100 nM, about 1 about 10 about 100 about 1 mM or about 10 mM. In some embodiments, the concentration of antigen in the modified antigen presenting cell is greater than about 10 mM. In some embodiments, the concentration of the agent that enhances the viability and/or function of the modified antigen presenting cell in the modified antigen presenting cell is any of between about 1 pM and about 10 pM, between about 10 pM and about 100 pM, between about 100 pM and about 1 nM, between about 1 nM and about 10 nM, between about 10 nM and about 100 nM, between about 100 nM and about 1 between about 1 μM and about 10 between about 10 μM and about 100 between about 100 μM and about 1 mM, or between 1 mM and about 10 mM.

In some embodiments, according to any of the method for modulating an immune response in an individual described herein, the molar ratio of the agent that enhances the viability and/or function of the modified antigen presenting cell to antigen in the modified antigen presenting cell is any of between about 10000:1 to about 1:10000. For example, in some embodiments, the molar ratio of the agent that enhances the viability and/or function of the modified antigen presenting cell to antigen in the modified antigen presenting cell is about any of 10000:1, about 1000:1, about 100:1, about 10:1, about 1:1, about 1:10, about 1:100, about 1:1000, or about 1:10000. In some embodiments, the molar ratio of the agent that enhances the viability and/or function of the modified antigen presenting cell to antigen in the modified antigen presenting cell is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the molar ratio of the agent that enhances the viability and/or function of the modified antigen presenting cell to adjuvant in the modified antigen presenting cell is any of between about 10000:1 to about 1:10000. For example, in some embodiments, the molar ratio of the agent to adjuvant in the modified antigen presenting cell is about any of 10000:1, about 1000:1, about 100:1, about 10:1, about 1:1, about 1:10, about 1:100, about 1:1000, or about 1:10000. In some embodiments, the molar ratio of the agent that enhances the viability and/or function of the modified antigen presenting cell to adjuvant in the modified antigen presenting cell is any of between about 10000:1 and about 1000:1, between about 1000:1 and about 100:1, between about 100:1 and about 10:1, between about 10:1 and about 1:1, between about 1:1 and about 1:10, between about 1:10 and about 1:100, between about 1:100 and about 1:1000, between about 1:1000 and about 1:10000. In some embodiments, the modified antigen presenting cell comprises a complex comprising: a) the agent that enhances the viability and/or function of the antigen presenting cell; b) the agent that enhances the viability and/or function of the antigen presenting cell and at least another agent that enhances the viability and/or function of the antigen presenting cell, c) the agent that enhances the viability and/or function of the antigen presenting cell and at least one antigen, d) the agent that enhances the viability and/or function of the antigen presenting cell and at least one adjuvant, and/or e) the agent that enhances the viability and/or function of the antigen presenting cell, at least one antigen and at least one adjuvant.

In some embodiments, according to any of the methods for modulating an immune response in an individual described herein employing a modified antigen presenting cell, the modified antigen presenting cell further comprises an additional agent that enhances the viability and/or function of the modified antigen presenting cell as compared to a corresponding modified antigen presenting cell that does not comprise the additional agent. In some embodiments, the additional agent is a stabilizing agent or a co-factor. In some embodiments, the agent is albumin. In some embodiments, the albumin is mouse, bovine, or human albumin. In some embodiments, the additional agent is a divalent metal cation, glucose, ATP, potassium, glycerol, trehalose, D-sucrose, PEG1500, L-arginine, L-glutamine, or EDTA.

In some embodiments, according to any of the methods for modulating an immune response in an individual described herein employing a modified antigen presenting cell, the modified antigen presenting cell comprises a further modification. In some embodiments, the modified antigen presenting cell comprises a further modification to modulate MHC class I expression. In some embodiments, the modified antigen presenting cell comprises a further modification to decrease MEW class I expression. In some embodiments, the modified antigen presenting cell comprises a further modification to increase MHC class I expression. In some embodiments, the modified T cell comprises a further modification to modulate MEW class II expression. In some embodiments, the modified antigen presenting cell comprises a further modification to decrease MEW class II expression. In some embodiments, the modified antigen presenting cell comprises a further modification to increase MHC class II expression. In some embodiments, an innate immune response mounted in an individual in response to administration, in an allogeneic context, of the modified antigen presenting cells is reduced compared to an innate immune response mounted in an individual in response to administration, in an allogeneic context, of corresponding modified antigen presenting cells that do not comprise the further modification. In some embodiments, the circulating half-life and/or in vivo persistence of the modified antigen presenting cells in an individual to which they were administered is increased compared to the circulating half-life and/or in vivo persistence of corresponding modified T cells that do not comprise the further modification in an individual to which they were administered.

In some embodiments, according to any of the methods for modulating an immune response in an individual employing a modified antigen presenting cell described herein, the method comprises administering the modified antigen presenting cell to the individual. In some embodiments, the modified antigen presenting cell is allogeneic to the individual. In some embodiments, the modified antigen presenting cell is autologous to the individual. In some embodiments, the individual is pre-conditioned to modulate inflammation and/or an immune response. In some embodiments, the individual is pre-conditioned to decrease inflammation and/or an immune response. In some embodiments, the individual is pre-conditioned to increase inflammation and/or an immune response. In some embodiments, administration of the modified antigen presenting cell to the individual results in activation and/or expansion of cytotoxic T lymphocytes (CTLs) specific for the antigen. In some embodiments, administration of the modified antigen presenting cell to the individual results in activation and/or expansion of helper T (Th) cells specific for the antigen. In some embodiments, the amount of the modified antigen presenting cell administered to the individual is between about 1×10⁶ and about 1×10¹² cells. In some embodiments, the amount of the modified antigen presenting cell administered to the individual is less than about any of 1×10⁶, 1×10⁷, 1×10⁸, 1×10⁹, 1×10¹⁰, 1×10¹¹ and about 1×10¹² cells. In some embodiments, the amount of the modified antigen presenting cell administered to the individual is between about any of 1×10⁶ and 1×10⁷, 1×10⁷ and 1×10⁸, 1×10⁸ and 1×10⁹, 1×10⁹ and 1×10¹⁰, 1×10¹⁰ and 1×10¹¹ and 1×10¹¹ and 1×10¹² cells. In some embodiments, the method comprises multiple administrations of the modified antigen presenting cell. In some embodiments, the method comprises any of about 2, 3, 4, 5, 6, 7, 8, 9, 10, or more than about 10 administrations. In some embodiments, the time interval between two successive administrations of the modified antigen presenting cell is between about 1 day and about 1 month. In some embodiments, the administration is daily, every 2 days, every 3 days, every 4 days, every 5 days, every 6 days, weekly, biweekly, or monthly. In some embodiments, successive administrations are given for up to one year or more.

In some embodiment according to any one of the methods described herein, the antigen presenting cell is isolated from the same individual. In some embodiments, the antigen presenting cell is autologous to the individual. In some embodiments, the antigen presenting cell is isolated from a second individual. In some embodiments, the antigen presenting cell is allogeneic to the individual. In some embodiments according to any one of the methods described herein, the modified antigen presenting cell is administered locally. In some embodiments, the modified antigen presenting cell is administered intratumorally or intranodally. In some embodiments according to any one of the methods described herein, the modified antigen presenting cell is administered systemically. In some embodiments, the modified antigen presenting cell is administered intravenously, intraarterially, subcutaneously, intramuscularly, or intraperitoneally.

In some embodiments, according to any of the methods for modulating an immune response in an individual employing a modified antigen presenting cell described herein, the method further comprises administering to the individual a second adjuvant. In some embodiments, the second adjuvant is administered systemically, e.g., intravenously. In some embodiments, the second adjuvant is administered locally, e.g., intratumorally. In some embodiments, the second adjuvant is not contained in a cell, e.g., the second adjuvant is free in solution. In some embodiments, the second adjuvant is IFN-α or a CpG ODN. In some embodiments, the adjuvant contained in the modified antigen presenting cell and the second adjuvant are the same compound. For example, in the embodiments, the modified antigen presenting cell comprises a CpG ODN, and the second adjuvant is also the CpG ODN. In some embodiments, the adjuvant contained in the modified antigen presenting cell and the second adjuvant are different compounds. For example, in some embodiments, the modified antigen presenting cell comprises a CpG ODN, and the second adjuvant is IFN-α. In some embodiments, the modified antigen presenting cell and the second adjuvant are administered concurrently or simultaneously. In some embodiments, the modified antigen presenting cell and the second adjuvant are administered sequentially. In some embodiments, the modified antigen presenting cell is administered prior to administering the second adjuvant. In some embodiments, the modified antigen presenting cell is administered following administration of the second adjuvant.

In some embodiments, according to any of the methods for modulating an immune response in an individual employing a modified antigen presenting cell described herein, the method further comprises administering an immune checkpoint inhibitor to the individual. In some embodiments, the modified antigen presenting cell and the immune checkpoint inhibitor are administered to the individual concurrently. In some embodiments, the modified antigen presenting cell and the immune checkpoint inhibitor are administered to the individual simultaneously. In some embodiments, the modified antigen presenting cell and the immune checkpoint inhibitor are administered to the individual sequentially. In some embodiments, the modified antigen presenting cell is administered to the individual following administration of the immune checkpoint inhibitor to the individual. In some embodiments, the modified antigen presenting cell is administered to the individual prior to administration of the immune checkpoint inhibitor to the individual. In some embodiments, the immune checkpoint inhibitor is targeted to any one of PD-1, PD-L1, CTLA-4, TIM-3, LAG3, VISTA, TIM1, B7-H4 (VTCN1) and BTLA. In some embodiments, the agent that enhances the viability and/or function of the modified antigen presenting cell is the same or similar as the immune checkpoint inhibitor further administered to the individual. For example, in some embodiments, the modified antigen presenting cell comprises an agent that inhibits PD-1, and the immune checkpoint inhibitor further administered also inhibits PD-1. In some embodiments, the agent that enhances the viability and/or function of the modified antigen presenting cell is not the same as the immune checkpoint inhibitor further administered to the individual. For example, in some embodiments, the modified antigen presenting cell comprises an agent that inhibits PD-1, and the immune checkpoint inhibitor further administered inhibits CTLA-4.

In certain aspects, there is provided a method for modulating an immune response in an individual, comprising: administering to the individual a modified antigen presenting cell associated with an agent that enhances the viability and/or function of the modified antigen presenting cell, wherein the modified antigen presenting cell is prepared by a process comprising the steps of: a) incubating an input antigen presenting cell with i) an agent that enhances the viability and/or function of the modified antigen presenting cell, ii) an agent that enhances the viability and/or function of the modified antigen presenting cell and an antigen, iii) an agent that enhances the viability and/or function of the modified antigen presenting cell and an adjuvant, or iv) an agent that enhances the viability and/or function of the modified antigen presenting cell, an antigen and an adjuvant, for a sufficient time to allow the agent that enhances the viability and/or function of the modified antigen presenting cell, the antigen and/or the adjuvant to associate with the cell surface of the input antigen presenting cell, thereby generating a modified antigen presenting cell; and b) administering the modified antigen presenting cell to the individual.

In certain aspects, there is provided a method for modulating an immune response in an individual, comprising: administering to the individual a modified antigen presenting cell associated with an agent that enhances the viability and/or function of the modified antigen presenting cell, wherein the modified antigen presenting cell is prepared by a process comprising the steps of: a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the agent that enhances the viability and/or function of the antigen presenting cell, the antigen and the adjuvant to pass through to form a perturbed input antigen presenting cell; and b) incubating the perturbed input antigen presenting cell with the agent that enhances the viability and/or function of the antigen presenting cell, the agent that enhances the viability and/or function of the antigen presenting cell, the antigen and the adjuvant for a sufficient time to allow the antigen and the adjuvant to enter the perturbed input antigen presenting cell; thereby generating the modified antigen presenting cell comprising the agent that enhances the viability and/or function of the antigen presenting cell, the antigen and the adjuvant. In some embodiments, the concentration of the agent that enhances the viability and/or function of the antigen presenting cell incubated with the perturbed input antigen presenting cell is between about 1 pM-10 mM, the concentration of the antigen incubated with the perturbed input antigen presenting cell is between about 1 pM-10 mM and the concentration of the adjuvant incubated with the perturbed input antigen presenting cell is between about 1 pM-10 mM. In some embodiments, the concentration of the agent that enhances the viability and/or function of the antigen presenting cell incubated with the perturbed input antigen presenting cell is between about 0.1 μM-10 mM, the concentration of the antigen incubated with the perturbed input antigen presenting cell is between about 0.1 μM-10 mM and the concentration of the adjuvant incubated with the perturbed input antigen presenting cell is between about 0.1 μM-10 mM. In some embodiments, the ratio of the agent to the antigen incubated with the perturbed input antigen presenting cell is between about 10000:1 to about 1:10000. In some embodiments, the ratio of the agent to the adjuvant incubated with the perturbed input antigen presenting cell is between about 10000:1 to about 1:10000. In some embodiments, the ratio of the antigen to the adjuvant incubated with the perturbed input antigen presenting cell is between about 10000:1 to about 1:10000.

In some embodiments, according to any of the method for modulating an immune response in an individual described herein, wherein the modified antigen presenting cell comprises an agent that enhances the viability and/or function of the modified antigen presenting cell, the input antigen presenting cell is a peripheral blood mononuclear cell (PBMC). In some embodiments, the antigen presenting cell is a mixed population of cells. In some embodiments, the antigen presenting cell is in a mixed population of cells, wherein the mixed population of cells is a population of PBMCs. In some embodiments, the PBMC is a T cell, a B cell, an NK cells, a monocyte, a macrophage and/or a dendritic cell.

In some embodiments, according to any of the method for modulating an immune response in an individual described herein employing a modified PBMC, the PBMC is engineered to present an antigen. In some embodiments, the agent enhances tumor homing of the antigen presenting cell. In some embodiments, the agent is an anti-apoptotic agent. In some embodiments, the agent enhances T-cell activation. In some embodiments, the agent enhances antigen processing. In some embodiments, the agent enhances antigen processing and loading into MHC-1. In some embodiments, the agent modulates immune activity. In some embodiments, the agent is a homing receptor. In some embodiments, the agent downregulates T cell inhibition.

In some embodiments, according to any of the methods for modulating an immune response in an individual employing a modified monocyte, or monocyte-dendritic progenitor or DC herein, the modified monocyte, or monocyte-dendritic progenitor or DC further comprises an antigen. In some embodiments, the antigen is delivered before, at the same time, or after the agent that promotes or inhibits DC formation is delivered to the cell. In some embodiments, the antigen is delivered to the monocyte, or monocyte-dendritic progenitor or DC by a method comprising: a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the antigen to pass into the monocyte, or monocyte-dendritic progenitor or DC; and b) incubating the perturbed input monocyte, or monocyte-dendritic progenitor or DC with the antigen for a sufficient time to allow the antigen to enter the perturbed input monocyte, or monocyte-dendritic progenitor or DC.

In some embodiments, according to any of the methods for modulating an immune response in an individual employing a modified monocyte, or monocyte-dendritic progenitor or DC herein, the modified monocyte, or monocyte-dendritic progenitor or DC further comprises an adjuvant. In some embodiments, the adjuvant is delivered before, at the same time, or after the antigen is delivered to the cell and/or before, at the same time, or after the agent that promotes or inhibits DC formation of the monocyte, or monocyte-dendritic progenitor or DC is delivered to the cell. In some embodiments, the adjuvant is delivered to the monocyte, or monocyte-dendritic progenitor or DC by a method comprising: a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the adjuvant to pass into the monocyte, or monocyte-dendritic progenitor or DC; and b) incubating the perturbed input monocyte, or monocyte-dendritic progenitor or DC with the adjuvant for a sufficient time to allow the adjuvant to enter the perturbed input monocyte, or monocyte-dendritic progenitor or DC.

Therefore in some embodiments, according to any of the methods for modulating an immune response in an individual employing a modified monocyte, or monocyte-dendritic progenitor or DC herein, the modified monocyte, or monocyte-dendritic progenitor or DC further comprises an antigen and/or an adjuvant. In some embodiments, the antigen is exogenous to the modified monocyte, or monocyte-dendritic progenitor or DC and comprises an immunogenic epitope, and the adjuvant is present intracellularly. Exogenous antigens are one or more antigens from a source outside the monocyte, or monocyte-dendritic progenitor or DC introduced into a cell to be modified. Exogenous antigens can include antigens that may be present in the monocyte, or monocyte-dendritic progenitor or DC (i.e. also present from an endogenous source), either before or after introduction of the exogenous antigen, and as such can thus be produced by the monocyte, or monocyte-dendritic progenitor or DC (e.g., encoded by the genome of the monocyte, or monocyte-dendritic progenitor or DC). For example, in some embodiments, the modified monocyte, or monocyte-dendritic progenitor or DC further comprises two pools of an antigen, a first pool comprising an endogenous source of the antigen, and a second pool comprising an exogenous source of the antigen produced outside of and introduced into the monocyte, or monocyte-dendritic progenitor or DC to be modified. In some embodiments, the antigen is ectopically expressed or overexpressed in a disease cell in an individual, and the modified monocyte, or monocyte-dendritic progenitor or DC is derived from the individual and comprises an exogenous source of the antigen, or an immunogenic epitope contained therein, produced outside of and introduced into the monocyte, or monocyte-dendritic progenitor or DC to be modified. In some embodiments, the antigen is a neoantigen (e.g., an altered-self protein or portion thereof) comprising a neoepitope, and the modified monocyte, or monocyte-dendritic progenitor or DC comprises an exogenous source of the antigen, or a fragment thereof comprising the neoepitope, produced outside of and introduced into the monocyte, or monocyte-dendritic progenitor or DC to be modified. In some embodiments, the adjuvant is exogenous to the modified monocyte, or monocyte-dendritic progenitor or DC. In some embodiments, the antigen and/or the adjuvant are present in multiple compartments of the modified monocyte, or monocyte-dendritic progenitor or DC. In some embodiments, the antigen and/or adjuvant are present in the cytosol and/or a vesicle of the modified monocyte, or monocyte-dendritic progenitor or DC. In some embodiments, the vesicle is an endosome. In some embodiments, the antigen or immunogenic epitope, and/or the adjuvant is bound to the surface of the modified monocyte, or monocyte-dendritic progenitor or DC.

In some embodiments, according to any of the methods for modulating an immune response in an individual employing a modified monocyte, or monocyte-dendritic progenitor or DC further comprising an antigen herein, the antigen is present in multiple compartments of the modified monocyte, or monocyte-dendritic progenitor or DC. In some embodiments, the antigen is present in the cytosol and/or a vesicle of the modified monocyte, or monocyte-dendritic progenitor or DC. In some embodiments, the vesicle is an endosome. In some embodiments, the antigen is bound to the surface of the modified monocyte, or monocyte-dendritic progenitor or DC. In some embodiments, the antigen or an immunogenic epitope contained therein is bound to the surface of the modified monocyte, or monocyte-dendritic progenitor or DC. In some embodiments, the antigen and/or the adjuvant are present in the cytosol and/or a vesicle of the monocyte, or monocyte-dendritic progenitor or DC.

In some embodiments, according to any of the methods for modulating an immune response in an individual employing a modified monocyte, or monocyte-dendritic progenitor or DC further comprising an adjuvant herein, the adjuvant is present in multiple compartments of the modified monocyte, or monocyte-dendritic progenitor or DC. In some embodiments, the adjuvant is present in the cytosol and/or a vesicle of the modified monocyte, or monocyte-dendritic progenitor or DC. In some embodiments, the vesicle is an endosome. In some embodiments, the adjuvant is bound to the surface of the modified monocyte, or monocyte-dendritic progenitor or DC. In some embodiments, the modified monocyte, or monocyte-dendritic progenitor or DC further comprises an antigen. In some embodiments, the antigen and/or the adjuvant are present in the cytosol and/or a vesicle of the monocyte, or monocyte-dendritic progenitor or DC.

In some embodiments, according to any of the methods for modulating an immune response in an individual employing a modified monocyte, or monocyte-dendritic progenitor or DC herein, the modified monocyte, or monocyte-dendritic progenitor or DC further comprises an adjuvant. In some embodiments, the adjuvant is a CpG oligodeoxynucleotide (ODN), IFN-α, STING agonists, RIG-I agonists, poly I:C, imiquimod, and/or resiquimod. In some embodiments, the adjuvant is a CpG ODN. In some embodiments, the CpG ODN is no greater than about 50 (such as no greater than about any of 45, 40, 35, 30, 25, 20, or fewer) nucleotides in length. In some embodiments, the CpG ODN is a Class A CpG ODN, a Class B CpG ODN, or a Class C CpG ODN. In some embodiments, the CpG ODN comprises the nucleotide sequences as disclosed in US provisional application U.S. 62/641,987. In some embodiments, the modified monocyte, or monocyte-dendritic progenitor or DC comprises a plurality of different CpG ODNs. For example, in some embodiments, the modified monocyte, or monocyte-dendritic progenitor or DC comprises a plurality of different CpG ODNs selected from among Class A, Class B, and Class C CpG ODNs.

In some embodiments, according to any of the methods for modulating an immune response in an individual employing a modified monocyte, or monocyte-dendritic progenitor or DC further comprising an antigen herein, the antigen is a disease-associated antigen. In further embodiments, the antigen is a tumor antigen. In some embodiments, the antigen is derived from a lysate. In some embodiments, the lysate is derived from a biopsy of an individual. In some embodiments, the lysate is derived from a biopsy of an individual being infected by a pathogen, such as a bacteria or a virus. In some embodiments, the lysate is derived from a biopsy of an individual bearing tumors (i.e. tumor biopsy lysates). Thus in some embodiments, the lysate is a tumor lysate.

In some embodiments, according to any of the methods for modulating an immune response in an individual employing a modified monocyte, or monocyte-dendritic progenitor or DC herein, the modified monocyte, or monocyte-dendritic progenitor or DC comprises an antigen comprising an immunogenic epitope. In some embodiments, the immunogenic epitope is derived from a disease-associated antigen. In some embodiments, the immunogenic epitope is derived from peptides or mRNA isolated from a diseased cell. In some embodiments, the immunogenic epitope is derived from a protein ectopically expressed or overexpressed in a diseased cell. In some embodiments, the immunogenic epitope is derived from a neoantigen, e.g., a cancer-associated neoantigen. In some embodiments, the immunogenic epitope comprises a neoepitope, e.g., a cancer-associated neoepitope. In some embodiments, the immunogenic epitope is derived from a non-self antigen. In some embodiments, the immunogenic epitope is derived from a mutated or otherwise altered self antigen. In some embodiments, the immunogenic epitope is derived from a tumor antigen, viral antigen, bacterial antigen, or fungal antigen. In some embodiments, the antigen comprises an immunogenic epitope fused to heterologous peptide sequences. In some embodiments, the antigen comprises a plurality of immunogenic epitopes. In some embodiments, some of the plurality of immunogenic epitopes are derived from the same source. For example, in some embodiments, some of the plurality of immunogenic epitopes are derived from the same viral antigen. In some embodiments, all of the plurality of immunogenic epitopes are derived from the same source. In some embodiments, none of the plurality of immunogenic epitopes are derived from the same source. In some embodiments, the modified monocyte, or monocyte-dendritic progenitor or DC comprises a plurality of different antigens.

In some embodiments, according to any of the methods for modulating an immune response in an individual employing a modified monocyte, or monocyte-dendritic progenitor or DC herein, the modified monocyte, or monocyte-dendritic progenitor or DC further comprises an antigen, wherein the antigen comprises an immunogenic epitope. In some embodiments, the antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the immunogenic peptide epitope fused to the N-terminal flanking polypeptide and/or the C-terminal flanking polypeptide is a non-naturally occurring sequence. In some embodiments, the N-terminal and/or C-terminal flanking polypeptides are derived from an immunogenic synthetic long peptide (SLP). In some embodiments, the N-terminal and/or C-terminal flanking polypeptides are derived from a disease-associated immunogenic SLP.

In some embodiments, according to any of the methods for modulating an immune response in an individual employing a modified monocyte, or monocyte-dendritic progenitor or DC herein, the modified monocyte, or monocyte-dendritic progenitor or DC further comprises an antigen, wherein the antigen is capable of being processed into an MHC class I-restricted peptide and/or an MHC class II-restricted peptide. In some embodiments, the antigen is capable of being processed into an MHC class I-restricted peptide. In some embodiments, the antigen is capable of being processed into an MHC class II-restricted peptide. In some embodiments, the antigen comprises a plurality of immunogenic epitopes, and is capable of being processed into an MHC class I-restricted peptide and an MHC class II-restricted peptide. In some embodiments, some of the plurality of immunogenic epitopes are derived from the same source. In some embodiments, all of the plurality of immunogenic epitopes are derived from the same source. In some embodiments, none of the plurality of immunogenic epitopes are derived from the same source.

In some embodiments, according to any of the methods for modulating an immune response in an individual employing a modified monocyte, or monocyte-dendritic progenitor or DC herein, the modified monocyte, or monocyte-dendritic progenitor or DC comprises a plurality of antigens that comprise a plurality of immunogenic epitopes. In some embodiments, following administration to an individual of the modified monocyte, or monocyte-dendritic progenitor or DC comprising the plurality of antigens that comprise the plurality of immunogenic epitopes, none of the plurality of immunogenic epitopes decreases an immune response in the individual to any of the other immunogenic epitopes.

In some embodiments, according to any of the methods for modulating an immune response in an individual employing a modified monocyte, or monocyte-dendritic progenitor or DC herein, the modified monocyte, or monocyte-dendritic progenitor or DC herein is prepared by a method that comprises a process employing a cell-deforming constriction through which an input monocyte, or monocyte-dendritic progenitor or DC is passed. In some embodiments, the diameter of the constriction is less than the diameter of the input monocyte, or monocyte-dendritic progenitor or DC. In some embodiments, the diameter of the constriction is about 20% to about 99% of the diameter of the input monocyte, or monocyte-dendritic progenitor or DC. In some embodiments, the diameter of the constriction is about 20% to about 60% of the diameter of the input monocyte, or monocyte-dendritic progenitor or DC. In some embodiments, the cell-deforming constriction is contained in a microfluidic channel, such as any of the microfluidic channels described herein. The microfluidic channel may be contained in any of the microfluidic devices described herein, such as described in the section titled Microfluidic Devices below. Thus, in some embodiments, according to any of the methods described herein prepared by a process employing a microfluidic channel including a cell-deforming constriction through which an input monocyte, or monocyte-dendritic progenitor or DC is passed, the process comprises passing the input monocyte, or monocyte-dendritic progenitor or DC through a microfluidic channel including a cell-deforming constriction contained in any of the microfluidic systems described herein. In some embodiments, a deforming force is applied to the input monocyte, or monocyte-dendritic progenitor or DC as it passes through the constriction, thereby causing the perturbations of the input monocyte, or monocyte-dendritic progenitor or DC.

### Antigens

In some embodiments, the invention employs delivery of antigens to antigen presenting cells to modulate an immune response, wherein the antigen is delivered to an antigen presenting cell by any of the methods described herein. In some embodiments, the antigen presenting cell comprises one or more agents that enhance viability or function of antigen presenting cell. In some embodiments, the antigen is a single antigen. In some embodiments, the antigen is a mixture of antigens. An antigen is a substance that stimulates a specific immune response, such as a cell or antibody-mediated immune response. Antigens bind to receptors expressed by immune cells, such as T cell receptors (TCRs), which are specific to a particular antigen. Antigen-receptor binding subsequently triggers intracellular signaling pathways that lead to downstream immune effector pathways, such as cell activation, cytokine production, cell migration, cytotoxic factor secretion, and antibody production.

In some embodiments, the antigen is a polypeptide antigen. In some embodiments, the antigen is a disease-associated antigen. In some embodiments, antigens are derived from foreign sources, such as bacteria, fungi, viruses, or allergens. In some embodiments, antigens are derived from internal sources, such as self-proteins (i.e. self-antigens) or a portion of a self-protein. In some embodiments, the antigen is a mutated or otherwise altered self-antigen. In some embodiments, the antigen is a tumor antigen. In some embodiments, the antigen is in a cell lysate. Self-antigens are antigens present on or in an organism's own cells. Self-antigens do not normally stimulate an immune response, but may in the context of autoimmune diseases, such as Type I Diabetes or Rheumatoid Arthritis, or when overexpressed or expressed aberrantly/ectopically.

In some embodiments, the antigen is associated with a virus. In some embodiments, the antigen is a viral antigen. Exemplary viral antigens include HPV antigen, SARS-CoV antigens, and influenza antigens.

In some embodiments, the antigen is associated with a microorganism; for example, a bacterium. In some embodiments, the modulated immune response comprises an increased pathogenic immune response to the microorganism; for example, a bacterium.

In certain aspects, the invention employs methods for further delivering an antigen into an antigen presenting cell comprising an agent that enhances the viability and/or function of the modified antigen presenting cell, the method comprising passing a cell suspension comprising the antigen presenting cell through a constriction, wherein said constriction deforms the antigen presenting cell, thereby causing a perturbation of the cell such that the antigen enters the cell, wherein said cell suspension is contacted with the antigen. In some embodiments, the antigen is delivered to the antigen presenting cell in vitro, ex vivo, or in vivo. In some embodiments, the antigen is delivered to the antigen presenting cell before, at the same time, or after the agent that enhances the viability and/or function of the modified antigen presenting cell is delivered to the cell.

In some embodiments, the antigen to deliver is purified. In some embodiments, the antigen is at least about 60% by weight (dry weight) the antigen of interest. In some embodiments, the purified antigen is at least about 75%, 90%, or 99% the antigen of interest. In some embodiments, the purified antigen is at least about 90%, 91%, 92%, 93%, 94%, 95%, 98%, 99%, or 100% (w/w) the antigen of interest. Purity is determined by any known methods, including, without limitation, column chromatography, thin layer chromatography (TLC), high-performance liquid chromatography (HPLC), nuclear magnetic resonance (NMR) spectroscopy, mass spectrometry, or SDS-PAGE gel electrophoresis. Purified DNA or RNA is defined as DNA or RNA that is free of exogenous nucleic acids, carbohydrates, and lipids.

### Adjuvants

Adjuvants can be used to boost an immune cell response (e.g. T cell response), such as an immune response to an antigen. Multiple adjuvants can also be used to enhance an immune response, and can be used in conjunction with antigens, for example to enhance an antigen-specific immune response as compared to the immune response to the antigens alone. In some embodiments, the invention employs delivery of adjuvants to enhance an immune response, wherein the adjuvant is delivered to an antigen presenting cell by any of the methods described herein. In some embodiments, the adjuvant enhances an immune response to an antigen. In some embodiments, the adjuvant promotes immunogenic presentation of the antigen by an antigen-presenting cell. In some embodiments, the adjuvant is introduced simultaneously with the antigen. In some embodiments, the adjuvant and antigen are introduced sequentially. In some embodiments, the adjuvant is introduced prior to introduction of the antigen. In some embodiments, the adjuvant is introduced following introduction of the antigen. In some embodiments, the adjuvant alters antigen presenting cell homing (e.g., antigen presenting cell homing to a target tissue, such as a tumor) as compared to antigen presenting cell homing in the absence of the adjuvant. In some embodiments, the adjuvant increases antigen presenting cell proliferation as compared to antigen presenting cell proliferation in the absence of the adjuvant.

In certain aspects, the invention employs methods for generating a modified antigen presenting cell further comprising an antigen, wherein the input antigen presenting cell is passed through a constriction, wherein said constriction deforms the input antigen presenting cell thereby causing a perturbation of the cell such that an agent that enhances the viability and/or function of the antigen presenting cell and the antigen to enter the input antigen presenting cell, thereby generating an enhanced antigen presenting cell further comprising the antigen. In some embodiments, the input antigen presenting cell is engineered to present the delivered antigen.

In certain aspects, the invention employs methods for further delivering an adjuvant into an antigen presenting cell comprising an agent that enhances the viability and/or function of the modified antigen presenting cell, the method comprising passing a cell suspension comprising the antigen presenting cell through a constriction, wherein said constriction deforms the antigen presenting cell, thereby causing a perturbation of the antigen presenting cell such that the adjuvant enters the cell, wherein said cell suspension is contacted with the adjuvant. In some embodiments, the adjuvant is delivered into the antigen presenting cell in vitro, ex vivo, or in vivo. In some embodiments, the antigen is delivered to the antigen presenting cell before, at the same time, or after the agent that enhances the viability and/or function of the modified antigen presenting cell is delivered to the cell.

### Microfluidic Systems and Components Thereof

### Microfluidic Channels to Provide Cell-Deforming Constrictions

In some embodiments, the invention provides methods for modulating an immune response by passing a cell suspension comprising an antigen presenting cell through a constriction, wherein the constriction deforms the antigen presenting cell thereby causing a perturbation of the an antigen presenting cell such that an agent that enhances the viability and/or function of the antigen presenting cell enters the antigen presenting cell, wherein the constriction is contained within a microfluidic channel. In some embodiments, multiple constrictions can be placed in parallel and/or in series within the microfluidic channel. Exemplary microfluidic channels containing cell-deforming constrictions for use in the methods disclosed herein are described in WO2013059343. Exemplary surfaces having pores for use in the methods disclosed herein are described in WO2017041050.

In some embodiments, the microfluidic channel includes a lumen and is configured such that PBMC suspended in a buffer can pass through, wherein the microfluidic channel includes a constriction. The microfluidic channel can be made of any one of a number of materials, including silicon, metal (e.g., stainless steel), plastic (e.g., polystyrene), ceramics, glass, crystalline substrates, amorphous substrates, or polymers (e.g., Poly-methyl methacrylate (PMMA), PDMS, Cyclic Olefin Copolymer (COC), etc.). Fabrication of the microfluidic channel can be performed by any method known in the art, including dry etching, wet etching, photolithography, injection molding, laser ablation, or SU-8 masks.

In some embodiments, the constriction within the microfluidic channel includes an entrance portion, a centerpoint, and an exit portion. In some embodiments, the length, depth, and width of the constriction within the microfluidic channel can vary. In some embodiments, the diameter of the constriction within the microfluidic channel is a function of the diameter of the antigen presenting cell. In some embodiments, the diameter of the constriction within the microfluidic channel is about 20%, to about 99% of the diameter of the antigen presenting cell. In some embodiments, the constriction size is about 20%, about 30%, about 40%, about 50%, about 60%, about 70%, about 80%, about 90%, or about 99% of the antigen presenting cell diameter. In some embodiments, the constriction size is about 20%, about 30%, about 40%, about 50%, about 60%, about 70%, about 80%, about 90%, or about 99% of the minimum cross-sectional distance of the antigen presenting cell. In some embodiments, the channel comprises a constriction width of between about 2 μm and about 10 μm or any width or range of widths therebetween. For example, the constriction width can be any one of about 2 μm, about 3 μm, about 4 μm, about 5 μm, about 6 μm, or about 7 μm. In some embodiments, the channel comprises a constriction length of about 10 μm and a constriction width of about 4 μm. The cross-section of the channel, the entrance portion, the centerpoint, and the exit portion can also vary. For example, the cross-sections can be circular, elliptical, an elongated slit, square, hexagonal, or triangular in shape. The entrance portion defines a constriction angle, wherein the constriction angle is optimized to reduce clogging of the channel and optimized for enhanced delivery of a compound into the antigen presenting cell. The angle of the exit portion can vary as well. For example, the angle of the exit portion is configured to reduce the likelihood of turbulence that can result in non-laminar flow. In some embodiments, the walls of the entrance portion and/or the exit portion are linear. In other embodiments, the walls of the entrance portion and/or the exit portion are curved.

In some embodiments according to any one of the methods, compositions or modified antigen presenting cells described herein, the diameter of the constriction is about 2 μm to about 15 μm. In some embodiments, the diameter of the constriction is about 3 μm to about 10 μm. In some embodiments, the diameter of the constriction is about 3 μm to about 6 μm. In some embodiments, the diameter of the constriction is about 3.5 μm to about 4.5 μm. In some embodiments, the diameter of the constriction is about 4 μm to about 10 μm. In some embodiments, the diameter of the constriction is about 4.2 μm to about 6 μm. In some embodiments, the diameter of the constriction is about 4.2 μm to about 4.8 μm. In some embodiments, the diameter of the constriction is any one of about 2 μm to about 14 μm, about 4 μm to about 12 μm-about 6 μm to about 9 μm, about 4 μm to about 6 μm, about 4 μm to about 5 μm-about 3.5 μm to about 7 μm, about 3.5 μm to about 6.3 μm-about 3.5 μm to about 5.6 μm-about 3.5 μm to about 4.9 μm-about 4.2 μm to about 6.3 μm, about 4.2 μm to about 5.6 μm, or about 4.2 μm to about 4.9 μm. In some embodiments, the diameter of the constriction is any one of about 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm. In some embodiments, the diameter of the constriction is any one of about 4.0 μm, 4.1 μm, 4.2 μm, 4.3 μm, 4.4 μm, 4.5 μm, 4.6 μm, 4.7 μm, 4.8 μm, 4.9 μm, or 5.0 μm In some embodiments, the diameter of the constriction is about 4.5 μm. In some embodiments, the diameter of the constriction is any one of about 3.0 μm, 3.1 μm, 3.2 μm, 3.3 μm, 3.4 μm, 3.5 μm, 3.6 μm, 3.7 μm, 3.8 μm, 3.9 μm, or 4.0 μm In some embodiments, the diameter of the constriction is about 3.5 μm. In some embodiments, the diameter of the constriction is about 4.0 μm.

In some embodiments according to any one of the methods or modified antigen presenting cells described herein, the constriction comprises a length and the length of the constriction is about 2 μm to about 50 μm. In some embodiments, the diameter of the constriction is about 5 μm to about 40 μm. In some embodiments, the length of the constriction is about 10 μm to about 30 μm. In some embodiments, the length of the constriction is about 8 μm to about 12 μm. In some embodiments, the length of the constriction is about 13 μm to about 15 μm. In some embodiments, the length of the constriction is about 18 μm to about 22 μm. In some embodiments, the length of the constriction is about 23 μm to about 27 μm. In some embodiments, the length of the constriction is about 28 μm to about 32 μm. In some embodiments, the length of the constriction is any one of about 2 μm, 5 μm, 8 μm, 9 μm, 10 μm, 11 μm, 12 μm, 13 μm, 14 μm, 15 μm, 16 μm, 17 μm, 18 μm, 19 μm, 20 μm, 22 μm, 24 μm, 25 μm, 26 μm, 28 μm, or 30 μm. In some embodiments, the length of the constriction is about 10 μm. In some embodiments, the length of the constriction is about 20 μm. In some embodiments, the length of the constriction is about 30 μm.

In some embodiments according to any one of the methods or modified antigen presenting cells described herein, the constriction comprises a depth and the depth of the constriction is about 1 μm to about 200 μm. In some embodiments, the depth of the constriction is about 20 μm to about 120 μm. In some embodiments, the depth of the constriction is about 20 μm to about 80 μm. In some embodiments, the depth of the constriction is about 40 μm to about 60 μm. In some embodiments, the depth of the constriction is about 60 μm to about 80 μm. In some embodiments, the depth of the constriction is about 35 μm to about 45 μm. In some embodiments, the depth of the constriction is about 55 μm to about 65 μm. In some embodiments, the depth of the constriction is about 75 μm to about 85 μm. In some embodiments, the depth of the constriction is any one of about 1 μm, 5 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 175 μm, or 200 μm. In some embodiments, the depth of the constriction is any one of about 40 μm, 45 μm, 50 μm, 55 μm, 60 μm, 65 μm, 70 μm, 75 μm, 80 μm, 90 μm, or 100 μm In some embodiments, the depth of the constriction is about 40 μm. In some embodiments, the depth of the constriction is about 80 μm. In some embodiments, the depth of the constriction is about 60 μm.

In some embodiments, the cross-sectional shape of the constriction is selected from the group consisting of: circular, elliptical, round, square, rectangular, star-shaped, triangular, polygonal, pentagonal, hexagonal, heptagonal, and octagonal. In some embodiments, the cross-sectional shape of the constriction is a slit. In some embodiments, the slit comprises a width of about 3 μm-5 μm and/or a depth of about 20 μm-120 μm. In some embodiments, the slit comprises a width of about 3.5 μm and/or a depth of about 80 μm. In some embodiments, the input antigen presenting cell are passed through multiple constrictions wherein the multiple constrictions are arranged in series and/or in parallel. In some embodiments, the constriction comprises an entrance portion and an exit portion, wherein the entrance portion defines an entrance angle and the entrance angle is between about 0 degree to about 90 degrees. In some embodiments, the entrance angle is between about 20 degrees to about 22 degrees. In some embodiments, the exit portion defines an exit angle and the exit angle is between about 0 degree to about 90 degrees. In some embodiments, the exit angel is between about 20 degrees to about 22 degrees.

In some embodiments, the input antigen presenting cell is passed through the constriction at a flow rate between about 100 mm/sec to about 10 m/sec. In some embodiments, the input antigen presenting cell is passed through the constriction at a flow rate between about 2 m/sec to about 10 m/sec. In some embodiments, the input antigen presenting cell is passed through the constriction at a flow rate between about 0.001 mL/cm²/sec to about 200 L/cm²/sec. In some embodiments, the input antigen presenting cell is passed through the constriction at a flow rate of about 100 L/cm²/sec. In some embodiments, the input antigen presenting cell is passed through the constriction at a temperature ranging from about 0° C. to about 37° C.

In some embodiments according to any one of the methods, compositions or modified antigen presenting cells, monocytes, or monocyte-dendritic progenitor cells described herein, the input antigen presenting cell is passed through the constriction at a temperature ranging from about 0° C. to about 37° C. In some embodiments, the input antigen presenting cell, monocyte or monocyte-dendritic progenitor cell is passed through the constriction at a temperature ranging from about 0° C. to about 10° C. In some embodiments, the input antigen presenting cell, monocyte or monocyte-dendritic progenitor cell is passed through the constriction at a temperature ranging from about 2° C. to about 8° C. In some embodiments, the input antigen presenting cell, monocyte or monocyte-dendritic progenitor cell is passed through the constriction at a temperature ranging from any one of about 2° C. to about 6° C., about 5° C. to about 10° C., about 10° C. to about 15° C., about 15° C. to about 20° C., about 20° C. to about 25° C., about 25° C. to about 30° C., about 30° C. to about 35° C., or about 35° C. to about 37° C. In some embodiments, the input antigen presenting cell, monocyte or monocyte-dendritic progenitor cell is passed through the constriction at a temperature of any one of about 0° C., 1° C., 2° C., 3° C., 4° C., 5° C., 6° C., 7° C., 8° C., 9° C., 10° C., 15° C., 20° C., 25° C., 30° C. or 37° C.

In some embodiments according to any one of the methods, compositions or modified antigen presenting cells, monocytes or monocyte-dendritic progenitor cells described herein, subsequent to passing through the constriction the modified antigen presenting cell, monocyte or monocyte-dendritic progenitor cell is incubated at a temperature of 37° C. for a sufficient time to allow the modified cell to normalize to 37° C. In some embodiments, subsequent to passing through the constriction the modified antigen presenting cell, monocyte or monocyte-dendritic progenitor cell is incubated at a temperature of 25° C. for a sufficient time to allow the modified cell to normalize to 25° C.

In some embodiments according to any one of the methods, compositions or modified antigen presenting cells, monocytes or monocyte-dendritic progenitor cells described herein, the input antigen presenting cell, monocyte or monocyte-dendritic progenitor cell is passed through the constriction at a flow rate between about 100 mm/sec to about 10 m/sec. In some embodiments, the flow rate is between about 100 mm/sec to about 1 cm/sec, about 1 cm/sec to about 10 cm/sec, about 10 cm/sec to about 100 cm/sec, about 100 cm/sec to about 1 m/sec, or between 1 m/sec to about 10 m/sec. In some embodiments, the flow rate is between about 2 m/sec to about 5 m/sec. In some embodiments, the flow rate is between about 0.1 m/sec to about 0.5 m/sec, 0.5 m/sec to about 1 m/sec, about 1 m/sec to about 1.5 m/sec, about 1.5 m/sec to about 2 m/sec, about 2 m/sec to about 2.5 m/sec, about 2.5 m/sec to about 3 m/sec, about 3 m/sec to about 3.5 m/sec, about 3.5 m/sec to about 4 m/sec, about 4 m/sec to about 4.5 m/sec, about 4.5 m/sec to about 5 m/sec, about 5 m/sec to about 6 m/sec, about 6 m/sec to about 7 m/sec, about 7 m/sec to about 8 m/sec, about 8 m/sec to about 9 m/sec, or about 9 m/sec to about 10 m/sec. In some embodiments, the input antigen presenting cell, monocyte or monocyte-dendritic progenitor cell is passed through the constriction at a flow rate of about any one of: 1 m/sec, 2 m/sec, 3 m/sec, 4 m/sec, 5 m/sec, 6 m/sec, 7 m/sec, 8 m/sec, 9 m/sec, or 10 m/sec.

In some embodiments according to any one of the methods, compositions or modified antigen presenting cells, monocytes or monocyte-dendritic progenitor cells described herein, the input antigen presenting cell, monocyte or monocyte-dendritic progenitor cell is passed through the constriction at a flow rate between about 0.001 mL/min to about 200 mL/min or any rate or range of rates therebetween. In some embodiments, the flow rate is between about 0.001 mL/min to about 175 mL/min, about 0.001 mL/min to about 150 mL/min, about 0.001 mL/min to about 125 mL/min, about 0.001 mL/min to about 100 mL/min, about 0.001 mL/min to about 50 mL/min, about 0.001 mL/min to about 25 mL/min, about 0.001 mL/min to about 10 mL/min, about 0.001 mL/min to about 7.5 mL/min, about 0.001 mL/min to about 5.0 mL/min, about 0.001 mL/min to about 2.5 mL/min, about 0.001 mL/min to about 1 mL/min, about 0.001 mL/min to about 0.1 mL/min or about 0.001 mL/min to about 0.01 mL/min. In some embodiments, the flow rate is between about 0.001 mL/min to about 200 mL/min, about 0.01 mL/min to about 200 mL/min, about 0.1 mL/min to about 200 mL/min, about 1 mL/min to about 200 mL/min, about 10 mL/min to about 200 mL/min, about 50 mL/min to about 200 mL/min, about 75 mL/min to about 200 mL/min, about 100 mL/min to about 200 mL/min, about 150 mL/min to about 200 mL/min, about 0.5 mL/min to about 200 mL/min, about 1 mL/min to about 200 mL/min, about 2.5 mL/min to about 200 mL/min, about 5 mL/min to about 200 mL/min, about 7.5 mL/min to about 200 mL/min, about 10 mL/min to about 200 mL/min, about 25 mL/min to about 200 mL/min, or about 175 mL/min to about 200 mL/min. In some embodiments, the input antigen presenting cell, monocyte or monocyte-dendritic progenitor cell is passed through the constriction at a flow rate of about any one of: 1 mL/min, 10 mL/min, 20 mL/min, 30 mL/min, 40 mL/min, 50 mL/min, 60 mL/min, 70 mL/min, 80 mL/min, 90 mL/min, 100 mL/min, 110 mL/min, 120 mL/min, 130 mL/min, 140 mL/min, 150 mL/min, 160 mL/min, 170 mL/min, 180 mL/min, 190 mL/min, or 200 mL/min. In some embodiments, the input antigen presenting cell, monocyte or monocyte-dendritic progenitor cell is passed through the constriction at a flow rate between about 10 mL/min to about 200 mL/min. In some embodiments, input antigen presenting cell, monocyte or monocyte-dendritic progenitor cell is passed through the constriction at a flow rate of about 100 mL/min.

In some embodiments according to any one of the methods, compositions or modified antigen presenting cells, monocytes or monocyte-dendritic progenitor cells described herein, the constriction can have any shape known in the art; e.g. a 3-dimensional shape or a 2-dimensional shape. The 2-dimensional shape, such as the cross-sectional shape, of the constriction can be, without limitation, circular, elliptical, round, square, star-shaped, triangular, polygonal, pentagonal, hexagonal, heptagonal, or octagonal. The 3-dimensional shape of the constriction can be, without limitation, cylindrical, conical, or cuboidal. In some embodiments, the cross-sectional shape of the constriction is a rectangle. In some embodiments, the cross-sectional shape of the constriction is a slit. In some embodiments, the cross-sectional shape of the constriction is a slit comprising a width of about 2 μm to about 10 μm and/or a depth of about 1 μm to about 200 μm. In some embodiments, the cross-sectional shape of the constriction is a slit comprising a width of about 2.5 μm to about 6 μm and/or a depth of about 20 μm to about 120 μm. In some embodiments, the cross-sectional shape of the constriction is a slit comprising a width of about 3 μm to about 5 μm and/or a depth of about 40 μm to about 100 μm. In some embodiments, the cross-sectional shape of the constriction is a slit comprising a width of about 3 μm to about 4 μm and/or a depth of about 40 μm to about 100 μm. In some embodiments, the cross-sectional shape of the constriction is a slit comprising a width of about 3.5 μm to about 4.5 μm and/or a depth of about 40 μm to about 100 μm. In some embodiments, the cross-sectional shape of the constriction is a slit comprising a width of about 3.3 μm to about 3.7 μm and/or a depth of about 20 μm to about 80 μm. In some embodiments, the cross-sectional shape of the constriction is a slit comprising a width of about 3.5 μm and/or a depth of about 80 μm. In some embodiments, the slit comprises a length of about 10 μm to about 30 μm. In some embodiments, the slit comprises a length of about 2 μm to about 50 μm. In some embodiments, the slit comprises a length of any one of about 2 μm to about 5 μm, about 5 μm to about 10 μm, about 10 μm to about 15 μm, about 15 μm to about 20 μm, about 20 μm to about 25 μm, about 25 μm to about 30 μm, about 30 μm to about 35 μm, about 35 μm to about 40 μm, about 40 μm to about 45 μm, or about 45 μm to about 50 μm. In some embodiments, the slit comprises a length of about 10 μm. In some embodiments, the cross-sectional shape of the constriction is a slit comprising a width of about 3 μm to about 5 μm, a length of about 10 μm to about 30 μm and/or a depth of about 20 μm to about 120 μm. In some embodiments, the cross-sectional shape of the constriction is a slit comprising a width of about 3.5 μm, a length of about 30 μm and/or a depth of about 80 μm.

In some embodiments, the constriction comprises an entrance portion and an exit portion. The entrances and exits of the constriction may have a variety of angles. In some embodiments, the constrictions have identical entrance and exit angles. In some embodiments, the constrictions have different entrance and exit angles. The constriction angle can be selected to minimize clogging of the constriction while input antigen presenting cells, monocytes or monocyte-dendritic progenitor cells are passing through. In some embodiments, the flow rate through the surface is between about 100 mm/sec to about 10 m/sec. In some embodiments, the follow rate is between about 2 m/sec to about 5 m/sec. In some embodiments the flow rate through the surface is between about 0.001 mL/min to about 100 mL/min or any rate or range of rates therebetween. In some examples, the angle of the entrance and/or exit portion can be between about 0 and about 90 degrees. In some embodiments, the entrance and/or exit portion can be greater than 90 degrees. In some embodiments, the entrance portion defines an entrance angle and the entrance angle is between about 0 degree to about 90 degrees. In some embodiments, the entrance angle is between any one of about 10 degrees to about 40 degrees, about 12 degrees to about 45 degrees, between about 15 degrees to about 30 degrees. In some embodiments, the entrance angle is between about 20 degrees to about 22 degrees. In some embodiments, the exit portion defines an exit angle and the exit angle is between about 0 degree to about 90 degrees. In some embodiments, the exit angle is between any one of about 10 degrees to about 40 degrees, about 12 degrees to about 45 degrees, between about 15 degrees to about 30 degrees. In some embodiments, the exit angle is between about 20 degrees to about 22 degrees. In some embodiments, the entrance portion defines an entrance angle and the entrance angle is between about 20 degrees to about 22 degrees, and the exit portion defines an exit angle and the exit angle is between about 20 degrees to about 22 degrees.

In some embodiments according to any one of the methods, compositions or modified antigen presenting cells, monocytes or monocyte-dendritic progenitor cells described herein, the constriction edge is smooth, e.g. rounded or curved. A smooth constriction edge has a continuous, flat, and even surface without bumps, ridges, or uneven parts. In some embodiments, the constriction edge is sharp. A sharp constriction edge has a thin edge that is pointed or at an acute angle. In some embodiments, the constriction passage is straight. A straight constriction passage does not contain curves, bends, angles, or other irregularities. In some embodiments, the constriction passage is curved. A curved constriction passage is bent or deviates from a straight line. In some embodiments, the constriction passage has multiple curves, e.g. about 2, 3, 4, 5, 6, 7, 8, 9, 10 or more curves.

In some embodiments according to any one of the methods, compositions or modified antigen presenting cells, monocytes or monocyte-dendritic progenitor cells described herein, the cell suspension comprising the input antigen presenting cell, monocyte or monocyte-dendritic progenitor cell is passed through multiple constrictions, wherein the multiple constrictions are arranged in series and/or in parallel. In some embodiments, the multiple constrictions are arranged in series. In some embodiments, the multiple constrictions are arranged in parallel. In some embodiments, the multiple constrictions are arranged in series and/or in parallel. In some embodiments, the multiple constrictions arranged in series comprise about any one of 2, 3, 4, 5, 6, 7, 8, 9, 10, 50, 75, 100, 500, 1,000 or more constrictions in series. In some embodiments, the multiple constrictions arranged in parallel may comprise about any one of 2, 5, 10, 50, 75, 100, 500, 1,000 or more constrictions in series.

### Surface Having Pores to Provide Cell Deforming Constrictions

In some embodiments, the invention provides methods for modulating an immune response by passing a cell suspension comprising an antigen presenting cell through a constriction, wherein the constriction deforms the antigen presenting cell thereby causing a perturbation of the antigen presenting cell such that agent that enhances the viability and/or function of the antigen presenting cell enters the antigen presenting cell, wherein the constriction is a pore or contained within a pore. In some embodiments, the pore is contained in a surface. Exemplary surfaces having pores for use in the methods disclosed herein are described in WO2017041050.

The surfaces as disclosed herein can be made of any one of a number of materials and take any one of a number of forms. In some embodiments, the surface is a filter. In some embodiments, the surface is a membrane. In some embodiments, the filter is a tangential flow filter. In some embodiments, the surface is a sponge or sponge-like matrix. In some embodiments, the surface is a matrix.

In some embodiments the surface is a tortuous path surface. In some embodiments, the tortuous path surface comprises cellulose acetate. In some embodiments, the surface comprises a material selected from, without limitation, synthetic or natural polymers, polycarbonate, silicon, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, and ceramic.

The surface disclosed herein can have any shape known in the art; e.g. a 3-dimensional shape. The 2-dimensional shape of the surface can be, without limitation, circular, elliptical, round, square, star-shaped, triangular, polygonal, pentagonal, hexagonal, heptagonal, or octagonal. In some embodiments, the surface is round in shape. In some embodiments, the surface 3-dimensional shape is cylindrical, conical, or cuboidal.

The surface can have various cross-sectional widths and thicknesses. In some embodiments, the surface cross-sectional width is between about 1 mm and about 1 m or any cross-sectional width or range of cross-sectional widths therebetween. In some embodiments, the surface has a defined thickness. In some embodiments, the surface thickness is uniform. In some embodiments, the surface thickness is variable. For example, in some embodiments, portions of the surface are thicker or thinner than other portions of the surface. In some embodiments, the surface thickness varies by about 1% to about 90% or any percentage or range of percentages therebetween. In some embodiments, the surface is between about 0.01 μm to about 5 mm thick or any thickness or range of thicknesses therebetween.

In some embodiments, the constriction is a pore or contained within a pore. The cross-sectional width of the pores is related to the type of antigen presenting cell to be treated. In some embodiments, the pore size is a function of the diameter of the antigen presenting cell or cluster of antigen presenting cells to be treated. In some embodiments, the pore size is such that an antigen presenting cell is perturbed upon passing through the pore. In some embodiments, the pore size is less than the diameter of the antigen presenting cell. In some embodiments, the pore size is about 10% to about 99% of the diameter of the antigen presenting cell. In some embodiments, the pore size is about 10%, about 15%, about 20%, about 30%, about 40%, about 50%, about 60%, about 70%, about 80%, about 90%, or about 99% of the antigen presenting cell diameter. Optimal pore size or pore cross-sectional width can vary based upon the application and/or PBMC cell type. In some embodiments, the pore size is about 2 μm to about 14 μm. In some embodiments, the pore size is about 2 μm, about 3 μm, about 4 μm, about 5 μm, about 8 μm, about 10 μm, about 12 μm, or about 14 μm. In some embodiments, the cross-sectional width is about 2 μm to about 14 μm. In some embodiments, the pore cross-sectional is about 2 μm, about 3 μm, about 4 μm, about 5 μm, about 8 μm, about 10 μm, about 12 μm, or about 14 μm.

The entrances and exits of the pore passage may have a variety of angles. The pore angle can be selected to minimize clogging of the pore while antigen presenting cells are passing through. In some embodiments the flow rate through the surface is between about 0.001 mL/cm²/sec to about 100 L/cm²/sec or any rate or range of rates therebetween. For example, the angle of the entrance or exit portion can be between about 0 and about 90 degrees. In some embodiments, the entrance or exit portion can be greater than 90 degrees. In some embodiments, the pores have identical entrance and exit angles. In some embodiments, the pores have different entrance and exit angles. In some embodiments, the pore edge is smooth, e.g. rounded or curved. A smooth pore edge has a continuous, flat, and even surface without bumps, ridges, or uneven parts. In some embodiments, the pore edge is sharp. A sharp pore edge has a thin edge that is pointed or at an acute angle. In some embodiments, the pore passage is straight. A straight pore passage does not contain curves, bends, angles, or other irregularities. In some embodiments, the pore passage is curved. A curved pore passage is bent or deviates from a straight line. In some embodiments, the pore passage has multiple curves, e.g. about 2, 3, 4, 5, 6, 7, 8, 9, 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some embodiments, the cross-section of the pore is round in shape. In some embodiments, the 3-dimensional shape of the pore is cylindrical or conical. In some embodiments, the pore has a fluted entrance and exit shape. In some embodiments, the pore shape is homogenous (i.e. consistent or regular) among pores within a given surface. In some embodiments, the pore shape is heterogeneous (i.e. mixed or varied) among pores within a given surface.

The surfaces described herein can have a range of total pore numbers. In some embodiments, the pores encompass about 10% to about 80% of the total surface area. In some embodiments, the surface contains about 1.0×10⁵ to about 1.0×10³⁰ total pores or any number or range of numbers therebetween. In some embodiments, the surface comprises between about 10 and about 1.0×10¹⁵ pores/mm² surface area.

The pores can be distributed in numerous ways within a given surface. In some embodiments, the pores are distributed in parallel within a given surface. In one such example, the pores are distributed side-by-side in the same direction and are the same distance apart within a given surface. In some embodiments, the pore distribution is ordered or homogeneous. In one such example, the pores are distributed in a regular, systematic pattern or are the same distance apart within a given surface. In some embodiments, the pore distribution is random or heterogeneous. In one such example, the pores are distributed in an irregular, disordered pattern or are different distances apart within a given surface. In some embodiments, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of compounds into different antigen presenting cell types.

In some embodiments, an individual pore has a uniform width dimension (i.e. constant width along the length of the pore passage). In some embodiments, an individual pore has a variable width (i.e. increasing or decreasing width along the length of the pore passage). In some embodiments, pores within a given surface have the same individual pore depths. In some embodiments, pores within a given surface have different individual pore depths. In some embodiments, the pores are immediately adjacent to each other. In some embodiments, the pores are separated from each other by a distance. In some embodiments, the pores are separated from each other by a distance of about 0.001 μm to about 30 mm or any distance or range of distances therebetween.

In some embodiments, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, or transmembrane proteins. In some embodiments, the surface is coated with polyvinylpyrrolidone (PVP). In some embodiments, the material is covalently attached to the surface. In some embodiments, the material is non-covalently attached or adsorbed to the surface. In some embodiments, the surface molecules are released as the antigen presenting cells pass through the pores.

In some embodiments, the surface has modified chemical properties. In some embodiments, the surface is polar. In some embodiments, the surface is hydrophilic. In some embodiments, the surface is non-polar. In some embodiments, the surface is hydrophobic. In some embodiments, the surface is charged. In some embodiments, the surface is positively and/or negatively charged. In some embodiments, the surface can be positively charged in some regions and negatively charged in other regions. In some embodiments, the surface has an overall positive or overall negative charge. In some embodiments, the surface can be any one of smooth, electropolished, rough, or plasma treated. In some embodiments, the surface comprises a zwitterion or dipolar compound. In some embodiments, the surface is plasma treated.

In some embodiments, the surface is contained within a larger module. In some embodiments, the surface is contained within a syringe, such as a plastic or glass syringe. In some embodiments, the surface is contained within a plastic filter holder. In some embodiments, the surface is contained within a pipette tip.

### Cell Perturbations

In some embodiments, the invention provides methods for modulating an immune response by passing a cell suspension comprising an antigen presenting cell through a constriction, wherein the constriction deforms the antigen presenting cell thereby causing a perturbation of the antigen presenting cell such that an agent that enhances the viability and/or function of the antigen presenting cell enters the antigen presenting cell, wherein the perturbation in the antigen presenting cell is a breach in the antigen presenting cell that allows material from outside the antigen presenting cell to move into the antigen presenting cell (e.g., a hole, tear, cavity, aperture, pore, break, gap, perforation). The deformation can be caused by, for example, mechanical strain and/or shear forces. In some embodiments, the perturbation is a perturbation within the antigen presenting cell membrane. In some embodiments, the perturbation is transient. In some embodiments, the antigen presenting cell perturbation lasts from about 1.0×10⁻⁹ seconds to about 2 hours, or any time or range of times therebetween. In some embodiments, the antigen presenting cell perturbation lasts for about 1.0×10⁻⁹ second to about 1 second, about 1 second to about 1 minute, or about 1 minute to about 1 hour. In some embodiments, the antigen presenting cell perturbation lasts for between any one of about 1.0×10⁷ to about 1.0×10⁻³, about 1.0×10⁶ to about 1.0×10⁻², about 1.0×10⁵ to about 1.0×10⁻², about 1.0×10⁴ to about 1.0×10⁻², about 1.0×10³ to about 1.0×10⁻², about 1.0×10² to about 1.0×10⁻², about 1.0×10¹ to about 1.0×10⁻², or about 1.0×10⁰ to about 1.0×10⁻¹ seconds. In some embodiment, the antigen presenting cell perturbation lasts for any one of about 1.0×10⁷ to about 1.0×10⁻¹, about 1.0×10⁶ to about 1.0×10⁻¹, about 1.0×10⁵ to about 1.0×10⁻¹, about 1.0×10⁴ to about 1.0×10⁻¹, about 1.0×10³ to about 1.0×10⁻¹, about 1.0×10² to about 1.0×10⁻¹, or about 1.0×10¹ to about 1.0×10⁻¹ seconds. The antigen presenting cell perturbations (e.g., pores or holes) created by the methods described herein are not formed as a result of assembly of protein subunits to form a multimeric pore structure such as that created by complement or bacterial hemolysins.

As the antigen presenting cell passes through the constriction, the constriction temporarily imparts injury to the antigen presenting cell membrane that allows for passive diffusion of material through the perturbation. In some embodiments, the antigen presenting cell is only deformed for a brief period of time, on the order of 100 μs to minimize the chance of activating apoptotic pathways through cell signaling mechanisms, although other durations are possible (e.g., ranging from nanoseconds to hours). In some embodiments, the antigen presenting cell is deformed for about 1.0×10⁻⁹ seconds to about 2 hours, or any time or range of times therebetween. In some embodiments, the antigen presenting cell is deformed for about 1.0×10⁻⁹ second to about 1 second, about 1 second to about 1 minute, or about 1 minute to about 1 hour. In some embodiments, the antigen presenting cell is deformed for between any one of about 1.0×10⁻⁹ to about 1.0×10⁻¹, about 1.0×10⁻⁹ to about 1.0×10⁻², about 1.0×10⁻⁹ to about 1.0×10⁻³, about 1.0×10⁻⁹ to about 1.0×10⁻⁴, about 1.0×10⁻⁹ to about 1.0×10⁻⁵, about 1.0×10⁻⁹ to about 1.0×10⁻⁶, about 1.0×10⁻⁹ to about 1.0×10⁻⁷, or about 1.0×10⁻⁹ to about 1.0×10⁻⁸ seconds. In some embodiment, the antigen presenting cell is deformed for any one of about 1.0×10⁻⁸ to about 1.0×10⁻¹, about 1.0×10⁻⁷ to about 1.0×10⁻¹, about 1.0×10⁻⁶ to about 1.0×10⁻¹, about 1.0×10⁻⁵ to about 1.0×10⁻¹, about 1.0×10⁻⁴ to about 1.0×10⁻¹, about 1.0×10⁻³ to about 1.0×10⁻¹, or about 1.0×10⁻² to about 1.0×10⁻¹ seconds. In some embodiments, deforming the antigen presenting cell includes deforming the antigen presenting cell for a time ranging from, without limitation, about 1 μs to at least about 750 μs, e.g., at least about 1 μs, 10 μs, 50 μs, 100 μs, 500 μs, or 750 μs.

In some embodiments, the passage of the agent that enhances the viability and/or function of the antigen presenting cell into the antigen presenting cell occurs simultaneously with the antigen presenting cell passing through the constriction and/or the perturbation of the antigen presenting cell. In some embodiments, passage of the compound into the antigen presenting cell occurs after the antigen presenting cell passes through the constriction. In some embodiments, passage of the compound into the antigen presenting cell occurs on the order of minutes after the antigen presenting cell passes through the constriction. In some embodiments, the passage of the compound into the antigen presenting cell occurs from about 1.0×10⁻² seconds to at least about 30 minutes after the antigen presenting cell passes through the constriction. For example, the passage of the compound into the antigen presenting cell occurs from about 1.0×10⁻² seconds to about 1 second, about 1 second to about 1 minute, or about 1 minute to about 30 minutes after the antigen presenting cell passes through the constriction. In some embodiments, the passage of the compound into the antigen presenting cell occurs about 1.0×10⁻² seconds to about 10 minutes, about 1.0×10⁻² seconds to about 5 minutes, about 1.0×10⁻² seconds to about 1 minute, about 1.0×10⁻² seconds to about 30 seconds, about 1.0×10⁻² seconds to about 10 seconds, about 1.0×10⁻² seconds to about 1 second, or about 1.0×10⁻² seconds to about 0.1 second after the antigen presenting cell passes through the constriction. In some embodiments, the passage of the compound into the antigen presenting cell occurs about 1.0×10⁻¹ seconds to about 10 minutes, about 1 second to about 10 minutes, about 10 seconds to about 10 minutes, about 50 seconds to about 10 minutes, about 1 minute to about 10 minutes, or about 5 minutes to about 10 minutes after the antigen presenting cell passes through the constriction. In some embodiments, a perturbation in the antigen presenting cell after it passes through the constriction is corrected within the order of about five minutes after the antigen presenting cell passes through the constriction.

In some embodiments, the cell viability after passing through a constriction is about 5% to about 100%. In some embodiments, the cell viability after passing through the constriction is at least about 5%, 10%, 20%, 30%, 40%, 50%, 60%, 70%, 75%, 80%, 85%, 90%, 95%, or 99%. In some embodiments, the cell viability is measured from about 1.0×10⁻² seconds to at least about 10 days after the antigen presenting cell passes through the constriction. For example, the cell viability is measured from about 1.0×10⁻² seconds to about 1 second, about 1 second to about 1 minute, about 1 minute to about 30 minutes, or about 30 minutes to about 2 hours after the antigen presenting cell passes through the constriction. In some embodiments, the cell viability is measured about 1.0×10⁻² seconds to about 2 hours, about 1.0×10⁻² seconds to about 1 hour, about 1.0×10⁻² seconds to about 30 minutes, about 1.0×10⁻² seconds to about 1 minute, about 1.0×10⁻² seconds to about 30 seconds, about 1.0×10⁻² seconds to about 1 second, or about 1.0×10⁻² seconds to about 0.1 second after the antigen presenting cell passes through the constriction. In some embodiments, the cell viability is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the antigen presenting cell passes through the constriction. In some embodiments, the cell viability is measured about 2 hours to about 5 hours, about 5 hours to about 12 hours, about 12 hours to about 24 hours, or about 24 hours to about 10 days after the antigen presenting cell passes through the constriction.

### Delivery Parameters

A number of parameters may influence the delivery of an agent to an antigen presenting cell for modulating an immune response by the methods described herein. In some embodiments, the cell suspension is contacted with the agent that enhances the viability and/or function of the antigen presenting cell before, concurrently, or after passing through the constriction. The antigen presenting cell may pass through the constriction suspended in a solution that includes the compound to deliver, although the compound can be added to the cell suspension after the antigen presenting cells pass through the constriction. In some embodiments, the compound to be delivered is coated on the constriction.

Examples of parameters that may influence the delivery of the compound into the antigen presenting cell include, but are not limited to, the dimensions of the constriction, the entrance angle of the constriction, the surface properties of the constrictions (e.g., roughness, chemical modification, hydrophilic, hydrophobic, etc.), the operating flow speeds (e.g., cell transit time through the constriction), the antigen presenting cell concentration, the concentration of the compound in the cell suspension, and the amount of time that the antigen presenting cell recovers or incubates after passing through the constrictions can affect the passage of the delivered compound into the antigen presenting cell. Additional parameters influencing the delivery of the compound into the antigen presenting cell can include the velocity of the antigen presenting cell in the constriction, the shear rate in the constriction, the viscosity of the cell suspension, the velocity component that is perpendicular to flow velocity, and time in the constriction. Such parameters can be designed to control delivery of the compound. In some embodiments, the antigen presenting cell concentration ranges from about 10 to at least about 10¹² cells/mL or any concentration or range of concentrations therebetween. In some embodiments, delivery compound concentrations can range from about 10 ng/mL to about 1 g/mL or any concentration or range of concentrations therebetween. In some embodiments, delivery compound concentrations can range from about 1 ng/mL to about 10 g/mL or any concentration or range of concentrations therebetween. In some embodiments, delivery compound concentrations can range from about 1 pM to at least about 2 M or any concentration or range of concentrations therebetween.

The temperature used in the methods of the present disclosure can be adjusted to affect compound delivery and cell viability. In some embodiments, the method is performed between about −5° C. and about 45° C. For example, the methods can be carried out at room temperature (e.g., about 20° C.), physiological temperature (e.g., about 37° C.), higher than physiological temperature (e.g., greater than about 37° C. to 45° C. or more), or reduced temperature (e.g., about −5° C. to about 4° C.), or temperatures between these exemplary temperatures.

Various methods can be utilized to drive the antigen presenting cells through the constrictions. For example, pressure can be applied by a pump on the entrance side (e.g., compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some embodiments, the antigen presenting cells are passed through the constrictions by positive pressure or negative pressure. In some embodiments, the antigen presenting cells are passed through the constrictions by constant pressure or variable pressure. In some embodiments, pressure is applied using a syringe. In some embodiments, the pressure is positive pressure applied using a gas (e.g., from a gas cylinder). In some embodiments, pressure is applied using a pump. In some embodiments, the pump is a peristaltic pump or a diaphragm pump. In some embodiments, pressure is applied using a vacuum. In some embodiments, the antigen presenting cells are passed through the constrictions by g-force. In some embodiments, the antigen presenting cells are passed through the constrictions by centrifugal force. In some embodiments, the antigen presenting cells are passed through the constrictions by capillary pressure.

In some embodiments according to any one of the methods, compositions or modified antigen presenting cells described herein, the input antigen presenting cell is passed through the constriction under a pressure ranging from about 1 psi to about 120 psi. In some embodiments according to any one of the methods described herein, the input antigen presenting cell is passed through the constriction under a pressure ranging from about 30 psi to about 120 psi. In some embodiments, the input antigen presenting cell is passed through the constriction under a pressure ranging from about 45 psi to about 105 psi. In some embodiments, the input antigen presenting cell is passed through the constriction under a pressure ranging from about 60 psi to about 100 psi. In some embodiments, the input antigen presenting cell is passed through the constriction under a pressure of about 90 psi. In some embodiments, the input antigen presenting cell is passed through the constriction under a pressure ranging from about 2 psi to about 10 psi. In some embodiments, the input antigen presenting cell is passed through the constriction under a pressure ranging from about 20 psi to about 200 psi. In some embodiments, the input antigen presenting cell is passed through the constriction under a pressure ranging from about 2 psi to about 10 psi, about 10 psi to about 20 psi, about 20 psi to about 30 psi, about 30 psi to about 40 psi, about 40 psi to about 50 psi, about 50 psi to about 60 psi, about 60 psi to about 70 psi, about 70 psi to about 80 psi, about 80 psi to about 90 psi, about 90 psi to about 100 psi, about 100 psi to about 110 psi, about 110 psi to about 120 psi. In some embodiments, the input antigen presenting cell is passed through the constriction under a pressure of about any one of 2 psi, 5 psi, 10 psi, 15 psi, 20 psi, 25 psi, 30 psi, 35 psi, 40 psi, 45 psi, 50 psi, 55 psi, 60 psi, 65 psi, 70 psi, 75 psi, 80 psi, 85 psi, 90 psi, 95 psi, 100 psi, 105 psi, 110 psi, 115 psi, or 120 psi.

In some embodiments according to any one of the methods, compositions or modified antigen presenting cells described herein, the input antigen presenting cell is passed through the constriction under a pressure ranging from about 130 kPa to about 2000 kPa. In some embodiments, the input antigen presenting cell is passed through the constriction under a pressure ranging from about 200 kPa to about 830 kPa. In some embodiments, the input antigen presenting cell is passed through the constriction under a pressure ranging from about 300 kPa to about 730 kPa. In some embodiments, the antigen presenting cell is passed through the constriction under a pressure ranging from about 415 kPa to about 690 kPa. In some embodiments, the antigen presenting cell is passed through the constriction under a pressure of about 620 kPa. In some embodiments, the input antigen presenting cell is passed through the constriction under a pressure ranging from any one of about 100 kPa to about 150 kPa, about 150 kPa to about 200 kPa, about 200 kPa to about 250 kPa, about 250 kPa to about 300 kPa, 300 kPa to about 350 kPa, about 350 kPa to about 400 kPa, 400 kPa to about 450 kPa, about 450 kPa to about 500 kPa, 500 kPa to about 550 kPa, about 550 kPa to about 600 kPa, 600 kPa to about 650 kPa, about 650 kPa to about 700 kPa, 700 kPa to about 750 kPa, about 750 kPa to about 800 kPa, 800 kPa to about 850 kPa, about 850 kPa to about 900 kPa, 900 kPa to about 950 kPa, about 950 kPa to about 1000 kPa, about 1000 kPa to about 1500 kPa, or about 1500 kPa to about 2000 kPa. In some embodiments, the input antigen presenting cell is passed through the constriction under a pressure of about any one of 200 kPa, 250 kPa, 300 kPa, 350 kPa, 400 kPa, 415 kPa, 450 kPa, 500 kPa, 550 kPa, 600 kPa, 620 kPa, 650 kPa, 700 kPa, 750 kPa, 800 kPa, 850 kPa, 900 kPa, or 1000 kPa.

In some embodiments, fluid flow directs the antigen presenting cells through the constrictions. In some embodiments, the fluid flow is turbulent flow prior to the antigen presenting cells passing through the constriction. Turbulent flow is a fluid flow in which the velocity at a given point varies erratically in magnitude and direction. In some embodiments, the fluid flow through the constriction is laminar flow. Laminar flow involves uninterrupted flow in a fluid near a solid boundary in which the direction of flow at every point remains constant. In some embodiments, the fluid flow is turbulent flow after the antigen presenting cells pass through the constriction. The velocity at which the antigen presenting cells pass through the constrictions can be varied. In some embodiments, the antigen presenting cells pass through the constrictions at a uniform cell speed. In some embodiments, the antigen presenting cells pass through the constrictions at a fluctuating cell speed.

In other embodiments, a combination treatment is used to modulate an immune response by passing a cell suspension comprising an antigen presenting cell through a constriction, wherein the constriction deforms the antigen presenting cell thereby causing a perturbation of the antigen presenting cells such that an agent that enhances the viability and/or function of the modified antigen presenting cell enters the antigen presenting cell, e.g., the methods described herein, followed by exposure to an electric field downstream of the constriction. In some embodiments, the antigen presenting cell is passed through an electric field generated by at least one electrode after passing through the constriction. In some embodiments, the electric field assists in delivery of compounds to a second location inside the antigen presenting cell such as the antigen presenting cell nucleus. For example, the combination of a cell-deforming constriction and an electric field delivers a plasmid encoding a transcription factor into the antigen presenting cell (e.g., the cell nucleus), resulting in the de novo production of a transcription factor. In some embodiments, one or more electrodes are in proximity to the cell-deforming constriction to generate an electric field. In some embodiments, the electric field is between about 0.1 kV/m to about 100 MV/m, or any number or range of numbers therebetween. In some embodiments, an integrated circuit is used to provide an electrical signal to drive the electrodes. In some embodiments, the antigen presenting cells are exposed to the electric field for a pulse width of between about 1 ns to about 1 s and a period of between about 100 ns to about 10 s or any time or range of times therebetween.

### Cell Suspensions for Delivery to Antigen Presenting Cells

The cell suspension may be a mixed or purified population of antigen presenting cells. In some embodiments, the cell suspension is a mixed cell population, such as whole blood. In some embodiments, the cell suspension is a mixed cell population, such as PBMCs. In some embodiments, the cell suspension is a purified cell population, such as a purified population of any one of: T cells, B cells, NK cells, monocytes, macrophages or dendritic cells.

The composition of the cell suspension (e.g., osmolarity, salt concentration, serum content, cell concentration, pH, etc.) can impact delivery of the agent that enhances the viability and/or function of the modified antigen presenting cell. In some embodiments, the suspension comprises whole blood. In some embodiments, the suspension comprises PBMCs. Alternatively, the cell suspension is a mixture of cells in a physiological saline solution or physiological medium other than blood. In some embodiments, the cell suspension comprises an aqueous solution. In some embodiments, the aqueous solution comprises cell culture medium, phosphate buffered saline (PBS), salts, metal ions, sugars, growth factors, animal derived products, bulking materials, surfactants, lubricants, lipids, vitamins, amino acids, proteins, cell cycle inhibitors, and/or an agent that impacts actin polymerization. In some embodiments, the cell culture medium is DMEM, Opti-MEM®, IMDM, RPMI, X-Vivo 10, or X-Vivo 15.

Additionally, solution buffer can include one or more lubricants (Pluronics® or other surfactants) that can be designed, for example, to reduce or eliminate clogging of the constriction or pore and improve cell viability. Exemplary surfactants include, without limitation, poloxamer, polysorbates, sugars or sugar alcohols such as mannitol, sorbitol, animal derived serum, and albumin protein.

In some configurations with certain types of antigen presenting cells, the antigen presenting cells can be incubated in one or more solutions that aid in the delivery of the agent that enhances the viability and/or function of the modified antigen presenting cell to the interior of the antigen presenting cell. In some embodiments, the aqueous solution comprises an agent that impacts actin polymerization. In some embodiments, the agent that impacts actin polymerization is Latrunculin A, Cytochalasin, and/or Colchicine. For example, the antigen presenting cells can be incubated in a depolymerization solution such as Lantrunculin A (0.1 μg/mL) for 1 hour prior to delivery to depolymerize the actin cytoskeleton. As an additional example, the antigen presenting cells can be incubated in 10 μM Colchicine (Sigma) for 2 hours prior to delivery to depolymerize the microtubule network.

In some embodiments, the cell population is enriched prior to use in the disclosed methods. For example, cells are obtained from a bodily fluid, e.g., peripheral blood, and optionally enriched or purified to concentrate antigen presenting cells. Cells may be enriched by any methods known in the art, including without limitation, magnetic cell separation, fluorescent activated cell sorting (FACS), or density gradient centrifugation.

The viscosity of the cell suspension can also impact the methods disclosed herein. In some embodiments, the viscosity of the cell suspension ranges from about 8.9×10⁻⁴ Pa·s to about 4.0×10⁻³ Pa·s or any value or range of values therebetween. In some embodiments, the viscosity ranges between any one of about 8.9×10⁻⁴ Pa·s to about 4.0×10⁻³ Pa·s, about 8.9×10⁻⁴ Pa·s to about 3.0×10⁻³ Pa·s, about 8.9×10⁻⁴ Pa·s to about 2.0×10⁻³ Pa·s, or about 8.9×10⁻³ Pa·s to about 1.0×10⁻³ Pa·s. In some embodiments, the viscosity ranges between any one of about 0.89 cP to about 4.0 cP, about 0.89 cP to about 3.0 cP, about 0.89 cP to about 2.0 cP, or about 0.89 cP to about 1.0 cP. In some embodiments, a shear thinning effect is observed, in which the viscosity of the cell suspension decreases under conditions of shear strain. Viscosity can be measured by any method known in the art, including without limitation, viscometers, such as a glass capillary viscometer, or rheometers. A viscometer measures viscosity under one flow condition, while a rheometer is used to measure viscosities which vary with flow conditions. In some embodiments, the viscosity is measured for a shear thinning solution such as blood. In some embodiments, the viscosity is measured between about −5° C. and about 45° C. For example, the viscosity is measured at room temperature (e.g., about 20° C.), physiological temperature (e.g., about 37° C.), higher than physiological temperature (e.g., greater than about 37° C. to 45° C. or more), reduced temperature (e.g., about −5° C. to about 4° C.), or temperatures between these exemplary temperatures.

### Systems and Kits

In some aspects, the invention provides a system comprising one or more of a constriction, an antigen presenting cell suspension, one or more agents that enhances the viability and/or function of the modified antigen presenting cell according to any of the embodiments described herein, such as for use in any of the methods described herein. In some embodiments, the system further comprises antigens and/or adjuvants. The system can include any embodiment described for the compositions of matter and methods disclosed herein, including those disclosed in the above section titled “Microfluidic systems and components thereof” In some embodiment, the cell-deforming constrictions are sized for delivery to antigen presenting cells. In some embodiments, the delivery parameters, such as operating flow speeds, cell and compound concentration, temperature, velocity of the cell in the constriction, and the composition of the cell suspension (e.g., osmolarity, salt concentration, serum content, cell concentration, pH, etc.) are optimized for maximum response of a compound for modulating an immune response.

Also provided are kits or articles of manufacture for use in modulating an immune response in an individual. In some embodiments, the kit comprises a modified antigen presenting cell comprising one or more agents that enhances the viability and/or function of the modified antigen presenting cell, including any of the modified antigen presenting cells described herein. In some embodiments, the system further comprises an antigen and/or an adjuvant. In some embodiments, the kit comprises one or more of a constriction, an antigen presenting cell suspension, agents that enhance the viability and/or function of the modified antigen presenting cell for use in generating modified antigen presenting cells with enhanced viability and/or function of antigen presenting cells, such as enhanced tumor homing, enhanced viability, enhanced antigen processing and/or loading onto MHC molecules, modulated immune activity, enhanced homing receptors, enhanced T cell activating capability, downregulated T cell inhibition, and altered differentiation for use in modulating an immune response in an individual. In some embodiments, the kits comprise components described herein (e.g. a microfluidic channel or surface containing pores, cell suspensions, and/or compounds) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The invention also provides kits comprising components of the methods described herein and may further comprise instructions for performing said methods to modulate an immune response in an individual and/or instructions for introducing an antigen and/or an adjuvant into an antigen presenting cell. The kits described herein may further include other materials, including buffers, diluents, filters, needles, syringes, and package inserts with instructions for performing any of the methods described herein; e.g., instructions for modulating an immune response in an individual or instructions for modifying an antigen presenting cell to contain an antigen and/or an adjuvant.

### EXEMPLARY EMBODIMENTS

Embodiment 1. A method for enhancing tumor homing of an antigen presenting cell, the method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances tumor homing of the antigen presenting cell to pass into the antigen presenting cell; and

b) incubating the perturbed input antigen presenting cell with the agent that enhances tumor homing of the antigen presenting cell for a sufficient time to allow the agent to enter the perturbed input antigen presenting cell, thereby generating an enhanced antigen presenting cell.

Embodiment 2. The method of embodiment 1, wherein the agent that enhances tumor homing of the antigen presenting cell upregulates expression of one or more of CXCR3, CCR5, VLA-4 or LFA-1.

Embodiment 3. The method of embodiment 2, wherein the agent that upregulates expression of one or more of CXCR3, CCR5, VLA-4 or LFA-1 is a nucleic acid, a protein or a nucleic acid-protein complex.

Embodiment 4. The method of embodiment 3, wherein the nucleic acid is a DNA, an mRNA, an siRNA, an shRNA or an miRNA.

Embodiment 5. The method of embodiment 3, wherein the nucleic acid-protein complex is a gene-editing complex with or without an ssODN for homologous recombination.

Embodiment 6. A method for enhancing the viability and/or function of an antigen presenting cell, the method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an anti-apoptotic agent to pass into the antigen presenting cell; and

b) incubating the perturbed input antigen presenting cell with the anti-apoptotic agent for a sufficient time to allow the agent to enter the perturbed input antigen presenting cell, thereby generating an enhanced antigen presenting cell.

Embodiment 7. The method of embodiment 6, wherein the anti-apoptotic agent upregulates expression of one or more of XIAP, cIAP1/2, survivin, livin, cFLIP, Hsp72, or Hsp90.

Embodiment 8. The method of embodiment 7, wherein the agent that upregulates expression of one or more of XIAP, cIAP1/2, survivin, livin, cFLIP, Hsp72 or Hsp90 is a nucleic acid, a protein or a nucleic acid-protein complex.

Embodiment 9. The method of embodiment 8, wherein the nucleic acid is a DNA, an mRNA, an siRNA, an shRNA or an miRNA.

Embodiment 10. The method of embodiment 8, wherein the nucleic acid-protein complex is a gene-editing complex with or without an ssODN for homologous recombination.

Embodiment 11. A method for enhancing the function of an antigen presenting cell, the method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing to pass into the antigen presenting cell; and

b) incubating the perturbed input antigen presenting cell with the agent that enhances antigen processing for a sufficient time to allow the agent to enter the perturbed input antigen presenting cell, thereby generating an enhanced antigen presenting cell.

Embodiment 12. The method of embodiment 11, wherein the agent that enhances antigen processing upregulates expression of one or more of LMP2, LMP7, MECL-1 or β5t.

Embodiment 13. The method of embodiment 12, wherein the agent that upregulates expression of one or more of LMP2, LMP7, MECL-1 or β5t is a nucleic acid, a protein or a nucleic acid-protein complex.

Embodiment 14. The method of embodiment 13, wherein the nucleic acid is a DNA, an mRNA, an siRNA, an shRNA or an miRNA.

Embodiment 15. The method of embodiment 13, wherein the nucleic acid-protein complex is a gene-editing complex with or without an ssODN for homologous recombination.

Embodiment 16. A method for enhancing the function of an antigen presenting cell, the method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing and/or loading onto MHC molecules to pass into the antigen presenting cell; and

b) incubating the perturbed input antigen presenting cell with the agent that enhances antigen processing and/or loading onto MHC molecules for a sufficient time to allow the agent to enter the perturbed input antigen presenting cell, thereby generating an enhanced antigen presenting cell.

Embodiment 17. The method of embodiment 16, wherein the agent that enhances antigen processing and/or loading onto MHC molecules upregulates expression of one or more of TAP, Tapasin, ERAAP, Calreticulin, Erp57 or PDI.

Embodiment 18. The method of embodiment 17, wherein the agent that upregulates expression of one or more of TAP, Tapasin, ERAAP, Calreticulin, Erp57 or PDI is a nucleic acid, a protein or a nucleic acid-protein complex.

Embodiment 19. The method of embodiment 18, wherein the nucleic acid is a DNA, an mRNA, an siRNA, an shRNA or an miRNA.

Embodiment 20. The method of embodiment 18, wherein the nucleic acid-protein complex is a gene-editing complex with or without an ssODN for homologous recombination.

Embodiment 21. A method for modulating immune activity of an antigen presenting cell, the method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that modulates immune activity to pass into the antigen presenting cell; and

b) incubating the perturbed input antigen presenting cell with the agent that modulates immune activity for a sufficient time to allow the agent to enter the perturbed input antigen presenting cell, thereby generating an enhanced antigen presenting cell.

Embodiment 22. The method of embodiment 21, wherein the agent that modulates immune activity upregulates expression of one or more of type I interferon, type II interferon, or type III interferon.

Embodiment 23. The method of embodiment 22, wherein the agent that upregulates expression of one or more of type I interferon, type II interferon, or type III interferon is a nucleic acid, a protein or a nucleic acid-protein complex.

Embodiment 24. The method of embodiment 23, wherein the nucleic acid is a DNA, an mRNA, an siRNA, an shRNA or an miRNA.

Embodiment 25. The method of embodiment 21, wherein the agent that modulates immune activity downregulates expression of interferon beta.

Embodiment 26. The method of embodiment 25, wherein the agent that downregulates expression of interferon beta is a nucleic acid, a protein, a peptide, a nucleic acid-protein complex or a small molecule.

Embodiment 27. The method of embodiment 23, wherein the nucleic acid is a DNA, an mRNA, an siRNA, an shRNA or an miRNA.

Embodiment 28. The method of embodiment 23, wherein the nucleic acid-protein complex is a gene-editing complex with or without an ssODN for homologous recombination.

Embodiment 29. A method for enhancing the viability of an antigen presenting cell, the method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances viability of the antigen presenting cell to pass into the antigen presenting cell; and

b) incubating the perturbed input antigen presenting cell with the agent that enhances viability of the antigen presenting cell for a sufficient time to allow the agent to enter the perturbed input antigen presenting cell, thereby generating an enhanced antigen presenting cell.

Embodiment 30. The method of embodiment 29, wherein the agent that enhances viability of the antigen presenting cell upregulates expression of a serpin.

Embodiment 31. The method of embodiment 30, wherein the agent that upregulates expression a serpin is a nucleic acid, a protein or a nucleic acid-protein complex.

Embodiment 32. The method of embodiment 31 wherein the nucleic acid is a DNA, an mRNA, an siRNA, an shRNA or an miRNA.

Embodiment 33. The method of embodiment 31, wherein the nucleic acid-protein complex is a gene-editing complex with or without an ssODN for homologous recombination.

Embodiment 34. A method for enhancing the function of an antigen presenting cell, the method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances homing receptors of the antigen presenting cell to pass into the antigen presenting cell; and

b) incubating the perturbed input antigen presenting cell with the agent that enhances homing receptors of the antigen presenting cell for a sufficient time to allow the agent to enter the perturbed input antigen presenting cell, thereby generating an enhanced antigen presenting cell.

Embodiment 35. The method of embodiment 34, wherein the agent that enhances homing receptors of the antigen presenting cell upregulates expression of a CCL2.

Embodiment 36. The method of embodiment 35, wherein the agent that upregulates expression of CCL2 is a nucleic acid, a protein or a nucleic acid-protein complex.

Embodiment 37. The method of embodiment 34, wherein the agent that enhances homing and/or triggers alternative homing upregulates expression of one or more of: CD62L, CCR2, CCR7, CX3CR1, or CXCR5.

Embodiment 38. The method of embodiment 37, wherein the agent that upregulates expression of one or more of: CD62L, CCR2, CCR7, CX3CR1, or CXCR5 comprises one or more of: a nucleic acid, a protein or a nucleic acid-protein complex.

Embodiment 39. The method of embodiment 37 or 38, wherein the agent enhances homing of the enhanced antigen presenting cell to lymph nodes.

Embodiment 40. The method of embodiment 39, wherein the antigen presenting cell is a dendritic cell.

Embodiment 41. The method of any one of embodiments 36 and 38-40, wherein the nucleic acid is a DNA, an mRNA, an siRNA, an shRNA or an miRNA.

Embodiment 42. The method of any one of embodiments 36 and 38-40, wherein the nucleic acid-protein complex is a gene-editing complex with or without an ssODN for homologous recombination.

Embodiment 43. A method for enhancing the viability and/or function of an antigen presenting cell, the method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that activates T cells to pass into the antigen presenting cell; and

b) incubating the perturbed input antigen presenting cell with the agent that activates T cells for a sufficient time to allow the agent to enter the perturbed input antigen presenting cell, thereby generating an enhanced antigen presenting cell.

Embodiment 44. The method of embodiment 43, wherein the agent that activates T cells upregulates expression of one or more of CD27, CD28, CD40, CD122, 4-1BB (CD137), OX40(CD134)/OX40L(CD252), GITR or ICOS.

Embodiment 45. The method of embodiment 44, wherein the agent that upregulates expression of one or more of CD27, CD28, CD40, CD122, 4-1BB (CD137), OX40(CD134)/OX40L(CD252), GITR or ICOS is a nucleic acid, a protein or a nucleic acid-protein complex.

Embodiment 46. The method of embodiment 43, wherein the agent that activates T cells upregulates expression of one or more of CD70, CD80, CD86, CD40L, 4-1BBL (CD137L), OX40L(CD252), GITRL or ICOSL.

Embodiment 47. The method of embodiment 46, wherein the agent that upregulates expression of one or more of CD70, CD80, CD86, CD40L, 4-1BBL (CD137L), OX40L(CD252), GITRL or ICOSL is a nucleic acid, a protein or a nucleic acid-protein complex.

Embodiment 48. The method of embodiment 45 or 47, wherein the nucleic acid is a DNA, an mRNA, an siRNA, an shRNA or an miRNA.

Embodiment 49. The method of embodiment 45 or 47, wherein the nucleic acid-protein complex is a gene-editing complex with or without an ssODN for homologous recombination.

Embodiment 50. A method for enhancing the viability and/or function of an antigen presenting T cell, the method comprising:

a) passing a cell suspension comprising an input antigen presenting T cell through a cell-deforming constriction, wherein a diameter of the constriction is a function of a diameter of the input antigen presenting cell in the suspension, thereby causing perturbations of the input antigen presenting cell large enough for an agent that downregulates T cell inhibition to pass into the antigen presenting cell; and

b) incubating the perturbed input antigen presenting cell with the agent that downregulates T cell inhibition for a sufficient time to allow the agent to enter the perturbed input antigen presenting cell, thereby generating an enhanced antigen presenting T cell.

Embodiment 51. The method of embodiment 50, wherein the agent that downregulates T cell inhibition downregulates expression of one or more of LAG3, VISTA, TIM1, B7-H4 (VTCN1) or BTLA.

Embodiment 52. The method of embodiment 51, wherein the agent that downregulates expression of one or more of LAG3, VISTA, TIM1, B7-H4 (VTCN1) or BTLA is a nucleic acid, a protein, a peptide, a nucleic acid-protein complex or a small molecule.

Embodiment 53. The method of embodiment 52, wherein the nucleic acid is an siRNA, an shRNA or an miRNA.

Embodiment 54. The method of embodiment 52, wherein the nucleic acid-protein complex is a gene-editing complex with or without an ssODN for homologous recombination.

Embodiment 55. A method for promoting DC formation from a monocyte, the method comprising:

a) passing a cell suspension comprising an input monocyte through a cell-deforming constriction, wherein a diameter of the constriction is a function of a diameter of the input monocyte in the suspension, thereby causing perturbations of the input monocyte large enough for an agent that promotes formation of DCs to pass into the monocyte; and

b) incubating the perturbed input monocyte with the agent that promotes formation of DCs for a sufficient time to allow the agent to enter the perturbed input monocyte.

Embodiment 56. The method of embodiment 55, wherein the agent that promotes formation of DCs upregulates expression of one or more of PU.1, Flt3, Flt3L or GMCSF.

Embodiment 57. The method of embodiment 56, wherein the agent that upregulates expression of one or more of PU.1, Flt3, Flt3L or GMCSF is a nucleic acid, a protein or a nucleic acid-protein complex.

Embodiment 58. The method of embodiment 57, wherein the nucleic acid is a DNA, an mRNA, an siRNA, an shRNA or an miRNA.

Embodiment 59. The method of embodiment 57, wherein the nucleic acid-protein complex is a gene-editing complex with or without an ssODN for homologous recombination.

Embodiment 60. A method for promoting plasmacytoid DC (pDC) formation from a monocyte or monocyte-dendritic progenitor cell, the method comprising:

a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of pDCs to pass into the monocyte or monocyte-dendritic progenitor cell; and

b) incubating the perturbed input monocyte or monocyte-dendritic progenitor cell with the agent that promotes formation of pDCs for a sufficient time to allow the agent to enter the perturbed input monocyte or monocyte-dendritic progenitor cell.

Embodiment 61. The method of embodiment 60, wherein the agent that promotes formation of pDCs upregulates expression of E2-2.

Embodiment 62. The method of embodiment 61, wherein the agent that upregulates expression of E2-2 is a nucleic acid, a protein or a nucleic acid-protein complex.

Embodiment 63. The method of embodiment 62, wherein the nucleic acid is a DNA, an mRNA, an siRNA, an shRNA or an miRNA.

Embodiment 64. The method of embodiment 62, wherein the nucleic acid-protein complex is a gene-editing complex with or without an ssODN for homologous recombination.

Embodiment 65. A method for promoting CD8a+/CD10+ DC formation from a monocyte or monocyte-dendritic progenitor cell, the method comprising:

a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that promotes formation of CD8a+/CD10+ DCs to pass into the monocyte; and

b) incubating the perturbed input monocyte or monocyte-dendritic progenitor cell with the agent that promotes formation of CD8a+/CD10+ DCs for a sufficient time to allow the agent to enter the perturbed input monocyte or monocyte-dendritic progenitor cell.

Embodiment 66. The method of embodiment 65, wherein the agent that promotes formation of CD8a+/CD10+ DCs upregulates expression of one or more of Batf3, IRF8 or Id2.

Embodiment 67. The method of embodiment 66, wherein the agent that upregulates expression of one or more of Batf3, IRF8 or Id2 is a nucleic acid, a protein or a nucleic acid-protein complex.

Embodiment 68. The method of embodiment 67, wherein the nucleic acid is a DNA, an mRNA, an siRNA, an shRNA or an miRNA.

Embodiment 69. The method of embodiment 67, wherein the nucleic acid-protein complex is a gene-editing complex with or without an ssODN for homologous recombination.

Embodiment 70. A method for promoting CD11b+ DC formation from a monocyte or monocyte-dendritic progenitor cell, the method comprising:

a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of CD11b+ DCs to pass into the monocyte or monocyte-dendritic progenitor cell; and

b) incubating the perturbed input monocyte or monocyte-dendritic progenitor cell with the agent that promotes formation of CD11b+ DCs for a sufficient time to allow the agent to enter the perturbed input monocyte or monocyte-dendritic progenitor cell.

Embodiment 71. The method of embodiment 70, wherein the agent that promotes formation of CD11b+ DCs upregulates expression of one or more of IRF4, RBJ, MgI or Mtg16.

Embodiment 72. The method of embodiment 71, wherein the agent that upregulates expression of one or more of IRF4, RBJ, MgI or Mtg16 is a nucleic acid, a protein or a nucleic acid-protein complex.

Embodiment 73. The method of embodiment 72, wherein the nucleic acid is a DNA, an mRNA, an siRNA, an shRNA or an miRNA.

Embodiment 74. The method of embodiment 72, wherein the nucleic acid-protein complex is a gene-editing complex with or without an ssODN for homologous recombination.

Embodiment 75. A method for inhibiting formation of pDCs and classical DCs from a monocyte or monocyte-dendritic progenitor cell, the method comprising:

a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that inhibits formation of pDCs and classical DCs to pass into the monocyte or monocyte-dendritic progenitor cell; and

b) incubating the perturbed input monocyte or monocyte-dendritic progenitor cell with the agent that inhibits formation of pDCs and classical DCs for a sufficient time to allow the agent to enter the perturbed input monocyte or monocyte-dendritic progenitor cell.

Embodiment 76. The method of embodiment 75, wherein the agent that inhibits formation of pDCs and classical DCs downregulates expression of STAT3 and/or Xbp1.

Embodiment 77. The method of embodiment 76, wherein the agent that downregulates expression of STAT3 and/or Xbp1 is a nucleic acid, a protein, a peptide, a nucleic acid-protein complex or a small molecule.

Embodiment 78. The method of embodiment 77, wherein the nucleic acid is an siRNA, an shRNA or an miRNA.

Embodiment 79. The method of embodiment 77, wherein the nucleic acid-protein complex is a gene-editing complex with or without an ssODN for homologous recombination.

Embodiment 80. The method of any one of embodiments 55-79, wherein the monocyte or monocyte-dendritic progenitor cell comprising the agent differentiates into a dendritic cell (DC).

Embodiment 81. The method of embodiment 80, wherein the DC is a pDC, a CD8a+/CD10+ DC, and/or a CD11b+ DC.

Embodiment 82. The method of any one of embodiments 1-54, wherein the antigen presenting cell further comprises an antigen.

Embodiment 83. The method of embodiment 82, wherein the antigen is delivered before, at the same time, or after the agent that enhances the viability and/or function of the antigen presenting cell is delivered to the cell.

Embodiment 84. The method of embodiment 83, wherein the antigen is delivered to the antigen presenting cell by a method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the antigen to pass into the antigen presenting cell; and

b) incubating the perturbed input antigen presenting cell with the antigen for a sufficient time to allow the antigen to enter the perturbed input antigen presenting cell.

Embodiment 85. The method of any one of embodiments 1-54, wherein the antigen presenting cell further comprises an adjuvant.

Embodiment 86. The method of embodiment 85, wherein the adjuvant is delivered before, at the same time, or after the antigen is delivered to the cell and/or before, at the same time, or after the agent that enhances the viability and/or function of the antigen presenting cell is delivered to the cell.

Embodiment 87. The method of embodiment 86, wherein the adjuvant is delivered to the antigen presenting cell by a method comprising:

a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the adjuvant to pass into the antigen presenting cell; and

b) incubating the perturbed input antigen presenting cell with the adjuvant for a sufficient time to allow the adjuvant to enter the perturbed input antigen presenting cell.

Embodiment 88. The method of any one of embodiments 85-87, wherein the adjuvant is a CpG ODN, IFN-α, STING agonists, RIG-I agonists, poly I:C, imiquimod, and/or resiquimod.

Embodiment 89. The method of any one of embodiments 82-86, wherein the antigen is capable of being processed into an MEW class I-restricted peptide and/or an MEW class II-restricted peptide.

Embodiment 90. The method of any one of embodiments 1-54 and 82-89, wherein the diameter of the constriction is less than the diameter of the input antigen presenting cell.

Embodiment 91. The method of embodiment 90, wherein the diameter of the constriction is about 20% to about 99% of the diameter of the input antigen presenting cell.

Embodiment 92. The method of embodiment 91, wherein the diameter of the constriction is about 20% to about 60% of the diameter of the input antigen presenting cell.

Embodiment 93. The method of any one of embodiments 86-92, wherein the antigen and/or adjuvant are present in the cytosol and/or a vesicle of the antigen presenting cell.

Embodiment 94. The method of any one of embodiments 82-93, wherein the antigen is bound to the surface of the antigen presenting cell.

Embodiment 95. The method of any one of embodiments 82-94, wherein the antigen is a disease associated antigen.

Embodiment 96. The method of any one of embodiments 82-95, wherein the antigen is a tumor antigen.

Embodiment 97. The method of any one of embodiments 82-96, wherein the antigen is derived from a lysate.

Embodiment 98. The method of embodiment 97, wherein the lysate is a tumor lysate.

Embodiment 99. The method of any one of embodiments 1-39 and 41-54, wherein the antigen presenting cell is a peripheral blood mononuclear cell (PBMC).

Embodiment 100. The method of any one of embodiments 1-39 and 41-54, wherein the antigen presenting cell is in a mixed population of cells.

Embodiment 101. The method of embodiment 100, wherein the mixed population of cells is a population of PBMCs.

Embodiment 102. The method of embodiment 99 or 101, wherein the PBMC is a T cell, a B cell, an NK cells, a monocyte, a macrophage and/or a dendritic cell.

Embodiment 103. The method of embodiment 99, 101 or 102, wherein the PBMC is engineered to present an antigen.

Embodiment 104. The method of any one of embodiments 55-81, wherein the monocyte, or monocyte-dendritic progenitor or DC further comprises an antigen.

Embodiment 105. The method of embodiment 104, wherein the antigen is delivered before, at the same time, or after the agent that promotes or inhibits DC formation is delivered to the cell.

Embodiment 106. The method of embodiment 105, wherein the antigen is delivered to the monocyte, or monocyte-dendritic progenitor or DC by a method comprising:

a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the antigen to pass into the monocyte, or monocyte-dendritic progenitor or DC; and

b) incubating the perturbed input monocyte, or monocyte-dendritic progenitor or DC with the antigen for a sufficient time to allow the antigen to enter the perturbed input monocyte, or monocyte-dendritic progenitor or DC.

Embodiment 107. The method of any one of embodiments 55-81 or 104-106, wherein the monocyte, or monocyte-dendritic progenitor or DC further comprises an adjuvant.

Embodiment 108. The method of embodiment 107, wherein the adjuvant is delivered before, at the same time, or after the antigen is delivered to the cell and/or before, at the same time, or after the agent that promotes DC formation is delivered to the cell.

Embodiment 109. The method of embodiment 108, wherein the adjuvant is delivered to the monocyte, or monocyte-dendritic progenitor or DC by a method comprising:

a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the adjuvant to pass into the monocyte, or monocyte-dendritic progenitor or DC; and

b) incubating the perturbed input monocyte, or monocyte-dendritic progenitor or DC with the adjuvant for a sufficient time to allow the adjuvant to enter the perturbed input monocyte, or monocyte-dendritic progenitor or DC.

Embodiment 110. The method of any one of embodiments 107-109, wherein the adjuvant is a CpG ODN, IFN-α, STING agonists, RIG-I agonists, poly I:C, imiquimod, and/or resiquimod.

Embodiment 111. The method of any one of embodiments 106-110, wherein the antigen is capable of being processed into an MEW class I-restricted peptide and/or an MEW class II-restricted peptide.

Embodiment 112. The method of any one of embodiments 55-81 and 104-111, wherein the diameter of the constriction is less than the diameter of the input monocyte, or monocyte-dendritic progenitor or DC.

Embodiment 113. The method of embodiment 112, wherein the diameter of the constriction is about 20% to about 99% of the diameter of the input monocyte, or monocyte-dendritic progenitor or DC.

Embodiment 114. The method of embodiment 113, wherein the diameter of the constriction is about 20% to about 60% of the diameter of the input monocyte, or monocyte-dendritic progenitor or DC.

Embodiment 115. The method of any one of embodiments 104-114, wherein the antigen and/or adjuvant are present in the cytosol and/or a vesicle of the monocyte, or monocyte-dendritic progenitor or DC.

Embodiment 116. The method of any one of embodiments 104-115, wherein the antigen is bound to the surface of the monocyte, or monocyte-dendritic progenitor or DC.

Embodiment 117. The method of any one of embodiments 104-116, wherein the antigen is a disease associated antigen.

Embodiment 118. The method of any one of embodiments 104-117, wherein the antigen is a tumor antigen.

Embodiment 119. The method of any one of embodiments 104-117, wherein the antigen is derived from a lysate.

Embodiment 120. The method of embodiment 119, wherein the lysate is a tumor lysate.

Embodiment 121. A modified antigen presenting cell comprising an agent that enhances the viability and/or function of an antigen presenting cell, wherein the cell is prepared by the method of any one of embodiments 1-54 and 82-103.

Embodiment 122. A modified monocyte, or monocyte-dendritic progenitor or DC, wherein the monocyte, or monocyte-dendritic progenitor or DC is prepared by the method of any one of embodiments 55-81 and 104-120.

Embodiment 123. A method for modulating an immune response in an individual, comprising: administering to the individual an antigen presenting cell, wherein the antigen presenting cell is prepared by a process according to any one of embodiments 1-54 and 82-103.

Embodiment 124. A method for modulating an immune response in an individual, comprising: administering to the individual a dendritic cell, wherein the dendritic cell is prepared by a process according to of any one of embodiments 55-81 and 104-120.

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Example 1

In order to determine if the ability of antigen presenting cell to activate an antigen-specific T cell response can be enhanced by overexpression (or upregulation) of certain co-stimulatory molecules, primary human mixed PBMC populations will be loaded with agents that upregulate CD80 and/or CD86, and responder cell IFN-γ secretion will be measured by ELISA.

Primary human mixed PBMC populations are isolated from multiple human donors (10M cells/mL). Specifically, 10-50 μM of each of OVA protein, and CD80 and CD86 mRNA will be delivered intracellularly by SQZ, and the level of IFN-γ, as measured by ELISA, will be compared between the SQZ conditions and a control wherein the CD80 and CD86 mRNAs are incubated with the PBMC_{APC} in the absence of SQZing (Endo). CD80 and CD86 upregulation can be assayed by flow cytometry. PBMC_{APC}s will then be co-cultured with OVA-specific CD8+ responder cells in a stimulator:effector ratio of 1:1 and cultured in the absence or presence of IL-2 (100 U/mL). After 18 h, supernatant is harvested from each condition and the level of IFN-γ production can be assessed by IFN-γ ELISA (Biolegend).

In alternative experiments, in lieu of CD80 and CD86 mRNA, the upregulation can be achieved by loading of CD80 and CD86 plasmid DNAs, and/or using CRISPR homology directed repair by loading a gene editing complex coupled with a single-stranded oligonucleotide donor templates for CD80 and CD86, using SQZ. Further experiments will be conducted to assess if the ability of antigen presenting cell to activate an antigen-specific T cell response can be further enhanced by upregulation of IL-2 using similar methods, i.e. loading of IL-2 mRNA, plasmid DNA and/or using CRISPR homology directed repair by loading a gene editing complex coupled with a single-stranded oligonucleotide donor templates for IL-2, using SQZ.

### Example 2

To determine if the antigen-specific immune response elicited by mixed PBMCA_{PCS} can be further enhanced by promotion of M1 macrophage phenotype in subpopulations of monocytes in the mixed PBMCs, primary human mixed PBMC populations will be loaded with agents that upregulate the expression of TLR4 (the target of LPS), IFN-γ and IL-12, and antigen-specific immune response can be measured by IFN-γ production, tetramer staining, or flow cytometry for antigen-specific T-cell cytotoxicity.

Primary human mixed PBMC populations are isolated from multiple human donors (10M cells/mL). Specifically, 10-50 μM of each of OVA protein, and mRNAs of TLR4, IFN-γ, and/or IL-12 will be delivered intracellularly by SQZ, and the level of antigen-specific immune response, as measured by IFN-γ production, tetramer assay or T cell-mediated cytotoxicity will be compared between the SQZ conditions and a control wherein the TLR4, IFN-γ, and/or IL-12 mRNAs are incubated with the PBMC_{APC} in the absence of SQZing (Endo). TLR4, IFN-γ, and/or IL-12 upregulation can be assayed by flow cytometry (TLR4, IFN-γ intracellular staining) or ELISA (IFN-γ secretion, IL-12). PBMC_{APC}s can then be co-cultured with OVA-specific CD8+ responder cells in a stimulator:effector ratio of 1:1 and cultured in the absence or presence of IL-2 (100 U/mL). After 18 h, supernatant is harvested from each condition and the level of IFN-γ production can be assessed by IFN-γ ELISA (Biolegend).

In alternative experiments, in lieu of TLR4, IFN-γ, and/or IL-12 mRNAs, the upregulation of TLR4, IFN-γ, and IL-12 can be achieved by loading of TLR4, IFN-γ, and/or IL-12 proteins directly using SQZ.

### Example 3

In order to determine if the ability of antigen presenting cell to activate an antigen-specific T cell and induce an antigen-specific T cell toxicity can be enhanced by the inhibition or downregulation of certain immune checkpoint regulators, primary human mixed PBMC populations will be loaded with agents that inhibit or downregulate PD-1, and antigen-specific T cell cytotoxicity will be measured by flow cytometry after co-culture.

Primary human mixed PBMC populations are isolated from multiple human donors (10M cells/mL). Specifically, 10-50 μM of each of OVA protein, and shRNA against PD-1 will be delivered intracellularly by SQZ, and the level of T cell-mediated cytotoxicity, as measured by flow cytometry, will be compared between the SQZ conditions and a control wherein the PD-1 shRNA are incubated with the PBMC_{APC} in the absence of SQZing (Endo). PD-1 downregulation can be assayed by flow cytometry. PBMC_{APC}s will then be co-cultured with OVA-specific CD8+ responder cells in a stimulator:effector ratio of 1:1 and cultured in the absence or presence of IL-2 (100 U/mL). After 18 h, the effect of PBMC_{APC}s in activating antigen-specific T cells and antigen-specific T cell toxicity can be assayed with tetramer staining and flow cytometry.

In alternative experiments, in lieu of PD-1 shRNA, the inhibition of PD-1 can be achieved by SQZ-loading of small molecule inhibitors, or the downregulation of PD-1 can be achieved by loading of one or more of PD-1 siRNA, or gene-editing enzymes or complexes such as CRIPSR, ZFN and TALENS using SQZ.

### Example 4

This example demonstrates, in part, that the ability of an antigen presenting cell to activate an in vitro antigen-specific T cell response can be enhanced by overexpression (or upregulation) of certain co-stimulatory molecules.

### Materials and Methods

To determine whether the ability of an antigen presenting cell to activate an antigen-specific T cell response can be enhanced by overexpression of co-stimulatory molecules, OVA antigen was delivered either with IL-2 mRNA or with IL-12 mRNA to dendritic cells using SQZ, followed by co-culture with OVA-specific OT-I cells and subsequent measurement of IFN-γ secretion using ELISA. Specifically, on Day −8, bone-marrow derived murine DCs (BMDCs) were harvested from C56BL/6J mice and maintained in culture media containing full-growth RMPI 1640+2-mercaptoethanol (55 recombinant murine GM-CSF (20 ng/mL) and recombinant mouse IL-4 (10 ng/mL). On Day −5, GM-CSF and IL-4 were replenished by adding half volume RPMI carrying twice the concentration of 2-mercaptoethanol, GM-CSF and IL-4 (supplementation). The GM-CSF and IL-4 supplementation was repeated on Day −1. On Day 0, BMDCs were collected, and matured in LPS (100 EU/mL) and IFN-γ (100 ng/mL) for 1 h at 37° C., with agitation every 15 mins. Subsequently, matured BMDCs were either incubated with Ova protein at 10 μg/mL (Ova Endocytosis), SQZ-loaded with Ova only (5 μg/mL), SQZ-loaded with IL-2 mRNA only (50 μg/mL), SQZ-loaded with IL-12 mRNA only (50 μg/mL), or SQZ-loaded with either (i) Ova and mouse IL-2 mRNA, or (ii) OVA and mouse IL-12 mRNA (50 μg/mL). As a positive control, BMDCs were pulsed with a peptide containing Ova minimal epitope (SIINFEKL pulse). The BMDCs processed as above were then co-cultured with purified OT-I cells at 1:10 ratios in triplicates. After 1 day of co-culture, the supernatant was collected and IFN-γ secretion was measured by ELISA, the results of which indicate the amount of in vitro antigen-specific T cell response stimulated by the antigen-loaded BMDCs with or without overexpression of co-stimulatory molecules.

### Results

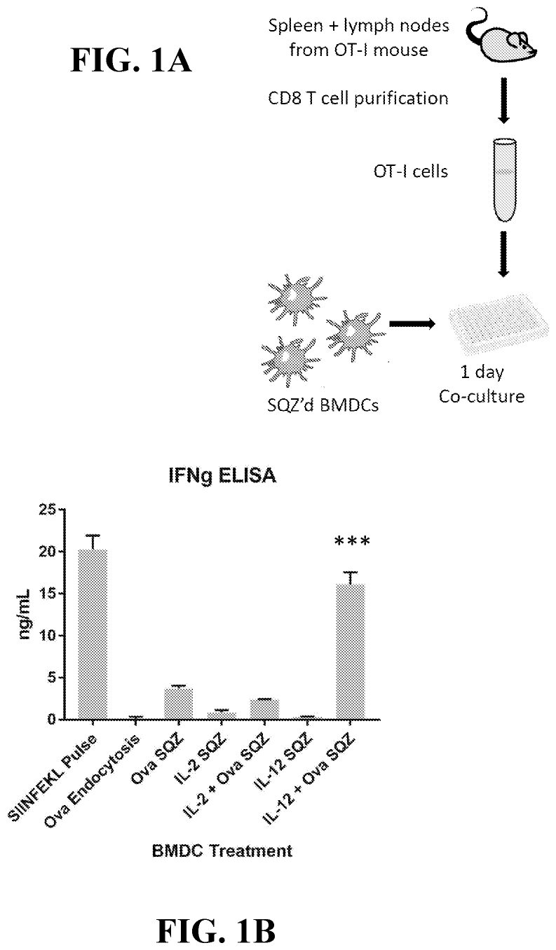

IFN-γ ELISA results showed that while there was a small increase in the Ova-specific response induced by the BMDCs with Ova delivered by SQZ (Ova SQZ) as compared to BMDCs incubated with Ova; the Ova-specific response was significantly higher in BMDCs with Ova and IL-12 mRNA co-delivered by SQZ (˜4-fold) compared to BMDCs with only Ova loaded (***p<0.001) (FIG. 1B). Taken together, these data show that in vitro antigen-specific T cell responses triggered by antigen presenting cells can be further enhanced when SQZ-loading certain co-stimulatory molecules (such as IL-12) in addition to the SQZ-loading of an antigen (such as OVA). Surprisingly, the increase in Ova-specific response is not significantly different between BMDCs SQZ-loaded with Ova and IL-2 mRNA SQZ and BMDCs with only Ova loaded (FIG. 1).

### Example 5

This example demonstrates, in part, that the ability of an antigen presenting cell to activate CD8+ T cell response in vivo can be enhanced by overexpression (or upregulation) of co-stimulatory molecules.

### Materials and Methods

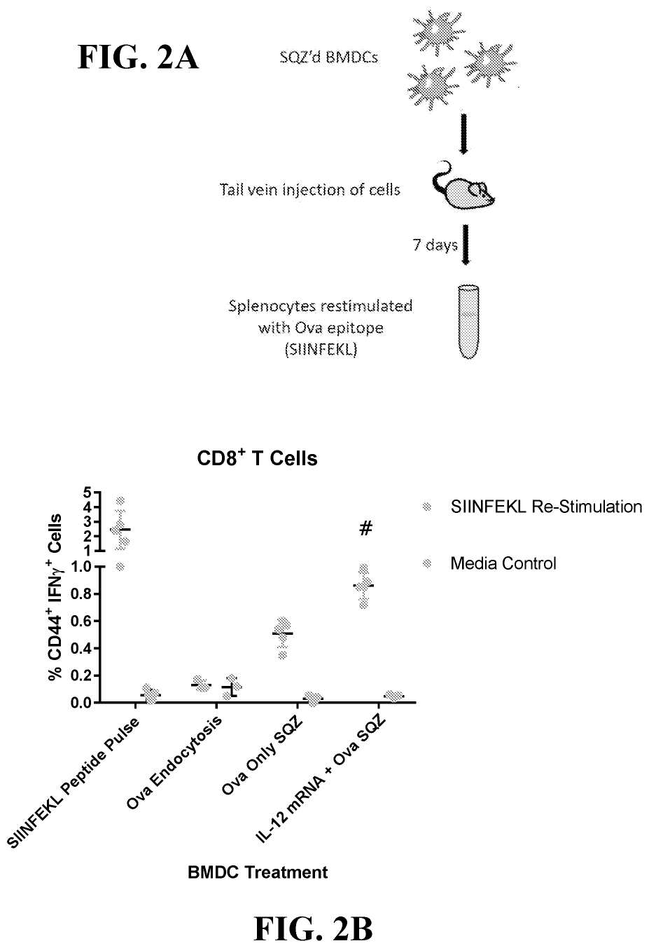

To determine whether the ability of an antigen presenting cell to activate CD8+ T cell response can be enhanced by overexpression of co-stimulatory molecules, OVA antigen and an mRNA encoding IL-12 were co-delivered to dendritic cells using SQZ, followed by injection into mice and subsequently analysis for CD8+ T cell responses using intracellular cytokine staining (ICS) and flow cytometry. Specifically, on Day −8, bone-marrow derived murine DCs (BMDCs) were harvested from C56BL/6J mice and maintained in culture media containing full-growth RMPI 1640+2-mercaptoethanol (55 recombinant murine GM-CSF (20 ng/mL) and recombinant mouse IL-4 (10 ng/mL). On Day −5, GM-CSF and IL-4 were replenished by adding half volume RPMI carrying twice the concentration of 2-mercaptoethanol, GM-CSF and IL-4 (supplementation). The GM-CSF and IL-4 supplementation was repeated on Day −1. On Day 0, BMDCs were collected, and matured in LPS (100 EU/mL) and IFN-γ (100 ng/mL) for 1 h at 37° C., with agitation every 15 mins. Subsequently, matured BMDCs were either incubated with Ova protein at 10 μg/mL (Ova Endocytosis), SQZ-loaded with Ova only (5 μg/mL), or SQZ-loaded with Ova and mouse IL-12 mRNA (50 μg/mL). As a positive control, BMDCs were pulsed with a peptide containing Ova minimal epitope (SIINFEKL pulse). The processed BMDCs were then injected into respective recipient mice (3E7 cells/mouse; 5 mice/group). After 7 days, splenocytes were harvested, re-challenged with Ova minimal epitope (SIINFEKL), and IFN-γ was measured by intracellular cytokine staining (ICS) and quantified using flow cytometry (FIG. 2A). The quantification of IFN-γ ICS indicates the amount of in vivo CD8+ T cell response stimulated by the antigen-loaded BMDCs with or without overexpression of co-stimulatory molecules.

### Results

IFN-γ ICS analysis showed that while there was a small increase in the CD8+ T cell response induced by the BMDCs with Ova loaded by SQZ (Ova Only SQZ) compared to BMDC incubated with Ova; the increase in response is higher in BMDCs with Ova and IL-12 mRNA loaded by SQZ (˜2-fold) compared to BMDCs with only Ova loaded (#P<0.005) (FIG. 2B). Taken together, these data show that CD8+ T cell responses triggered by antigen presenting cells can be further enhanced when SQZ-loading a co-stimulatory molecule (such as IL-12) in addition to SQZ-loading of an antigen (such as OVA).

### Example 6

Dendritic cells (DCs) prime T cell responses most efficiently in lymph nodes (LNs), where DCs have the highest probability of encountering their cognate T cell. For this reason, DCs SQZ-loaded with antigen may prime more potent T cell responses with improved trafficking of DCs to LNs post-vaccination. To evaluate this hypothesis, SQZ-loaded DCs were administered intravenously (IV) or intranodally (iLN), and the magnitude of T cell responses were compared between the two routes of administration.

### Materials and Methods

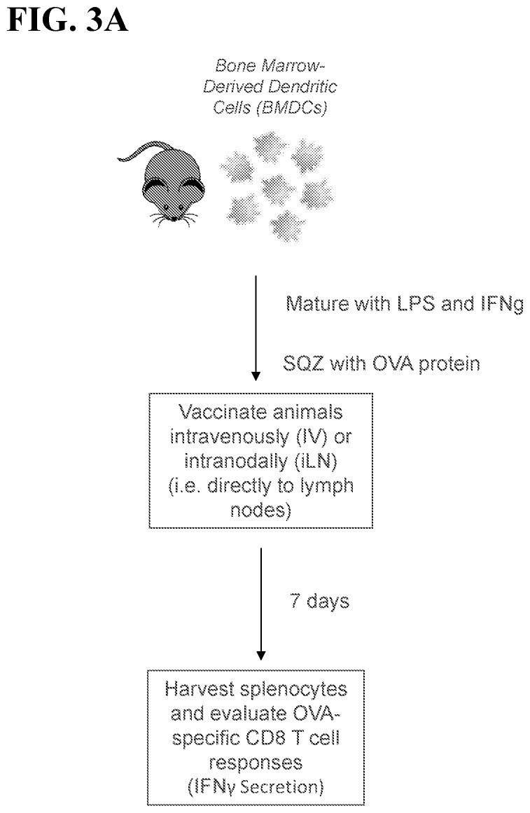

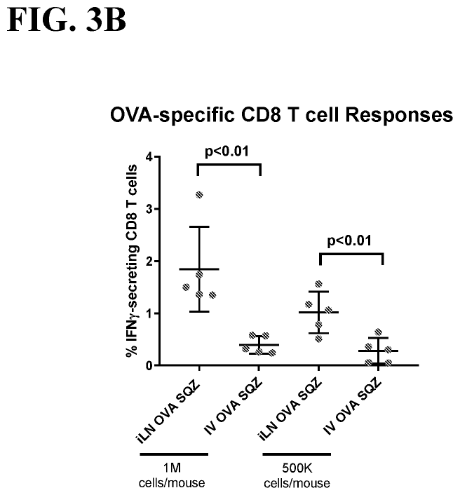

DCs were differentiated from murine bone marrow in GM-CSF and IL-4 for 8 days. On day 8 of differentiation, DCs were matured in LPS and IFNg for 1 hr and then SQZ-loaded with 5 ug/mL ovalbumin protein (OVA). These SQZ-loaded DCs were then administered to C57BL/6J mice at two different doses (1M/mouse or 500 k/mouse) either by IV or iLN injection. Seven days later, spleens were harvested from vaccinated mice and a single cell suspension of splenocytes was generated (FIG. 3A). These splenocytes were then re-stimulated ex vivo with 1 ug/mL SIINFEKL, the known H-2 kb-restricted CD8 T cell epitope associated with OVA. After 1 hour of re-stimulation, protein transport inhibitors (GOLGIPLUG™ and GOLGISTOP™) were added to prevent secretion of cytokines and to allow their accumulation within the cell after stimulation. After 4 additional hours of culture, the splenocytes were then harvested and processed for intracellular cytokine staining to allow for identification of IFN-γ-positive CD8 T cells and detection of IFN-γ responses within this cell population.

### Results

As shown in FIG. 3B, at both doses, iLN administration of SQZ-loaded DCs resulted in more antigen-specific CD8 T cells than IV administration did. Responses achieved with iLN administration ranged from 3.6- to 4.7-fold higher than those achieved with IV administration. These results suggest that improved trafficking of DCs to LNs could enable more potent T cell responses primed by DCs SQZ-loaded with antigen.

### Example 7

Dendritic cells (DCs) prime T cell responses most efficiently in lymph nodes (LNs), where DCs have the highest probability of encountering their cognate T cell. For this reason, DCs loaded with antigen may prime more potent T cell responses with improved trafficking of DCs to LNs post-vaccination. Overexpression of certain homing molecules, such as CD62L and/or CCR7, may help improve trafficking to LNs. CD62L allows lymphocytes to enter secondary lymphoid tissues from the blood via high endothelial venues, while CCR7 allows lymphocytes to traffic to the T cell zones of the spleen and LNs. In this study, DCs were SQZ-loaded with CD62L mRNA or CCR7 mRNA, respectively, to investigate whether SQZ-mediated loading could facilitate higher expression levels of these homing molecules.

### Materials and Methods

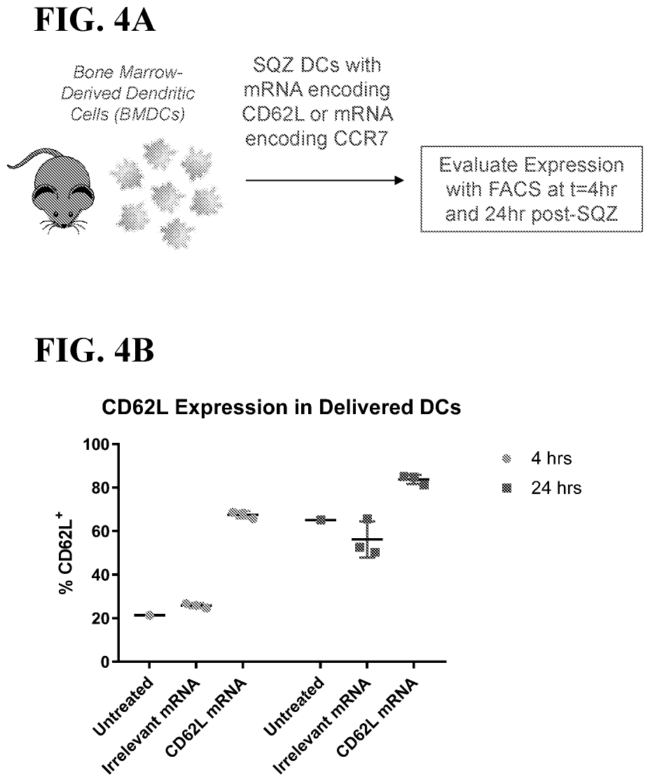

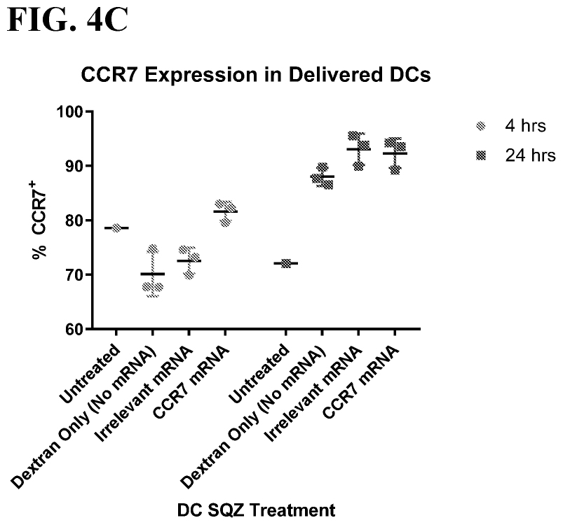

DCs were differentiated from murine bone marrow in GM-CSF and IL-4 for 8 days. On day 8 of differentiation, DCs were SQZ-loaded with 100 ug/mL of CD62L-encoding mRNA or CCR7-encoding mRNA. Using flow cytometry, surface expression of CD62L and CCR7 was evaluated at 4 hrs and 24 hrs post-SQZ (FIG. 4A).

### Results

DCs SQZ-loaded with CD62L mRNA showed higher expression of CD62L than untreated DCs and DCs that were SQZ-loaded with an irrelevant mRNA construct (FIG. 4B). At 4 hrs post-SQZ, CD62L expression was tripled in the CD62L mRNA SQZ group, when compared to the other negative control groups. At 24 hrs, CD62L seemed to naturally increase in expression in the untreated and irrelevant mRNA-treated DCs. Regardless, the DCs SQZ-loaded with CD62L mRNA still showed a ˜1.5-fold enhancement in CD62L expression compared to the controls. These results demonstrate that SQZ-mediated loading can be used to achieve enhanced expression of homing molecules via mRNA delivery (FIG. 4B). On the other hand, enhanced CCR7 expression with SQZ-loading of CCR7 mRNA was observed over the untreated and irrelevant mRNA controls at only the 4-hour time point post-SQZ (FIG. 4C). By 24 hrs, all SQZ groups, regardless of cargo, showed similar surface expression of CCR7 (FIG. 4C).

### Example 8

Maturation of antigen presenting cells such as dendritic cells (DCs) is accompanied by phenotypic maturation ligands such as CD80, CD86, CD83, which are co-stimulatory molecules that play important roles in activation of T lymphocytes. 4-1BB Ligand (4-1BBL, or CD137L) is a costimulatory ligand which mediates activation of T cells. Interferons, such as IFN-α2 play an important role in differentiation and maturation of antigen presenting cells such as dendritic cells. In this study, PBMCs were SQZ-loaded with CD86 mRNA and IFN-α2 mRNA, respectively, to investigate whether SQZ-mediated loading could facilitate higher expression levels of these molecules in different subsets of PBMCs.

### Materials and Methods

Primary human PBMC populations were isolated from multiple human donors (10M cells/mL). The PBMCs were either left untreated (NC); SQZ-processed with empty payload (Empty SQZ) or SQZ-loaded with mRNA encoding CD86 (100 ug/mL) or mRNA encoding IFN-α2 (100 ug/mL) at room temperature. 4 hours subsequent to SQZ processing, the loaded PBMCs were analyzed for the composition of B cells (CD19⁺), T cells (CD86⁺), NK cells (CD56⁺) and monocytes (CD14⁺), as well as respective surface expression of CD86 via flow cytometry. To measure expression of IFN-α2, cells were incubated for 4 hours with GOLGIPLUG™ or GOLGISTOP™ to inhibit secretion. The accumulated IFN-α2 was then analyzed by intracellular staining.

### Results

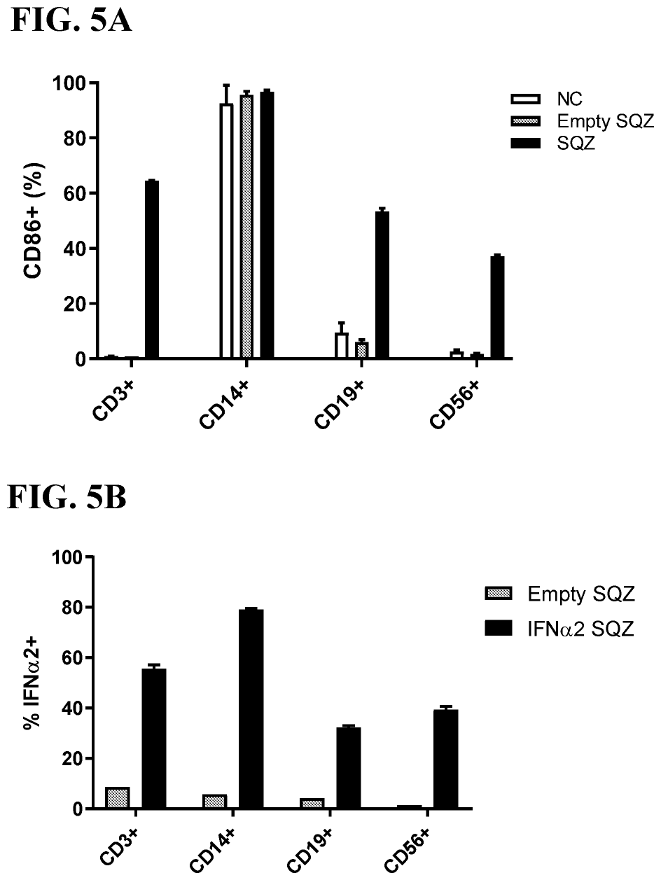

As shown in FIG. 5A, SQZ-loading of CD86 mRNA in PBMCs significantly increased the amount of cells displaying surface CD86 expression in B cells (CD19⁺), T cells (CD86⁺), NK cells (CD56⁺) compared to that of untreated PBMCs and PBMCs SQZ-processed with empty payload. Monocytes (CD14⁺) inherently expresses CD86 and SQZ-loading of CD86 mRNA did not significantly modulate surface expression (FIG. 5A). As shown in FIG. 5B, SQZ-loading of CD86 mRNA in PBMCs significantly increased the amount of cells displaying intracellular IFN-α2 expression in all subsets of B cells (CD19⁺), T cells (CD86⁺), NK cells (CD56⁺) and monocytes (CD14+) compared to that of untreated PBMCs and PBMCs SQZ-processed with empty payload.

### Example 9

Maturation of antigen presenting cells such as dendritic cells (DCs) is accompanied by phenotypic maturation ligands such as CD80, CD86, CD83, which are co-stimulatory molecules that play important roles in activation of T lymphocytes. 4-1BB Ligand (4-1BBL, or CD137L) is a costimulatory ligand which mediates activation of T cells. When overexpressed, these co-stimulatory molecules (e.g. CD86, 4-1BBL) may improve maturation and/or function of an antigen presenting cell. In this study, PBMCs were SQZ-loaded with CD86 and 4-1BBL mRNA, respectively, to investigate the surface expression level over time after the mRNA encoding these co-stimulatory molecules were delivered by SQZ-processing.

### Materials and Methods

Primary human PBMC populations were isolated from multiple human donors (10M cells/mL). The PBMCs were either SQZ-processed with empty payload (Empty SQZ) or SQZ-loaded with either mRNA encoding CD86 or mRNA encoding 4-1BBL (100 ug/mL) at room temperature. Subsequent to SQZ-processing, the PBMCs were analyzed for surface expression of CD86 or 4-1BBL over time (4 hours, 24 hours, 48 hours, and 72 hours) via flow cytometry.

### Results

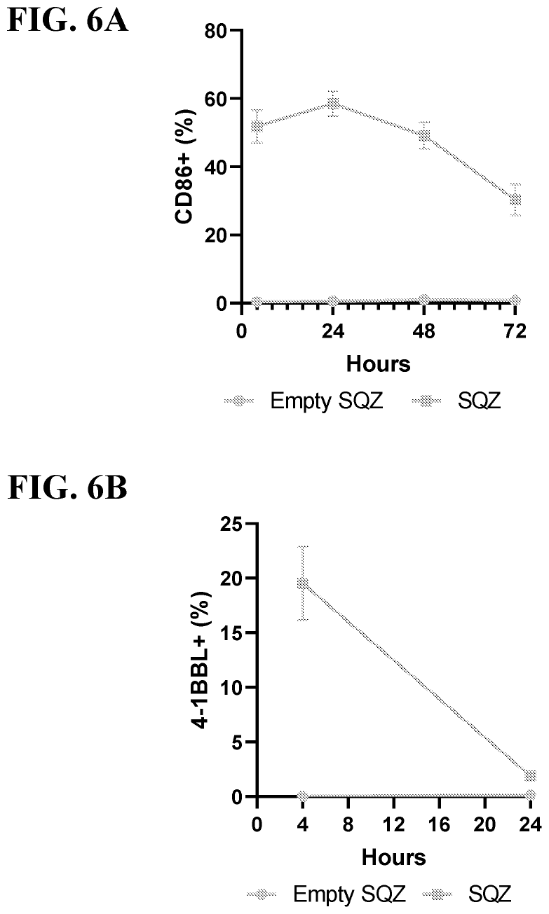

As shown in FIG. 6A, SQZ-loading of CD86 mRNA in PBMCs significantly increased the amount of the T cell subset (CD3⁺) displaying surface CD86 expression (>50%) compared to that of PBMCs SQZ-processed with empty payload (0%) at 4 hours and 24 hours post SQZ-processing. The amount of CD86⁺ cells in the SQZ-loaded T cell subset slightly tapered off after 24 hours and at 72 hours post SQZ-processing, about 30% of PBMCs still displayed surface CD86 expression. As shown in FIG. 6B, SQZ-loading of 4-1BBL mRNA in PBMCs increased the amount of the T cell subset (CD3⁺) displaying surface CD86 expression (>20%) compared to that of PBMCs SQZ-processed with empty payload (0%) at 4 hours post SQZ-processing. However, at 72 hours post SQZ-processing, less than 2% of PBMCs displayed surface 4-1BBL. These results indicate that the degree of and duration of protein expression induced by the SQZ-loading of mRNAs varied for different candidate mRNAs.

### Example 10

To determine if modification of mRNA could affect translation efficiency subsequent to mRNA delivery by SQZ-loading, human PBMCs were SQZ-loaded with unmodified eGFP or an eGFP modified with a 5-metoxyuridine backbone (5mou).

### Materials and Methods

Primary human PBMC populations were isolated from multiple human donors (10M cells/mL). The PBMCs were either SQZ-processed with either mRNA encoding unmodified eGFP or mRNA encoding 5mou-modified eGFP at various mRNA concentrations (0 to 200 ug/mL) at room temperature. Subsequent to SQZ-processing, the PBMCs were analyzed for eGFP expression via mean fluorescence intensity (MFI) using flow cytometry.

### Results

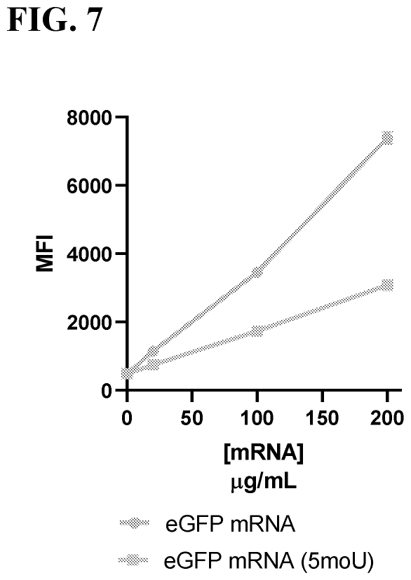

As shown in FIG. 7, SQZ-loading of eGFP or 5mou-eGFP mRNA in PBMCs increased the MFI in T cell subset (CD3⁺). For either eGFP or 5mou-eGFP, the MFI increased as the mRNA concentration used in SQZ-processing increased. However, at the concentrations tested, the increase in MFI effected by SQZ-loading of eGFP is higher than that by SQZ-loading of 5mou-eGFP, indicating that 5mou modification of mRNA did not enhance translation subsequent to SQZ-mediated delivery.

### Example 11

To study whether SQZ-loading of cytokines in antigen presenting cells can increase the expression and/or secretion cytokines, PBMCs were SQZ-loaded with IL-2, IFNα or IL-12a mRNA, respectively.

### Materials and Methods

Primary human PBMC populations were isolated from multiple human donors (10M cells/mL). The PBMCs were either left untreated (NC), SQZ-processed with empty payload (Empty SQZ) or SQZ-loaded with mRNA encoding IL-12 (50 ug/mL IL-12α mRNA+50 ug/mL IL-12(3), mRNA encoding IFNα (100 ug/mL) or mRNA encoding IL-2 (100 ug/mL) at room temperature. Subsequent to SQZ-processing, the PBMCs were incubated at 37° C. for four hours. Supernatants were collected and expression of IL-12, IFNα, or IL-2 were measured by ELISA.

### Results

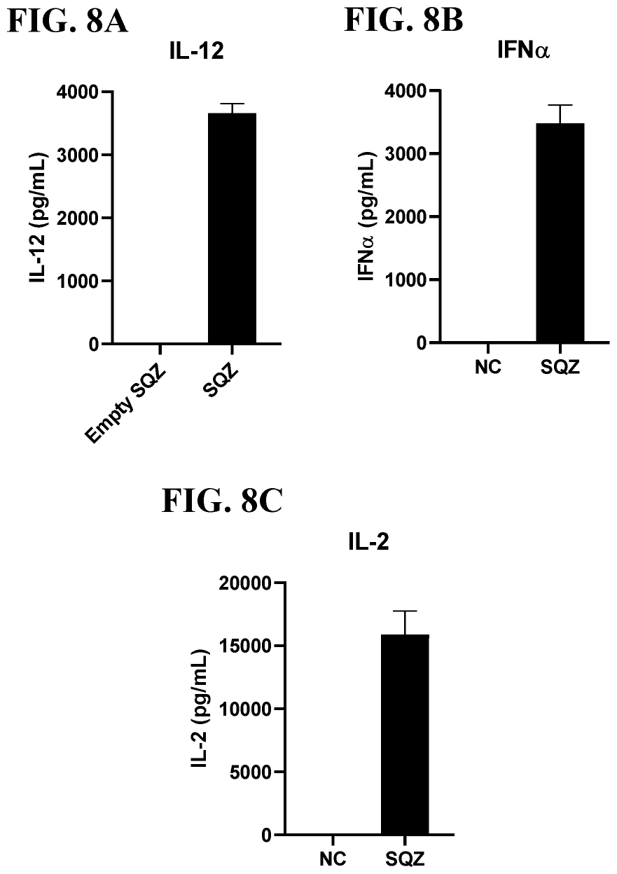

As shown in FIGS. 8A, 8B and 8C, SQZ-loading of IL-2, IFNα or IL-12a mRNA in PBMCs significantly increased the secretion of IL-2, IFNα or IL-12a by SQZ-processed PBMCs into the respective supernatants. These results indicated that SQZ-mediated delivery of mRNA in PBMCs could be used to increase expression and secretion of cytokines.

What is claimed is:

1. A method for enhancing tumor homing of an antigen presenting cell, the method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances tumor homing of the antigen presenting cell to pass into the antigen presenting cell; and
b) incubating the perturbed input antigen presenting cell with the agent that enhances tumor homing of the antigen presenting cell for a sufficient time to allow the agent to enter the perturbed input antigen presenting cell, thereby generating an enhanced antigen presenting cell.

2. The method of claim 1, wherein the agent that enhances tumor homing of the antigen presenting cell upregulates expression of one or more of CXCR3, CCR5, VLA-4 or LFA-1.

3. The method of claim 2, wherein the agent that upregulates expression of one or more of CXCR3, CCR5, VLA-4 or LFA-1 is a nucleic acid, a protein or a nucleic acid-protein complex.

4. The method of claim 3, wherein the nucleic acid is a DNA, an mRNA, an siRNA, an shRNA or an miRNA.

5. The method of claim 3, wherein the nucleic acid-protein complex is a gene-editing complex with or without an ssODN for homologous recombination.

6. A method for enhancing the viability and/or function of an antigen presenting cell, the method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an anti-apoptotic agent to pass into the antigen presenting cell; and
b) incubating the perturbed input antigen presenting cell with the anti-apoptotic agent for a sufficient time to allow the agent to enter the perturbed input antigen presenting cell, thereby generating an enhanced antigen presenting cell.

7. The method of claim 6, wherein the anti-apoptotic agent upregulates expression of one or more of XIAP, cIAP1/2, survivin, livin, cFLIP, Hsp72, or Hsp90.

8. The method of claim 7, wherein the agent that upregulates expression of one or more of XIAP, cIAP1/2, survivin, livin, cFLIP, Hsp72 or Hsp90 is a nucleic acid, a protein or a nucleic acid-protein complex.

9. The method of claim 8, wherein the nucleic acid is a DNA, an mRNA, an siRNA, an shRNA or an miRNA.

10. The method of claim 8, wherein the nucleic acid-protein complex is a gene-editing complex with or without an ssODN for homologous recombination.

11. A method for enhancing the function of an antigen presenting cell, the method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing to pass into the antigen presenting cell; and
b) incubating the perturbed input antigen presenting cell with the agent that enhances antigen processing for a sufficient time to allow the agent to enter the perturbed input antigen presenting cell, thereby generating an enhanced antigen presenting cell.

12. The method of claim 11, wherein the agent that enhances antigen processing upregulates expression of one or more of LMP2, LMP7, MECL-1 or β5t.

13. The method of claim 12, wherein the agent that upregulates expression of one or more of LMP2, LMP7, MECL-1 or β5t is a nucleic acid, a protein or a nucleic acid-protein complex.

14. The method of claim 13, wherein the nucleic acid is a DNA, an mRNA, an siRNA, an shRNA or an miRNA.

15. The method of claim 13, wherein the nucleic acid-protein complex is a gene-editing complex with or without an ssODN for homologous recombination.

16. A method for enhancing the function of an antigen presenting cell, the method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances antigen processing and/or loading onto MHC molecules to pass into the antigen presenting cell; and
b) incubating the perturbed input antigen presenting cell with the agent that enhances antigen processing and/or loading onto MHC molecules for a sufficient time to allow the agent to enter the perturbed input antigen presenting cell, thereby generating an enhanced antigen presenting cell.

17. The method of claim 16, wherein the agent that enhances antigen processing and/or loading onto MHC molecules upregulates expression of one or more of TAP, Tapasin, ERAAP, Calreticulin, Erp57 or PDI.

18. The method of claim 17, wherein the agent that upregulates expression of one or more of TAP, Tapasin, ERAAP, Calreticulin, Erp57 or PDI is a nucleic acid, a protein or a nucleic acid-protein complex.

19. The method of claim 18, wherein the nucleic acid is a DNA, an mRNA, an siRNA, an shRNA or an miRNA.

20. The method of claim 18, wherein the nucleic acid-protein complex is a gene-editing complex with or without an ssODN for homologous recombination.

21. A method for modulating immune activity of an antigen presenting cell, the method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that modulates immune activity to pass into the antigen presenting cell; and
b) incubating the perturbed input antigen presenting cell with the agent that modulates immune activity for a sufficient time to allow the agent to enter the perturbed input antigen presenting cell, thereby generating an enhanced antigen presenting cell.

22. The method of claim 21, wherein the agent that modulates immune activity upregulates expression of one or more of type I interferon, type II interferon, or type III interferon.

23. The method of claim 22, wherein the agent that upregulates expression of one or more of type I interferon, type II interferon, or type III interferon is a nucleic acid, a protein or a nucleic acid-protein complex.

24. The method of claim 23, wherein the nucleic acid is a DNA, an mRNA, an siRNA, an shRNA or an miRNA.

25. The method of claim 21, wherein the agent that modulates immune activity downregulates expression of interferon beta.

26. The method of claim 25, wherein the agent that downregulates expression of interferon beta is a nucleic acid, a protein, a peptide, a nucleic acid-protein complex or a small molecule.

27. The method of claim 23, wherein the nucleic acid is a DNA, an mRNA, an siRNA, an shRNA or an miRNA.

28. The method of claim 23, wherein the nucleic acid-protein complex is a gene-editing complex with or without an ssODN for homologous recombination.

29. A method for enhancing the viability of an antigen presenting cell, the method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances viability of the antigen presenting cell to pass into the antigen presenting cell; and
b) incubating the perturbed input antigen presenting cell with the agent that enhances viability of the antigen presenting cell for a sufficient time to allow the agent to enter the perturbed input antigen presenting cell, thereby generating an enhanced antigen presenting cell.

30. The method of claim 29, wherein the agent that enhances viability of the antigen presenting cell upregulates expression of a serpin.

31. The method of claim 30, wherein the agent that upregulates expression of a serpin is a nucleic acid, a protein or a nucleic acid-protein complex.

32. The method of claim 31 wherein the nucleic acid is a DNA, an mRNA, an siRNA, an shRNA or an miRNA.

33. The method of claim 31, wherein the nucleic acid-protein complex is a gene-editing complex with or without an ssODN for homologous recombination.

34. A method for enhancing the function of an antigen presenting cell, the method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that enhances homing and/or triggers alternative homing to pass into the antigen presenting cell; and
b) incubating the perturbed input antigen presenting cell with the agent that enhances homing and/or triggers alternative homing for a sufficient time to allow the agent to enter the perturbed input antigen presenting cell, thereby generating an enhanced antigen presenting cell.

35. The method of claim 34, wherein the agent that enhances homing and/or triggers alternative homing upregulates expression of a CCL2.

36. The method of claim 35, wherein the agent that upregulates expression of CCL2 is a nucleic acid, a protein or a nucleic acid-protein complex.

37. The method of claim 34, wherein the agent that enhances homing and/or triggers alternative homing upregulates expression of one or more of: CD62L, CCR2, CCR7, CX3CR1, or CXCR5.

38. The method of claim 37, wherein the agent that upregulates expression of one or more of: CD62L, CCR2, CCR7, CX3CR1, or CXCR5 comprises one or more of: a nucleic acid, a protein or a nucleic acid-protein complex.

39. The method of claim 37 or 38, wherein the agent enhances homing of the enhanced antigen presenting cell to lymph nodes.

40. The method of claim 39, wherein the antigen presenting cell is a dendritic cell.

41. The method of any one of claims 36 and 38-40, wherein the nucleic acid is a DNA, an mRNA, an siRNA, an shRNA or an miRNA.

42. The method of any one of claims 36 and 38-40, wherein the nucleic acid-protein complex is a gene-editing complex with or without an ssODN for homologous recombination.

43. A method for enhancing the viability and/or function of an antigen presenting cell, the method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that activates T cells to pass into the antigen presenting cell; and
b) incubating the perturbed input antigen presenting cell with the agent that activates T cells for a sufficient time to allow the agent to enter the perturbed input antigen presenting cell, thereby generating an enhanced antigen presenting cell.

44. The method of claim 43, wherein the agent that activates T cells upregulates expression of one or more of CD27, CD28, CD40, CD122, 4-1BB (CD137), OX40(CD134)/OX40L(CD252), GITR or ICOS.

45. The method of claim 44, wherein the agent that upregulates expression of one or more of CD27, CD28, CD40, CD122, 4-1BB (CD137), OX40(CD134)/OX40L(CD252), GITR or ICOS is a nucleic acid, a protein or a nucleic acid-protein complex.

46. The method of claim 43, wherein the agent that activates T cells upregulates expression of one or more of CD70, CD80, CD86, CD40L, 4-1BBL (CD137L), OX40L(CD252), GITRL or ICOSL.

47. The method of claim 46, wherein the agent that upregulates expression of one or more of CD70, CD80, CD86, CD40L, 4-1BBL (CD137L), OX40L(CD252), GITRL or ICOSL is a nucleic acid, a protein or a nucleic acid-protein complex.

48. The method of claim 45 or 47, wherein the nucleic acid is a DNA, an mRNA, an siRNA, an shRNA or an miRNA.

49. The method of claim 45 or 47, wherein the nucleic acid-protein complex is a gene-editing complex with or without an ssODN for homologous recombination.

50. A method for enhancing the viability and/or function of an antigen presenting cell, the method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for an agent that downregulates T cell inhibition to pass into the antigen presenting cell; and
b) incubating the perturbed input antigen presenting cell with the agent that downregulates T cell inhibition for a sufficient time to allow the agent to enter the perturbed input antigen presenting cell, thereby generating an enhanced antigen presenting cell.

51. The method of claim 50, wherein the agent that downregulates T cell inhibition downregulates expression of one or more of LAG3, VISTA, TIM1, B7-H4 (VTCN1) or BTLA.

52. The method of claim 51, wherein the agent that downregulates expression of one or more of LAG3, VISTA, TIM1, B7-H4 (VTCN1) or BTLA is a nucleic acid, a protein, a peptide, a nucleic acid-protein complex or a small molecule.

53. The method of claim 52, wherein the nucleic acid is an siRNA, an shRNA or an miRNA.

54. The method of claim 52, wherein the nucleic acid-protein complex is a gene-editing complex with or without an ssODN for homologous recombination.

55. A method for promoting DC formation from a monocyte or monocyte-dendritic progenitor cell, the method comprising:
a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that promotes formation of DCs to pass into the monocyte or monocyte-dendritic progenitor cell; and
b) incubating the perturbed input monocyte with the agent that promotes formation of DCs for a sufficient time to allow the agent to enter the perturbed input monocyte or monocyte-dendritic progenitor cell.

56. The method of claim 55, wherein the agent that promotes formation of DCs upregulates expression of one or more of PU.1, Flt3, Flt3L or GMCSF.

57. The method of claim 56, wherein the agent that upregulates expression of one or more of PU.1, Flt3, Flt3L or GMCSF is a nucleic acid, a protein or a nucleic acid-protein complex.

58. The method of claim 57, wherein the nucleic acid is a DNA, an mRNA, an siRNA, an shRNA or an miRNA.

59. The method of claim 57, wherein the nucleic acid-protein complex is a gene-editing complex with or without an ssODN for homologous recombination.

60. A method for promoting plasmacytoid DC (pDC) formation from a monocyte or monocyte-dendritic progenitor cell, the method comprising:
a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of pDCs to pass into the monocyte or monocyte-dendritic progenitor cell; and
b) incubating the perturbed input monocyte or monocyte-dendritic progenitor cell with the agent that promotes formation of pDCs for a sufficient time to allow the agent to enter the perturbed input monocyte or monocyte-dendritic progenitor cell.

61. The method of claim 60, wherein the agent that promotes formation of pDCs upregulates expression of E2-2.

62. The method of claim 61, wherein the agent that upregulates expression of E2-2 is a nucleic acid, a protein or a nucleic acid-protein complex.

63. The method of claim 62, wherein the nucleic acid is a DNA, an mRNA, an siRNA, an shRNA or an miRNA.

64. The method of claim 62, wherein the nucleic acid-protein complex is a gene-editing complex with or without an ssODN for homologous recombination.

65. A method for promoting CD8a+/CD10+ DC formation from a monocyte or monocyte-dendritic progenitor cell, the method comprising:
a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that promotes formation of CD8a+/CD10+ DCs to pass into the monocyte; and
b) incubating the perturbed input monocyte or monocyte-dendritic progenitor cell with the agent that promotes formation of CD8a+/CD10+ DCs for a sufficient time to allow the agent to enter the perturbed input monocyte or monocyte-dendritic progenitor cell.

66. The method of claim 65, wherein the agent that promotes formation of CD8a+/CD10+ DCs upregulates expression of one or more of Batf3, IRF8 or Id2.

67. The method of claim 66, wherein the agent that upregulates expression of one or more of Batf3, IRF8 or Id2 is a nucleic acid, a protein or a nucleic acid-protein complex.

68. The method of claim 67, wherein the nucleic acid is a DNA, an mRNA, an siRNA, an shRNA or an miRNA.

69. The method of claim 67, wherein the nucleic acid-protein complex is a gene-editing complex with or without an ssODN for homologous recombination.

70. A method for promoting CD11b+ DC formation from a monocyte or monocyte-dendritic progenitor cell, the method comprising:
a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte or monocyte-dendritic progenitor cell large enough for an agent that promotes formation of CD11b+ DCs to pass into the monocyte or monocyte-dendritic progenitor cell; and
b) incubating the perturbed input monocyte or monocyte-dendritic progenitor cell with the agent that promotes formation of CD11b+ DCs for a sufficient time to allow the agent to enter the perturbed input monocyte or monocyte-dendritic progenitor cell.

71. The method of claim 70, wherein the agent that promotes formation of CD11b+ DCs upregulates expression of one or more of IRF4, RBJ, MgI or Mtg16.

72. The method of claim 71, wherein the agent that upregulates expression of one or more of IRF4, RBJ, MgI or Mtg16 is a nucleic acid, a protein or a nucleic acid-protein complex.

73. The method of claim 72, wherein the nucleic acid is a DNA, an mRNA, an siRNA, an shRNA or an miRNA.

74. The method of claim 72, wherein the nucleic acid-protein complex is a gene-editing complex with or without an ssODN for homologous recombination.

75. A method for inhibiting formation of pDCs and classical DCs from a monocyte or monocyte-dendritic progenitor cell, the method comprising:
a) passing a cell suspension comprising an input monocyte or monocyte-dendritic progenitor cell through a cell-deforming constriction, wherein a diameter of the constriction is a function of a diameter of the input monocyte or monocyte-dendritic progenitor cell in the suspension, thereby causing perturbations of the input monocyte large enough for an agent that inhibits formation of pDCs and classical DCs to pass into the monocyte or monocyte-dendritic progenitor cell; and
b) incubating the perturbed input monocyte or monocyte-dendritic progenitor cell with the agent that inhibits formation of pDCs and classical DCs for a sufficient time to allow the agent to enter the perturbed input monocyte or monocyte-dendritic progenitor cell.

76. The method of claim 75, wherein the agent that inhibits formation of pDCs and classical DCs downregulates expression of STAT3 and/or Xbp1.

77. The method of claim 76, wherein the agent that downregulates expression of STAT3 and/or Xbp1 is a nucleic acid, a protein, a peptide, a nucleic acid-protein complex or a small molecule.

78. The method of claim 77, wherein the nucleic acid is an siRNA, an shRNA or an miRNA.

79. The method of claim 77, wherein the nucleic acid-protein complex is a gene-editing complex with or without an ssODN for homologous recombination.

80. The method of any one of claims 55-79, wherein the monocyte or monocyte-dendritic progenitor cell comprising the agent differentiates into a dendritic cell (DC).

81. The method of claim 80, wherein the DC is a pDC, a CD8a+/CD10+ DC, and/or a CD11b+ DC.

82. The method of any one of claims 1-54, wherein the antigen presenting cell further comprises an antigen.

83. The method of claim 82, wherein the antigen is delivered before, at the same time, or after the agent that enhances the viability and/or function of the antigen presenting cell is delivered to the cell.

84. The method of claim 83, wherein the antigen is delivered to the antigen presenting cell by a method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the antigen to pass into the antigen presenting cell; and
b) incubating the perturbed input antigen presenting cell with the antigen for a sufficient time to allow the antigen to enter the perturbed input antigen presenting cell.

85. The method of any one of claims 1-54, wherein the antigen presenting cell further comprises an adjuvant.

86. The method of claim 85, wherein the adjuvant is delivered before, at the same time, or after the antigen is delivered to the cell and/or before, at the same time, or after the agent that enhances the viability and/or function of the antigen presenting cell is delivered to the cell.

87. The method of claim 86, wherein the adjuvant is delivered to the antigen presenting cell by a method comprising:
a) passing a cell suspension comprising an input antigen presenting cell through a cell-deforming constriction, wherein a diameter of the constriction is a function of a diameter of the input antigen presenting cell in the suspension, thereby causing perturbations of the input antigen presenting cell large enough for the adjuvant to pass into the antigen presenting cell; and
b) incubating the perturbed input antigen presenting cell with the adjuvant for a sufficient time to allow the adjuvant to enter the perturbed input antigen presenting cell.

88. The method of any one of claims 85-87, wherein the adjuvant is a CpG ODN, IFN-α, STING agonists, RIG-I agonists, poly I: C, imiquimod, and/or resiquimod.

89. The method of any one of claims 82-88, wherein the antigen is capable of being processed into an MHC class I-restricted peptide and/or an MHC class II-restricted peptide.

90. The method of any one of claims 1-52 and 82-89, wherein the diameter of the constriction is less than the diameter of the input antigen presenting cell.

91. The method of claim 90, wherein the diameter of the constriction is about 20% to about 99% of the diameter of the input antigen presenting cell.

92. The method of claim 91, wherein the diameter of the constriction is about 20% to about 60% of the diameter of the input antigen presenting cell.

93. The method of any one of claims 85-92, wherein the antigen and/or adjuvant are present in the cytosol and/or a vesicle of the antigen presenting cell.

94. The method of any one of claims 82-93, wherein the antigen is bound to the surface of the antigen presenting cell.

95. The method of any one of claims 82-94, wherein the antigen is a disease associated antigen.

96. The method of any one of claims 82-95, wherein the antigen is a tumor antigen.

97. The method of any one of claims 82-96, wherein the antigen is derived from a lysate.

98. The method of claim 97, wherein the lysate is a tumor lysate.

99. The method of any one of claims 1-39 and 41-54, wherein the antigen presenting cell is a peripheral blood mononuclear cell (PBMC).

100. The method of any one of claims 1-39 and 41-54, wherein the antigen presenting cell is in a mixed population of cells.

101. The method of claim 100, wherein the mixed population of cells is a population of PBMCs.

102. The method of claim 99 or 101, wherein the PBMC is a T cell, a B cell, an NK cells, a monocyte, a macrophage and/or a dendritic cell.

103. The method of claim 99, 101 or 102, wherein the PBMC is engineered to present an antigen.

104. The method of any one of claims 55-81, wherein the monocyte, or monocyte-dendritic progenitor or DC further comprises an antigen.

105. The method of claim 104, wherein the antigen is delivered before, at the same time, or after the agent that promotes or inhibits DC formation is delivered to the cell.

106. The method of claim 105, wherein the antigen is delivered to the monocyte, or monocyte-dendritic progenitor or DC by a method comprising:
a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the antigen to pass into the monocyte, or monocyte-dendritic progenitor or DC; and
b) incubating the perturbed input monocyte, or monocyte-dendritic progenitor or DC with the antigen for a sufficient time to allow the antigen to enter the perturbed input monocyte, or monocyte-dendritic progenitor or DC.

107. The method of any one of claims 55-81 and 104-106, wherein the monocyte, or monocyte-dendritic progenitor or DC further comprises an adjuvant.

108. The method of claim 107, wherein the adjuvant is delivered before, at the same time, or after the antigen is delivered to the cell and/or before, at the same time, or after the agent that promotes DC formation is delivered to the cell.

109. The method of claim 108, wherein the adjuvant is delivered to the monocyte, or monocyte-dendritic progenitor or DC by a method comprising:
a) passing a cell suspension comprising an input monocyte, or monocyte-dendritic progenitor or DC through a cell-deforming constriction, wherein a diameter of the constriction is a function of a diameter of the input monocyte, or monocyte-dendritic progenitor or DC in the suspension, thereby causing perturbations of the input monocyte, or monocyte-dendritic progenitor or DC large enough for the adjuvant to pass into the monocyte, or monocyte-dendritic progenitor or DC; and
b) incubating the perturbed input monocyte, or monocyte-dendritic progenitor or DC with the adjuvant for a sufficient time to allow the adjuvant to enter the perturbed input monocyte, or monocyte-dendritic progenitor or DC.

110. The method of any one of claims 107-109, wherein the adjuvant is a CpG ODN, IFN-α, STING agonists, RIG-I agonists, poly I:C, imiquimod, and/or resiquimod.

111. The method of any one of claims 106-110, wherein the antigen is capable of being processed into an MHC class I-restricted peptide and/or an MHC class II-restricted peptide.

112. The method of any one of claims 55-81 and 104-111, wherein the diameter of the constriction is less than the diameter of the input monocyte, or monocyte-dendritic progenitor or DC.

113. The method of claim 112, wherein the diameter of the constriction is about 20% to about 99% of the diameter of the input monocyte, or monocyte-dendritic progenitor or DC.

114. The method of claim 113, wherein the diameter of the constriction is about 20% to about 60% of the diameter of the input monocyte, or monocyte-dendritic progenitor or DC.

115. The method of any one of claims 104-114, wherein the antigen and/or adjuvant are present in the cytosol and/or a vesicle of the monocyte, or monocyte-dendritic progenitor or DC.

116. The method of any one of claims 104-115, wherein the antigen is bound to the surface of the monocyte, or monocyte-dendritic progenitor or DC.

117. The method of any one of claims 104-116, wherein the antigen is a disease associated antigen.

118. The method of any one of claims 104-117, wherein the antigen is a tumor antigen.

119. The method of any one of claims 104-117, wherein the antigen is derived from a lysate.

120. The method of claim 119, wherein the lysate is a tumor lysate.

121. A modified antigen presenting cell comprising an agent that enhances the viability and/or function of an antigen presenting cell, wherein the cell is prepared by the method of any one of claims 1-54 and 82-103.

122. A modified monocyte, or monocyte-dendritic progenitor or DC, wherein the monocyte, or monocyte-dendritic progenitor or DC is prepared by the method of any one of claims 55-81 and 104-120.

123. A method for modulating an immune response in an individual, comprising:
administering to the individual an antigen presenting cell, wherein the antigen presenting cell is prepared by a process according to any one of claims 1-54 and 82-103.

124. A method for modulating an immune response in an individual, comprising:
administering to the individual a dendritic cell, wherein the dendritic cell is prepared by a process according to of any one of claims 80-81 and 104-120.
